SEQUENCE LISTING (76 sequences)

SEQ ID NO: 1 - DNA, 1436 bp, Bacteroides fragilis
atgaacgctagctacaggcttaacacatgcaagtcgaggggcatcaggaagaaagcttgctttctttgctggcgaccggcgcacgggtgagtaacacgtatccaacctgccctttactcggggatagcctttcgaaagaaagattaatacccgatagcataatgattccgcatggtttcattattaaaggattccggtaaaggatggggatgcgttccattaggttgttggtgaggtaacggctcaccaagccttcgatggataggggttctgagaggaaggtcccccacattggaactgagacacggtccaaactcctacgggaggcagcagtgaggaatattggtcaatgggcgctagcctgaaccagccaagtagcgtgaaggatgaaggctctatgggtcgtaaacttcttttatataagaataaagtgcagtatgtatactgttttgtatgtattatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctagcgggtgaccgtatgctaatcccaaaatcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgtaaccgcaaggatcgtcctagggtaaaac

SEQ ID NO: 2 - DNA, 1395 bp, Bacteroides ovatus
  misc_feature (51)..(51): n is a, c, g, or t
  misc_feature (125)..(125): n is a, c, g, or t
  misc_feature (154)..(154): n is a, c, g, or t
  misc_feature (168)..(168): n is a, c, g, or t
  misc_feature (170)..(170): n is a, c, g, or t
  misc_feature (177)..(177): n is a, c, g, or t
  misc_feature (180)..(180): n is a, c, g, or t
  misc_feature (393)..(393): n is a, c, g, or t
  misc_feature (435)..(435): n is a, c, g, or t
  misc_feature (720)..(721): n is a, c, g, or t
  misc_feature (902)..(902): n is a, c, g, or t
  misc_feature (973)..(973): n is a, c, g, or t
  misc_feature (999)..(999): n is a, c, g, or t
  misc_feature (1082)..(1082): n is a, c, g, or t
  misc_feature (1232)..(1232): n is a, c, g, or t
  misc_feature (1235)..(1235): n is a, c, g, or t
atgaacgctagctacaggcttaacacatgcaagtcgaggggcagcattttngtttgcttgcaaactgaagatggcgaccggcgcacgggtgagtaacacgtatccaacctgccgataactccggnatagcctttcgaaagaaagattaataccngatagcatacgaanancgcatgntanttttattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatgggcgagagcctgaaccagccaagtagcgtgaaggatganggccctatgggtcgtaaacttcttttatatgggaataaagtnttccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgnnactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggccngcacaagcggaggaacatgtggtttaattcgatgatacgcgaggaaccttacccgggcttaaattgcaacngaatatattggaaacagtatagccgnaaggctgttgtgaaggtgctgcatggttgtcgtcagctcgtgccgtgaggtgtcggcttaagtgccataacgagcgcaacccntatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctaccnggngacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggt

SEQ ID NO: 3 - DNA, 1510 bp, Bacteroides vulgatus
tattacaatgaagagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaag

SEQ ID NO: 4 - DNA, 1467 bp, Bilophila wadsworthia
  misc_feature (789)..(789): n is a, c, g, or t
cttaacacatgcaagtcgaacgtgaaagtccttcgggatgagtaaaagtggcgcacgggtgagtaacgcgtggataatctacccttaagatggggataacggctggaaacggtcgctaataccgaatacgctcccgattttatcattggggggaaagatggcctctgcttgcaagctatcgcttaaggatgagtccgcgtcccattagctagttggcggggtaacggcccaccaaggcaacgatgggtagccggtctgagaggatgaccggccacactggaactggaacacggtccagactcctacgggaggcagcagtggggaatattgcgcaatgggcgaaagcctgacgcagcgacgccgcgtgagggatgaaggttctcggatcgtaaacctctgtcaggggggaagaaaccccctcgtgtgaataatgcgagggcttgacggtacccccaaaggaagcaccggctaactccgtgccagcagccgcggtaatacggagggtgcaagcgttaatcggaatcactgggcgtaaagcgcacgtacgcggcttggtaagtcaggggtgaaatcccacagcccaactgtggaactgcctttgatactgccacgcttgagtaccggagagggtggcggaattccaggtgtaggagtgaaatccgtagatatctggaggaacaccggtggcgaaggcggccacctggacggtaactgacgctgaggtgcgaaagcgtgggtagcaaacaggattagataccctggtagtccacgctgtaaacgatgggtgcngggtgctgggatgtatgtctcggtgccgtagctaacgcgataagcaccccgcctggggagtacggtcgcaaggctgaaactcaaagaaattgacgggggcccgcacaagcggtggagtatgtggtttaattcgatgcaacgcgaagaaccttacccaggcttgacatctagggaacccttcggaaatgaaggggtgcccttcggggagccctaagacaggtgctgcatggctgtcgtcagctcgtgccgtgaggtgttgggttaagtcccgcaacgagcgcaacccctatcttcagttgccagcaggtaaggctgggcactctggagagaccgccccggtcaacggggaggaaggtggggacgacgtcaagtcatcatggcccttacgcctggggctacacacgtactacaatggcgcgcacaaagggtagcgagaccgcgaggtggagccaatcccaaaaaacgcgtcccagtccggattggagtctgcaactcgactccatgaagtcggaatcgctagtaattcgagatcagcatgctcgggtgaatgcgttcccgggccttgtacacaccgcccgtcacaccacgaaagtcggttttacccgaagccggtgagctaactcgcaagaggagcagccgtctacggtagggccgatgattggggtgaagtcgtaacaa

SEQ ID NO: 5 - DNA, 1476 bp, Clostridium bifermentans
  misc_feature (1449)..(1449): n is a, c, g, or t
catrgctcaggatgaacgctggcggcgtgcctaacacatgcaagtcgagcgatctcttcggagagagcggcggacgggtgagtaacgcgtgggtaacctgccctgtacacacggataacataccgaaaggtatactaatacgggataacatatgaaagtcgcatggcttttgtatcaaagctccggcggtacaggatggacccgcgtctgattagctagttggtaaggtaatggcttaccaaggcaacgatcagtagccgacctgagagggtgatcggccacactggaactgagacacggtccagactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatatgtagggggctagcgttatccggaattactgggcgtaaagggtgcgtaggtggttttttaagtcagaagtgaaaggctacggctcaaccgtagtaagcttttgaaactagagaacttgagtgcaggagaggagagtagaattcctagtgtagcggtgaaatgcgtagatattaggaggaataccagtagcgaaggcggctctctggactgtaactgacactgaggcacgaaagcgtggggagcaaacaggattagataccctggtagtccacgccgtaaacgatgagtactaggtgtcgggggttacccccctcggtgccgcactaacgcattaagtactccgcctgggaagtacgctcgcaagagtgaaactcaaaggaatttdcggggacccgcacaagtagcggagcatgtggtttaattcgaagcaacgcgaagaaccttacctaagcttgacatcccactgacctctccctaatcggagatttcccttcggggacagtggtgacaggtggtgcatggttgtcgtcagctcgtgtcgtgagatgttgggttaagtcccgcaacgagcgcaacccttgcctttagttgccagcattaagttgggcactctagagggactgccgaggataactcggaggaaggtggggatgacgtcaaatcatcatgccccttatgcttagggctacacacgtgctacaatgggtggtacagagggttgccaagccgcgaggtggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatggaagttgggggcgcccgaagccggttagctaaccttttaggaagcggccgtcgaaggtgaacaaatgactggggtgaagtcgtaacaaggtanccgtatcggaaggtgcggcbggatcaa

SEQ ID NO: 6 - DNA, 1393 bp, Clostridium hiranonis
acatgcaagtcgagcgattctcttcggagaagagcggcggacgggtgagtaacgcgtgggtaacctgccctgtacacacggataacataccgaaaggtatgctaatacgggataatatataagagtcgcatgacttttatatcaaagatttttcggtacaggatggacccgcgtctgattagcttgttggcggggtaacggcccaccaaggcgacgatcagtagccgacctgagagggtgatcggccacattggaactgagacacggtccaaactcctacgggaggcagcagtggggaatattgcacaatgggcgcaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggatttactgggcgtaaagggtgcgtaggcggtctttcaagtcaggagttaaaggctacggctcaaccgtagtaagctcctgatactgtctgacttgagtgcaggagaggaaagcggaattcccagtgtagcggtgaaatgcgtagatattgggaggaacaccagtagcgaaggcggctttctggactgtaactgacgctgaggcacgaaagcgtggggagcaaacaggattagataccctggtagtccacgctgtaaacgatgagtactagttgtcggaggttaccccttcggtgccgcagctaacgcattaagtactccgcctggggagtacgcacgcaagtgtgaaactcaaaggaattgacggggacccgcacaagtagcggagcatgtggtttaattcgaagcaacgcgaagaaccttacctaggcttgacatccttctgaccgaggactaatctcctctttccctccggggacagaagtgacaggtggtgcatggttgtcgtcagctcgtgtcgtgagatgttgggttaagtcccgcaacgagcgcaacccttgtctttagttgccatcattaagttgggcactctagagagactgccagggataacctggaggaaggtggggatgacgtcaaatcatcatgccccttatgcctagggctacacacgtgctacaatgggtggtacagagggcagccaagccgtgaggtggagcaaatcccttaaagccattctcagttcggattgtaggctgaaactcgcctacatgaagctggagttactagtaatcgcagatcagaatgctgcggtgaatgcgttcccgggtcttgtacacaccgcccgtcacaccatgggagttggagacacccgaagccgactatctaaccttttgggagaagtcgtccccctcgaatcaatacccc

SEQ ID NO: 7 - DNA, 1465 bp, Clostridium leptum
  misc_feature (1)..(84): n is a, c, g, or t
  misc_feature (233)..(233): n is a, c, g, or t
  misc_feature (271)..(271): n is a, c, g, or t
  misc_feature (337)..(338): n is a, c, g, or t
  misc_feature (389)..(389): n is a, c, g, or t
  misc_feature (482)..(485): n is a, c, g, or t
  misc_feature (523)..(523): n is a, c, g, or t
  misc_feature (659)..(659): n is a, c, g, or t
  misc_feature (670)..(670): n is a, c, g, or t
  misc_feature (812)..(813): n is a, c, g, or t
  misc_feature (822)..(822): n is a, c, g, or t
  misc_feature (827)..(828): n is a, c, g, or t
  misc_feature (906)..(908): n is a, c, g, or t
  misc_feature (932)..(932): n is a, c, g, or t
  misc_feature (945)..(946): n is a, c, g, or t
  misc_feature (968)..(968): n is a, c, g, or t
  misc_feature (1017)..(1017): n is a, c, g, or t
  misc_feature (1079)..(1080): n is a, c, g, or t
  misc_feature (1095)..(1095): n is a, c, g, or t
  misc_feature (1186)..(1187): n is a, c, g, or t
  misc_feature (1224)..(1224): n is a, c, g, or t
  misc_feature (1365)..(1366): n is a, c, g, or t
  misc_feature (1461)..(1462): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnttggatttaacttagtggcggacgggtgagtaacgcgtgagtaacctgcctttcagagggggataacgttctgaaaagaacgctaataccgcataacatcaatttatcgcatgataggttgatcaaaggagcaatccgctggaagatgnactcgcgtccgattagccagttggcggggtaacggccnaccaaagcgacgatcggtagccggactgagaggttgaacggccacattgggactgagacacggccnngactcctacgggaggcagcagtgggggatattgcacaatgggggaaacccngatgcagcaacgccgcgtgagggaagaaggttttcggattgtaaacctctgttcttagtgacgataatgacggtagctaaggagaaagctccnnnnaactacgtgccagcagccgcggtaatacgtagggagcnagcgttgtccggatttactgggtgtaaagggtgcgtaggcggcgaggcaagtcaggcgtgaaatctatgggcttaacccataaactgcgcttgaaactgtcttgcttgagtgaagtagaggtaggcggaattcccngtgtagcggtnaaatgcgtagagatcgggaggaacaccagtggcgaaggcggcctactgggctttaactgacgctgaagcacgaaagcatgggtagcaaacaggattagataccctggtagtccatgccgtaaacgatgattactaggtgtgnngggggtctnacccnntccgtgccgcagttaacacaataagtaatccacctggggagtacggccgcaaggttgaaactcaaaggaattgacggnnncccgcacaagcagtggagtatgtngtttaattcgaannaacgcgaagaaccttaccaggncttgacatccgtctaacgaagcagagatgcattaggtgcccttcggggnaaggcgagacaggtggtgcatggttgtcgtcagctcgtgtcgtgagatgttgggttaagtcnngcaacgagcgcaacncttgtttctagttgctacgcaagagcactctagagagactgccgttgacaaaacggaggaaggtggggacgacgtcaaatcatcatgcccnntatgacctgggccacacacgtactacaatggctgtanacagagggaagcaaagccgcgaggtggagcaaaaccctaaaagcagtcccagttcggatcgcaggctgcaacccgcctgcgtgaagtcggaattgctagtaatcgcggatcagcatgccgcggtgaatacgttcccgggcnntgtacacaccgcccgtcacaccatgggagccggtaatacccgaagccagtagttcaaccgcaaggagagcgctgtcgaaggtaggattggcgacnnggg

SEQ ID NO: 8 - DNA, 1530 bp, Clostridium ramosum
  misc_feature (70)..(70): n is a, c, g, or t
  misc_feature (541)..(541): n is a, c, g, or t
  misc_feature (1372)..(1372): n is a, c, g, or t
  misc_feature (1423)..(1423): n is a, c, g, or t
  misc_feature (1471)..(1471): n is a, c, g, or t
  misc_feature (1476)..(1482): n is a, c, g, or t
  misc_feature (1502)..(1502): n is a, c, g, or t
  misc_feature (1508)..(1515): n is a, c, g, or t
  misc_feature (1530)..(1530): n is a, c, g, or t
acaatggagagtttgatcctggctcaggatgaacgctggcggcgtgcctaatacatgcaagtcgaacgcnagcacttgtgcttcgagtggcgaacgggtgagtaatacataagtaacctgccctagacagggggataactattggaaacgatagctaagaccgcataggtacggacactgcatggtgaccgtattaaaagtgcctcaaagcactggtagaggatggacttatggcgcattagctagttggcggggtaacggcccaccaaggcgacgatgcgtagccgacctgagagggtgaccggccacactgggactgagacacggcccagactcctacgggaggcagcagtagggaattttcggcaatgggggaaaccctgaccgagcaacgccgcgtgaaggaagaaggttttcggattgtaaacttctgttataaaggaagaacggcggctacaggaaatggtagccgagtgacggtactttattagaaagccacggctaactacgtgccagcagccgcggtaatacgtaggtggcnagcgttatccggaattattgggcgtaaagagggagcaggcggcagcaagggtctgtggtgaaagcctgaagcttaacttcagtaagccatagaaaccaggcagctagagtgcaggagaggatcgtggaattccatgtgtagcggtgaaatgcgtagatatatggaggaacaccagtggcgaaggcgacgatctggcctgcaactgacgctcagtcccgaaagcgtggggagcaaataggattagataccctagtagtccacgccgtaaacgatgagtactaagtgttggatgtcaaagttcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatactcataaaggctccagagatggagagatagctatatgagatacaggtggtgcatggttgtcgtcagctcgtgtcgtgagatgttgggttaagtcccgcaacgagcgcaacccttatcgttagttaccatcattaagttggggactctagcgagactgccagtgacaagctggaggaaggcggggatgacgtcaaatcatcatgccccttatgacctgggctacacacgtgctacaatggatggtgcagagggaagcgaagccgcgaggtgaagcaaaacccataaaaccattctcagttcggattgtagtctgcaactcgactacatgaagttggaatcgctagtaatcgcgaatcagcatgtcgcggtgaatacgttctcgngccttgtacacaccgcccgtcacaccacgagagttgataacacccgaagcnggtggcctaaccgcaaggaaggagctgtctaaggtgggattgatgatnggggnnnnnnngtaacaaggtatccctacgngaacgnnnnnnnngatcacctcctttcn

SEQ ID NO: 9 - DNA, 1519 bp, Clostridium sardiniense
tttaaattgagagtttgatcctggctcaggacgaacgctggcggcgtgcctaacacatgcaagtcgagcgatgaagtttccttcgggaaacggattagcggcggacgggtgagtaacacgtgggtaacctgcctcatagaggggaatagccttccgaaaggaagattaataccgcataacattgcagtttcgcatgaaacagcaattaaaggagcaatccgctatgagatggacccgcggcgcattagctagttggtaaggtaatggcttaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgacggtcttcggattgtaaagctctgtctttggggacgataatgacggtacccaaggaggaagccacggctaactacgtgccagcagccgcggtaatacgtaggtggcaagcgttgtccggatttactgggcgtaaagggagcgtaggcggatttttaagtgggatgtgaaatacccgggctcaacctgggtgctgcattccaaactgggaatctagagtgcaggaggggagagtggaattcctagtgtagcggtgaaatgcgtagagattaggaagaacaccagtggcgaaggcgactctctggactgtaactgacgctgaggctcgaaagcgtggggagcaaacaggattagataccctggtagtccacgccgtaaacgatgaatactaggtgtaggggtttcgatacctctgtgccgccgctaacgcattaagtattccgcctggggagtacggtcgcaagattaaaactcaaaggaattgacgggggcccgcacaagtagcggagcatgtggtttaattcgaagcaacgcgaagaaccttacctagacttgacatcttctgcattacccttaatcggggaagtcctttcggggacagaatgacaggtggtgcatggttgtcgtcagctcgtgtcgtgagatgttgggttaagtcccgcaacgagcgcaacccctattgttagttgctaccattaagttgagcactctagcgagactgcccgggttaaccgggaggaaggtggggatgacgtcaaatcatcatgccccttatgtctagggctacacacgtgctacaatggcaagtacagagagatgcaataccgtgaggtggagctaaacttcaaaacttgtctcagttcggattgtaggctgaaactcgcctacatgaagctggagttactagtaatcgcgaatcagcatgtcgcggtgaatacgttcccgggccttgtacacaccgcccgtcacaccatgagagttggcaatacccaaagttcgtgagctaacgcgtaagcgaggcagcgacctaaggtagggtcagcgattggggtgaagtcgtaacaaggtagccgtaggagaacctgcggctggatcacctcctttct

SEQ ID NO: 10 - DNA, 1529 bp, Clostridium scindens
gagagtttgatcctggctcaggatgaacgctggcggcgtgcctaacacatgcaagtcgaacgaagcgcctggccccgacttcttcggaacgaggagccttgcgactgagtggcggacgggtgagtaacgcgtgggcaacctgccttgcactgggggataacagccagaaatggctgctaataccgcataagaccgaagcgccgcatggcgcggcggccaaagccccggcggtgcaagatgggcccgcgtctgattaggtagttggcggggtaacggcccaccaagccgacgatcagtagccgacctgagagggtgaccggccacattgggactgagacacggcccagactcctacgggaggcagcagtggggaatattgcacaatgggggaaaccctgatgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcgatgcaagccagatgtgaaagcccggggctcaaccccgggactgcatttggaactgcgtggctggagtgtcggagaggcaggcggaattcctagtgtagcggtgaaatgcgtagatattaggaggaacaccagtggcgaaggcggcctgctggacgatgactgacgttgaggctcgaaagcgtggggagcaaacaggattagataccctggtagtccacgccgtaaacgatgactactaggtgtcgggtggcaaggccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgatcttgacatcccgatgccaaagcgcgtaacgcgctctttcttcggaacatcggtgacaggtggtgcatggttgtcgtcagctcgtgtcgtgagatgttgggttaagtcccgcaacgagcgcaacccctatcttcagtagccagcattttggatgggcactctggagagactgccagggagaacctggaggaaggtggggatgacgtcaaatcatcatgccccttatgaccagggctacacacgtgctacaatggcgtaaacaaagggaggcgaacccgcgagggtgggcaaatcccaaaaataacgtctcagttcggattgtagtctgcaactcgactacatgaagttggaatcgctagtaatcgcgaatcagaatgtcgcggtgaatacgttcccgggtcttgtacacaccgcccgtcacaccatgggagtcagtaacgcccgaagccggtgacccaacccgtaagggagggagccgtcgaaggtgggaccgataactggggtgaagtcgtaacaaggtagccgtatcggaaggtgcggctggatcacctccttc

SEQ ID NO: 11 - DNA, 844 bp, Escherichia coli
gcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaac

SEQ ID NO: 12 - DNA, 512 bp, Klebsiella oxytoca
cctgaaagaacctgcctttgtagtgctcacacagattgtctgatgaaaaataagcagtaagaaaatctctgcaggcttgtagctcaggtggttagagcgcacccctgataagggtgaggtcggtggttcaagtccactcaggcctaccaaatttctgctgatgctgcgttgcggcgacactcacatactttaagtatgtttcgtgtcaccacgccttgcctcaacagaaattaaggttgatgagattttaactacgatggggctatagctcagctgggagagcgcctgctttgcacgcaggaggtctgcggttcgatcccgcatagctccaccatcattactgccaaaaacaagaaaacttcagagtgtacctgaaaaggttcactgcgaagttttgctctttaaaaatctggatcaagctgaaaattgaaacgacacacagctaatgtgtgttcgagtctctcaaattttcgcgacacgatgatgtttcacgaaacatcttcgggttgtga

SEQ ID NO: 13 - DNA, 1497 bp, Parabacteroides distasonis
caatttaaacaacgaagagtttgatcctggctcaggatgaacgctagcgacaggcttaacacatgcaagtcgaggggcagcggggtgtagcaatacaccgccggcgaccggcgcacgggtgagtaacgcgtatgcaacttgcctatcagagggggataacccggcgaaagtcggactaataccgcatgaagcagggatcccgcatgggaatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccattactgacgctgatgcacgaaagcgtggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggttaggctgaggactctggtgggactgccagcgtaagctgcgaggaaggcggggatgacgtcaaatcagcacggcccttacatccggggcgacacacgtgttacaatggcgtggacaaagggaggccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgaaaggatcggcctagggtaaaactggtgactggggctaagtcgtaacaag

SEQ ID NO: 14 - DNA, 1506 bp, Prevotella melaninogenica
  misc_feature (36)..(36): n is a, c, g, or t
gatgaacgctagctacaggcttaacacatgcaagtngaggggaaacggcattgagtgcttgcactctttggacgtcgaccggcgcacgggtgagtaacgcgtatccaaccttcccattactgtgggataacctgccgaaaggcagactaataccgcatagtcttcgatgacggcatcagatttgaagtaaagatttatcggtaatggatggggatgcgtctgattagcttgttggcggggtaacggcccaccaaggcaacgatcagtaggggttctgagaggaaggtcccccacattggaactgagacacggtccaaactcctacgggaggcagcagtgaggaatattggtcaatggacggaagtctgaaccagccaagtagcgtgcaggatgacggccctatgggttgtaaactgcttttgtatggggataaagttagggacgtgtccctatttgcaggtaccatacgaataaggaccggctaattccgtgccagcagccgcggtaatacggaaggtccaggcgttatccggatttattgggtttaaagggagcgtaggctggagattaagtgtgttgtgaaatgtagacgctcaacgtctgaattgcagcgcatactggtttccttgagtacgcacaacgttggcggaattcgtcgtgtagcggtgaaatgcttagatatgacgaagaactccgattgcgaaggcagctgacgggagcgcaactgacgcttaagctcgaaggtgcgggtatcaaacaggattagataccctggtagtccgcacagtaaacgatggatgcccgctgttggtacctggtatcagcggctaagcgaaagcattaagcatcccacctggggagtacgccggcaacggtgaaactcaaaggaattgacgggggcccgcacaagcggaggaacatgtggtttaattcgatgatacgcgaggaaccttacccgggcttgaattgcagaggaaggatttagagataatgacgcccttcggggtctctgtgaaggtgctgcatggttgtcgtcagctcgtgccgtgaggtgtcggcttaagtgccataacgagcgcaacccctctcttcagttgccatcaggttaagctgggcactctggagacactgccaccgtaaggtgtgaggaaggtggggatgacgtcaaatcagcacggcccttacgtccggggctacacacgtgttacaatggccggtacagagggacggtgtaatgcaaattgcatccaatcttgaaagccggtcccagttcggactggggtctgcaacccgaccccacgaagctggattcgctagtaatcgcgcatcagccatggcgcggtgaatacgttcccgggccttgtacacaccgcccgtcaagccatgaaagccgggggtgcctgaagtccgtgaccgcaaggatcggcctagggcaaaactggtgattggggctaagtcgtaacaaggtagccgtaccggaaggtgcggctggaacacctcctttct

SEQ ID NO: 15 - DNA, 1497 bp, Proteus mirabilis
tgatcctggctcagattgaacgctggcggcaggcctaacacatgcaagtcgagcggtaacaggagaaagcttgctttcttgctgacgagcggcggacgggtgagtaatgtatggggatctgcccgatagagggggataactactggaaacggtggctaataccgcataatgtctacggaccaaagcaggggctcttcggaccttgcactatcggatgaacccatatgggattagctagtaggtggggtaaaggctcacctaggcgacgatctctagctggtctgagaggatgatcagccacactgggactgagacacggcccagactcctacgggaggcagcagtggggaatattgcacaatgggcgcaagcctgatgcagccatgccgcgtgtatgaagaaggccttagggttgtaaagtactttcagcggggaggaaggtgataaggttaatacccttatcaattgacgttacccgcagaagaagcaccggctaactccgtgccagcagccgcggtaatacggagggtgcaagcgttaatcggaattactgggcgtaaagcgcacgcaggcggtcaattaagtcagatgtgaaagccccgagcttaacttgggaattgcatctgaaactggttggctagagtcttgtagaggggggtagaattccatgtgtagcggtgaaatgcgtagagatgtggaggaataccggtggcgaaggcggccccctggacaaagactgacgctcaggtgcgaaagcgtggggagcaaacaggattagataccctggtagtccacgctgtaaacgatgtcgatttagaggttgtggtcttgaaccgtggcttctggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagcgaatcctttagagatagaggagtgccttcgggaacgctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcagatacaaagagaagcgacctcgcgagagcaagcggaactcataaagtctgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtagc

SEQ ID NO: 16 - DNA, 1428 bp, Subdoligranulum variabile
  misc_feature (1189)..(1189): n is a, c, g, or t
tgcaagtcgaacggagttatttcggttgaagttttcggatggatactggtttaacttagtggcgaacgggtgagtaacgcgtgagtaacctgccctggagtgggggacaacagttggaaacgactgctaataccgcataagcccacgatccggcatcggattgagggaaaaggatttattcgcttcaggatggactcgcgtccaattagctagttggtgaggtaacggcccaccaaggcgacgattggtagccggactgagaggttgaacggccacattgggactgagacacggcccagactcctacgggaggcagcagtgggggatattgcacaatgggggaaaccctgatgcagcgacgccgcgtggaggaagaaggttttcggattgtaaactcctgtcgttagggacgaatcttgacggtacctaacaagaaagcaccggctaactacgtgccagcagccgcggtaaaacgtagggtgcaagcgttgtccggaattactgggtgtaaagggagcgcaggcggaccggcaagttggaagtgaaatctatgggctcaacccataaattgctttcaaaactgctggccttgagtagtgcagaggtaggtggaattcccggtgtagcggtggaatgcgtagatatcgggaggaacaccagtggcgaaggcgacctactgggcaccaactgacgctgaggctcgaaagcatgggtagcaaacaggattagataccctggtagtccatgccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccgatgcatagtgcagagatgcatgaagtccttcgggacatcgagacaggtggtgcatggttgtcgtcagctcgtgtcgtgagatgttgggttaagtcccgcaacgagcgcaacccttattgccagttactacgcaagaggactctggcgagactgccgttgacaaaacggaggaaggtggggatgacgtcaaatcatcatgccctttatgacctgggctacacacgtactacaatggcgtttaacaaagagangcaagaccgcgaggtggagcaaaactcaaaaacaacgtctcagttcagattgcaggctgcaactcgcctgcatgaagtcggaattgctagtaatcgcggatcagcatgccgcggtgaatacgttcccgggccttgtacacaccgcccgtcacaccatgagagccgggggggacccgaagtcggtaagtaagtctaaccgcaaggaggacgccgccgaaggtaaaactggtgattgggtg

SEQ ID NO: 17 - DNA, 26 bp, Artificial Sequence
  synthetic oligonucleotide
gaacgtttgaagtatcaggaggacaa

SEQ ID NO: 18 - DNA, 27 bp, Artificial Sequence
  synthetic oligonucleotide
actcaaagggctgcaatagctagagga

SEQ ID NO: 19 - DNA, 26 bp, Artificial Sequence
  synthetic oligonucleotide
gatttcctgtaggttcatcggctaat

SEQ ID NO: 20 - DNA, 28 bp, Artificial Sequence
  synthetic oligonucleotide
gctcttaatgaattaccatgtgcaacaa

SEQ ID NO: 21 - DNA, 26 bp, Artificial Sequence
  synthetic oligonucleotide
aggctcgtttaccagcctgcatatct

SEQ ID NO: 22 - DNA, 24 bp, Artificial Sequence
  synthetic oligonucleotide
acaacaattgagcgggttattcct

SEQ ID NO: 23 - DNA, 22 bp, Artificial Sequence
  synthetic oligonucleotide
ggcagttcggcaagtatctgtt

SEQ ID NO: 24 - DNA, 24 bp, Artificial Sequence
  synthetic oligonucleotide
tgcatgacatcgggaaaggcaggc

SEQ ID NO: 25 - DNA, 22 bp, Artificial Sequence
  synthetic oligonucleotide
gcaatctatgcagcggcaattc

SEQ ID NO: 26 - DNA, 27 bp, Artificial Sequence
  synthetic oligonucleotide
ttctatggctttgtatgaaactcttgc

SEQ ID NO: 27 - DNA, 25 bp, Artificial Sequence
  synthetic oligonucleotide
tcaccaccaggatggttgtttggca

SEQ ID NO: 28 - DNA, 30 bp, Artificial Sequence
  synthetic oligonucleotide
agtcatatatactctgtatagagcaaaccc

SEQ ID NO: 29 - DNA, 21 bp, Artificial Sequence
  synthetic oligonucleotide
aatactgactccggcgcaaat

SEQ ID NO: 30 - DNA, 24 bp, Artificial Sequence
  synthetic oligonucleotide
aagctgaacgccggctacaaaggt

SEQ ID NO: 31 - DNA, 22 bp, Artificial Sequence
  synthetic oligonucleotide
cgccttggtgtttggctttatg

SEQ ID NO: 32 - DNA, 23 bp, Artificial Sequence
  synthetic oligonucleotide
tgttcaagctgctataggatcag

SEQ ID NO: 33 - DNA, 25 bp, Artificial Sequence
  synthetic oligonucleotide
acttgcggctggactaaattgtgga

SEQ ID NO: 34 - DNA, 19 bp, Artificial Sequence
  synthetic oligonucleotide
gctccaagtggtgcagtta

SEQ ID NO: 35 - DNA, 22 bp, Artificial Sequence
  synthetic oligonucleotide
aacctagccaggcattgtgaac

SEQ ID NO: 36 - DNA, 25 bp, Artificial Sequence
  synthetic oligonucleotide
accctgcctcaagggaaacacatgc

SEQ ID NO: 37 - DNA, 27 bp, Artificial Sequence
  synthetic oligonucleotide
cgtacttcttccgaaaccggattatag

SEQ ID NO: 38 - DNA, 20 bp, Artificial Sequence
  synthetic oligonucleotide
caccggacaattcgggataa

SEQ ID NO: 39 - DNA, 25 bp, Artificial Sequence
  synthetic oligonucleotide
cgaatctgtagctgtctcgctccaa

SEQ ID NO: 40 - DNA, 21 bp, Artificial Sequence
  synthetic oligonucleotide
cgtttccgagcaggaatatca

SEQ ID NO: 41 - DNA, 26 bp, Artificial Sequence
  synthetic oligonucleotide
cttgtcccgtaatcttcaccgtattc

SEQ ID NO: 42 - DNA, 24 bp, Artificial Sequence
  synthetic oligonucleotide
aaccctgcacctgtttgctctgca

SEQ ID NO: 43 - DNA, 25 bp, Artificial Sequence
  synthetic oligonucleotide
ggtattgtcttgtttgccgttagga

SEQ ID NO: 44 - DNA, 22 bp, Artificial Sequence
  synthetic oligonucleotide
gtgattatcggcggaggcattt

SEQ ID NO: 45 - DNA, 29 bp, Artificial Sequence
  synthetic oligonucleotide
acgggaacaggtctggaaattcgtgatga

SEQ ID NO: 46 - DNA, 24 bp, Artificial Sequence
  synthetic oligonucleotide
ggcatccggccttatttcctaatc

SEQ ID NO: 47 - DNA, 23 bp, Artificial Sequence
  synthetic oligonucleotide
gtgccaagtggactgacatcttt

SEQ ID NO: 48 - DNA, 27 bp, Artificial Sequence
  synthetic oligonucleotide
acagcagcaagaaacctatctgtggcc

SEQ ID NO: 49 - DNA, 24 bp, Artificial Sequence
  synthetic oligonucleotide
caacgaggtaaggacgcaacatag

SEQ ID NO: 50 - DNA, 23 bp, Artificial Sequence
  synthetic oligonucleotide
cggtagagcctactgcaggataa

SEQ ID NO: 51 - DNA, 23 bp, Artificial Sequence
  synthetic oligonucleotide
atcaccgttgcacccgaaccgtt

SEQ ID NO: 52 - DNA, 24 bp, Artificial Sequence
  synthetic oligonucleotide
ccggagttaaaggtatcggctatg

SEQ ID NO: 53 - DNA, 23 bp, Artificial Sequence
  synthetic oligonucleotide
gtagtgtgttggcggctcaaata

SEQ ID NO: 54 - DNA, 24 bp, Artificial Sequence
  synthetic oligonucleotide
tgccaacgcaccgatgtaagagcc

SEQ ID NO: 55 - DNA, 23 bp, Artificial Sequence
  synthetic oligonucleotide
gcccgcagagggaatatacaaag

SEQ ID NO: 56 - DNA, 22 bp, Artificial Sequence
  synthetic oligonucleotide
gcgcttatgatccaaggcatga

SEQ ID NO: 57 - DNA, 24 bp, Artificial Sequence
  synthetic oligonucleotide
aggaacagggcacgctcgtctaca

SEQ ID NO: 58 - DNA, 24 bp, Artificial Sequence
  synthetic oligonucleotide
catgaagacgttggagacgaacag

SEQ ID NO: 59 - DNA, 19 bp, Artificial Sequence
  synthetic oligonucleotide
ttaccgaccgaactctcct

SEQ ID NO: 60 - DNA, 25 bp, Artificial Sequence
  synthetic oligonucleotide
cgccgcaggatgtggtgaataaggt

SEQ ID NO: 61 - DNA, 21 bp, Artificial Sequence
  synthetic oligonucleotide
gatcgactggccttccataat

SEQ ID NO: 62 - DNA, 747 bp, Artificial Sequence
  synthetic polynucleotide
atggaaattttaaaaattaataatgtatctaaaacttatgaagggaaggtatcttatcaagccttaaaaaatataaacttgtctatagaagaaggtgaatttgttgctgtaatggggccaagtggtagtggaaagtcaactttattaaatgttatatctacaatagatagaccaacttcaggtgaagtaatattaaactctaaaaacccgcatgaattaaaaggccaggatttagcaaatttcagaagaaatgaacttgggtttgtatttcaaaactttaacttgttagatactttaacaattggtgaaaatatagtacttcctttaacattagatggagcttcagtaaaagatatgaataatagattaaatgaaatatctaaaaagctaggtatagaacagataataaacaaaagaacgtttgaagtatcaggaggacaaactcaaagggctgcaatagctagaggaataataaataaaccatcaattttattagccgatgaacctacaggaaatctagattcaaaatctacagatgatgttatggatttatttacaaaaataaatactgaaaataaaatgactacgcttatggtaacgcatgagccttatactgcaagtttttgtaacagaatcatttttataaaagacggagaaatttacaaagaacttaataaaaaaggtaatagagaagacttctatgaagaaatactattagtgctatctcaaataggaggtgctagatag

SEQ ID NO: 63 - DNA, 453 bp, Artificial Sequence
  synthetic polynucleotide
aaggatcaatgtttcaatcataattaaacagtaactagttttttaccctgatcagcaacagcatcgatcttctcttttaataacgtttcatcacctaaatagtaatgttttattactttaaattcatcatcaaattcatatactaaaggaattccagttggaatattaatattcatgattgcttcgttacttaacttatcaaagtatttaaccaacgctcttaatgaattaccatgtgcaacaattaaggctcgtttaccagcctgcatatctttttttactgtttcattaaaataaggaataacccgctcaattgttgtttttaagctctcacctgctggtaaaagagcagagtcaatatttcgatacattgcctgcttttgcgcgctgcgtttatcatttatatttaaagctggtggcagtacatcaaatgaacgacgccaaattttcacc

SEQ ID NO: 64 - DNA, 513 bp, Artificial Sequence
  synthetic polynucleotide
tcatattctaatctccttattctttttttcagtactccagttacagtccgtttctgccggacaggcgaaacaggcgcgagaggccttgtcgctttcatataatagtgcttccagaggttcagcgttctcgcgaagcttccggtgtccacggattgccgtaagaatctgttcgttttcattcgaagtaaacataagttcttccggcagttcggcaagtatctgttcagcaatatcagcgcttgcaagttcatgtgggattcctgactcatactgcctgcctttcccgatgtcatgcagaattgccgctgcatagattgcttctttggaaattccaattcctctttcaaggctcagaatgtaagcaattctggctgtgtccagcagatgattcatctgatggcagcagaagatacgttcttgctccagttcttgcagtctttggtaagaagatatatataggggatgcttgcggatataagaaattcttaacatcgtgttctccatatgattcat

SEQ ID NO: 65 - DNA, 501 bp, Artificial Sequence
  synthetic polynucleotide
atgaaaaatatatttaaggtaaatgacaaatttaagcttataccattgatcatatcaatattaattccagtaggtggaggcgtattggtaggatatattacaaaagattctatggctttgtatgaaactcttgcaaaacctaaattttcaccaccaggatggttgtttggcattgtatggcctattctatatatcattatagggtttgctctatacagagtatatatgactcttaaagaagaaaagaggagttatggaattttaatagtgtattttattcagcttttgattaatttcttatggcctatattattctttaatttaaaattgtatggattatcagcaattaatataatcatacttataattttaataataatatgtataataaaatttataaagattgataagatatcatcagtattattaattccatatttaatatggtgcggttatgcagcttatttaaatatagtaatttggatgataaatgaaatgtaa

SEQ ID NO: 66 - DNA, 809 bp, Artificial Sequence
  synthetic polynucleotide
ttgacgcttctgtctgatagcgtcaaaggcttttttattggttacagctgttatacagcatacagtgatttgctcaatagatttgacaaggaacgaagaagtcctctattgctgcgcgcctgcctgcgcggcatgaagatggctgaaatcgatttccttttcaatgttatgccgatgcatataaacgctttccacaattccgacgccaaggtacagacacgccacggaggagcctcctgagctgaaaaagggaagagtaatgccgattaccgggagcagacccaggcacatgcctacgttgatgacagtctgaaccgccagcatagcgaaaaagccaaaacaaaggaatttacccaggtcatccttggcggaaagagcgttcatgacgcagcggagcatcagcagcaaaagcagtcccagcaggacgacgcacccgataaagcccagctcttctccggctacagagaaaataaaatcattttcctgatagggaaccgaccctgccgccacccgggggccttcatagtagccccggccatacatttcgccggaggcgatagaaatttttccttggagctgctggtagccgaacccattgggatcggattccaaattaaacagagtccaaaaccgcattttttgatcctcatttaatacagaattccataaaatcggaatactgactccggcgcaaatgattaaagctaaatagtatctaagctgaacgccggctacaaaggtcataatcaggaacataaagccaaacaccaaggcggtgccgtcgtcgcccataaaatggattaatccaa

SEQ ID NO: 67 - DNA, 1188 bp, Artificial Sequence
  synthetic polynucleotide
atgttagtatcacttgctttaatttttttagttggaatgagcttggcatctatatgcgaaaaaattaaaattccgagaattataggaatgcttgtaactgggataattttaggtccttatgtactagattttttagatagctcaattctaaacatatcatcagagcttagaaaaatggctcttatcatcatcttaatcaaagctgggttatcactagacttaaaggacttaaaaaaagttgggagacctgcgcttttaatgtcgtttcttccagctacatttgaaataattgcttatgcaatatttgctccaattttatttggtgtcagcagagtagaggctgcactaattggagcagtacttagtgcagtatctccagcagttgtagttcctagaatggttgatttaatggacaataatctcgggacaaagaagggaatacctcagatgattttagctggggcatcttttgacgatgtatttgtaattgtgctattcagtacatttttagctatgaatcaaggagaaggtgttaatctttctagttttgcagacataccaatttcgatagtatctggaattttaattgggtctgttgttggattaattctttatagattctttgagtatagatacaacaaagaacatctgataagaaacagcacaaaagtcataattatcttggctgtatcgttcttacttgttgcacttgaagattatttaaaaggaagagttgctatgtccggacttcttgctgtaacaagtatggcattagtacttgctatgaagagtacaaatattgtaaaggtcagacttcaagagaaattcggtaaaatttggatagccgcagaagttgttttattcgtacttgttggggctgctgtcgatataagatatacaatgggagctggatttacagcagttattatgatatttatcgcacttgcaattcgttcaataggtgtatttatttgcatgattggaacagaattaaacactaaagaaagattattctgtgtgttctcatatcttccaaaggcaactgttcaagctgctataggatcagtaccacttgcggctggactaaattgtggaaaacttgtactatcaattgcagtacttgcaataataataactgcaccacttggagcatttttaatagatttctcaaaagaaaaattattatag

SEQ ID NO: 68 - DNA, 1200 bp, Artificial Sequence
  synthetic polynucleotide
caacatgggcagtgaaccgggtatggcctggaactggtgcataaacctagccaggcattgtgaactatacattatcactgaaggtgaattcagagacaaaatagaggcagtgctccctaccctgcctcaagggaaacacatgcacttttactataatccggtttcggaagaagtacggaagatgtgttggaatcaaggagattggcgtttctataaacactataagaaatggcaatggaagacttacgagatggcacaggaaataatagtcaaacaacatatagatattgtacaccaattaaatatgattggctttagagaacccggatacctttggaaactagataagccatttgtttggggaccggtagatgctaaagaaaaatttccgacagcatatctaagagatgcagggataaaagcaaacttattcatcagattaaaaaatcacataaccggtttacagttacgatattcacaacgagtaaaaaaagctgtaaaaaaagcctctgtagtaacatccgcatcttctgaatctcagaagagtttcaagaaatattttcatattgacgctcccttattaaatgaaacagggtgttatcctaaaacaacaataataaacagtacaaaagaaaaaggtgatttaaatttgctttgggtaggtaaattagatttcagaaaacaattgcctttagcgataaaggccatagcacgactggctaatccacatataaaactccatatcgtaggtggtaacaataattcctatcaaaagttagcgatggaattgaacatatcacatcaatgtatctggcatggggttatctcacataatgaagttcaggaactcatgcagaaagcagatattttcttttttaccagcatagctgaaggaactccacatgttgttttagaagccatcaacaataaccttcctgttatctgtttcgacatatgtggacatggtgactcaattaatgaacaagtagggataaaaattcccctatctactccgcaacaatccatcaacgattttgcggagaagataacatatctgtttaaccatagagacgtacttaagcaaatgtctgaaaattgcagagtccgtcaagaggaactatcgtgggacaataaagccaaacagatggtcagtctatataaaaaagtattgtcacaaaaatgagtaaaagatacaagcta

SEQ ID NO: 69 - DNA, 807 bp, Artificial Sequence
  synthetic polynucleotide
ttatctgcgtaacggagtccgttcatcaataattccgcggcctccgtaaagtctcatcgtaacaggacttccggcttctatggtaatatcctgatctgcgatgataggttcttccttatcgtctacaattacagaaccaatcttctcaccagccttgattatctgtggaatcaagttagtcaggataccttcggaaattttccggatactgatatattccggggttgcattcttgtcagcttcgattttaccgttgaaaagatcctgcacacctttgttcttcaagaaaagtttaacgattgctttaccttcgtacgggttgcctttttcatcaagtagaataacgggcgcagtcattcgcttgaatgtaagggctacattgctagaacctttgctggcttgggctgtgccaaacaaatattctaaaccgcctttgatgtcacggtagcgactaaagagggatctagtcgttacatcaccggacaattcgggataatgagcatagaaagttacagcttccgaatctgtagctgtctcgctccaattttggttatctactcccaaagtaaaaggctgatattcctgctcggaaacggacattgaaatgacgtctccagctgaaaaactactttttaaagggagtgacaggttactgctgctacgagtaacagcgtcagtgatactttcattgtcgacagtagtaagaaagttgataccttctttactgggatcttgcagttcttcttgctgttgacaactgaacaggcatactgatgccataacaaataaaaggctttttcttttcat

SEQ ID NO: 70 - DNA, 3078 bp, Artificial Sequence
  synthetic polynucleotide
ttatttgaatgataaattaataccaaatattacattgcgagtcattgcatccgatgtattgatgctgacattcaactgttcaggatcaaccacatcctttgcacagaaaataaatggattctgaactgttgcatataaacgcaaattgcctaatttcattttctcaagaaccgatggtttaaaagtatatcccaaagtaatataagaaatcttaaggaaattactggaaaacatcgtatgtgacatttccgattttgcatttccccatgatcctgccttgcttcgataagtacccatgttactaggctgtgcagaagcattggtaggattttccggagtccaataatcttttcttaaattattaaaattgagattgttgttttccaatgcatacgaaacatagaactgatttctagctcttgcccctgcttggaaagctgcttgaaatgacaaatcaaattccttatatgtaaatgtattagtcataccacctgtccaatccggagtacgtttaccatcaatcacacgatctttatcgtcaatcacaccatcctcattcaaatccaaaggtttatattgaccaggtttacaaccatatttagctgcttcttcagcttcatccagctgccatactcccaatgtcatcaaattataatttatatcaataggctgaccaataatccaaagattactataatcaccactcataccagctaaagacaatccacgggaagtcaaattctccttatactgtaaatctacaattttattcttattataggcaaaattcaaactagttctccatgaaaaatctttagtacggatattatccgaattaatatttacctcaaacccttcatttctaacagaccctacatttgcttttacagaagaataaccggtagtaacaggtactgtcttattcataatcaagtcttctgtcaaacgattataatattccacactacccgaaatccgattattgaaaaatccgaagtcaagacctacattatattctgtagtacgttcccatcccaaatccaaattccgtaaattattgggaacatatccaatagactcactagaaccgaatgtataatatttggcaccactaatagatccttgcgtctgatacgcacttacattatcattaccggtctgaccataacttaaacgtaacttcaaattactcaaccaagacaagctcttcataaattcttctcctgacactctccatgctacagctgctgatggaaaacttccccacttattgccttcagctaattttgacgaaccatcaaaacgaatacttgctgttacaaaatacttgtccttatacacataattggcacgtgctaaataagacatcaaattcgttttggtaaaaccggaagaagaagtattacttgccgatcctcctgccaaattataccacaaagagttatatgacaatccattaccaattccttttaacttttcatcttgtgattgctgcatagagaacactccggtcaaatctacgcgatgatccttcaattcaaagttataatttacaagattatcccaaacccaatctacatagctatttttcgcataattactggttgccttattttgccctttattagctttagtgtatttacctctgtactggccaatttcttccaaatttatatccggcgagaaagtggtcttcaaagtcaatcctttaattggagttattgccagataaatgttactcagcaaattgtactttttggttttattcagttcattcttcatggtagttaacgcattatactgaccattagaataagcccacatttcttctcctgtcaccaaatcagtcggatgataagtcggacgcagacgaaaagcatcctgtaacaagtcagagttacccgtatcacgtactgaatgggttccatacatattaattccaaatttcatgtatttgctaggttctacatccacaacagcacgcagattataacgggaatattcttgaggctccaacataccatcttcaaaataatatccggcagataaagcataagtagccatctcattacctccggtagccgatatagtatgatttgtcataaaggccggactagaaacagcatccacccaatcaaaataatttccatcttgaatagcctttaactctgatgctgtgaaaatctgactatcatccacatatttattattatttccggctcttttagcctctcgtgccaattgcacatattcttcaccagacatcatatcgggcatattcgtatatttcctatatccggcatatccactataatctatttttactttacctatcttacctcttttagtagtaaccatgactacaccattagtagcacgggaaccatagattgcggtagatgatgcatctttcaaaatatcaattttttctatatcatccggattcaggttagataaagaagctcctggcacaccatctacaacaattaagggagatgtacttccgctaattgtattcaaaccacgaatcagaatattatattcccctcctggtttactattagaacgttgaatttgcacacctggcaaagccccttgcatagctcctactgcatccgttctaccacttcttatcaaatcctgagtagaaacagaagctacagcacctgtcaaatccgatttctttaccgaaccataacctaccactacaacttcgtccaacagctcagtatcttcttttagaacaatcttgaaagaacggttagaaccaatattcaattcttgagtttgaaaaccaacgtaagatatctgtaacttatgagaaggaaaaacagtcaaactaaaattaccatccaaatcagttacaataccgttggtagttcctttctccattactgtagcacctattatcggttctccatttggatcaataacttgtcccgtaatcttcaccgtattctgcaatacttcttgattaatcggagagtacaaccctgcacctgtttgctctgcaaccgcatttcctaacggcaaacaagacaataccataagtaataaaaatttgtctttctgtttcat

SEQ ID NO: 71 - DNA, 927 bp, Artificial Sequence
  synthetic polynucleotide
atgaaatacgctatcgcattagacattggaggaacaagcataaaatatactcttgtgaaccagaatggcgacattctttacgaatcgtcggaaaccacacaatcaaaagagaatccacgcccattatccgataccataaaaagtatcgtacggaaaatgacagactacgcccgttcccgggactgggggatttacggaattggcataggtgtaccttccgtagtagataagggggtggttctctttgccaataatcttcctgaactggacaaccaacaattggatcttgcgttagcggaatttaatctaccggtattcatcgacaacgacgcaaaccttatggggttgggcgaggtgatatatggggcggctaagggcctctccgatatcgttttcttgacggtgggtaccggcatagggggcgccttgttcttgaacggccggctttatggcggctaccggaaccggggcaccgaacttgggcacttaattattcatggtctgaatgggaatcaatgtacttgcggagcgtccggttgcctagaggcacacgcttccgtaagtgcgttgatcgccttatatcggcaattattggagaagaacggacgggagataccttcccgtatcgatggaaaatatatagtagagcgctataaagctcaagagaaagaggccgtactcgctatggaggatcatttccggaatctgtccctaggcgtagcgagccttatcaacattttcgctccgcaaaaggtgattatcggcggaggcatttcggaatccggagatttctacattaataatatacgggaacaggtctggaaattcgtgatgaaggaaacctcgtacttcacgactatcgaacttgcccgattaggaaataaggccggatgcctcggggcggcggcattggtgtttaatcattga

SEQ ID NO: 72 - DNA, 900 bp, Artificial Sequence
  synthetic polynucleotide
ttacctttggtacttccctcagcatgacattctaaggagaggattaagggtaggagaaaaaggatgctgcagctaattcagctcacttctcctaaagtcctttctcatgatatgtaacacgatgctctacttatataattcactacatgttcaacagcatttcatcaaagaaactaagctttggtacgttcttctgtctatatattgcacagatggtaccgtcatcctttcttatgacagccttacaggttatcatgagggaaggacaatatagtcttgcaaccattgggttgctaaaccttgtccgagttccatggacgataaagttcttatggtcgccttttgttgaccgccactgcgtaacagtgcgtgactacaaacgtacgattatcgctacagagttgatatatgccgttgcactcttagccacagggctgattaatgtgcgttcggaaatcatgcttgtagtcattctggcgtttatctctatgcttgcttcggctacgcaagacattgctacagatgcgcttgccatcctttcttttaagaagcacgaccatagtatgcttaatagtatgcagtctatgggagctttcggaggtgctgttattggtggaggagtcttgcttatcttactgaaaagctatggctggaatgttgtagtaccctgcttagccttgtttgtttgtatgatgattattcctttaatgtttaatcctcatatcaagatagagaatgagaaaccaagagagcgtgccaagtggactgacatctttagcttctttggacgtaaggagatatggccacagataggtttcttgctgctgtattatatgggaatcatcggtattctttctatgttgcgtccttacctcgttgataagggatacgatatgaagg

SEQ ID NO: 73 - DNA, 1074 bp, Artificial Sequence
  synthetic polynucleotide
ttattgataagaaaaagtgagactggctaatccgtttgctttacccggttttagtgtcccagtcgtgatatagcgagttgacaggtttaatatgtagttttcgttgttagaagttgttatatggagtaaacgttggttaggttgtcctatcgcactgatatcagggccatacaccacgggagttggattatcattaaagaaaaattgaataccaataccagaagcatcagaatctgatgtcaatgtgaggatatctgtattattagaataggtagactgatcggtcatgctggcatataaatgcacattactatcgcatgtaacagagatgggtttagcctgactttttgatgttgaaccaatggctgccatatctcgcattgagatatcacctaattcataggtgagatttttggtatttacggtacagcctcgagctttaatagtaatagtgacggggttaattgctacgatagaggtattattgcctttatggcctccaccatattcagaccatgtattggcgataactgtatatggaatgttataaacgccagtctcaagatgtttatctgtcaccacaaagacgaccttgcctttcagtgaaacgcgatccacattatggttaacggtagagcctactgcaggataaatcaccgttgcacccgaaccgttatcaacatcaataggtacgtaagcaacactatcattgttatcttttaaaccaaaagcatagccgatacctttaactccggggagtgaataaacaggataacgattactaccttcgtaatagtagatcccgctaatttttgaaccagtagcattagcatagagggtttcaacccaacatttttgcaaaaatttcttctcacatttgaaaccattatggacagaggtttcacctatccaagttgacgtgatgggcgttcctgcgggagtaccatcggcggcaccatcaaaattcataggtggaggggttaccactaatggatctgcaatagcccatgccatttggctcgggatacaaagaagagaaaccagcaatgtgaagaaagaaaaagagcgtgtcat

SEQ ID NO: 74 - DNA, 1440 bp, Artificial Sequence
  synthetic polynucleotide
ggcgatatttcttcaggctgtgggacgtatgcgtcggttggtaatcgctggcaggttggccatgaagatgaaatttttgcctttgcactcaccaacgccattaccagtactggtaaaggcgttaatctacaggggctacaattttgcaaactcattgataaaagctcaccgctactgtctaatgccatcaatcagaatgagcggttatttattgaaatcgatttgtatcgtataaataaaagcgggcgctgggaacggtattattatattcagctaagaaatgcttcattaactgctattcatgtaaacatttctgacaataatcttcctaccgaatgtgtaaatgttaattatgactacatattatgcaaacatctaatagccaatacggaatttgactggttggcctttcctgctggctataatagcttatttattccacctaaaaacccacctgccagtaatcttaaccctgagccgctaccagttgttaaccttccactctctccaccagcggttaaaccggtctatgccaaatcctgtctgaaggagaagggatgtacagatgccggaacggcagaagaacccgctgaaaacttcgggcaagtagcgatttttgctctgccagtggttgatgactgctgtggataccaccatccggaggctaacgatgttgggcaacccgcagaagctcaaaccatgctactgtttccgggtagtgtgttggcggctcaaatatggggaaaatggtcgctcagtggcatactcagtgcaacccgcggctcttacatcggtgcgttggcatctgctttgtatattccctctgcgggcgagggcagtgctcgtgtgcctggacgtgatgagttctggtatgaggaagaactgcgccagaaagcgcttgcaggcagtaccgccactacccgggtgcgttttttctggggaactgacattcacagcaagccccaggtatatggtgttcatacgggtgaaggtacgccgtatgaaaacgtccgcgtggcgaacatgctgtggaacgaggagaggcagcgttatgaatttacccccgctcacgatgtcgatggccccctgattacctggacgccggaaaatccggaacatgggaatgttccgggccataccggtaacgacaggccgccgctggatcagcccaccattctggtgacgccgattccggacggcaccgatacctataccacgccgccattcccggttcctgatccgaaagaattcaacgattatattctggtttttccggcgggatccggtattaagcccatctatgtttacctgaaggaggatccgcgaaagctgcctggtgttgtaacagggcgcggcgtcctgctttcaccaggaactcgctggctggatatgtcggtatccaataacggcaacggcgcaccaatcccggcgcatattgct

SEQ ID NO: 75 - DNA, 1494 bp, Artificial Sequence
  synthetic polynucleotide
atgttcgatatgttcatgacagccttcagttcggcctgtagtctggaagccctcgtcgccaacttcatcggcgtggcgctcggcatcgtcttcggcgcgctgcccggcctcaccgccgtcatgggcgtggccctgctcattcccttgaccttcggcttccccgccgtcatcgccttttcctccctgctcggcatgtactgcggggccatctacgcgggcagcatcaccgccatcctcgtcggcactccgggcacggcggccgccgccgcgaccatgctcgaagggccgcagttcaccgcccgcggggaatcgctcaaggcgctcgaaatgaccaccatcgcctccttcatcggcggcatcttctcctgccttgtgctggccacggtcgccccccagctcgcacacttcgccctcgacttcagcgccccggaatatttctccctcggcatcttcggcctgaccattgtggcgacgctgtccgaaggtgcgctgctcaagggctgcatcgcggcgctgctcggcatgctcatctccatgatcggcatggacccgctttccggcaatctgcgcatgaccttcgactcgcccgacctcatcaacggcgtatccctcgtcccggcgctcgtcggcctgtacgccctgtcgcaagtgctgatcacggttgaagacgtgttcatgggccgcaagctctccactgccgaaatctcgcgcaagcgtatgcccctttccgaaatctggacaaaccgggccgccctgctccgcggctcgatcatcggcacgttcatcggcatcgtcccggccacgggctcgggcacggcctcattcgcggcctacagcgaaaccaagcgccattcaaagcatcccgaacttttcggcaagggctccattgaaggcatcgcggccacggaatccgccaacaacgccgtcaccggcggcgcgctcatccccttgctgaccctcggcgtgcccggcgacgtggtcacggcgatcatgctcggcgcgcttatgatccagggcatgactcccggtccgctgctgttccaggaacagggcacgctcgtctacagcattttcatcgccctgttcgtctccaacgtcttcatgctgcttctgggctactacgcggtgcgcctgttcgccaaggtcgtgctcattcccggcggcatcctcatgcccctcgtcaccaccctgtgcgtggtgggcggctacgccctgaacaactccaacttcgacctcgccgtcatggccggtttcgggttgctcggctacatcatgaccaaggcgcgcttcccgctcgcccccctgctcctcgccatgatcctctccggaatcatcgaaaccaacttccgcagggcgctcagcatctccaatcaggatttctccgtattcttcacccgtcctgtctgcgcggcgttcctcgccatcagcctcttcatcctgttcaacctgctctggaaggaatggaagaagtaccgcgccgccagcgccgcctga

SEQ ID NO: 76 - DNA, 888 bp, Artificial Sequence
  synthetic polynucleotide
atgagtcagacagaatccagttcgctccctaacggcatcggccttgccccctggcttcgcatgaagcaggaaggaatgaccgaaaacgagagccgtatcgtcgaatggttgcttacccccggcaatctcagcgatgcgccggcaatcaaggacgtcgcggaagcgctgtcggtatcagaagccatgatcgttaaggtttctaagctgctggggtttagcggttttcgtaacctgcgcagcgcgctggaggcctacttttcgcagtctgaacaggtgttaccgaccgaactctcctttgatgatgcgccgcaggatgtggtgaataaggtgttcaacatcaccctgcgcaccattatggaaggccagtcgatcgtgaacgttgacgaaattcaccgcgcggcgcgcttttttgcccaagccaaccagcgcgacctgtacggcgcgggcggctccaacgccatctgcgccgacgtgcagcataagtttttacgcatcggcgtacgctgccaggcctacccggacgcgcatatcatgatgatgtccgcctcgctgctgaaggaaggcgatgtcgtgctggtggtctctcactccgggcgcaccagcgatatcaaatcagcggtggagctggcgaaaaagaacggggcgaaaattatctgtatcacccatagctatcattcgccgattgccaagttagctgattttattatttgttcgccagcaccagagacgcctttattagggcgtaacgcctcagcgcgtatattacaactcacattattagatgcgttttttgtttccgttgcacagctcaacattgagcaagcgaatttaaatatgcaaaaaactggcgcgattgttaatttcttttcacccggcgcgcttaaataa

(19) United States
(12) Patent Application Publication
(10) Pub. No.: US 2022/0193151 A1
(43) Pub. Date: Jun. 23, 2022

(54) THERAPEUTIC MICROBIOTA FOR THE TREATMENT AND/OR PREVENTION OF DYSBIOSIS

(71) Applicants: THE CHILDREN'S MEDICAL CENTER CORPORATION, Boston, MA (US); THE BRIGHAM AND WOMEN'S HOSPITAL, INC., Boston, MA (US)

(72) Inventors: Talal A. CHATILA, Belmont, MA (US); Azza ABDEL-GADIR, Cambridge, MA (US); Emmanuel STEPHEN VICTOR, Mysuru (IN); Rima RACHID, Belmont, MA (US); Lynn BRY, Jamaica Plain, MA (US); Georg GERBER, Newton, MA (US)

(73) Assignees: THE CHILDREN'S MEDICAL CENTER CORPORATION, Boston, MA (US); THE BRIGHAM AND WOMEN'S HOSPITAL, INC., Boston, MA (US)

(21) Appl. No.: 17/442,904

(22) PCT Filed: Mar. 26, 2020
(86) PCT No.: PCT/US2020/025008

§ 371 Date: Sep. 24, 2021

Related U.S. Application Data
(60) Provisional application No. 62/823,866, filed on Mar. 26, 2019.

(51) Int. Cl. A61K 35/741; A61P 37/08; A61P 1/14

(52) U.S. Cl. CPC A61K 35/741; A61P 1/14; A61P 37/08

(57) ABSTRACT

Disclosed are methods and compositions for the prevention and treatment of dysbiosis and associated conditions. In particular, described herein are microbial consortia, including monotherapies or minimal microbial consortia, that can prevent and/or cure dysbiosis and associated conditions. In certain embodiments, the therapy comprises Subdoligranulum variabile. In certain embodiments, the microbial consortia comprise certain members of the taxa Clostridiales, Bacteroidetes, Prevotella, and/or Parabacteroides.

### CROSS-REFERENCE TO RELATED APPLICATIONS

This application claims benefit under 35 U.S.C. § 119(e) of U.S. Provisional Application No. 62/823,866 filed Mar. 26, 2019, the contents of which are incorporated herein by reference in their entirety.

### GOVERNMENT SUPPORT

This invention was made with Government Support under Grant Nos. 1R56AI117983 and 1R01AI126915 awarded by the National Institutes of Health. The Government has certain rights in the invention.

### SEQUENCE LISTING

The instant application contains a Sequence Listing which has been submitted in ASCII format via EFS-Web and is hereby incorporated by reference in its entirety. Said ASCII copy, created on Mar. 26, 2020, is named 701039-094520WOPT.txt and is 66,904 bytes in size.

### FIELD OF THE DISCLOSURE

The present disclosure relates to the treatment and/or prevention of dysbiosis and associated conditions.

### BACKGROUND

Growing evidence indicates that the microbial flora is a key environmental influence on a myriad of physiological signaling mechanisms in the body. Furthermore, the microbial flora can influence the development of disease.

As a non-limiting example, food allergies are a growing public health problem in both developed and rapidly developing countries and affects large numbers of children and adults. The incidence of food allergy has increased dramatically in the last few decades. This increase can be associated with sensitization to multiple foods in up to 50% of subjects. Growing evidence indicates that the microbial flora is a key environmental influence in programming oral tolerance.

### SUMMARY

Provided herein are methods and compositions for the treatment and/or prevention of dysbiosis and associated diseases or disorders, including but not limited to inflammatory diseases or disorders, metabolic diseases or disorders, and atopic diseases or disorders. The methods and compositions described herein are based, in part, on the discovery that altered intestinal microbiota (e.g., from antibiotic treatments, C-section births, diet etc.) can promote dysbiosis and inflammatory and atopic diseases while some combinations of microbes can prevent and/or ameliorate or cure dysbiosis, and inflammatory and/or atopic diseases.

In one aspect, described herein is a pharmaceutical composition comprising a species of at least one viable gut bacteria, and a pharmaceutically acceptable carrier, wherein the viable gut bacteria is comprised in a preparation selected from the group consisting of: (i) a preparation of a viable, culturable, anaerobic gut bacterial strain(s) that expresses exopolysaccharide, lipoteichoic acid (LTA), lipopolysaccharide (LPS) or other microbial adjuvant molecules that promote the development of regulatory T cells (Treg); (ii) a preparation of a viable, culturable, anaerobic gut bacterial strain(s) that produces butyrate and/or propionate fermentation products via fermentation of carbohydrates and other carbon sources in the gut lumen; (iii) a preparation of one or more viable, culturable, anaerobic gut bacterial strains that alone or in combination performs the full complement of bile acid transformations; (iv) a preparation of a viable, culturable, anaerobic gut bacterial strain that produces compounds capable of stimulating the aryl hydrocarbon receptor (AhR) receptor pathway in gut epithelial cells, antigen presenting cells and/or T cells to stimulate development of regulatory T cell responses; (v) a preparation of a viable, culturable, anaerobic gut bacterial strain(s) that produces compounds capable of stimulating the pregnane X receptor with beneficial effects upon gut barrier function and/or development of regulatory T cell responses; (vi) a preparation of a viable, culturable, anaerobic gut bacterial strain(s) that produces compounds capable of stimulating the RORgamma (RAR-related orphan receptor gamma) pathways to stimulate development of regulatory T cell responses via direct stimulation or RORgamma-activated pathways in gut antigen presenting cells and/or epithelial cells that then stimulate regulatory T cell responses; (vii) a preparation of viable, culturable, anaerobic gut bacterial strain(s) that stimulates host production of mucins and complex glycoconjugates that improve gut barrier function and colonization by protective commensal species; (viii) a preparation of a viable, culturable, anaerobic gut bacterial strain(s) that alters the gut luminal environment to reduce the deleterious activities of dysbiotic species promoting development of unhealthy allergic T cell responses to food antigens; (ix) a preparation of a viable, culturable, anaerobic gut bacterial strain(s) that alters the gut luminal environment to promote improved colonization by other members of the administered consortium for any of the above stated effects, and/or colonization by existing beneficial species in the patients underlying microbiota; (x) a preparation of a viable, culturable, anaerobic gut bacterial strain(s) that promotes the colonization or growth of a bacterial strain in a preparation of (i)-(ix) above, in vivo.

In one aspect described herein is a pharmaceutical composition comprising: (i) a preparation comprising a species of viable gut bacteria, in an amount sufficient to treat or prevent a dysbiosis when administered to an individual in need thereof, and (ii) a pharmaceutically acceptable carrier.

In some embodiments of any of the aspects, the species of viable gut bacteria is Subdoligranulum variabile.

In some embodiments of any of the aspects, the pharmaceutical composition is formulated to deliver the viable bacteria to the small intestine.

In some embodiments of any of the aspects, the pharmaceutically acceptable carrier comprises an enteric coating composition that encapsulates the species of viable gut bacteria.

In some embodiments of any of the aspects, the enteric-coating composition is in the form of a capsule, gel, pastille, tablet or pill.

In some embodiments of any of the aspects, the composition is formulated to deliver a dose of at least 5×10⁶ colony forming units per mL (CFU/mL)-2×10⁷ CFU/mL.

In some embodiments of any of the aspects, the composition is formulated to deliver at least 5×10⁶ CFU/mL-2×10⁷ CFU/mL in less than 30 capsules per one time dose.

In some embodiments of any of the aspects, the composition is frozen for storage.

In some embodiments of any of the aspects, the species of viable gut bacteria are encapsulated under anaerobic conditions.

In some embodiments of any of the aspects, anaerobic conditions comprise one or more of the following: (i) oxygen impermeable capsules, (ii) addition of a reducing agent including N-acetylcysteine, cysteine, or methylene blue to the composition, or (iii) use of spores for organisms that sporulate.

In some embodiments of any of the aspects, the composition comprises a 16S rDNA sequence at least 97% identical to a 16S rDNA sequence present in a reference strain operational taxonomic unit for Subdoligranulum variabile.

In some embodiments of any of the aspects, the enteric-coating comprises a polymer, nanoparticle, fatty acid, shellac, or a plant fiber.

In some embodiments of any of the aspects, the species of viable gut bacteria is encapsulated, lyophilized, formulated in a food item, or is formulated as a liquid, gel, fluid-gel, or nanoparticles in a liquid.

In some embodiments of any of the aspects, the pharmaceutical composition further comprises a pre-biotic composition.

In some embodiments of any of the aspects, the dysbiosis is associated with an inflammatory disease or a metabolic disorder.

In some embodiments of any of the aspects, the dysbiosis is associated with an atopic disease or disorder.

In some embodiments of any of the aspects, the atopic disease or disorder is selected from the group consisting of: food allergy, eczema, asthma, and rhinoconjunctivitis.

In one aspect described herein is a method for treating or preventing the onset of a dysbiosis in a subject, the method comprising: administering to a subject a pharmaceutical composition as described herein, thereby treating or preventing dysbiosis in the subject.

In one aspect described herein is a method for the treatment, or prevention of gut inflammation or a metabolic disease or disorder, the method comprising: administering to a subject a pharmaceutical composition as described herein, thereby treating, or preventing the gut inflammation or metabolic disease or disorder in the subject.

In one aspect described herein is a method for the treatment, or prevention of an atopic disease or disorder, the method comprising: administering to a subject a pharmaceutical composition as described herein, thereby treating, or preventing the atopic disease or disorder in the subject.

In some embodiments of any of the aspects, the atopic disease or disorder is selected from the group consisting of: food allergy, eczema, asthma, and rhinoconjunctivitis.

In some embodiments of any of the aspects, the pharmaceutical composition is administered by oral administration, enema, suppository, or orogastric tube.

In some embodiments of any of the aspects, the species of viable gut bacteria are isolated and/or purified from a subject known to be tolerant to a selected allergen.

In some embodiments of any of the aspects, the species of viable gut bacteria are prepared by culture under anaerobic conditions.

In some embodiments of any of the aspects, the species of viable gut bacteria are formulated to maintain anaerobic conditions.

In some embodiments of any of the aspects, anaerobic conditions are maintained by one or more of the following: (i) oxygen impermeable capsules, (ii) addition of a reducing agent including N-acetylcysteine, cysteine, or methylene blue to the composition, or (iii) use of spores for organisms that sporulate.

In some embodiments of any of the aspects, the method further comprising administering a pre-biotic composition.

In some embodiments of any of the aspects, the pharmaceutical composition is enteric-coated.

In some embodiments of any of the aspects, the treatment administered prevents and/or reverses T_{H}2 programming.

In some embodiments of any of the aspects, the subject is a human subject.

In some embodiments of any of the aspects, the subject is under the age of 2 years old.

In some embodiments of any of the aspects, the subject is age 2 to under 5 years old.

In some embodiments of any of the aspects, the subject is age 5 to under 12 years old

In some embodiments of any of the aspects, the subject is age 12 to under 18 years old.

In some embodiments of any of the aspects, the subject is age 18 to under 65 years old.

In some embodiments of any of the aspects, the subject is over age 65 years old.

In some embodiments of any of the aspects, the method further comprises a step of diagnosing the subject as having or likely to develop an inflammatory disease or an atopic disease or disorder.

In some embodiments of any of the aspects, the method further comprises a step of testing a fecal sample from the subject for the presence and/or levels of one or more of the bacteria in the pharmaceutical composition.

In some embodiments of any of the aspects, the atopic disease is a food allergy, and wherein the food allergy comprises allergy to soy, wheat, eggs, dairy, peanuts, tree nuts, shellfish, fish, mushrooms, stone fruits and/or other fruits.

In some embodiments of any of the aspects, the pharmaceutical composition is administered before the first exposure to a potential food allergen.

In some embodiments of any of the aspects, the pharmaceutical composition is administered upon clinical signs of atopic symptoms.

In some embodiments of any of the aspects, the pharmaceutical composition is administered to an individual with diagnosed with a food allergy.

In some embodiments of any of the aspects, the subject is pretreated with an antibiotic.

In one aspect described herein is a method for reducing or eliminating a subject's immune reaction to an allergen, the method comprising: administering to a subject a pharmaceutical composition as described herein, thereby reducing or eliminating a subject's immune reaction to the allergen.

In some embodiments of any of the aspects, the pharmaceutical composition is administered by oral administration, enema, suppository, or orogastric tube.

In some embodiments of any of the aspects, the treatment prevents and/or reverses T_{H}2 programming.

In some embodiments of any of the aspects, the subject is a human subject.

In some embodiments of any of the aspects, the subject is under the age of 2 years old.

In some embodiments of any of the aspects, the subject is age 2 to under 5 years old.

In some embodiments of any of the aspects, the subject is age 5 to under 12 years old

In some embodiments of any of the aspects, the subject is age 12 to under 18 years old.

In some embodiments of any of the aspects, the subject is age 18 to under 65 years old.

In some embodiments of any of the aspects, the subject is over age 65 years old.

In some embodiments of any of the aspects, the method further comprises a step of diagnosing the subject as having an IgE-mediated allergy.

In some embodiments of any of the aspects, the method further comprises a step of testing a fecal sample from the subject for the presence and/or levels of one or more of the bacteria in the pharmaceutical composition.

In some embodiments of any of the aspects, the IgE-mediated allergy is a food allergy selected from the group consisting of: allergy to soy, wheat, eggs, dairy, peanuts, tree nuts, shellfish, fish, mushrooms, stone fruits or other fruits.

In some embodiments of any of the aspects, the pharmaceutical composition is administered after an initial exposure and/or reaction to a potential allergen.

In some embodiments of any of the aspects, the biomass of each of the microbes in the administered compositions is greater than the biomass of each of the microbes relative to a reference.

In some embodiments of any of the aspects, the subject is pretreated with an antibiotic.

In some embodiments of any of the aspects, the subject is pretreated with a fasting period not longer than 24 hours.

A method of monitoring a subject's microbiome, the method comprising: determining the presence and/or biomass in a biological sample obtained from a subject, and wherein if at least one or more species selected from the group consisting of Subdoligranulum variabile, Bacteroides fragilis, Bacteroides ovatus, Bacteroides vulgatus, Parabacteroides distasonis, and Prevotella melaninogenica, are absent or low relative to a reference, the subject is treated with a pharmaceutical composition as described herein.

In some embodiments of any of the aspects, the method further comprises predicting that a subject will have an immune response to an allergen when the at least one member is absent, the biomass of the at least one member is low relative to a reference, or at least one member of a dysbiotic species is present or elevated relative to a reference.

In some embodiments of any of the aspects, the method is repeated at least one additional time.

In some embodiments of any of the aspects, the biological sample is a fecal sample.

In one aspect described herein is a method of treating atopic disease or disorder in an individual in need thereof, the method comprising administering a pharmaceutical composition as described herein to the individual.

In some embodiments of any of the aspects, the administration shifts the balance of Tₕ1/Tₕ2 cells towards Tₕ1 T cells.

In some embodiments of any of the aspects, the administration reduces the number or activity of Tₕ2 T cells.

In one aspect described herein is a method of reducing the number or activity of Th2 cells in a tissue of an individual in need thereof, the method comprising administering a pharmaceutical composition as described herein to the individual.

In some embodiments of any of the aspects, the tissue is a gut tissue.

In one aspect described herein is a pharmaceutical composition as described herein, for use in treating or preventing gut inflammation.

In one aspect described herein is a pharmaceutical composition for use in treating or preventing a metabolic disease or disorder.

In one aspect described herein is a pharmaceutical composition for use in treating or preventing an atopic disease or disorder

In one aspect described herein is a pharmaceutical composition for use in treating or preventing a food allergy.

In one aspect described herein is a pharmaceutical composition for use in treating or preventing eczema.

In one aspect described herein is a pharmaceutical composition for use in treating or preventing asthma.

In one aspect described herein is a pharmaceutical composition for use in treating or preventing rhinoconjunctivitis.

In one aspect described herein is use of a pharmaceutical composition for treating or preventing gut inflammation.

In one aspect described herein is use of a pharmaceutical composition for treating or preventing a metabolic disease or disorder.

In one aspect described herein is use of a pharmaceutical composition for treating or preventing an atopic disease or disorder

In one aspect described herein is use of a pharmaceutical composition for treating or preventing a food allergy.

In one aspect described herein is use of a pharmaceutical composition for treating or preventing eczema.

In one aspect described herein is use of a pharmaceutical composition for treating or preventing asthma.

In one aspect described herein is use of a pharmaceutical composition for treating or preventing rhinoconjunctivitis.

### BRIEF DESCRIPTION OF THE FIGURES

FIG. 1 shows a schematic representation of tolerance failure in atopic disease, such as food allergy, which is due to a failure of oral tolerance to food antigens. The pathophysiological mechanism of food allergy is associated with Tₕ2 immunity and allergen-specific IgE responses. T regulatory cells (T_{regs}) generally suppress type-2 innate lymphoid cells (ILC2), Tₕ2 cell activation, mast cell activation, and dendritic cell (DC) activation.

FIG. 2 shows a schematic representation of an atopic disease experimental model: Il4raF709 mutant mice, which are prone to development of food allergy. A mutation in the Type I IL-4 receptor, ITIM, (Y709 to F709) results in a gain of function of the IL-4 receptor.

FIG. 3 shows a schematic representation of an exemplary ovalbumin sensitization protocol. IL4raF709 mutant mice and WT mice were challenged with chicken egg ovalbumin (OVA) along with the mucosal adjuvant staphylococcal entero-toxin B (SEB), followed by a subsequent challenge with OVA. Mutant and WT mice were monitored for changes in core body temperature, total serum IgE, Ova-specific serum IgE, mucosal mast cell protease-1 (MMCP-1) levels, and mast cell counts.

FIGS. 4A-4C show ovalbumin (OVA)-induced food allergic reaction in Il4raF709 mice. FIG. 4A shows the core body temperature change of WT and Il4raF709 mice in response to saline or OVA-SEB over time (minutes). Il4raF709 mice treated with OVA-SEB exhibited a statistically significant drop in core body temperature, indicative of anaphylaxis. WT mice treated with OVA-SEB and WT mice and Il4raF709 mice treated with saline (PBS) did not have significant changes in core body temperature. FIG. 4B shows total serum IgE (i), number of (Nbr) mast cells/LPF levels (ii), OVA-specific IgE levels (iii), and MMCP-1 levels (iv) in WT and Il4raF709 mice treated with saline or OVA-SEB. FIG. 4C shows flow cytometry of immune cells isolated from WT and Il4raF709 mice. Il4raF709 mice treated with OVA-SEB exhibited increases in the percentage (%) of CD4+ IL-4+ T cells and the number (Nbr) of CD4+ IL-4+ T cells compared to WT mice treated with OVA-SEB.

FIGS. 5A-5C show allergen-specific T_{reg} cell deficiency in allergic Il4raF709 mice. FIG. 5A demonstrates representative flow cytometry plots of CD4+Foxp3+ T_{reg} cells. FIG. 5B demonstrates that the number of CD4+Fox3p+ T_{reg} cells in the small intestine (SI), mesenteric lymph nodes (MLN), and spleen are significantly decreased in Il4raF709 mice treated with OVA-SEB compared with WT mice treated with OVA-SEB. In the small intestine, Il4raF709 mice exhibited reductions in the number of CD4+Fox3p+ T_{reg} cells compared to WT mice treated with saline. FIG. 5C shows analysis of CD4+Foxp3+ T_{reg} cell proliferation following OVA-SEB or saline treatment in WT and Il4raF709 mice. CD4+Foxp3+ T_{reg} cell proliferation was reduced in Il4raF709 mice treated with OVA-SEB compared with WT mice treated with OVA-SEB.

FIG. 6 demonstrates that the oral allergic sensitization in F709 mutant mice is associated with dysbiosis. Il4raF709 mice exhibited reductions in several phyla of bacteria in the small intestine.

FIG. 7 provides a schematic representation of an exemplary protocol to test whether the microbiota of sensitized Il4raF709 mice transmit susceptibility to food allergy. Microbiota from WT or IL4raF704 mice are transferred to WT germ free (GF) mice followed by OVA challenge at 8-weeks post-transfer.

FIG. 8 shows that the microbiota of Il4raF709 mice promote allergic sensitization and anaphylaxis in germ free (GF) mice. The left graph shows that the core body temperature of WT germ free mice following OVA challenge and administered Il4raF709 flora exhibited a drop in core body temperature compared with WT mice that receive WT microbial flora. This result is similar to that observed in the Il4raF709 mice challenged with OVA. The right graph demonstrates that GF WT mice that were administered Il4raF709 flora exhibit a significant increase in mMCP-1 levels after re-challenge with OVA compared with GF WT mice that received WT flora.

FIG. 9 shows the microbiota of Il4raF709 mice promote allergic sensitization and anaphylaxis. The left graphs show analysis of CD3+CD4+ T cells in WT GF mice that received WT or Il4raF709 flora. The bar graphs (right) show that GF WT mice that were administered Il4raF709 flora exhibited a significant increase in the percentage of IL4+ cells compared with WT GF mice administered WT microbial flora.

FIG. 10 shows a schematic representation of an exemplary protocol to determine whether microbiota of food tolerant mice transmit protection against food allergy.

FIGS. 11A-11D demonstrate that the microbiota of food tolerant mice protect against allergic sensitization and anaphylaxis in a genetically susceptible host. FIG. 11A shows the change in core body temperature over time following OVA challenge in Il4raF709 mice that were administered WT or Il4raF709 microbial flora. Il4raF709 mice that were administered WT microbial flora exhibited protection from anaphylaxis. FIG. 11B shows total IgE levels (left) and OVA-specific IgE levels (right) for Il4raF709 mice that were administered WT or Il4raF709 microbial flora. FIG. 11C shows flow cytometry analysis for IL-4+ and IFNγ T cells isolated after OVA challenge from Il4raF709 mice that were administered WT or Il4raF709 microbial flora. FIG. 11D shows the percentage of OVA-specific CD4+IL-4+ T cells and CD4+IFNγ+ T cells isolated from Il4raF709 mice that were administered WT or Il4raF709 microbial flora. Il4raF709 mice that were administered WT microbial flora exhibited significant decreases in total IgE, OVA-IgE, and CD4+IL-4+ T cells compared with Il4raF709 mice that were administered Il4raF709 microbial flora.

FIGS. 12A-12D show the microbiota of food tolerant mice promotes the formation of allergen-specific T_{reg} cells. FIG. 12A shows flow cytometry analysis of CD4+Fox3p+ T_{reg} cells isolated from Il4raF709 mice that were administered WT or Il4raF709 microbial flora. FIG. 12B shows that the Il4raF709 mice that were administered WT microbial flora exhibited significant increases in the percentage and number (Nbr) of CD4+Fox3p+ T_{reg} cells when compared with Il4raF709 mice that were administered Il4raF709 microbial flora. FIGS. 12C-12D shows flow cytometry analysis of Fox3p+L_{egs} that were actively proliferating using violet proliferative dye. FIG. 12D confirms that Il4raF709 mice that were administered WT microbial flora exhibited an increase in the percentage of proliferating CD4+Fox3p+ T_{regs} compared with Il4raF709 mice that were administered Il4raF709 microbial flora.

FIG. 13 shows a graphical visualization of relative abundances of phyla following ovalbumin (OVA) treatment at 8 weeks for WT (top) and Il4raF709 mice (bottom).

FIG. 14 shows a table of selected OTUs demonstrating differences between WT and F709 mice challenged with OVA. The table shows OTUs in the duodenum, jejunum, and ileum.

FIG. 15 shows an exemplary protocol for determining whether treatment with defined bacterial mixes will protect against food allergy. Il4raF709 mutant mice were given antibiotics for 7 days prior to the start of the protocol.

FIGS. 16A-16D show that Clostridia and Bacteroidetes protect against development of allergen-specific responses and anaphylaxis. FIG. 16A shows change in core body temperature following OVA challenge of Il4raF709 mice treated with Clostridia, Proteobacteria, or Bacteroidetes compared with Il4raF709 mice that did not receive bacterial treatment. Mutant mice treated with Bacteroidetes and Clostridia were protected from a drop in core body temperature following OVA challenge. FIG. 16B shows the number of mast cells, and MMCP-1 levels in Il4raF709 mice administered Clostridia, Proteobacteria, or Bacteroidetes compared with no bacterial treatment. Mutant mice treated with Bacteroidetes and Clostridia exhibited reductions in the number of mast cells and MMCP-1 levels after Ova challenge compared with mutant mice that were not treated with bacteria or those administered Proteobacteria. FIG. 16C shows jejunal mast cells in OVA-challenged Il4raF709 mice treated with Clostridia, Proteobacteria, or Bacteroidetes compared with no bacterial treatment. FIG. 16D shows total IgE and OVA-specific IgE levels in OVA-challenged Il4raF709 mice treated with Clostridia, Proteobacteria, or Bacteroidetes compared with no bacterial treatment.

FIGS. 17A-17D show oral allergic sensitization is associated with T_{reg} cell Tₕ2 reprogramming. FIG. 17A shows flow cytometry analysis of MLN CD3+CD4+ T cells from OVA sensitized WT and Il4raF709 mice for IL-4 and Foxp3 markers. FIG. 17B shows the percentage (top) of IL-4+CD4+Foxp3+ T cells in WT and Il4raF709 mice sensitized with OVA-SEB or treated with PBS. FIG. 17C shows flow cytometry analysis of GATA3+ T cells in WT and Il4raF709 mice sensitized with OVA-SEB or controls treated with PBS. Number and percentage of GATA3+CD4+Foxp3+ cells are shown at right. FIG. 17D shows flow cytometry analysis of IRF-4+ T cells in WT and Il4raF709 mice sensitized with OVA-SEB or controls treated with PBS. Number and percentage of IRF-4+CD4+Foxp3+ cells are shown at right.

FIGS. 18A-18F shows deletion of Il4/Il13 in T_{reg} cells protects against food allergy. FIG. 18A shows fold change in IL-4 in CD4+FoxP3− versus CD4+FoxP3+ cells in Il4raF709 mice versus Il4raF709 IL4, IL-13^{-/-} mice. FIG. 18B change in body core temperature after OVA challenge over time for Il4raF709 mice Il4raF709 IL4, IL-13^{-/-} mice. FIG. 18C shows total IgE, OVA-specific IgE, MMCP-1 level, and mast cell number in Il4raF709 mice and Il4raF709 IL4, IL-13^{-/-} mice. FIG. 18D-FIG. 18E show number (bottom), and percentage (top) of CD4+Fox3pd+(18D), percentage of IRF-4 (18E, bottom) and GATA3+(18E, top) CD4+Foxp3+ cells in Il4raF709 versus Il4raF709 IL-13^{-/-} mice. FIG. 18F shows number (bottom) and percentage (top) of IL-4+CD4+Foxp3-cells in the Il4raF709 mice versus Il4raF709 IL4, IL-13^{-/-} mice.

FIG. 19 shows reduced Tₕ2-skewed T_{reg} phenotype indicates that Clostridia and Bacteroidetes have different molecular mechanisms of action. Left: number and percentage of CD3+CD4+Foxp3+T_{reg} cells in OVA-challenged Il4raF709 mice treated with no bacteria and mice treated with Clostridia, Proteobacteria, or Bacteroidetes. Center: FACs plots and graphical representation of CD3+CD4+Foxp3+GATA3+ cells in Il4raF709 mice treated with no bacteria and mice treated with Clostridia, Proteobacteria, or Bacteroidetes. Clostridia and Bacteroidetes each protect, but show significant differences in relative IL-4 and GATA3 expression in T_{regs}.

FIGS. 20A-20D demonstrate that short chain fatty acid (SCFA) therapy does not rescue food allergy in Il4raF709 mice. FIG. 20A shows SCFAs, isovalerate, valerate, acetate, propionate, and butyrate (mM) concentrations measured in WT and Il4raF709 mice administered saline or OVA-SEB. FIG. 20B shows change in core body temperature in WT and Il4raF709 mice treated with PBS or OVA-SEB with and without SCFA treatment. FIG. 20C shows total IgE and OVA-specific IgE in WT and Il4raF709 mice treated with OVA-SEB, PBS-SCFA, and OVA-SEB+SCFAs. FIG. 20D shows flow cytometry analysis for CD4+Foxp3+ cells isolated from the small intestine of WT and Il4raF709 mice treated with OVA—EB, PBS-SCFAs, and OVA-SEB-SCFAs.

FIG. 21A-21I demonstrate that a defined consortium of human Bacteroidales species prevents food allergy in specifically-associated germfree mice and in therapeutically treated conventional Il4raF709 mice. FIG. 21A shows a schematic representation of specific-association studies in germfree mice with the Bacteroidales microbial consortium versus gnotobiotic controls. FIG. 21A also shows core body temperature changes in GF Il4raF709 mice that were uncolonized or reconstituted with the Bacteroidales consortium, then either sham-(PBS) or OVA/SEB-sensitized and challenged with OVA. FIG. 21B shows total and OVA-specific serum IgE concentrations post-OVA challenge. FIG. 21C shows serum MMCP-1 concentrations post OVA challenge. FIG. 21D shows frequencies of MLN CD4+Foxp3+, IL-4+Foxp3+ and GATA3+Foxp3+ T cells. FIG. 21E shows frequencies of Helios-NRP1−Foxp3+ T cells. For FIGS. 21A-21E: N=5 to 10 mice/group. FIG. 21F shows a schematic representation of SPF mouse studies; Abx: antibiotics. FIG. 21F also shows core body temperature changes in OVA/SEB-sensitized and OVA-challenged Il4raF709 mice that were either untreated or treated with the Bacteroidales consortium. FIG. 21G shows total and OVA-specific IgE responses and serum MMCP-1 concentrations post OVA challenge. FIG. 21H shows frequencies of CD4+Foxp3+, IL-4+Foxp3+GATA3+Foxp3+, and FIG. 21I shows Helios—NRP1-Foxp3+ T cells in the MLN. For f-i: N=5-8 mice/group. *P<0.05, **P<0.01,***P<0.001, ****P<0.0001 by one-way ANOVA with Dunnett post hoc analysis. For core body temperature measurements ****P<0.0001 by repeat measures two-way ANOVA.

FIGS. 22A-22D demonstrates that treatment with the Clostridiales or Bacteroidales therapeutic consortia suppress established food allergy in conventional IL4raF709 mice. FIG. 22A shows an experimental scheme (left) and core body temperature changes in OVA/SEB sensitized and OVA-challenged Il4raF709 mice treated with the respective bacterial mixes (right). FIG. 22B shows total and OVA-specific IgE. FIG. 22C shows Jejunal mast cells (arrows), mast cell counts per low powered field and serum MMCP-1 concentrations post OVA challenge. FIG. 22D shows frequencies of CD4+Foxp3+, IL-4+CD4+Foxp3+, IL-4+CD4+Foxp3−, and GATA3+Foxp3+ T cells in the MLN. N=5-15 mice/group. **p<0.01, ***p<0.001, ****p<0.0001 by one-way ANOVA with Dunnett post hoc analysis. For core body temperature measurements ***P<0.001 by repeat measures two-way ANOVA.

FIGS. 23A-23F demonstrate Bacteroidales consortium persistence in vivo. FIG. 23A demonstrates the results of extracted stool DNA that was subject to qPCR with probes specific for each organism. No cross-reactivity was found in baseline stool from mice prior to administering the consortium (Panel A, right column). Ct values were compared against a standard curve of defined biomass of each organism spiked into conventional stool to obtain a normalized Log₁₀ CFU/g. FIGS. 23B-23F show normalized Log₁₀ CFU/g values of stool samples for each of the organisms administered in the dose. N=6 mice group; grey dotted line indicates the sensitivity of detection or each qPCR probe. For mice falling below the sensitivity of detection, data points are placed at log₁₀=1.

FIG. 24 shows a table of bacterial species and strain designations; growth conditions for the microbial consortium; and the respective 16S rRNA sequences (SEQ ID NOs: 1-15).

FIGS. 25A-25D demonstrate that FA infants exhibit an evolving dysbiosis of their gut microbiota. FIG. 25A-D show heat map representations of log 2 fold relative abundances of fecal bacterial taxa between FA and health control (HC) infants displayed across the different age groups: 1-6, 7-12, 3-18, 19-24, and 25-30 months. For detailed group description and subject characteristics, see e.g., FIG. 31 and TABLE 6. Taxa represented included those from the order Clostridiales, family Lachnospiraceae (FIG. 25A), order Clostridiales, other Families (FIG. 25B), order Bacteroidales (FIG. 25C) and other miscellaneous taxa (FIG. 25D). Taxonomic information is on the right side of the respective panel. Analysis was carried out using the DESeq2 software package as described in the Methods section. Data from FIG. 25A-25D are also shown in Table 7, wherein negative log 2 fold change values represent higher abundance in control subjects, and positive log 2 fold change values represent higher abundance in food allergic subjects.

FIGS. 26A-26I show that FA is associated with altered mucosal antibody responses to the gut commensal flora. FIGS. 26A-D show flow cytometric analysis and frequencies of human fecal bacteria of FA subjects and healthy control (HC) subjects stained with a PE-conjugated isotype control mAb or phycoerythrin (PE)-conjugated mouse anti-human IgA (FIG. 26A and FIG. 26B) or IgE mAb (FIG. 26C and FIG. 26D). Each symbol represents a result from one subject. N=15 HC subjects and N=13 FA subjects for FIG. 26B, and n=14 HC subjects and n=13 FA subjects for FIG. 26D. FIG. 26E shows core body temperature changes in WT (n=10 mice per group) and Il4ra^{F709} mice (n=7 mice per group) that have been either sham sensitized (PBS) or sensitized with OVA/SEB, as indicated, and challenged with OVA. FIGS. 26F-I show flow cytometric analysis and frequencies of IgA (FIG. 26F and FIG. 26G) and IgE (FIG. 26H and FIG. 26I) staining of fecal bacteria of WT and Il4ra^{F709} mice that were either sham sensitized (PBS) or sensitized with OVA/SEB. Fecal pellets of Rag2-deficient (Rag2^{-/-}) mice and IgE-deficient Il4ra^{F709} (Il4ra^{F709}Igh7^{-/-}) mice were used as negative controls for sIgA and IgE staining, respectively. Each symbol represents one mouse (n=7-14 mice per group). FIG. 26B, FIG. 26D: **P<0.01, ***P<0.001 by Student's unpaired two tailed t test. FIG. 26G, FIG. 26I: *P<0.05, **P<0.01, ***P<0.001 by one-way analysis of variance (ANOVA) with Dunnett post hoc analysis. For core body temperature measurements ***P<0.001 by repeat measures two-way ANOVA.

FIGS. 27A-27H show that a defined consortium of human Clostridiales species prevents FA in Il4ra^{F709} mice. FIG. 27A: left panel shows a Schema of GF Il4ra^{F709} mouse studies, and right panel shows core body temperature changes in GF Il4ra^{F709} mice either GF or colonized with the Clostridiales or Proteobacteria consortium, then sham-(PBS) or OVA/SEB-sensitized and challenged with OVA (n=5-10 mice per group). FIG. 27B shows total and OVA-specific serum IgE concentrations (n=5-13 mice per group). FIG. 27C shows jejunal mast cells histology (arrows) and counts per low powered field (LPF) and serum MMCP-1 concentrations post OVA challenge. (n=4-7 mice per group). FIG. 27D shows frequencies of the indicated MLN Treg cell populations (n5-6 mice per group). FIG. 27E: left panel shows a schema of SPF Il4ra^{F709} mouse studies; Abx: antibiotics. Right panel shows Core body temperature changes in OVA/SEB-sensitized and OVA-challenged Il4ra^{F709} mice treated with the respective consortia (n=6-7 mice per group). FIG. 27F shows total and OVA-specific IgE responses (n=6-17 mice per group), jejunal mast cell counts and serum MMCP-1 concentrations post OVA challenge (n=5 mice per group). FIG. 27G shows frequencies of the indicated MLN Treg cells population (n=4-7 mice per group). FIG. 27H shows flow cytometric analysis and frequencies of ROR-γt⁺Foxp3⁺ in the MLN (n=4-5 mice/group). ***P<0.001, ****P<0.0001 by one-way ANOVA with Dunnett post hoc analysis. For core body temperature measurements ****P<0.0001 by repeat measures two-way ANOVA. n.s.: non-significant.

FIGS. 28A-28D show that treatment with the Clostridiales and Bacteroidales consortia suppresses established FA in Il4ra^{F709} mice. FIG. 28A: left panel shows experimental scheme. Right panel shows core body temperature changes in OVA/SEB sensitized Il4ra^{F709} mice, sham sensitized Il4ra^{F709} mice treated with Clostridiales consortium, and OVA/SEB sensitized Il4ra^{F709} mice that were subsequently treated with the Clostridiales, Bacteroidales or Proteobacteria consortia, as indicated, then challenged with OVA (n=5-10 mice per group). FIG. 28B shows total and OVA-specific serum IgE concentrations for the groups listed in (n=4-10 mice per group for total IgE and n=5-11 mice per group for OVA-specific IgE). FIG. 28C shows jejunal mast cells (arrows), mast cell counts per LPF and serum MMCP-1 concentrations post OVA challenge (n=4-5 mice per group for mast cell counts and n=5-11 mice per group for MMCP1 measurements). FIG. 28D shows frequencies of MLN CD4⁺Foxp3⁺ T cells (n=5-14 mice per group), IL-4⁺CD4⁺Foxp3⁺ T cells (n=5-14 mice per group), IL-4⁺CD4⁺Foxp3⁻ T cells (n=5-10 mice per group), GATA3⁺Foxp3⁺ T cells (n=4-10 mice per group), and ROR-γt⁺Foxp3⁺ T cells (n=4-9 mice per group) in the respective treatment groups. *p<0.05, **p<0.01, ***p<0.001, ****p<0.0001 by one-way ANOVA with Dunnett post hoc analysis. For core body temperature measurements ***P<0.001 by repeat measures two-way ANOVA.

FIGS. 29A-29K show that ROR-γt⁺ Treg cell deficiency promotes FA. FIGS. 29A-C show flow cytometric analysis and frequencies of circulating ROR-γt⁺Foxp3⁺ Treg cells and ROR-γt⁺Foxp3⁻ T cells from FA, atopic (atopy) and healthy controls (HC) subjects (n=10 HC, n=11 Atopic and n=22 FA subjects respectively). For subject characteristics, see e.g., TABLE 10. FIG. 29D shows frequencies of MLN ROR-γt⁺Foxp3⁺ and ROR-γt⁺Foxp3⁻ T cells in Foxp3^{YFPCre} and Il4ra^{F709}Foxp3^{YFPCre} mice that were sham (PBS) or OVA/SEB-sensitized, as indicated, then challenged with OVA (n=5-9 mice per group). FIG. 29E shows core body temperature changes in Foxp3^{YFPCre}, Foxp3^{YFPCre}Rorc^{Δ/Δ} and Il4ra^{F709}Foxp3^{YFPCre} mice that were sensitized as indicated and challenged with OVA (n=5 mice per group). FIG. 29F shows total and OVA-specific IgE responses (n=5-7 mice per group). FIG. 29G shows jejunal mast cells (arrows). FIG. 29H shows mast cell numbers/LPF (n=5 mice per group) and serum MMCP1 concentrations post OVA challenge in the indicated mouse groups (n=4-5 mice per group). FIG. 29I shows frequencies of MLN CD4⁺Foxp3⁺ T cells in the respective mouse groups (n=5-6 mice per group). FIG. 29J shows frequencies of MLN IL-4⁺Foxp3⁺ and IL-4⁺Foxp3⁻ T cells, GATA3⁺Foxp3⁺ and GATA3⁺Foxp3⁻ T cells in the respective mouse groups (n5-6 mice per group). FIG. 29K shows frequencies of ROR-γt⁺Foxp3⁺ T cells in the MLN and small intestinal LPL of GF Il4ra^{F709} mice that underwent FMT with fecal microbiota from HC or FA subjects then subjected to OVA/SEB-sensitization and challenged with OVA (see e.g., FIGS. 31E-G) (n=7 mice/group, each mouse receiving FMT from one donor). *p<0.05, **p<0.01, ***p<0.001, ****p<0.0001 by one-way ANOVA with Dunnett post hoc analysis. For core body temperature measurements ***P<0.0001 by repeat measures two-way ANOVA.

FIGS. 30A-30J show that protection against FA by the bacterial consortia is dependent on ROR-γt⁺ Treg cells. FIGS. 30A-30B shows core body temperature changes (FIG. 30A) and total and OVA-specific serum IgE responses (FIG. 30B) in Il4ra^{F709} and Il4ra^{F709} Foxp3^{YFPCre}Rorc^{Δ/Δ} conventional SPF mice that were sensitized with OVA/SEB without or with additional treatment with the Clostridiales or Bacteroidales consortia (following the schema in FIG. 3E), then challenged with OVA. (n=5-13 mice per group for FIG. 30A and 5-9 mice per group for FIG. 30B). FIGS. 30C-30D show jejunal mast cells (arrows) (FIG. 30C) and serum MMCP-1 concentrations (FIG. 30D) post OVA challenge (n=5-10 mice per group). FIGS. 30E-30F show flow cytometric analysis and frequencies of MLN ROR-γt⁺Foxp3⁺ Treg cells in the indicated mouse groups (n=5-9 mice per group). FIG. 30G shows core body temperature changes in OVA/SEB-sensitized and OVA-challenged Il4ra^{F709}Foxp3^{YFPCre} mice treated with the Bacteroidales consortium, and in OVA/SEB-sensitized and OVA-challenged Il4ra^{F709}Foxp3^{YFPCre}Myd88^{Δ/Δ} mice otherwise untreated or treated with the Clostridiales or Bacteroidales consortia, as indicated (n=7-14 mice per group). FIG. 30H shows total and OVA-specific serum IgE responses and serum MMCP1 concentrations post OVA challenge (n=7-9 mice per group). FIGS. 301-30J show flow cytometric analysis and frequencies of MLN ROR-γt⁺Foxp3⁺ Treg cells in the groups detailed in FIG. 30G (n=8-9 mice per group). (*p<0.05, **p<0.01, ***p<0.001, ****p<0.0001 by one-way ANOVA with Dunnett post hoc analysis. For core body temperature measurements ****P<0.0001 by repeat measures two-way ANOVA.

FIGS. 31A-31C show a microbiota analysis in FA and healthy control infants. FIG. 31A shows the design of analyses of demographic variables of human FA and control subject groups. FA and control subjects were stratified into 5 age groups spaced at 6 month age intervals starting at age 1-6 months. Differential abundance analyses were carried out using DESeq2, and were controlled for variables including gender (G), mode of delivery (MD) and breast feeding (BF). A subgroup analysis was also performed on subjects consuming cow's milk proteins. FIG. 31B shows alpha diversity of gut microbiome of all age groups. Alpha diversity values were calculated using Shannon entropy to measure diversity in each sample. Bars represents average Shannon entropy values, and error bars represents S.E.M. Open bars represents control subjects, and gray represents FA subjects. FIG. 31C shows beta diversity of gut microbiome in all subjects. Beta-diversity values were calculated using the unweighted/weighted Unifrac dissimilarity measures, to assess differences in overall microbial community structure. Overall community structure differed significantly among age groups (adjusted P<0.001), but not between control and FA subjects, using Analysis of Molecular Variance for statistical hypothesis testing.

FIGS. 32A-32H show that FA infants tolerant to cow milk protein (CMP) exhibit dysbiosis. FIGS. 32A-32D show heat map representations of log 2 fold relative abundances of fecal bacterial taxa between FA (milk tolerant) and control (C) infants displayed across the different age groups: 7-12, 3-18, 19-24, and 25-30 months. All the infants in the 1-6 months group were milk-allergic and were accordingly excluded from the analysis. Taxa represented included those from the order Clostridiales, family Lachnospiraceae (FIG. 32A), order Clostridiales, other Families (FIG. 32B), order Bacteroidales (FIG. 32C) and other miscellaneous taxa (FIG. 32D). Taxonomic information is on the right side of the respective panel. Differential abundance analyses were carried out using the DESeq2 software package as described in the Methods section. Data from FIG. 32A-32D are also shown in Table 8, wherein negative log 2 fold change values represent higher abundance in control subjects, and positive log 2 fold change values represent higher abundance in food allergic subjects. FIG. 32E shows core body temperature changes in GF Il4ra^{F709} mice that were left uncolonized or reconstituted with FMT from HC or FA subjects, then sensitized with OVA/SEB and challenged with OVA (n=7 mice per group; each recipient mouse received FMT from one HC or FA subject). ****P<0.0001 by two-way ANOVA, *P<0.05, **P<0.01 with Sidak post hoc analysis.

FIGS. 32F-32G show total and OVA-specific serum IgE concentrations (n=7 mice per group, as in FIG. 32E). FIG. 32H shows serum MMCP-1 concentrations post OVA challenge (n=7 mice per group, as in FIG. 32E). For FIGS. 32F-32H, *P<0.05 by One-way ANOVA with Dunnett's post hoc analysis. Throughout, data represent mean±s.e.m. from two or three independent experiments.

FIGS. 33A-33F show that fecal matter transplant (FMT) from WT but not Il4ra^{F709} mice protects against FA in GF Il4ra^{F709} FIG. 33A shows core body temperature changes in GF Il4ra^{F709} mice that were left uncolonized or reconstituted with FMT from WT or Il4ra^{F709} mice, then sensitized with OVA/SEB and challenged with OVA (n=15 WT and 14 Il4ra^{F709} mice). ****P<0.0001 by two-way ANOVA. FIGS. 33B-33C show total and OVA-specific serum IgE concentrations. N=15 WT and 14 Il4ra^{F709} mice. FIG. 33D shows serum MMCP-1 concentrations post OVA challenge; n=6 mice per group. FIGS. 33E-F show flow cytometric analysis and cell frequencies of ROR-γt and GATA3 expression in MLN Helios⁻NRP1⁻ and Helios⁺NRP1⁺ Treg cells. N=6 mice per group. Each dot represents one mouse. Throughout, data represent mean±s.e.m. from two or three independent experiments. **P<0.01, ***p<0.001, ****p<0.0001 by student's unpaired two tailed t test with Welch correction.

FIGS. 34A-34F show gating strategy for analyzing IgA and IgE bound bacteria in human and mice fecal samples. FIG. 34A and FIG. 34C show representative FACS plots showing the gating strategy for human (FIG. 34A) and mouse (FIG. 34C) fecal bacteria. IgA- and IgE-bound fecal bacteria were analyzed by first gating on forward versus side scatter area (FSC-A versus SSC-A) on a log-log scale (Left most panels). Doublets were discriminated by gating on the forward scatter height (FSC-H) versus FSC-A and subsequently gating on SSC-H versus SSC-A (Second and third panels from the left, respectively). Bacteria present in the feces was further identified by gating on SYTO-BC⁺ events (right side panels). FIG. 34B and FIG. 34D show frequencies of IgA- and IgE-bound bacteria as assessed by gating on bacteria-bound with the respective PE-labelled anti-IgA and anti-IgE antibodies, as shown in FIG. 34A and FIG. 34C. FIGS. 34E-34F show flow cytometric analysis and frequencies of sIgA⁺ (FIG. 34E) and IgE⁺ (FIG. 34F) fecal bacteria of Il4ra^{F709} mice sensitized with OVA/SEB without or with additional bacterial therapy. Fecal pellets of Rag2^{-/-} and Igh7^{-/-}Il4ra^{F709} mice were used as negative controls for the respective antibody staining. Each symbol in the scatter plots represents one mouse (n=11 mice for the no treatment group, n=8 mice for Clostridiales consortium-treated group and n=7 (FIG. 34E) and n=9 (FIG. 34F) for Proteobacteria consortium-treated group). Throughout, data represent mean±s.e.m. from two independent experiments. ***P<0.001, ****P<0.0001 by one-way ANOVA with Dunnett post hoc analysis.

FIGS. 35A-35D show the impact of bacterial therapy on MLN Treg cell markers in FA Il4ra^{F709} mice. FIGS. 35A-35D show representative FACS plots showing the expression of Helios/GATA3 and Helios/ROR-γt in CD4⁺Foxp3⁺ Treg cells in SPF Il4ra^{F709} mice that were treated with antibiotics first then sensitized with OVA/SEB either without (FIG. 35A) or with additional treatment with the Clostridiales (FIG. 35B), Proteobacteria (FIG. 35C) or Bacteroidales (FIG. 35D) consortium, as indicated.

FIGS. 36A-36F show an analysis of small intestinal LPL in OVA/SEB-sensitized and Clostridiales consortium-treated SPF Il4ra^{F709} mice. FIGS. 36A-36B show cell frequencies of total CD4⁺Foxp3⁺, CD4⁺Foxp3⁻ (FIG. 36A) and IL-4⁺CD4⁺Foxp3⁺ (FIG. 36B) Treg cells in the LPL of OVA/SEB sensitized that were either untreated or treated with the Clostridiales consortium, as detailed in FIG. 27E (n=4-5 mice in the OVA/SEB-treated Il4ra^{F709} group and n=7 mice in the OVA/SEB-+Clostridiales-treated Il4ra^{F709} group). FIGS. 36C-36F show flow cytometric analysis and cell frequencies of GATA3⁺CD4⁺Foxp3⁺ and ROR-γt⁺CD4⁺Foxp3⁺ Treg cells in the respective mouse groups (n=4-5 mice in the OVA/SEB-treated Il4ra^{F709} groups and n=7 mice in the OVA/SEB-+Clostridiales-treated Il4ra^{F709} group). Each dot represents one mouse. Data represent mean±s.e.m. from two independent experiments. Unless otherwise indicated, *P<0.05, ***p<0.001 by Student's unpaired two tailed t test.

FIGS. 37A-37E show that antibiotic therapy potentiates the therapeutic efficacy of the Clostridiales consortium in Il4ra^{F709} mice. FIG. 37A shows core body temperature changes in SPF Il4ra^{F709} mice that were either sham or antibiotic-treated then sensitized with OVA/SEB while receiving either sham treatment or treatment with the Clostridales consortium, and thereafter challenged with OVA (n=5-6 mice per group). ****P<0.0001 by two-way ANOVA. FIGS. 37B-37C show total and OVA-specific serum IgE concentrations (n=4-5 mice per group). FIG. 37D shows serum MMCP-1 concentrations post OVA challenge (n=4-5 mice per group. FIG. 37E shows cell frequencies of total CD4⁺Foxp3⁺, Helios⁻NRP1⁻Foxp3⁺, ROR-γt⁺CD4⁺Foxp3⁺ and IL-4⁺CD4⁺Foxp3⁺Treg cells in the MLN of the respective mouse group (n=4-6 mice per group). Each dot represents one mouse. Throughout, data represent mean±s.e.m. from two or three independent experiments. Unless otherwise indicated, *P<0.05, **P<0.01, ***p<0.001, ****p<0.0001 by one-way ANOVA with Dunnett post hoc analysis.

FIGS. 38A-38G show that mono-bacteriotherapy therapy with Subdoligranulum variabile protects against FA in Il4ra^{F709} mice. FIG. 38A shows core body temperature changes in SPF Il4ra^{F709} mice that were antibiotic-treated then sensitized with OVA/SEB while receiving by gavage either sham treatment or treatment with the Subdoligranulum variabile, and thereafter challenged with OVA (n=8-11 mice per group). ****P<0.0001 by two-way ANOVA. FIGS. 38B-38C show total and OVA-specific serum IgE concentrations (n=8-11 mice per group). FIG. 38D shows serum MMCP-1 concentrations post OVA challenge (n=8-11 mice per group). FIGS. 38E-38F shows flow cytometric analysis and cell frequencies of ROR-γt⁺ and GATA3⁺cells among MLN Helios⁻NRP1⁻Foxp3⁺ Treg cells (n=5-8 mice per group). FIG. 38G shows flow cytometric analysis and cell frequencies of MLN IL-4⁺CD4⁺Foxp3⁺ Treg cells and IL-4⁺CD4⁺Foxp3− Teff cells (n=5-8 mice per group). Each dot represents one mouse. Throughout, data represent mean±s.e.m. from two or three independent experiments. For FIGS. 38B-38G, **P<0.01, by Student's unpaired two tailed t test.

FIGS. 39A-39E show that the Clostridiales consortium protects against percutaneous sensitization-mediated FA in WT BALB/c mice. FIG. 39A shows core body temperature changes in SPF WT BALB/c mice that were antibiotic-treated then percutaneously sensitized with OVA/SEB while receiving by gavage either sham treatment or treatment with the Clostridales consortium, and thereafter challenged with OVA (n=11-14 mice per group). ****P<0.0001 by two-way ANOVA. FIG. 39B-39C show total and OVA-specific serum IgE concentrations (n=7 mice per group). FIG. 39D show serum MMCP-1 concentrations post OVA challenge (n=7 mice per group). FIG. 39E show flow cytometric analysis and cell frequencies of MLN ROR-γt⁺CD4⁺Foxp3⁺ Treg cells (n=7 mice per group). Each dot represents one mouse. Data represent mean±s.e.m. from two independent experiments. For FIGS. 39B-39E, **P<0.01, ****P<0.0001 by Student's unpaired two tailed t test.

FIGS. 40A-40J show that a defined consortium of human Bacteroidales species prevents FA in Il4ra^{F709} mice. FIG. 40A: left panel shows schema of GF mouse studies. Right panel shows core body temperature changes in GF Il4ra^{F709} mice that were uncolonized or reconstituted with the Bacteroidales consortium, then either sham-(PBS) or OVA/SEB-sensitized and challenged with OVA (n=5 mice per group). FIG. 40B shows total and OVA-specific serum IgE concentrations (n=4-7 mice per group). FIG. 40C shows serum MMCP-1 concentrations post OVA challenge ((n=5-7 mice per group). FIG. 40D shows frequencies of MLN CD4⁺Foxp3⁺, IL-4⁺Foxp3⁺ and GATA3⁺Foxp3⁺ T cells (n=4-8 mice per group). FIG. 40E shows frequencies of Helios⁻Nrp1⁻Foxp3⁺ and ROR-γt⁺Foxp3⁺ T cells, respectively (n=5-7 mice per group). FIG. 40F: left panel shows a schema of SPF mouse studies; Abx: antibiotics. Right panel shows core body temperature changes in OVA/SEB-sensitized and OVA-challenged Il4ra^{F709} mice that were either untreated or treated with the Bacteroidales consortium (n=5-6 mice per group). FIG. 40G shows total and OVA-specific IgE responses and serum MMCP-1 concentrations post OVA challenge (n=5-10 mice per group). FIG. 40H shows frequencies of CD4⁺Foxp3⁺, IL-4⁺Foxp3⁺GATA3⁺Foxp3⁺, and FIG. 40I shows Helios⁻ Nrp1⁻Foxp3⁺ and ROR-γt⁺Foxp3⁺ T cells in the MLN (n=5-10 mice per group). FIG. 40J shows flow cytometric analysis and frequencies of IgE and IgA staining of fecal bacteria of Il4ra^{F709} mice that were sensitized with OVA/SEB and left uncolonized or reconstituted with the Bacteroidales consortium. Staining was carried out with PE-conjugated rat isotype control mAbs or rat anti-mouse IgA or IgE mAb, as indicated. Fecal pellets of Rag2-deficient (Rag2^{-/-}) mice and IgE-deficient Il4ra^{F709} (Il4ra^{F709}Igh7^{-/-}) mice were used as negative staining controls (n=8-11 mice per group). Each dot represents one mouse. Data represent mean±s.e.m. from two independent experiments. For core body temperature measurements (FIG. 40A, FIG. 40F), ****P<0.0001 by repeat measures two-way ANOVA. For FIGS. 40B-40E, *P<0.05, **P<0.01, ***P<0.001, ****P<0.0001 by one-way ANOVA with Dunnett post hoc analysis. For FIG. 40F-40J: ****P<0.01, ***P<0.001, ****P<0.0001 by Student's unpaired two tailed t test.

FIGS. 41A-41N shows that depletion of Treg cells ablates the protective effects of the microbiota mixes. FIG. 41A shows a schema of OVA sensitization, bacterial preventive therapy and Diphtheria Toxin (DT) treatment. FIG. 41B shows core body temperature changes in Il4ra^{F709}Foxp3^{EGFP} and Il4ra^{F709}Foxp3^{EGFP/DTR+} mice that have been sensitized with OVA/SEB without or with further treatment with the Clostridiales or Bacteroidales consortium and DT, as shown in FIG. 41A, then challenged with OVA (n=7-9 mice per group). FIG. 41C shows total and OVA-specific serum IgE post-sensitization (n=5-6 mice per group). FIG. 41D shows serum MMCP-1 concentrations post OVA challenge (n=8-12 mice per group). FIGS. 41E-41G show frequencies of MLN CD4⁺Foxp3⁺ and IL-4⁺Foxp3⁺ T cells (FIGS. 41E-41F) and of ROR-γt⁺Foxp3⁺ and GATA3⁺Foxp3⁺subpopulations among Foxp3⁺ T cells (FIG. 41G) in the respective mouse groups (n=6-10 mice per group). FIG. 41H shows a schema of OVA sensitization, curative therapy with the Clostridiales consortium and anti-CD25 (αCD25) or isotype Control (IC) mAb treatment. FIG. 41I shows core body temperature changes in Il4ra^{F709} mice that were either sham sensitized (PBS), or sensitized with OVA/SEB either without or with further treatment with the Clostridiales consortium and the indicated mAb, then challenged with OVA (n=5-9 mice per group). FIG. 41J shows total and OVA-specific serum IgE antibody concentrations (n=5-8 mice per group). FIG. 41K shows serum MMCP-1 concentrations post OVA challenge (n=5-7 mice per group). FIG. 41L-41N shows Frequencies of MLN CD4⁺Foxp3⁺ and IL-4⁺Foxp3⁺ T cells (FIG. 41L-41M) and of ROR-γt⁺Foxp3⁺ and GATA3⁺Foxp3⁺ subpopulations among Foxp3⁺ T cells (FIG. 41N) (n=5-8 mice per group). Each dot represents one mouse. Data represent mean±s.e.m. from two independent experiments. FIGS. 41C-F; For FIGS. 41J-M: ***P<0.001, ****P<0.0001 by one-way ANOVA with Dunnett post hoc analysis. For FIG. 41G and FIG. 41N: ****P<0.0001 by repeat measures two-way ANOVA.

FIGS. 42A-42H show that oral SCFA supplementation does not protect against FA. FIG. 42A shows concentrations of the short chain fatty acids isovalerate, valerate, acetate, propionate and butyrate in fecal pellet samples of WT and Il4ra^{F709} mice that were either sham (PBS) sensitized or sensitized with OVA/SEB (n=4-10 mice per group). FIG. 42B shows core body temperature changes in WT and Il4ra^{F709} mice that were kept without or with oral supplementation with short chain fatty acids (SCFAs) in their drinking water while being either sham-(PBS) or OVA/SEB-sensitized, as indicated, then challenged with OVA. (n=7-24 mice per group). FIG. 42C shows total and OVA-specific serum IgE antibody concentrations (n=4-8 mice per group). FIG. 42D shows representative flow plots of CD4⁺Foxp3⁺ T cells in WT and Il4ra^{F709} mice treated with SCFAs. FIG. 42E shows frequencies and numbers of small intestine (SI) CD4⁺Foxp3⁺ T cells (n=4-13 mice per group). FIG. 42F shows representative flow plots of KI67 expression on CD4⁺Foxp3⁺ T cells in WT and Il4ra^{F709} mice treated with SCFAs. FIG. 42G shows frequencies and numbers of KI67⁺ expressing small intestinal (SI) CD4⁺Foxp3⁺ T cells (n=4-9 mice per group). FIG. 42H shows frequencies of CD4⁺Foxp3⁺ROR-γt⁺ and CD4⁺Foxp3⁺ROR-γt⁺ T cells in the MLN of the respective mouse group (n=4-7 mice per group). Each dot represents one mouse. Data represent mean±s.e.m. from two independent experiments. For FIG. 42A, *p<0.05 by Student's unpaired two tailed t test. For FIGS. 42B-42H, ***P<0.001, ****p<0.0001 by one-way ANOVA with Dunnett post hoc analysis. For core body temperature measurements **P<0.01 by repeat measures two-way ANOVA.

FIGS. 43A-43B show the persistence of the Clostridiales consortium species in Il4ra^{F709} mice. FIG. 43A shows the abundance of the respective Clostridiales species in fecal pellets of Il4ra^{F709} mice at baseline and following a single gavage with the indicated species at 10⁸ colony forming units (CFU) and their stools serially sampled at the indicated times thereafter and analyzed for species abundance by RT-PCR. The results are normalized to Log 10 CFU/gram fecal matter (n=6 mice/group). FIG. 43B shows the abundance of the respective Clostridiales species in fecal pellets of Il4ra^{F709} mice that were treated with antibiotics (ABX) for one week then sensitized weekly with OVA/SEB for eight weeks either without or with additional treatment with the Clostridales consortium at 10⁷ CFU/species. The stools were collected at the end of ABX treatment and at the end of the sensitization period and analyzed for species abundance by RT-PCR. The results are normalized to Log 10 CFU/gram fecal matter (n=5 mice/group). Each dot represents one mouse.

FIGS. 44A-44C show the persistence of the Bacteroidales and Proteobacteria consortia species in Il4ra^{F709} mice. FIG. 44A shows the abundance of the respective Bacteroidales species in fecal pellets of Il4ra^{F709} mice at baseline and following a single gavage with the indicated species at 10⁸ colony forming units (CFU) and their stools serially sampled at the indicated times thereafter and analyzed for species abundance by RT-PCR. The results are normalized to Log 10 CFU/gram fecal matter (n=6 mice/group). FIGS. 44B-44C show the abundance of the respective Bacteroidales and Proteobacteria species in fecal pellets of Il4ra^{F709} mice that were treated with antibiotics (ABX) for one week then sensitized weekly with OVA/SEB for eight weeks either without or with additional treatment with the respective consortia at 10⁷ CFU/species. The stools were collected at the end of ABX treatment and at the end of the sensitization period and analyzed for species abundance by RT-PCR. The results are normalized to Log 10 CFU/gram fecal matter (n=5 mice/group). Each dot represents one mouse. Data represent mean±s.e.m. from one experiment.

FIGS. 45A-45F show that heat-inactivated Clostridiales consortium does not protect against FA in Il4ra^{F709} mice. FIG. 45A shows core body temperature changes in Il4ra^{F709} mice that were antibiotic-treated then orally sensitized with OVA/SEB while receiving by gavage either sham treatment or treatment with heat-inactivated Clostridales consortium species, and thereafter challenged with OVA (n=5-7 mice per group). ns=not significant, by two-way ANOVA. FIGS. 45B-C show total and OVA-specific serum IgE concentrations (n=5-7 mice per group). FIG. 45D shows serum MMCP-1 concentrations post OVA challenge (n=5-7 mice per group). FIGS. 45E-45F show flow cytometric analysis and cell frequencies of MLN ROR-γt⁺CD4⁺Foxp3⁺ Treg cells (n=5-7 mice per group). Each dot represents one mouse. Throughout, data represent mean±s.e.m. from two independent experiments. For FIGS. 45B-45F: ns=not significant by Student's unpaired two tailed t test.

FIGS. 46A-46G show an analysis of ROR-γt⁺ expression in human subjects and mutant mice. FIG. 46A shows the gating strategy for CD4⁺Foxp3⁺ (G1) and CD4⁺Foxp3⁻ T (G2) cells ex vivo. FIG. 46B shows the gating strategy for the expression of ROR-γt in Teff cells (G2) from FA patients, healthy controls (HC) and atopic subjects (atopy), as compared to an isotype control. FIG. 46C shows representative flow cytometric plots and frequencies of peripheral blood CD4⁺Foxp3⁺ROR-γt⁺ T cells in WT and Il4ra^{F709} mice (n=7 mice per group). FIG. 46D shows representative flow cytometric plots and frequencies of peripheral blood CD4⁺Foxp3⁺Helios⁻NRP1⁻ ROR-γt⁺ T cells in WT and Il4ra^{F709} mice (n=7 mice per group). FIGS. 46E-46F show representative flow cytometric plots and frequencies of MLN CD4⁺Foxp3⁺ROR-γt⁺ T cells from Foxp3^{YFPCre} mice sensitized with OVA/SEB, and Foxp3^{YFPCre}Rorc^{Δ/Δ} either sham sensitized (PBS) or sensitized with OVA/SEB, as indicated (n=5 mice per group). FIG. 46G show quantitative RT-PCR of Rorc gene expression in MLN CD4⁺Foxp3⁺ Treg and CD4⁺Foxp3⁻Teff cells from Foxp3^{YFPCre}, Foxp3^{YFPCre}Rorc^{Δ/Δ}, and Il4ra^{F709}Foxp3^{YFPCre}Rorc^{Δ/Δ} mice. Data were normalized to the endogenous Hprt transcripts (n=5 mice per group). Each dot represents one mouse. Results represent Means±S.E.M. collated from 2 independent experiments. For FIG. 46G, ****p<0.0001 by one-way ANOVA with Dunnett post hoc analysis.

FIGS. 47A-47H show that Treg cell-specific deletion of Rorc and Myd88 dysregulates the mucosal immune responses. FIGS. 47A-47D show flow cytometric analysis and frequencies of sIgA⁺ (FIGS. 47A-B) and IgE⁺ (FIGS. 47C-D) fecal bacteria in Foxp3^{YFPCre}, Il4ra^{F709}Foxp3^{YFPCre} and Foxp3^{YFPCre}Rorc^{Δ/Δ} mice sensitized with OVA/SEB. Fecal pellets of Rag2^{Δ/Δ} and Igh7^{-/-}Il4ra^{F709} mice were used as negative controls for the respective antibody staining (n=6-11 mice per group). FIGS. 47E-47F show flow cytometric analysis and frequencies of GATA3⁺Foxp3⁺ Treg cells in OVA/SEB-sensitized Il4ra^{F709}Foxp3^{YFPCre} mice, and in OVA/SEB-sensitized Il4ra^{F709}Foxp3^{YFPCre} and Il4ra^{F709}Foxp3^{YFPCre}Rorc^{Δ/Δ} mice treated with the Clostridiales or Bacteroidales consortia, as indicated (n=4-9 mice per group). FIGS. 47G-47H show flow cytometric analysis and frequencies of GATA3⁺Foxp3⁺ Treg cells in OVA/SEB-sensitized Il4ra^{F709}Foxp3^{YFPCre} mice treated with the Bacteroidales consortium, and in OVA/SEB-sensitized Il4ra^{F709}Foxp3^{YFPC re}Myd88^{Δ/Δ} mice otherwise untreated or treated with the Clostridiales or Bacteroidales consortia, as indicated (n=8-9 mice per group). Each symbol represents one mouse. Results represent Means±S.E.M. collated from 2 independent experiments. **P<0.01, ****P<0.0001 by one-way ANOVA with Dunnett post hoc analysis.

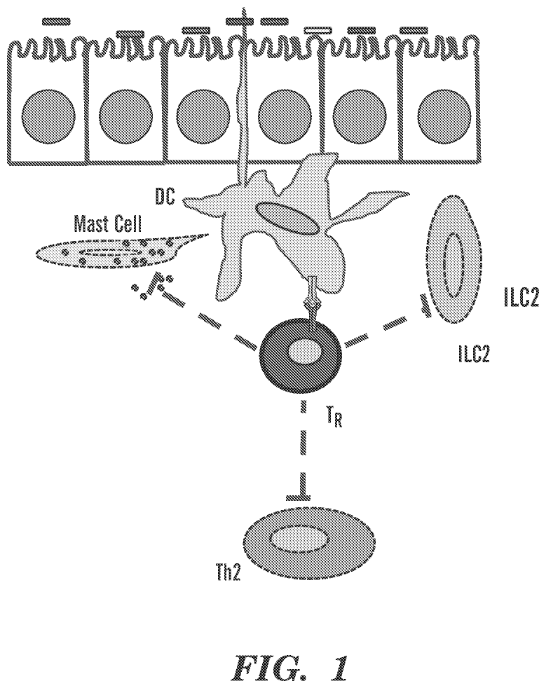

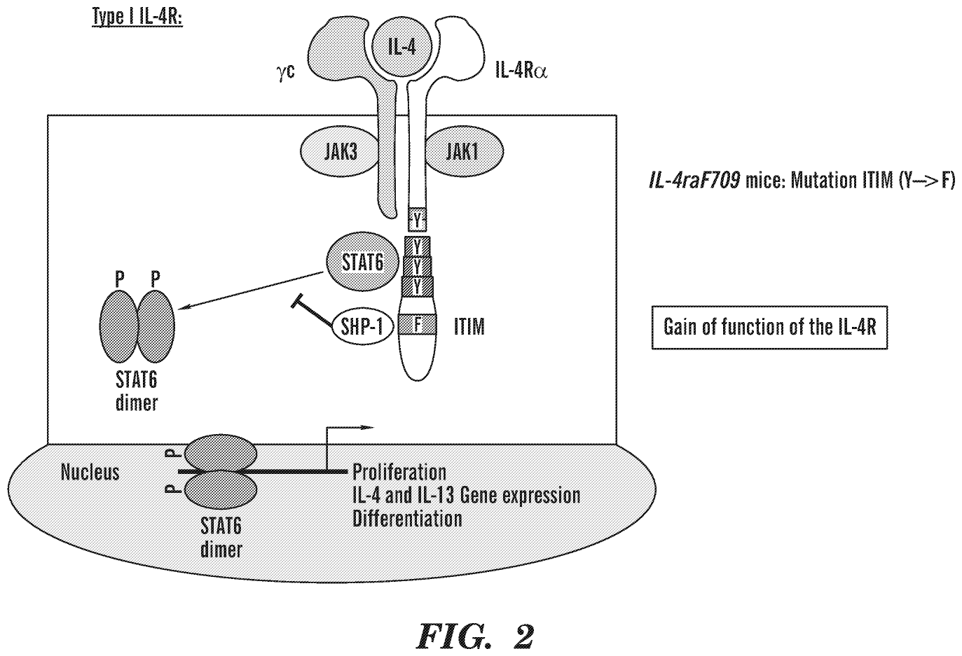

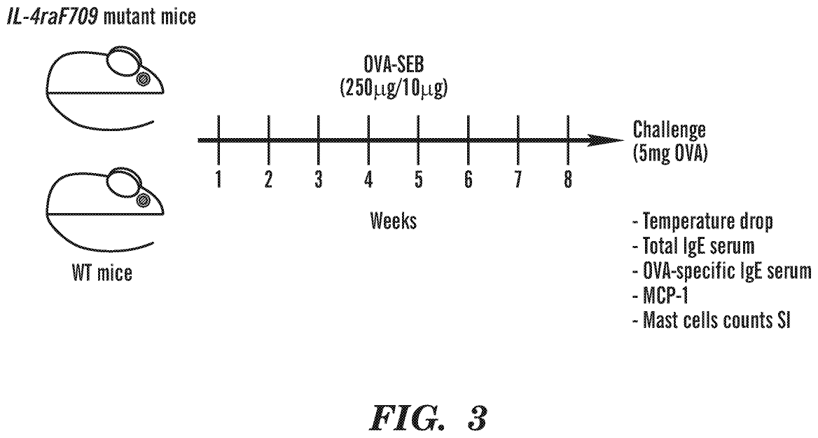

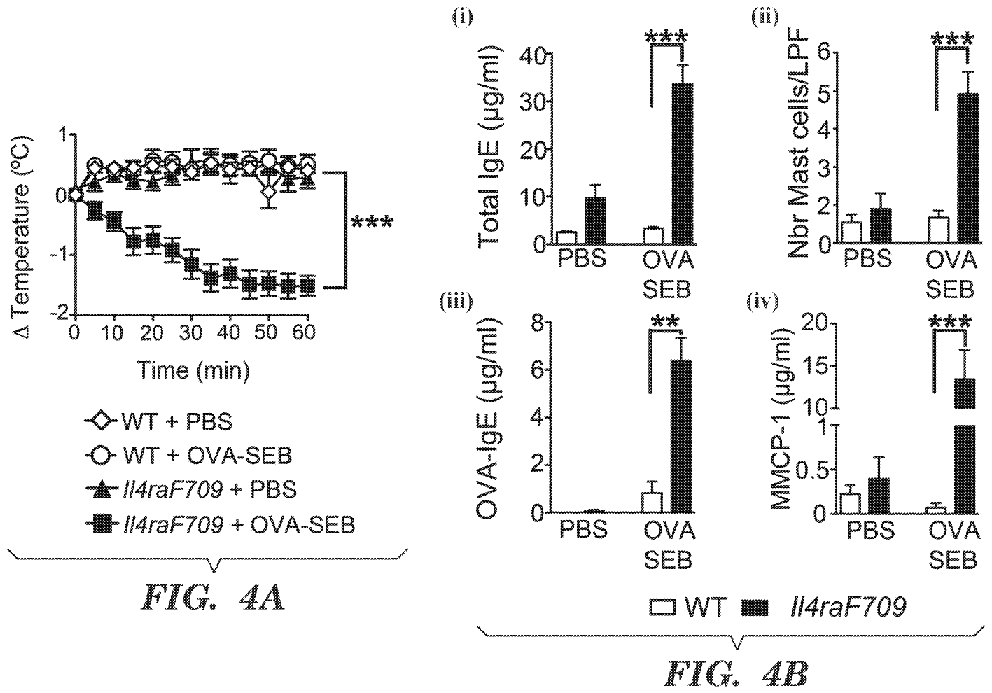

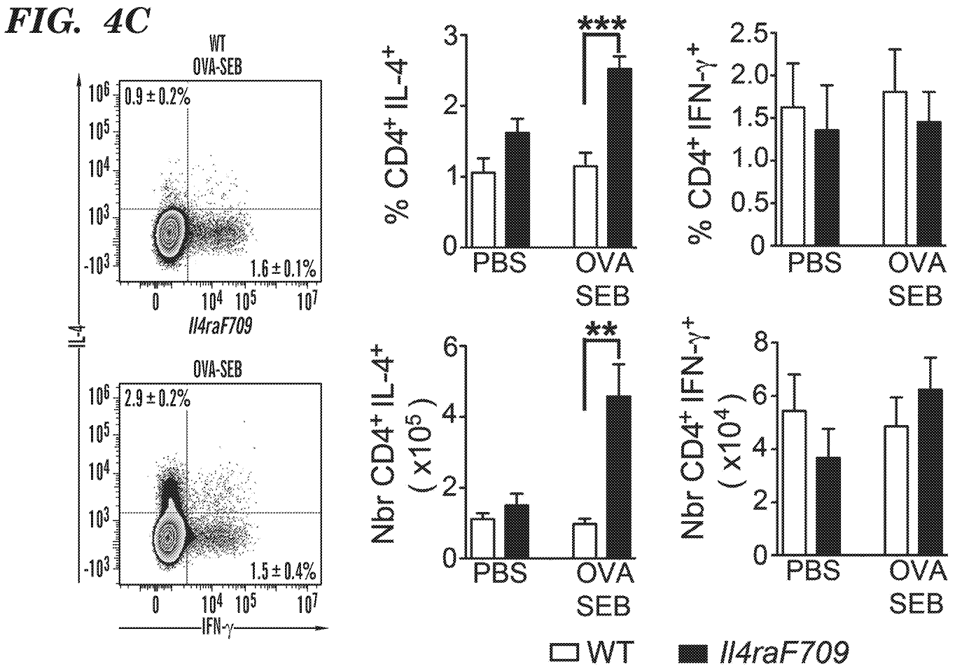

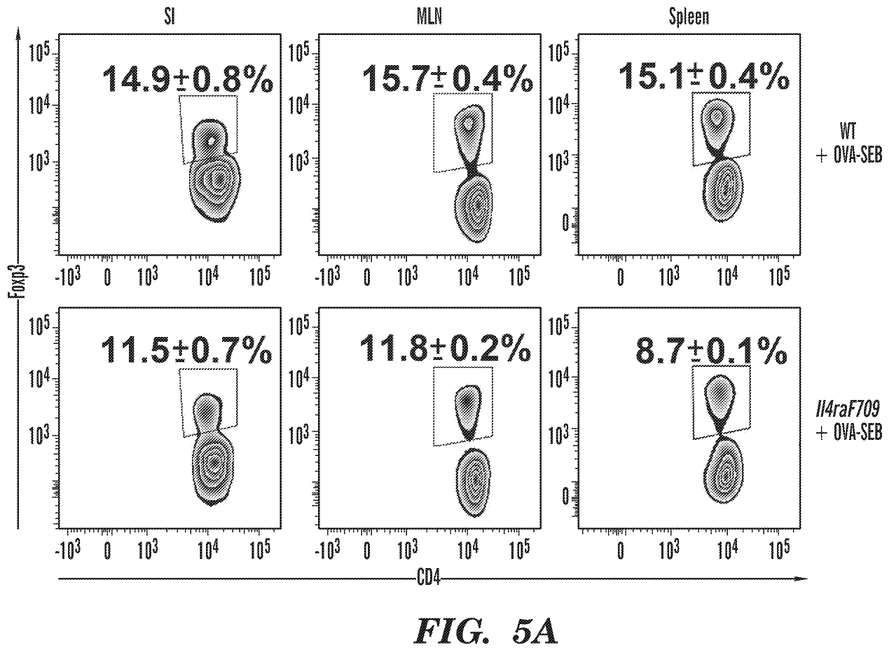

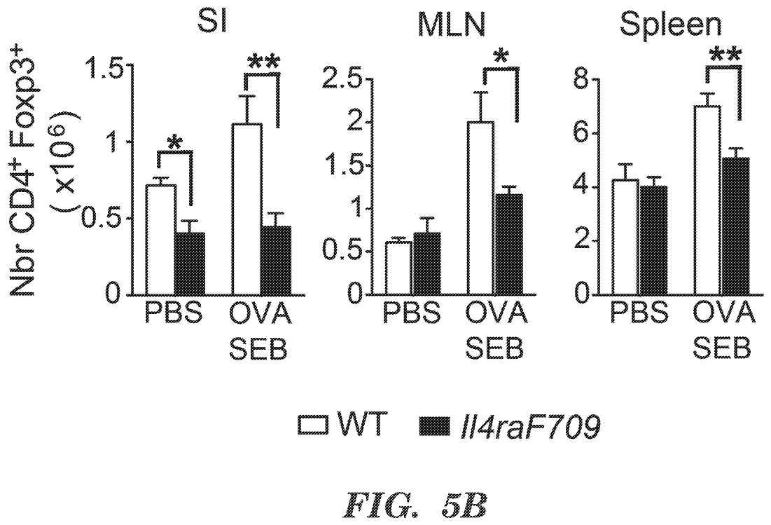

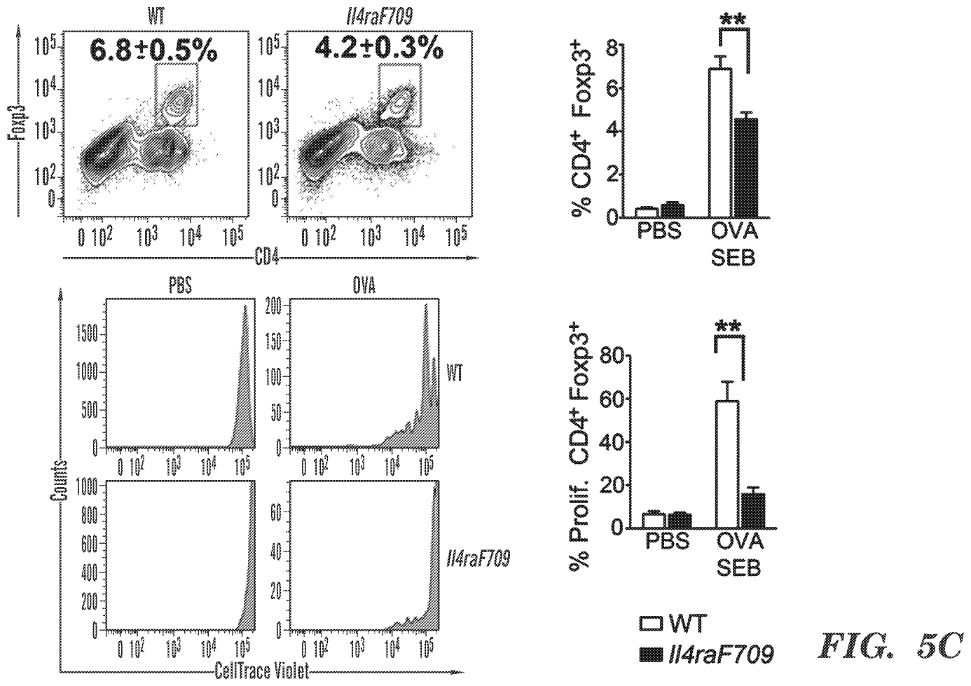

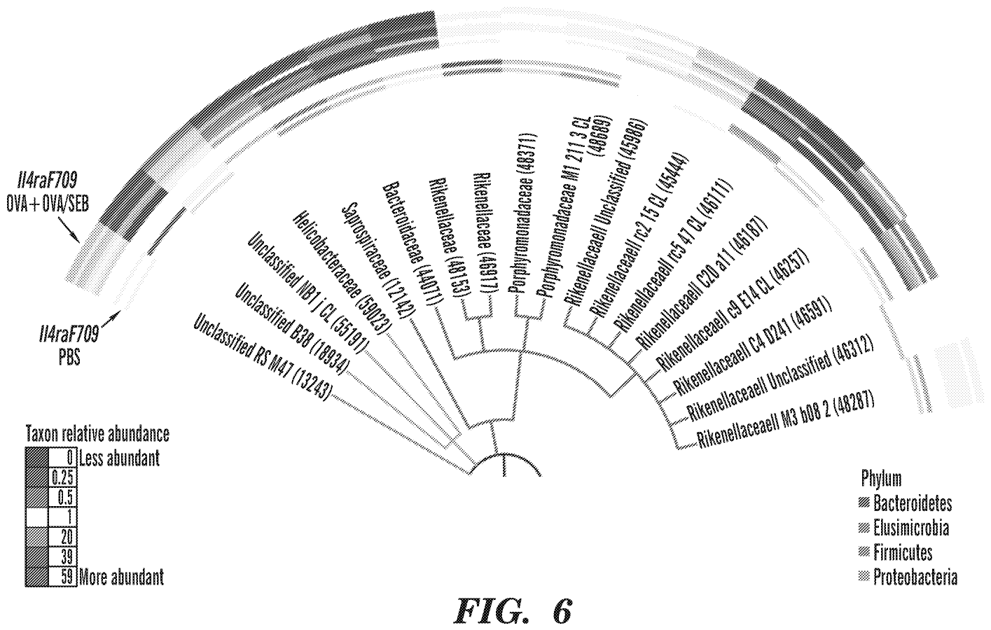

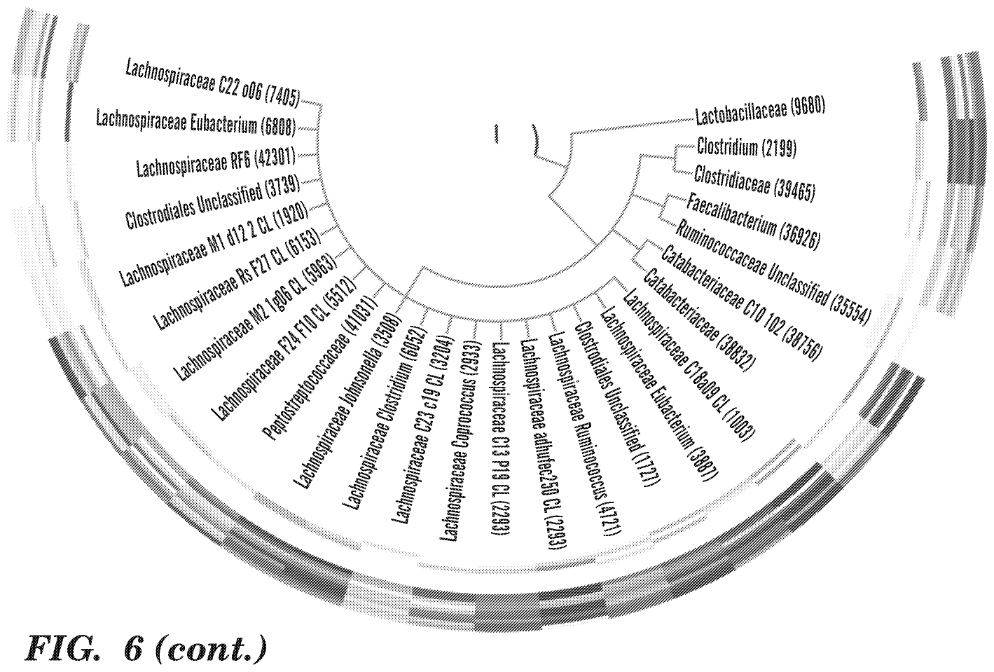

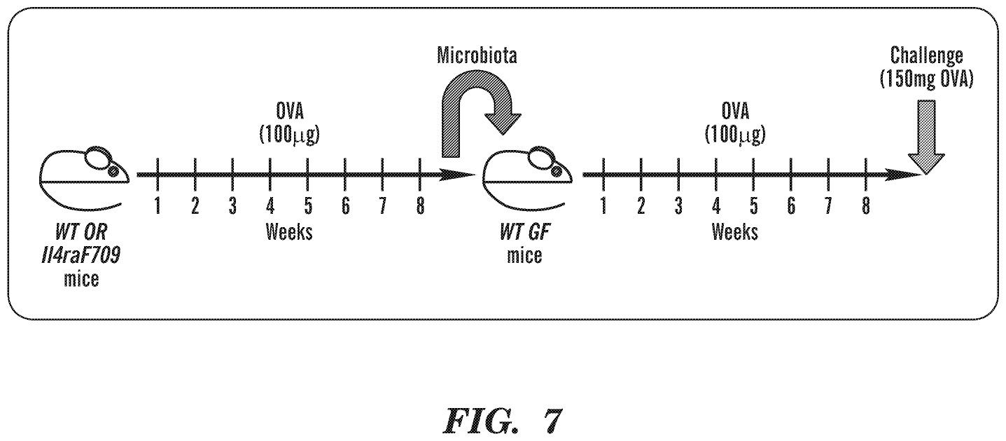

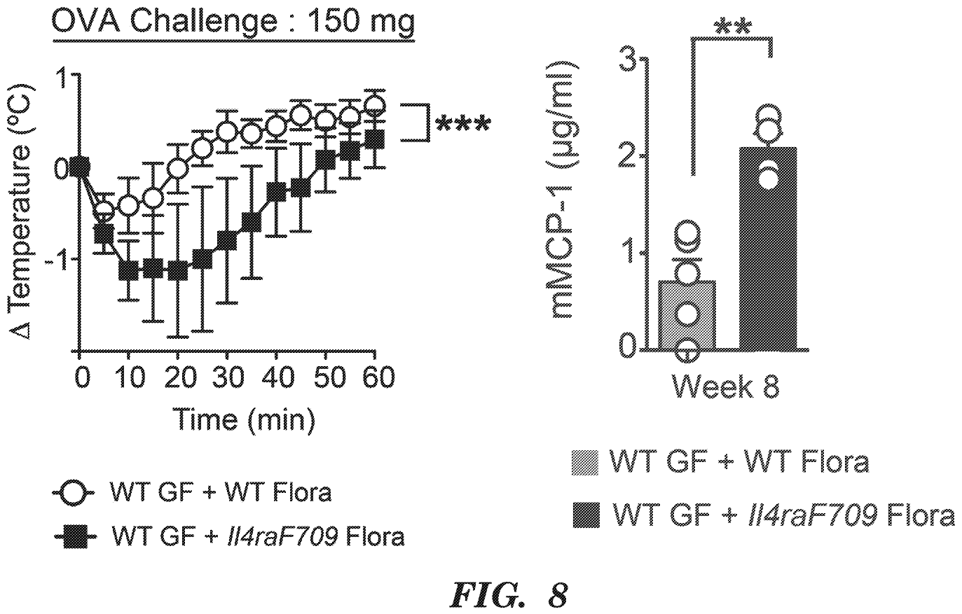

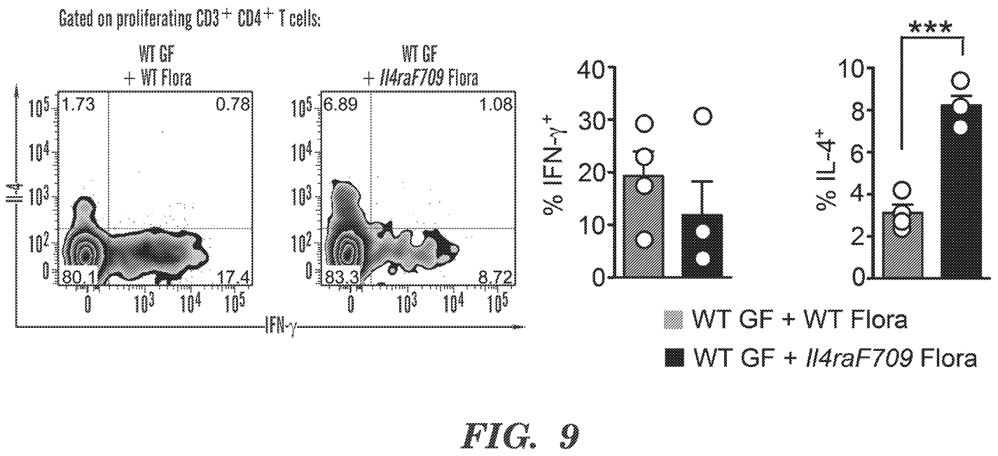

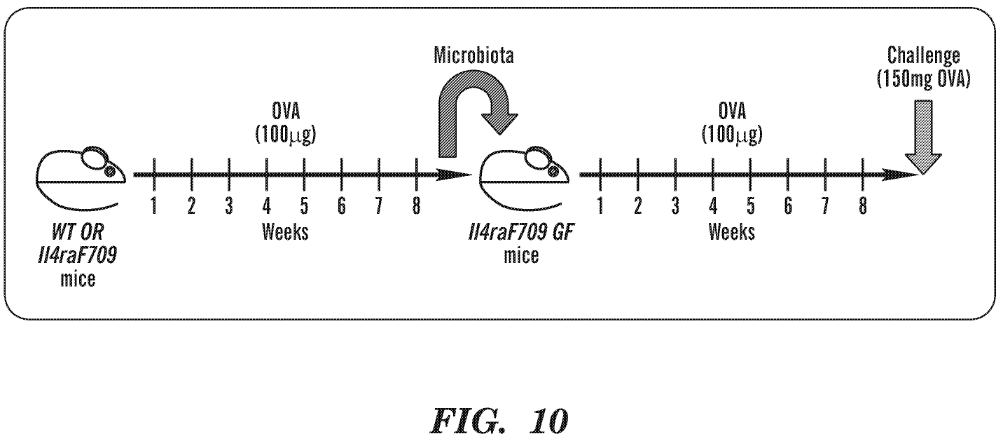

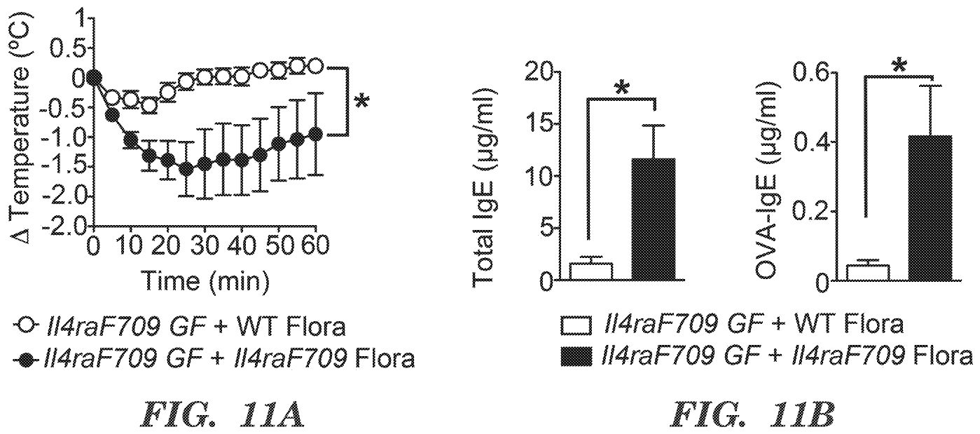

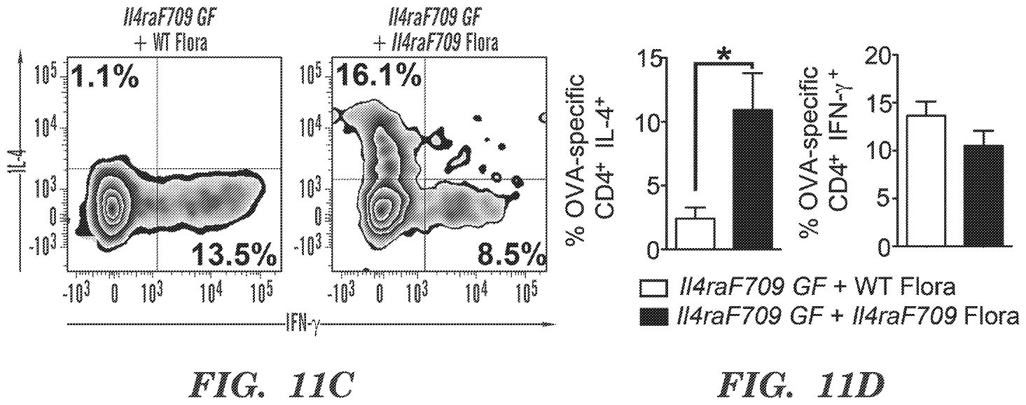

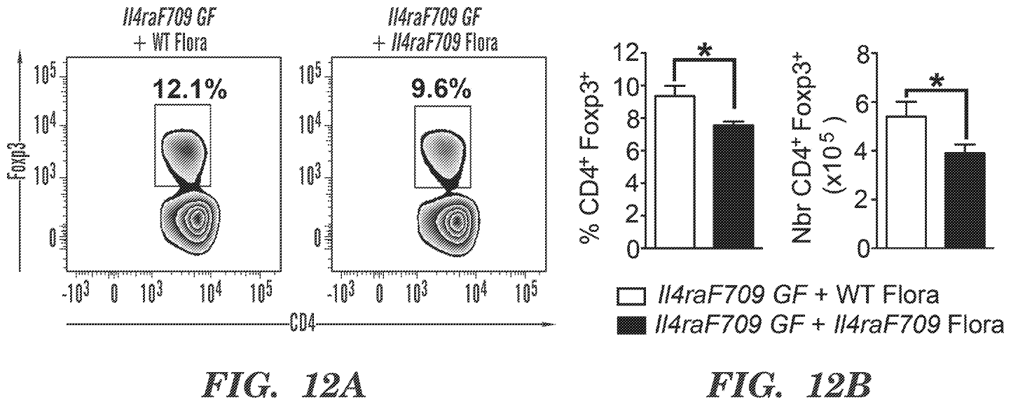

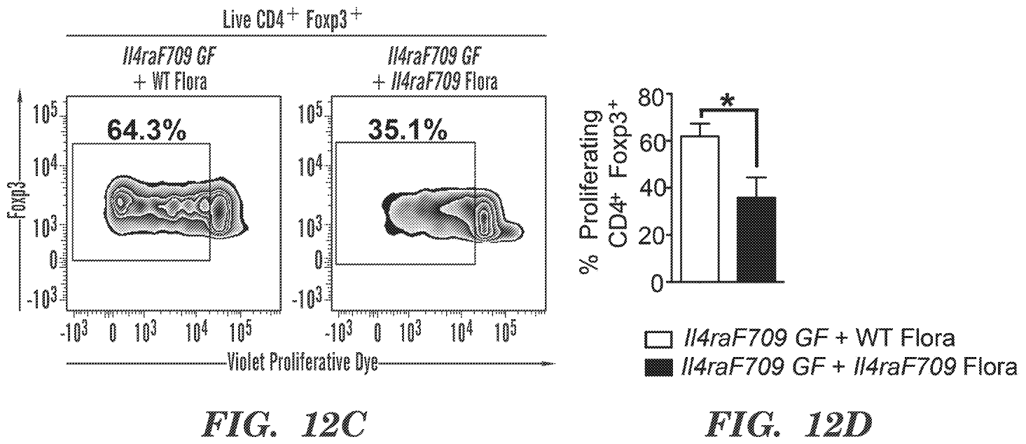

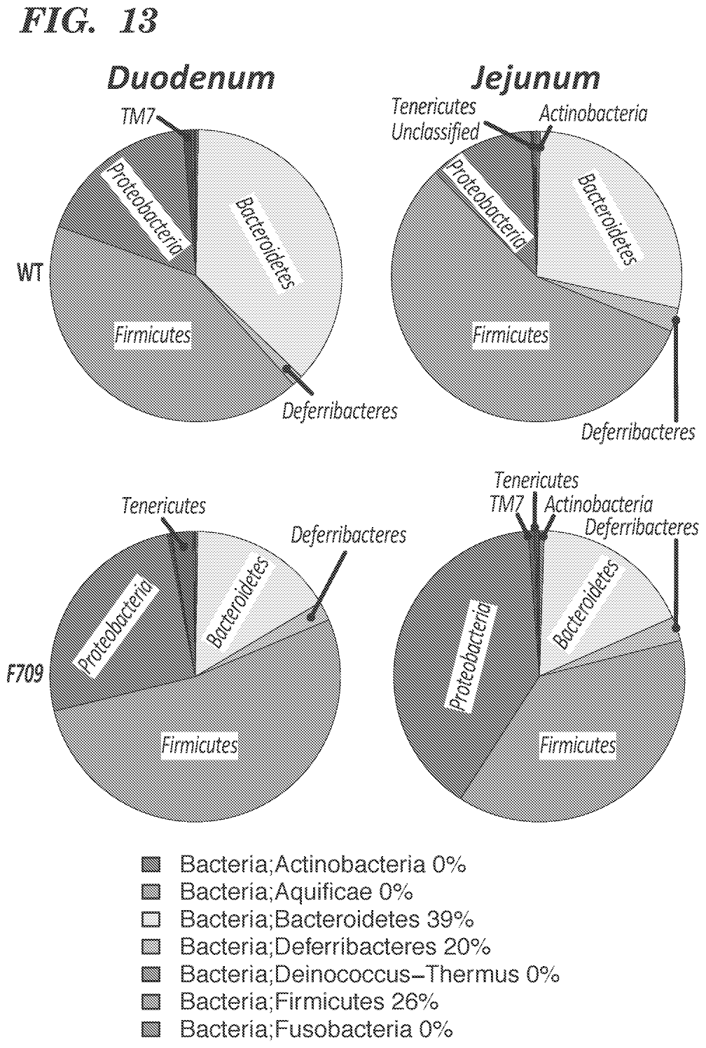

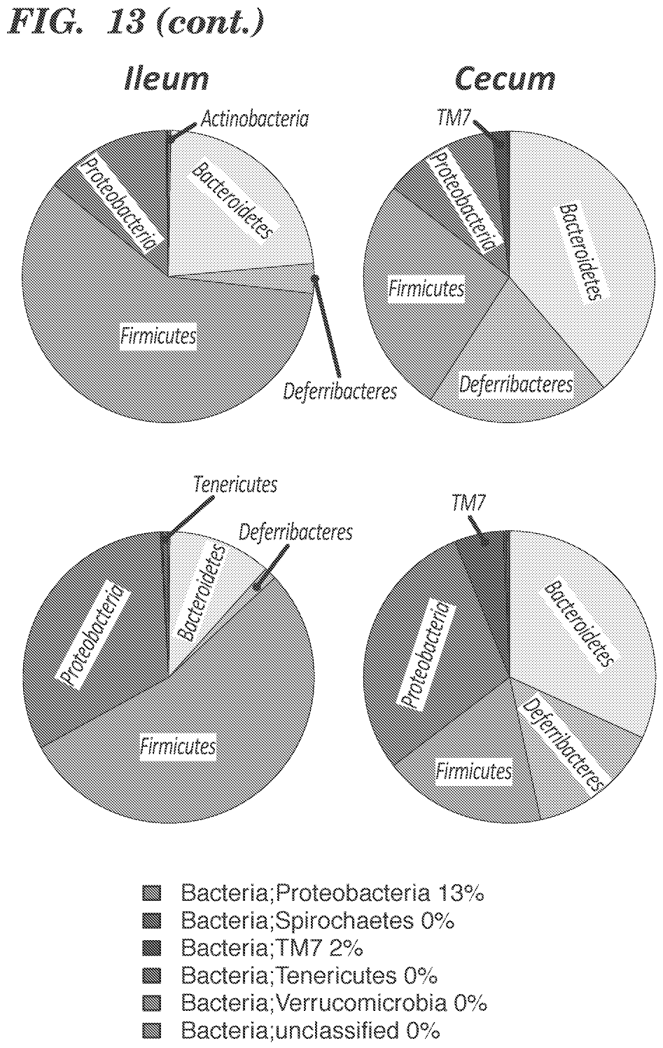

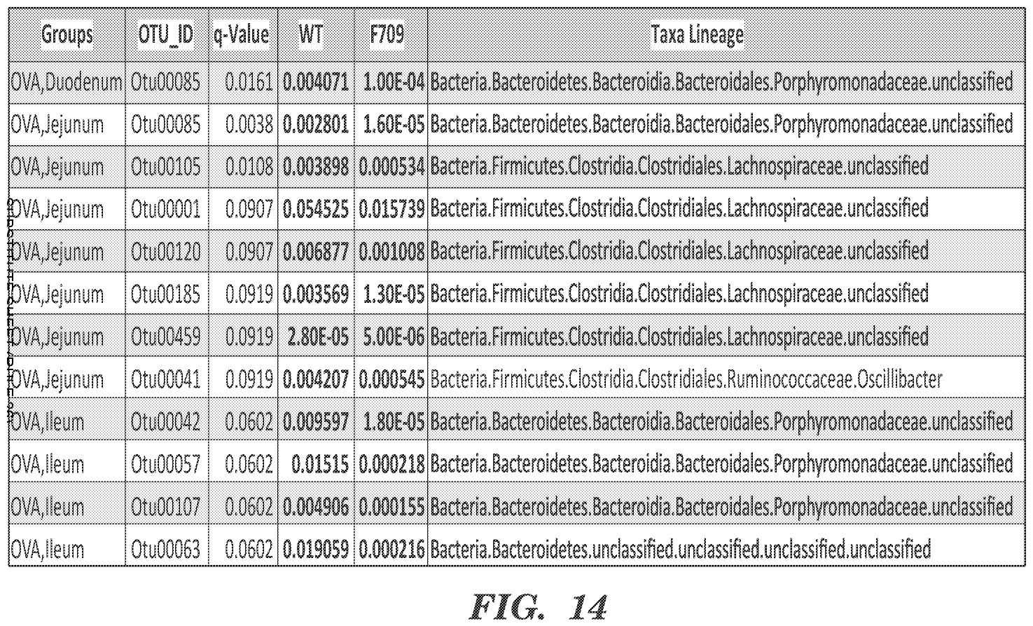

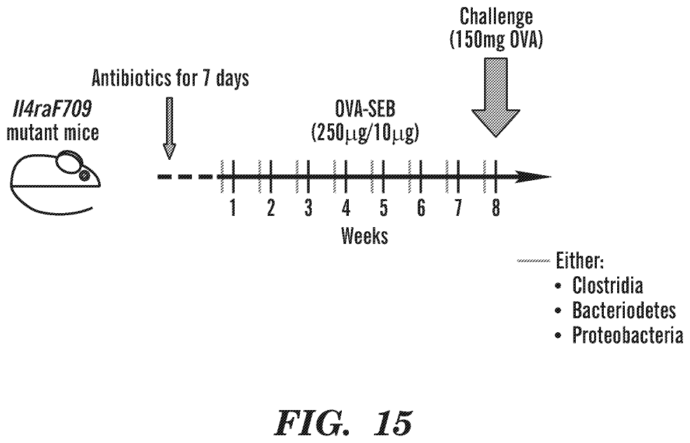

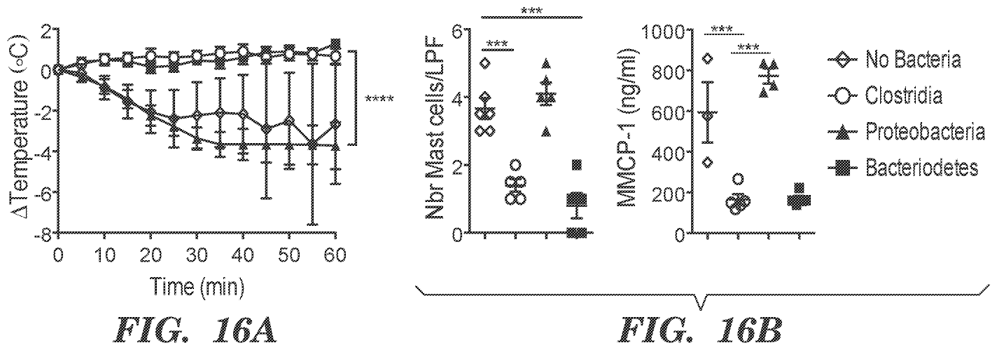

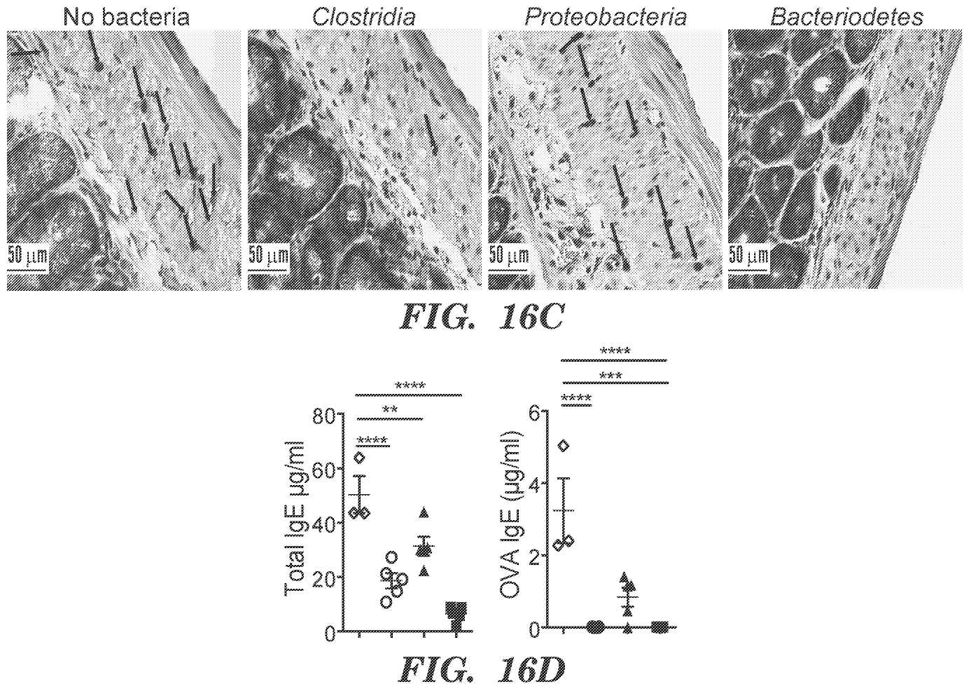

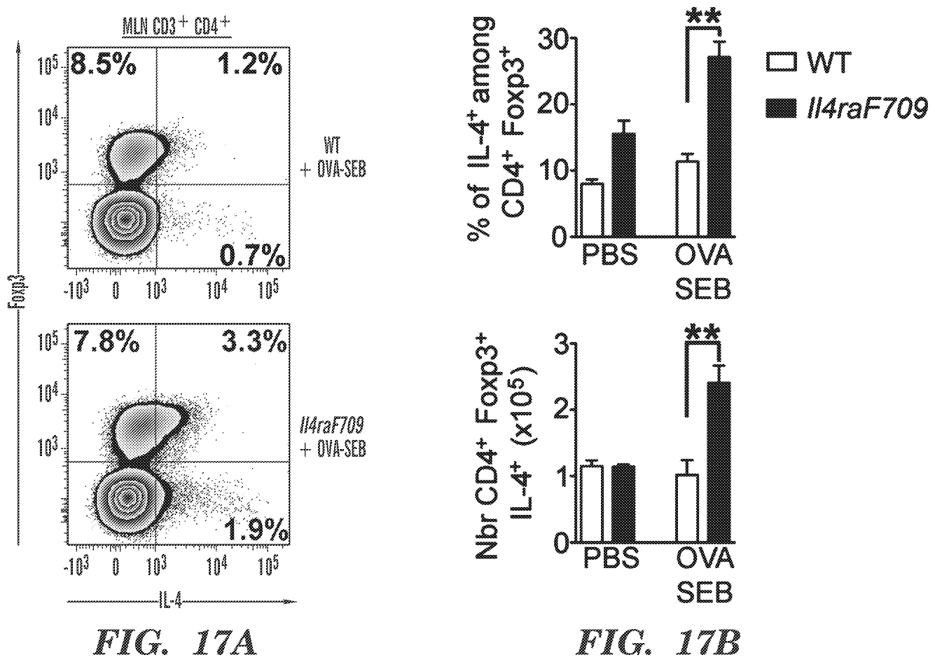

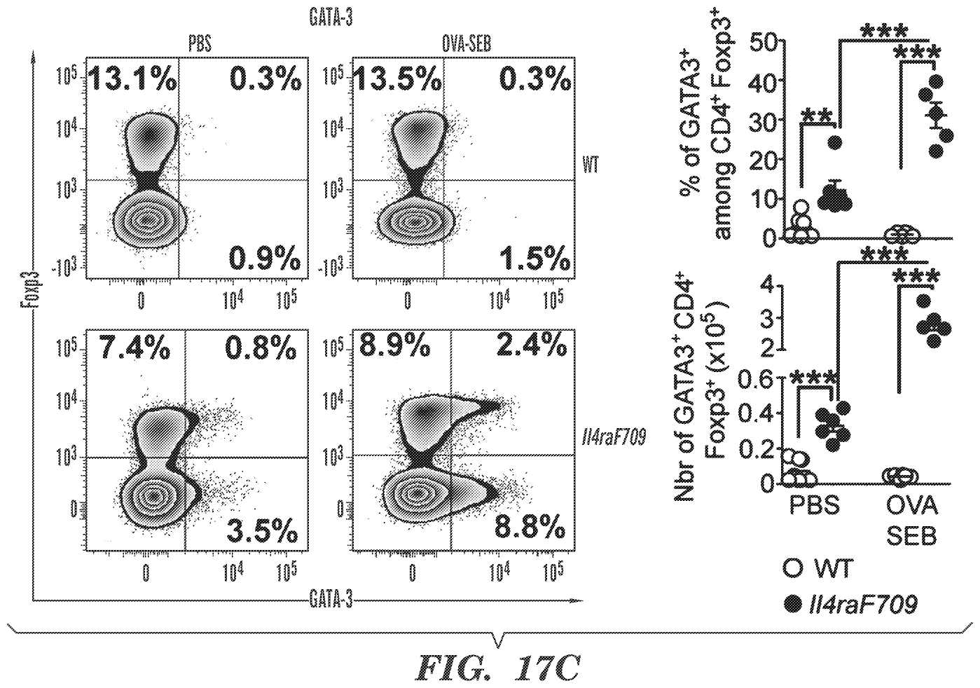

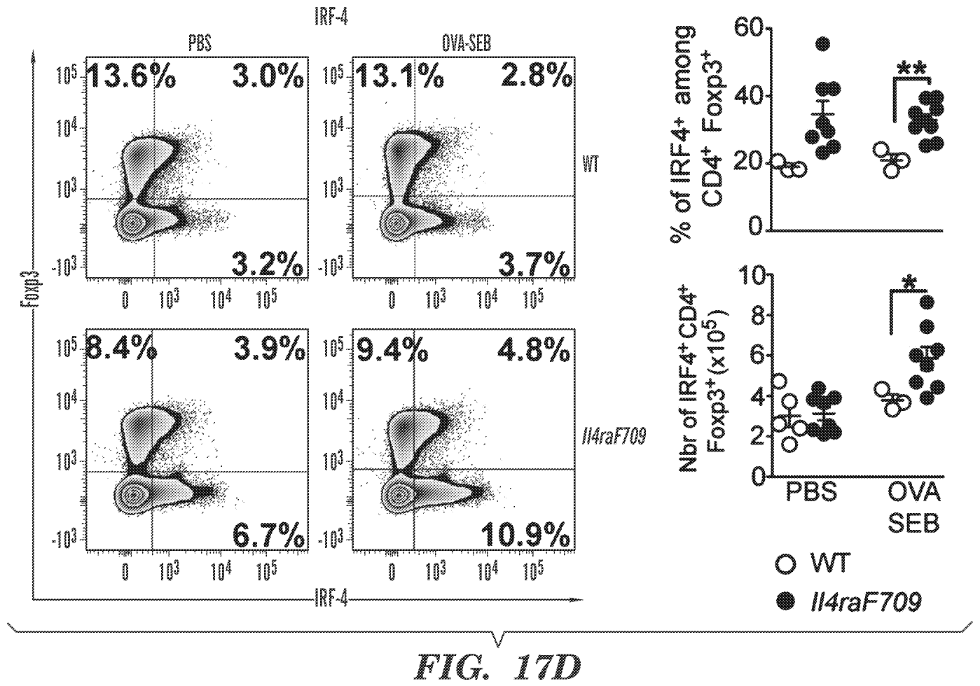

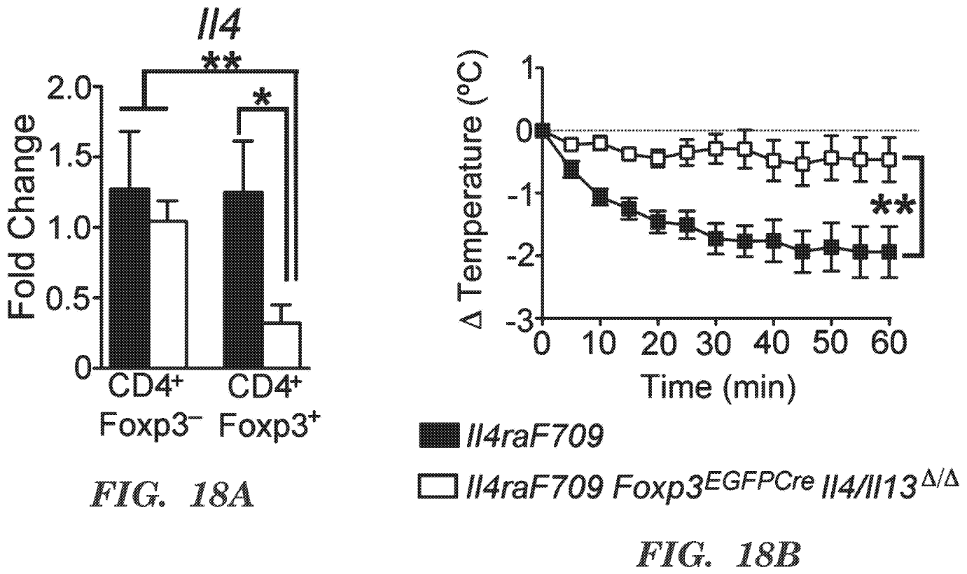

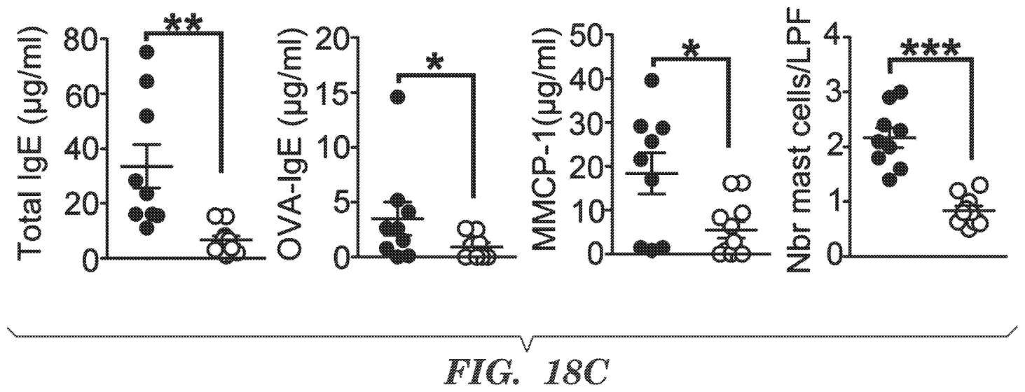

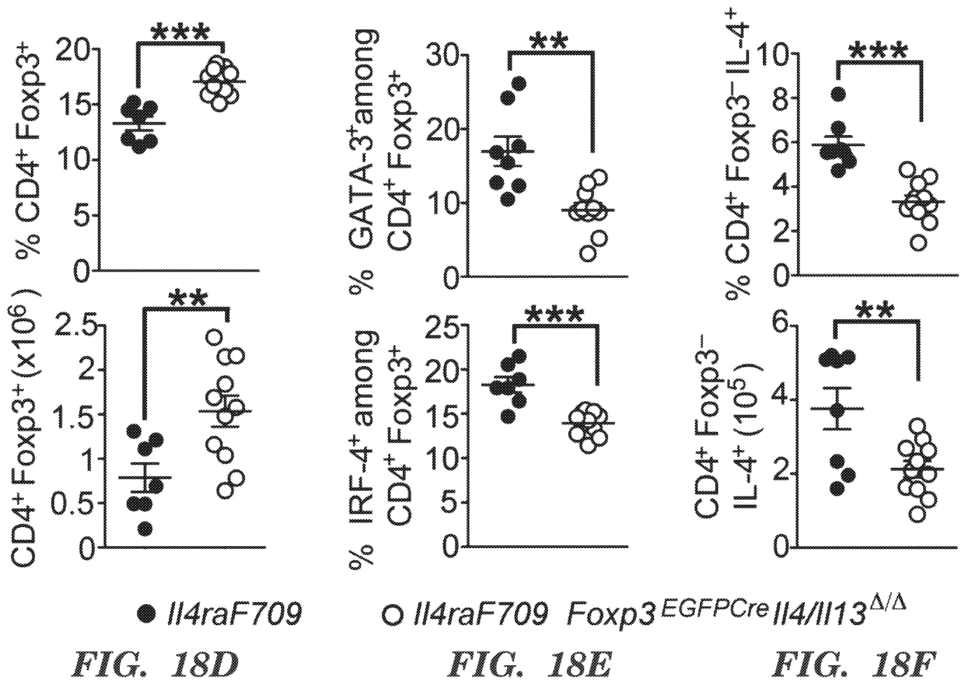

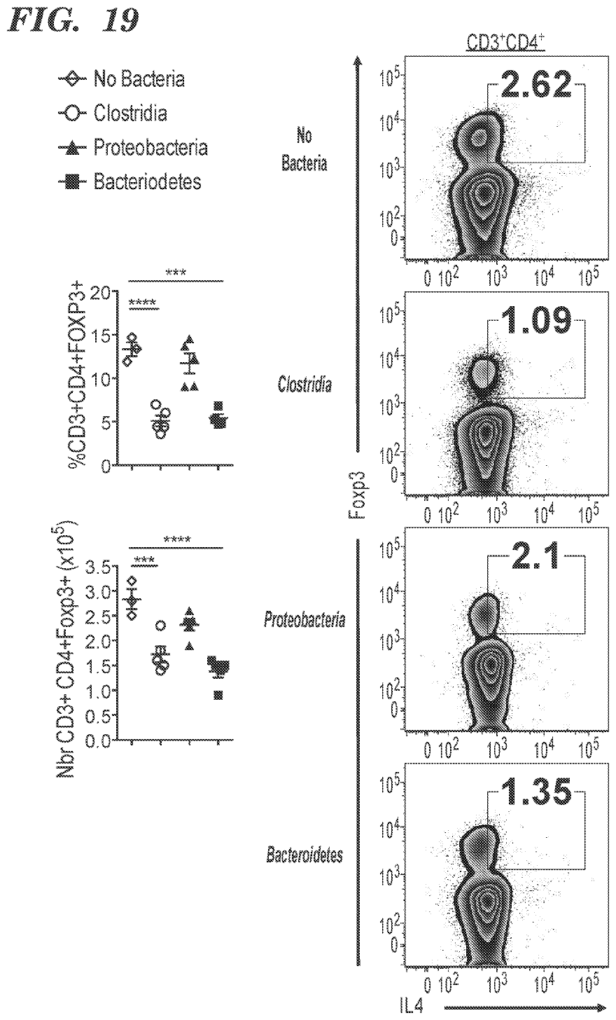

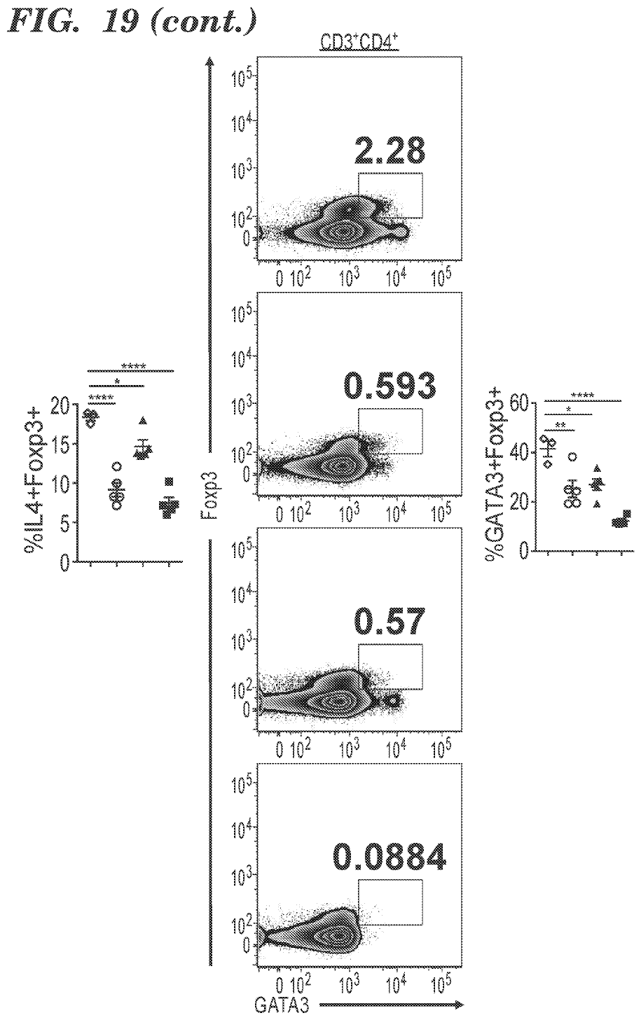

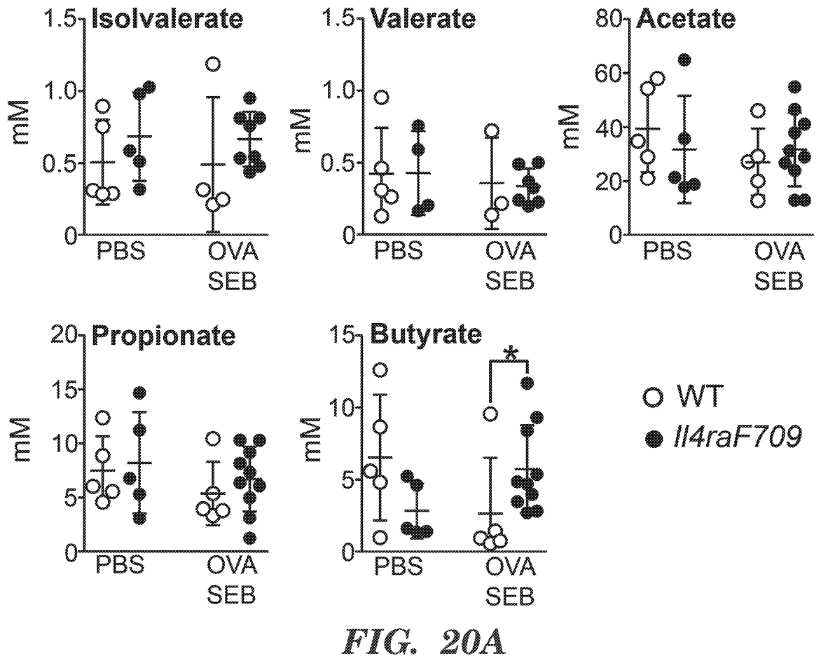

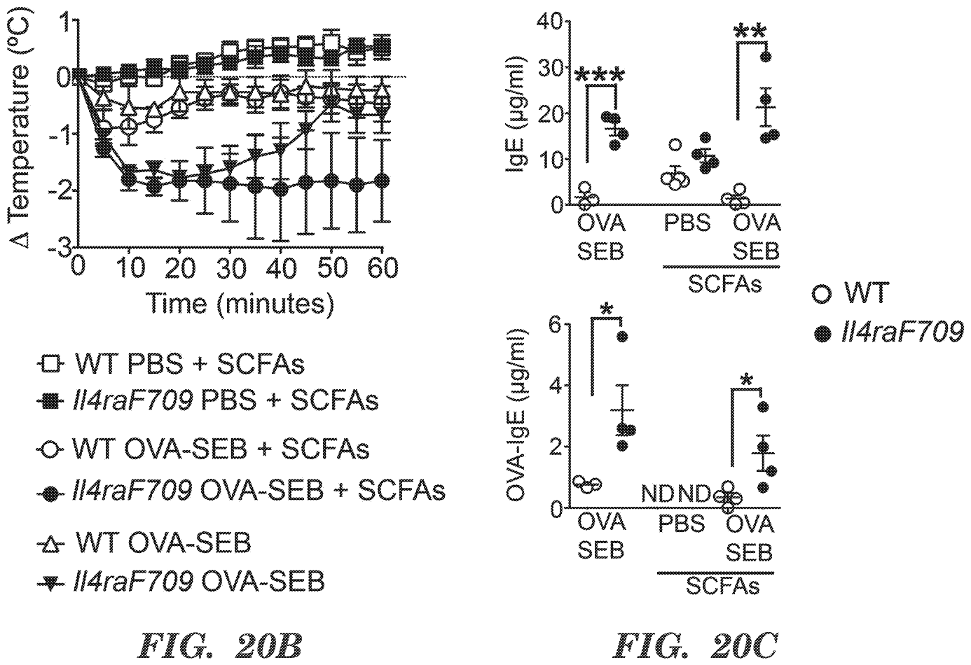

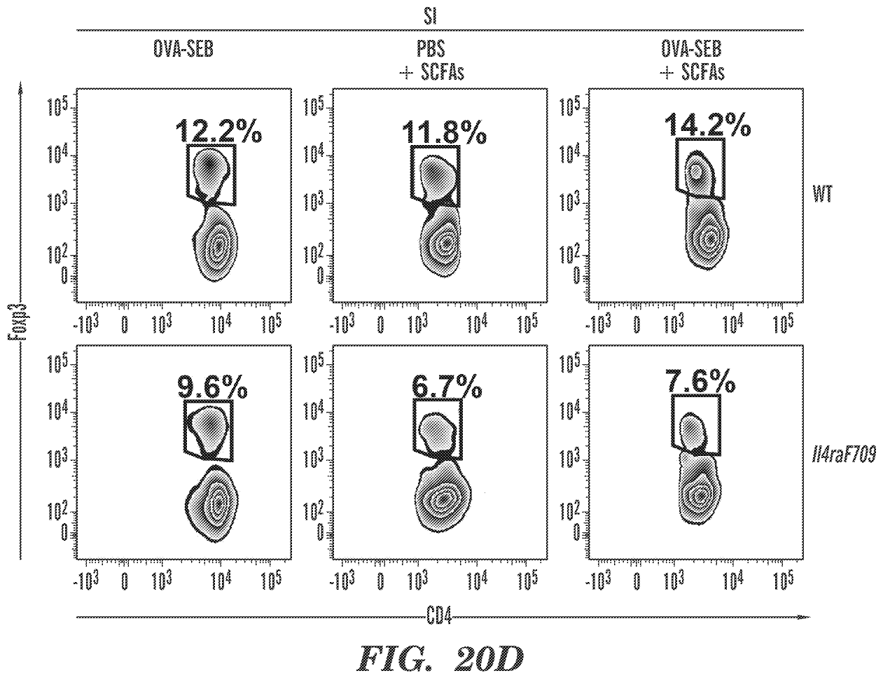

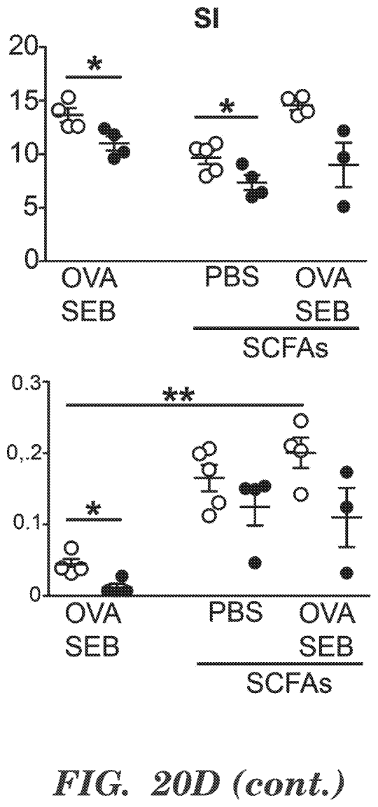

### DETAILED DESCRIPTION

### Definitions

As used herein, the term “food allergy” refers to a failure of oral tolerance to food antigens associated with Tₕ2 immunity and allergen-specific IgE responses. That is, an immune response is generated in response to particular food antigens and can lead to hives, gastrointestinal symptoms, abdominal pain, anaphylaxis and even death.

As used herein, the term “microbiota” can refer to the human microbiome, the human microbiota, or the human gut microbiota. The human microbiome (or human microbiota) may be understood as the aggregate of microorganisms that reside on the surface and in deep layers of skin, in the saliva and oral mucosa, in the conjunctiva, and in the genitourinary and gastrointestinal tracts of humans. The human microbiome is comprised of bacteria, fungi, viruses, and archaea. At least some of these organisms perform tasks that are useful for the human host. Under normal circumstances, these microorganisms do not cause acute disease to the human host, but instead cause no harm or participate in maintaining health. Hence, this population of organisms is frequently referred to as the “normal flora.” The population of microorganisms living in the human gastrointestinal tract is commonly referred to as “microbial flora”, “gut flora”, and/or “gut microbiota”. The microbial flora of the human gut encompasses a wide variety of microorganisms that aid in digestion, the synthesis of vitamins and other metabolites, and creating enzymes not produced by the human body.

As used herein, the term “minimal microbial consortium” refers to a mixed population of cells comprising at least two species of viable gut bacteria that do not promote acute disease in a subject. The microbial consortium is “minimal” when an additional bacterial species is added and there is no additional benefit (e.g., less than 5%) in avoiding or mitigating an allergic response. In some embodiments, the minimal microbial consortium comprises at least 3, at least 4, at least 5, at least 6, at least 7, at least 8, at least 9, at least 10, at least 11, or more different species of bacteria. In some embodiments, the minimal microbial consortium comprises at least one species of bacteria from the phyla Clostridia and/or Bacteroidetes.

“Operational taxonomic unit (OTU, plural OTUs)” refers to a terminal leaf in a phylogenetic tree and is defined by a specific genetic sequence and all sequences that share a specified degree of sequence identity to this sequence at the level of species. A “type” or a plurality of “types” of bacteria includes an OTU or a plurality of different OTUs, and also encompasses a strain, species, genus, family or order of bacteria. The specific genetic sequence may be the 16S rRNA sequence or a portion of the 16S rRNA sequence, or it may be a functionally conserved housekeeping gene found broadly across the eubacterial kingdom. OTUs generally share at least 95%, 96%, 97%, 98%, or 99% sequence identity. OTUs are frequently defined by comparing sequences between organisms. Sequences with less than the specified sequence identity (e.g., less than 97%) are not considered to form part of the same OTU.

“Clade” refers to the set of OTUs or members of a phylogenetic tree downstream of a statistically valid node in a phylogenetic tree. The clade comprises a set of terminal leaves in the phylogenetic tree that is a distinct monophyletic evolutionary unit.

In microbiology, “16S sequencing” or “16S rRNA” or “16S-rRNA” or “16S” refers to sequence derived by characterizing the nucleotides that comprise the 16S ribosomal RNA gene(s). The bacterial 16S rDNA is approximately 1500 nucleotides in length and is used in reconstructing the evolutionary relationships and sequence similarity of one bacterial isolate to a second isolate using phylogenetic approaches. 16S sequences are used for phylogenetic reconstruction as they are in general highly conserved, but contain specific hypervariable regions that harbor sufficient nucleotide diversity to differentiate genera and species of most bacteria, as well as fungi.

The “V1-V9 regions” of the 16S rRNA refers to the first through ninth hypervariable regions of the 16S rRNA gene that are used for genetic typing of bacterial samples. These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature. Brosius et al., Complete nucleotide sequence of a 16S ribosomal RNA gene from Escherichia coli, PNAS 75(10):4801-4805 (1978). In some embodiments, at least one of the V1, V2, V3, V4, V5, V6, V7, V8, and V9 regions are used to characterize an OTU. In one embodiment, the V1, V2, and V3 regions are used to characterize an OTU. In another embodiment, the V3, V4, and V5 regions are used to characterize an OTU. In another embodiment, the V4 region is used to characterize an OTU. A person of ordinary skill in the art can identify the specific hypervariable regions of a candidate 16S rRNA by comparing the candidate sequence in question to the reference sequence and identifying the hypervariable regions based on similarity to the reference hypervariable regions.

“Dysbiosis” refers to a state of the microbiota or microbiome of the gut or other body area, including mucosal or skin surfaces in which the normal diversity and/or function of the ecological network is disrupted. Any disruption from the preferred (e.g., ideal) state of the microbiota can be considered a dysbiosis, even if such dysbiosis does not result in a detectable decrease in health. This state of dysbiosis may be unhealthy, it may be unhealthy under only certain conditions, or it may prevent a subject from becoming healthier. Dysbiosis may be due to a decrease in diversity, the overgrowth of one or more pathogens or pathobionts, symbiotic organisms able to cause disease only when certain genetic and/or environmental conditions are present in a patient, or the shift to an ecological network that no longer provides a beneficial function to the host and therefore no longer promotes health.

The terms “patient”, “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. In another embodiment, the subject is a domesticated animal including companion animals (e.g., dogs, cats, rats, guinea pigs, hamsters etc.).

As used herein, the term “enteric coated drug delivery device” or “enteric coated composition” refers to any drug delivery method that can be administered orally but is not degraded or activated until the device enters the intestines. Such methods can utilize a coating or encapsulation that is degraded using e.g., pH dependent means, permitting protection of the delivery device and the microbial consortium to be administered or transplanted throughout the upper gastrointestinal tract until the device reaches the alkaline pH of the intestines. In one embodiment, the enteric coated drug delivery device comprises a capsule or a pill. Such drug delivery devices are known to those of skill in the art.

As used herein, a “prebiotic” refers to an ingredient that allows or promotes specific changes, both in the composition and/or activity in the gastrointestinal microbiota that may (or may not) confer benefits upon the host. In some embodiments, a prebiotic can include one or more of the following: fructooligosaccharide, galactooligosaccharides, hemicelluloses (e.g. , arabinoxylan, xylan, xyloglucan, and glucomannan), inulin, chitin, lactulose, mannan oligosaccharides, oligofructose-enriched inulin, gums (e.g. , guar gum, gum arabic and carrageenan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g. , soy fiber, sugarbeet fiber, pea fiber, corn bran, and oat fiber) and xylooligosaccharides.

As used herein, the terms “administering,” “introducing” and “transplanting” are used interchangeably in the context of the placement of cells, e.g. a microbial consortium, as described herein into a subject, by a method or route which results in at least partial localization of the introduced cells at a desired site, such as the intestines or a region thereof, such that a desired effect(s) is produced (e.g., tolerance to a food allergen). The cells can be administered by any appropriate route which results in delivery to a desired location in the subject where at least a portion of the delivered cells or components of the cells remain viable. The period of viability of the cells after administration to a subject can be as short as a few hours, e.g., twenty-four hours, to a few days, to as long as several years, i.e., long-term engraftment.

As used herein “preventing” or “prevention” refers to any methodology where the disease state does not occur due to the actions of the methodology (such as, for example, administration of a composition comprising a microbial consortium as described herein). In one aspect, it is understood that prevention can also mean that the disease is not established to the extent that occurs in untreated controls. For example, there can be a 5, 10, 15, 20, 25, 30, 35, 40, 50, 60, 70, 80, 90, or 100% reduction in the establishment of disease frequency relative to untreated controls. Accordingly, prevention of a disease encompasses a reduction in the likelihood that a subject will develop the disease, relative to an untreated subject (e.g. a subject who is not treated with a composition comprising a microbial consortium as described herein).

As used herein, the term “full complement of bile acid transformations” refers to the metabolism of primary bile acids to secondary bile acids. Bile acid transformations performed by gut microbes include deconjugation, deglucuronidation, oxidation of hydroxyl groups, reduction of oxo groups to yield epimeric hydroxyl bile acids, esterification and dehydroxylation. These reactions on bile acids are the full complement of bile acid transformations as the term is used herein.

“Synergy” or “synergistic interactions” refers to the interaction or cooperation of two or more microbes to produce a combined effect greater than the sum of their separate effects. For example, in one embodiment, “synergy” between two or more microbes can result from a first microbe secreting a waste product or metabolite that the second microbe uses to fuel growth or other processes.

As used herein, the term “persistence” refers to the maintenance of one or more members of the microbial consortium in the gastrointestinal tract at a number, biomass or activity that is at or above the threshold for treating and/or preventing food allergy. Persistence can be measured by obtaining a stool sample to determine the number, biomass, and/or activity of one or more members of the microbial consortium. In some embodiments, persistence can be measured by obtaining a ratio of the measured biomass of at least two members of the microbial consortium in the stool sample.

As used herein, the term “formulated to deliver the viable bacteria to the intestine” refers to a formulation that permits or facilitates the delivery of the bacteria in the pharmaceutical composition described herein to the intestine or small intestine in viable form. Such a formulation will protect the bacteria from the harsh acidic pH conditions of the stomach and thereby permit delivery to the intestine in viable form. Enteric coating or micro- or nano-particle formulations can facilitate such delivery as can, for example, buffer or other protective formulations.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase” or “enhance” or “activate” are all used herein to generally mean an increase of a property, level, or other parameter by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

The term “pharmaceutically acceptable” can refer to compounds and compositions which can be administered to a subject (e.g., a mammal or a human) without undue toxicity.

As used herein, the term “pharmaceutically acceptable carrier” can include any material or substance that, when combined with an active ingredient, allows the ingredient to retain biological activity and is non-reactive with the subject's immune system. Examples include, but are not limited to, any of the standard pharmaceutical carriers such as a phosphate buffered saline solution, emulsions such as oil/water emulsion, and various types of wetting agents. The term “pharmaceutically acceptable carriers” excludes tissue culture media.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

It should be understood that this invention is not limited to the particular methodologies, protocols, and reagents, etc., described herein and as such can vary therefrom. The terminology used herein is for the purpose of describing particular embodiments only, and is not intended to limit the scope of the present invention, which is defined solely by the claims.

### Microbial Flora

Each individual has a personalized gut microbiota including an estimated 500 to 5000 or more species of bacteria, fungi, viruses, archaea and other microorganisms, up to 100 trillion individual organisms, that reside in the digestive tract, providing a host of useful symbiotic functions, for example, including aiding in digestion, providing nutrition for the colon, producing vitamins, regulating the immune system, assisting in defense against exogenous bacteria, modulating energy metabolism, and the production of short chain fatty acids (SCFAs), e.g., via dietary carbohydrates, including resistant starches and dietary fiber, which are substrates for fermentation that produce SCFAs, primarily acetate, propionate, succinate, butyrate, 1,2 propanediol or 1,3 propanediol as end products.

An imbalance in the microbial flora found in and on the human body is known to be associated with a variety of disease states. For example, obesity in both humans and experimental mouse models is associated with alterations in the intestinal microbiota that appear to be pathogenic. In settings of “dysbiosis” or disrupted symbiosis, microbiota functions that can be lost or deranged, resulting in increased susceptibility to pathogens, include altered metabolic profiles, or induction of proinflammatory signals that can result in local or systemic inflammation or autoimmunity. In addition, in asthmatic subjects, both the bacterial burden and bacterial diversity were significantly higher as compared to control subjects, which were also correlated with bronchial hyper-responsiveness. Thus, the intestinal microbiota plays a significant role in the pathogenesis of many diseases and disorders, including a variety of pathogenic infections of the gut. For instance, patients become more susceptible to pathogenic infections when the normal intestinal microbiota has been disturbed due to use of broad-spectrum antibiotics. Many of these diseases and disorders are chronic conditions that significantly decrease a patient's quality of life and can be ultimately fatal.

Subdoligranulum variabile

In one embodiment, a pharmaceutical composition comprises a preparation of Subdoligranulum variabile, in an amount sufficient to treat or prevent a food allergy when administered to an individual in need thereof, and a pharmaceutically acceptable carrier. Monotherapy with Subdoligranulum variabile is shown herein to suppress food allergies in a mouse model (see e.g., Example 5).

Subdoligranulum variabile was first isolated during studies on the microflora of human feces, and described as a strictly anaerobic, non-spore-forming, Gram-negative staining organism which exhibits a somewhat variable coccus-shaped morphology. Comparative 16S ribosomal RNA gene sequencing studies showed the organism was phylogenetically a member of the Clostridium leptum supra-generic rRNA cluster and displayed a close affinity to some rDNA clones derived from human and pig feces. The nearest named relatives of the isolate corresponded to Faecalibacterium prausnitzii (formerly Fusobacterium prausnitzii) displaying a 16S rRNA sequence divergence of approximately 9%, with Anaerofilum agile and A. pentosovorans the next closest relatives of the unidentified bacterium (sequence divergence approximately 10%). Based on phenotypic and phylogenetic considerations, the unusual coccoid-shaped organism was classified as a new genus and species, Subdoligranulum variabile. The type strain of S. variabile is BI 114T, which can also be referred to as CCUG 47106T and/or DSM 15176T (see e.g., Holmstrom et al. Anaerobe 2004, 10(3): 197, 203, which is incorporated by reference herein in its entirety).

In some embodiments, the species of viable gut bacteria comprises a 16S rRNA sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 1-16.

In one aspect, described herein is a pharmaceutical composition comprising: a preparation comprising a species of viable gut bacteria, in an amount sufficient to treat or prevent a food allergy when administered to an individual in need thereof, and a pharmaceutically acceptable carrier. In some embodiments of any of the aspects, the species of viable gut bacteria is Subdoligranulum variabile. In some embodiments, the species of viable gut bacteria comprises a 16S rRNA sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 16.

In some embodiments of any of the aspects, the pharmaceutical composition is formulated to deliver the viable bacteria to the small intestine. In some embodiments of any of the aspects, the pharmaceutically acceptable carrier comprises an enteric coating composition that encapsulates the species of viable gut bacteria, wherein the enteric-coating composition is in the form of a capsule, gel, pastille, tablet or pill. In some embodiments of any of the aspects, the composition is formulated to deliver a dose of at least 5×10⁶ colony forming units per mL (CFU/mL)-2×10⁷ CFU/mL. In some embodiments of any of the aspects, the composition is formulated to deliver at least 5×10⁶ CFU/mL-2×10⁷ CFU/mL in less than 30 capsules per one time dose, and/or the composition is frozen for storage. In some embodiments of any of the aspects, the species of viable gut bacteria are encapsulated under anaerobic conditions, wherein anaerobic conditions comprise one or more of the following: (i) oxygen impermeable capsules, (ii) addition of a reducing agent including N-acetylcysteine, cysteine, or methylene blue to the composition, or (iii) use of spores for organisms that sporulate. In some embodiments of any of the aspects, the composition comprises a 16S rDNA sequence at least 97% identical to a 16S rDNA sequence present in a reference strain operational taxonomic unit for Subdoligranulum variabile. In some embodiments of any of the aspects, the enteric-coating comprises a polymer, nanoparticle, fatty acid, shellac, or a plant fiber. In some embodiments of any of the aspects, the species of viable gut bacteria is encapsulated, lyophilized, formulated in a food item, or is formulated as a liquid, gel, fluid-gel, or nanoparticles in a liquid. In some embodiments of any of the aspects, the composition further comprises a pre-biotic composition.

In some embodiments of any of the aspects, the composition comprising Subdoligranulum variabile can be administered as a monotherapy. The Subdoligranulum variabile monotherapy can be administered once or multiple times.

In some embodiments of any of the aspects, the composition comprising Subdoligranulum variabile can be administered as a combinatorial therapy together with a second species of viable gut bacteria and/or together with a microbial consortia described herein (e.g., the Clostridiales consortium and/or the Bacteroidales consortium). The combinatorial therapy, comprising a composition comprising Subdoligranulum variabile and at least one other species of viable gut bacteria and/or at least one microbial consortia described herein, can be provided as a single solution, package, pill, and/or syringe. The combinatorial therapy, comprising a composition comprising Subdoligranulum variabile and at least one other species of viable gut bacteria and/or at least one microbial consortia described herein, can be provided as at least two separate solutions, package, pill, and/or syringe. The combinatorial therapy can be administered concurrently or consecutively.

### Microbial Consortia

In one embodiment, a microbial consortium of isolated bacteria useful in the compositions and methods described herein comprises two to twenty, two to nineteen, two to eighteen, two to seventeen, two to sixteen, two to fifteen, two to fourteen, two to thirteen, two to twelve, two to eleven, two to ten, two to nine, two to eight, two to seven, two to six, two to five, two to four, or two to three species of viable gut bacteria. In another embodiment, the microbial consortium of isolated bacteria comprises no more than forty species, no more than 35 species, no more than 30 species, or no more than 25 species. In another embodiment, the microbial consortium comprises two to twenty-one species, two to twenty-two species, two to twenty-three species, two to twenty-four species, two to twenty-five species, two to twenty-six species, two to twenty-seven species, two to twenty-eight species, two to twenty-nine species, two to thirty species, two to thirty-one species, two to thirty-two species, two to thirty-three species, two to thirty-four species, two to thirty-five species, two to thirty-six species, two to thirty-seven species, two to thirty-eight species, tow to thirty-nine species or two to forty species

In some embodiments, a microbial consortium comprises at least 2, at least 3, at least 4, at least 5, at least 6, at least 7, at least 8, at least 9, at least 10, at least 11, at least 12 or more different viable, bacterial species, e.g., 15 or more, 20 or more, 25 or more, 30 or more, or even 40 species. In another embodiment, a minimal microbial consortium comprises 12 or less, 11 or less, 10 or less, 9 or less, 8 or less, 7 or less, 6 or less, 5 or less, 4 or less, or 3 or less different viable bacterial species. Also contemplated are consortia of 2 to 40 species, 4 to 30 species, 4 to 25 species, 4 to 20 species, 4 to 15 species, 4 to 11 species, 5 to 40 species, 5 to 30 species, 5 to 25 species, 5 to 20 species, 5 to 15 species, 5 to 11 species, 6 to 40 species, 6 to 30 species, 6 to 25 species, 6 to 20 species, 6 to 15 species, 6 to 11 species, 7 to 40 species, 7 to 30 species, 7 to 25 species, 7 to 20 species, 7 to 15 species, 7 to 11 species, 8 to 40 species, 8 to 30 species, 8 to 25 species, 8 to 20 species, 8 to 15 species, 8 to 11 species, 9 to 40 species, 9 to 30 species, 9 to 25 species, 9 to 20 species, 9 to 15 species, 9 to 11 species, 10 to 40 species, 10 to 30 species, 10 to 25 species, 10 to 20 species, 10 to 15 species, or 10 to 11 species.

In some embodiments of any of the aspects, a therapeutic microbial consortium for the treatment or prevention of an indication as described herein comprises Subdoligranulum variable and at least one bacterial strain identified herein as being elevated in a control group compared to a food allergy group (see e.g., Tables 2, 4, 7, or 8). In some embodiments of any of the aspects, a therapeutic microbial consortium for the treatment or prevention of an indication as described herein comprises Subdoligranulum variable and at least one bacterial strain selected from the group consisting of: Clostridium hathewayi, Clostridium nexile, Clostridium hylemonae, Clostridium glycyrrhizinilyticum, Clostridium scindens, Clostridium lavalense, Clostridium fimetarium, Clostridium symbiosum, Clostridium sporosphaeroides, Dialister proprionicifaciens, Dialister succinatiphilus, Parabacteroides distasonis, Parabacteroides goldsteinii, Parabacteroides merdae, Peptostreptococcus anaerobius, and Veilonella ratti.

In one embodiment, a therapeutic microbial consortium for the treatment or prevention of an indication as described herein comprises at least two bacterial strain(s) of viable gut bacteria selected from the group consisting of: Bacteroides fragilis, Bacteroides ovatus, Bacteroides vulgatus, Parabacteroides distasonis, and Prevotella melaninogenica. In another embodiment, the therapeutic microbial consortium for the treatment or prevention of an indication as described herein further comprises one or more, two or more, three or more, four or more, five or more, or all six of the species including: Clostridium ramosum, Clostridium scindens, Clostridium hiranonsis, Clostridium bifermentans, Clostridium leptum, and Clostridium sardiniensis.

In another embodiment, a therapeutic microbial consortium comprises at least three bacterial strain(s) of viable gut bacteria selected from the group consisting of: Bacteroides fragilis, Bacteroides ovatus, Bacteroides vulgatus, Parabacteroides distasonis, and Prevotella melaninogenica. In another embodiment, the therapeutic microbial consortium for the treatment or prevention of an indication as described herein further comprises one or more, two or more, three or more, four or more, five or more, or all six of the species including: Clostridium ramosum, Clostridium scindens, Clostridium hiranonsis, Clostridium bifermentans, Clostridium leptum, and Clostridium sardiniensis.

In another embodiment, a therapeutic microbial consortium comprises at least four bacterial strain(s) of viable gut bacteria selected from the group consisting of: Bacteroides fragilis, Bacteroides ovatus, Bacteroides vulgatus, Parabacteroides distasonis, and Prevotella melaninogenica. In another embodiment, the therapeutic microbial consortium for the treatment or prevention of an indication as described herein further comprises one or more, two or more, three or more, four or more, five or more, or all six of the species including: Clostridium ramosum, Clostridium scindens, Clostridium hiranonsis, Clostridium bifermentans, Clostridium leptum, and Clostridium sardiniensis.

In another embodiment, a therapeutic microbial consortium comprises each of the bacterial strain(s) of viable gut bacteria selected from the group consisting of: Bacteroides fragilis, Bacteroides ovatus, Bacteroides vulgatus, Parabacteroides distasonis, and Prevotella melaninogenica. In another embodiment, the therapeutic microbial consortium for the treatment or prevention of an indication as described herein further comprises one or more, two or more, three or more, four or more, five or more, or all six of the species including: Clostridium ramosum, Clostridium scindens, Clostridium hiranonsis, Clostridium bifermentans, Clostridium leptum, and Clostridium sardiniensis.

In another embodiment, the microbial consortium comprises at least two species of viable gut bacteria are selected from the group consisting of: Bacteroides fragilis, Bacteroides ovatus, Bacteroides vulgatus, Parabacteroides distasonis, Prevotella melaninogenica, and the microbial consortium further comprises one or more, two or more, three or more, four or more, five or more, or all six of Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides ovatus. In another embodiment, the microbial consortium further comprises at least one of the group consisting of: Bacteroides vulgatus, Parabacteroides distasonis, and Prevotella melaninogenica.

In another embodiment, the microbial consortium comprises Bacteroides fragilis and Bacteroides ovatus. In another embodiment, the microbial consortium further comprises at least two of the group consisting of: Bacteroides vulgatus, Parabacteroides distasonis, and Prevotella melaninogenica.

In another embodiment, the microbial consortium comprises Bacteroides fragilis and Bacteroides ovatus, and the microbial consortium further comprises each of the group consisting of: Bacteroides vulgatus, Parabacteroides distasonis, and Prevotella melaninogenica.

In another embodiment, the microbial consortium comprises Bacteroides fragilis and Bacteroides ovatus, and at least one of the group consisting of: Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides ovatus, and at least two of the group consisting of: Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides ovatus, and at least three of the group consisting of: Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides ovatus, and at least four of the group consisting of: Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides ovatus, and at least five of the group consisting of: Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides ovatus, and each of the group consisting of Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides vulgatus and at least one of the group consisting of: Bacteroides ovatus, Parabacteroides distasonis, and Prevotella melaninogenica.

In another embodiment, the microbial consortium comprises Bacteroides fragilis and Bacteroides vulgatus and at least two of the group consisting of: Bacteroides ovatus, Parabacteroides distasonis, and Prevotella melaninogenica.

In another embodiment, the microbial consortium comprises Bacteroides fragilis and Bacteroides vulgatus and further comprises Bacteroides ovatus, Parabacteroides distasonis, and Prevotella melaninogenica.

In another embodiment, the microbial consortium comprises Bacteroides fragilis and Bacteroides vulgatus and at least one of the group consisting of: Clostridium ramosum, Clostridium scindens, Clostridium hiranonsis, Clostridium bifermentans, Clostridium leptum, and Clostridium sardiniensis.

In another embodiment, the microbial consortium further comprises Bacteroides fragilis and Bacteroides vulgatus and at least two of the group consisting of: Clostridium ramosum, Clostridium scindens, Clostridium hiranonsis, Clostridium bifermentans, Clostridium leptum, and Clostridium sardiniensis.

In another embodiment, the microbial consortium further comprises Bacteroides fragilis and Bacteroides vulgatus and at least three of the group consisting of: Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides vulgatus and at least four of the group consisting of: Clostridium ramosum, Clostridium scindens, Clostridium hiranonsis, Clostridium bifermentans, Clostridium leptum, and Clostridium sardiniensis.

In another embodiment, the microbial consortium further comprises Bacteroides fragilis and Bacteroides vulgatus and at least five of the group consisting of: Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides vulgatus and further comprises Clostridium ramosum, Clostridium scindens, Clostridium hiranonsis, Clostridium bifermentans, Clostridium leptum, and Clostridium sardiniensis.

Bacterial species or bacterial strains in consortia or compositions described herein are not pathogenic in the human gut.

In one embodiment, the species of viable gut bacteria do not include Escherichia coli, Klebsiella pneumoniae, Proteus mirabilis, Enterobacter cloacae, Bilophila wadsworthia, Alistipes onderdonkii, Desulfovibrio species, Lactobacillus johnsoni, and Parasutterella excrementihominis.

In another embodiment, the consortium does not comprise bacteria of the Genera Bilophila, Enterobacter, Escherichia, Klebsiella, Proteus, Alistipes, Desulfovibrio, Blautia, or Parasutterella.

In another embodiment, the consortium does not comprise bacteria of the Families Desulfovibrionaceae, Enterobacteriaceae, Rikenellaceae, and Sutterellaceae.

In another embodiment, the consortium does not comprise bacteria of the Families Lactobacillaceae or Enterbacteriaceae.

In another embodiment, the consortium does not comprise bacteria of the Order Burkholdales, Desulfovibrionates, or Enterobacteriales.

Metabolic Features: Various features of gut microbes are beneficial for protection from or therapy for allergy, including food allergy. In the following, features and corresponding functions contemplated to render particular species or taxa of gut microbes well-suited for a protective or therapeutic microbial consortium as described herein are described. In practice, a consortium comprising four or more, e.g., five or more, six or more, seven or more, eight or more, nine or more or ten or more of these features and corresponding functions is considered a likely candidate for protection or therapy for food allergy.

In some embodiments, the microbial consortium comprises one or more types of microbes capable of producing butyrate in a mammalian subject. Butyrate-producing microbes can be identified experimentally, e.g., by NMR or gas chromatography analyses of microbial products or colorimetric assays (Rose I A. 1955. Methods Enzymol. 1: 591-5). Butyrate-producing microbes can also be identified computationally, e.g., by the identification of one or more enzymes involved in butyrate synthesis. Non-limiting examples of enzymes found in butyrate-producing microbes include butyrate kinase, phosphotransbutyrylase, and butyryl CoA:acetate CoA transferase (Louis P., et al. 2004. J Bact 186(7): 2099-2106). Butyrate-producing species include, but are not limited to, Clostridium sardiniensis, Clostridium hiranonsis, Facealibacterium prausnitzii, Butyrovibrio spp., Eubacterium rectale, and Roseburia intestinalis.

In some embodiments, a pharmaceutical composition comprises one or more types of microbes or bacterial species, wherein the at least two types of microbes are capable of producing butyrate in a mammalian subject. In other embodiments, the composition comprises two or more types of microbes, that cooperate (i.e., cross-feed) to produce an immunomodulatory short chain fatty acid (SCFA) (e.g., butyrate) in a mammalian subject. In one embodiment, the composition comprises at least one type of microbe (e.g., Bifidobacterium spp., Bacteroides vulgatus, Bacteroides fragilis or Clostridium ramosum) capable of metabolizing a prebiotic, including but not limited to, inulin, inulin-type fructans, fucose-containing glycoconjugates including the H1, H2, Lewis A, B, X, or Y antigens, or oligofructose, such that the resulting metabolic product can be converted by a second type of microbe (e.g., a butyrate-producing microbe such as Roseburia spp.) to an immunomodulatory SCFA such as butyrate (Falony G., ET al. 2006 Appl. Environ. Microbiol. 72(12): 7835-7841). In other aspects, the composition can comprise at least one acetate-consuming, butyrate-producing microbe (e.g., Faecalibacterium prausnitzii or Roseburia intestinalis).

In some embodiments, the composition comprises one or more types of microbe capable of producing propionate and/or succinate in a mammalian subject, optionally further comprising a prebiotic or substrate appropriate for propionate and/or succinate biosynthesis. Examples of prebiotics or substrates used for the production of propionate include, but are not limited to, L-rhamnose, D-tagalose, resistant starch, inulin, polydextrose, arabinoxylans, arabinoxylan oligosaccharides, mannooligosaccharides, and laminarans (Hosseini E., et al. 2011. Nutrition Reviews. 69(5): 245-258). Propionate-producing microbes can be identified experimentally, such as by NMR or gas chromatography analyses of microbial products or colorimetric assays (Rose I A. 1955. Methods Enzymol. 1: 591-5). Propionate-producing microbes can also be identified computationally, such as by the identification of one or more enzymes involved in propionate synthesis. Non-limiting examples of enzymes found in propionate-producing microbes include enzymes of the succinate pathway, including but not limited to phosphoenylpyruvate carboxykinase, pyruvate kinase, pyruvate carboxylase, malate dehydrogenase, fumarate hydratase, succinate dehydrogenase, succinyl CoA synthetase, methylmalonyl Coa decarboxylase, and propionate CoA transferase, as well as enzymes of the acrylate pathway, including but not limited to L-lactate dehydrogenase, propionate CoA transferase, lactoyl CoA dehydratase, acyl CoA dehydrogenase, phosphate acetyltransferase, and propionate kinase. For example, microbes that utilize the succinate pathway include certain species of the Bacteroides genus, such as Bacteroides fragilis, Clostridium sardiniensis and Clostridum hiranonsis. In one embodiment, the propionate-producing species is Bacteroides fragilis, Bacteroides thetaiotaomicron, or Bacteroides ovatus. In one embodiment, the succinate-producing species is Bacteroides fragilis, Bacteroides thetaiotaomicron, Bacteroides vulgatus, or Bacteroides ovatus.

Functional methods to define species that produce butyrate, propionate and/or succinate includes analysis of short-chain fatty acid (SCFA) production using gas-chromatography/liquid chromatography (GC/LC) to identify propionate, butyrate, and/or succinate or mass spectroscopy based methods to detect these SCFA, as well as 1,2-propanediol, and 1,3-propanediol. Studies can be performed in cultured supernatants from colonized gnotobiotic mice and from conventional patients and/or animal samples.

Additional methods for identifying species that produce butyrate comprise those species expressing butyryl-CoA:acetate CoA transferases (But genes) or butyrate kinases (Buk genes) for production of butyrate from anaerobic fermentation of sugars. In another embodiment, organisms producing butyrate (from amino acids such as lysine, glutarate or 4-aminobutyrate pathways) express enzymes including e.g., L2Hgdh, 2-hydroxyglutarate dehydrogenase; Gct, glutaconate CoA transferase (α, β subunits); HgCoAd, 2-hydroxy-glutaryl-CoA dehydrogenase (α, β, γ subunits); Gcd, glutaconyl-CoA decarboxylase (α, β subunits); Th1, thiolase; hbd, β-hydroxybutyryl-CoA dehydrogenase; Cro, crotonase; Bcd, butyryl-CoA dehydrogenase (including electron transfer protein α, β subunits); KamA, lysine-2,3-aminomutase; KamD,E, β-lysine-5,6-aminomutase (α, β subunits); Kdd, 3,5-diaminohexanoate dehydrogenase; Kce, 3-keto-5-aminohexanoate cleavage enzyme; Kal, 3-aminobutyryl-CoA ammonia lyase; AbfH, 4-hydroxybutyrate dehydrogenase; AbfD, 4-hydroxybutyryl-CoA dehydratase; Isom, vinylacetyl-CoA 3,2-isomerase (same protein as AbfD): 4Hbt, butyryl-CoA:4-hydroxybutyrate CoA transferase; But, butyryl-CoA:acetate CoA transferase; Ato, butyryl-CoA:acetoacetate CoA transferase (α, β subunits); Ptb, phosphate butyryltransferase; Buk, and butyrate kinase (see e.g., Vital et al. mBIO 5(2):e00889-14).

In some embodiments, a microbial consortium comprises at least one bacterial species that produces compounds capable of stimulating the aryl hydrocarbon (AhR) receptor in gut epithelial cells, antigen-presenting cells and/or T cells. Without wishing to be bound by theory, stimulation of the AhR receptor can aid in the development of regulatory T cell processes that can prevent and/or treat food allergy. Some non-limiting examples of compounds that stimulate host aryl hydrocarbon receptor pathways include (i) indole, (ii) intermediates from microbial synthesis of indole, tryptophan, tyrosine and histidine, (iii) microbial synthesis of flavonoids, phenazines and/or quinones or (iv) compounds or intermediates of metabolism of host ingested flavonoids, phenazines and/or quinones. In one example, a viable, culturable, anaerobic gut bacterial strain produces aryl hydrocarbon receptor agonists sufficient to stimulate host aryl hydrocarbon receptor pathways comprises at least one gene associated with the synthesis of tryptophan or the synthesis of quinone molecules. In an additional example, a viable, culturable, anaerobic gut bacterial strain that produces aryl hydrocarbon receptor agonists sufficient to stimulate host aryl hydrocarbon receptor pathways by microbial synthesis of flavonoids, phenazines, and/or quinones. Thus microbes that express or encode biosynthetic enzymes that participate in the synthesis of flavonoids, phenazines and/or quinones are identified as microbes that produce host aryl hydrocarbon receptor agonists. In one embodiment, the biosynthetic enzymes include the last enzyme in the pathway that catalyzes the final biosynthetic reaction producing e.g., flavonoids, phenazine or quinone compounds.

In some embodiments, a microbial consortium comprises at least one bacterial species that produces compounds capable of stimulating the pregnane X receptor that e.g., has beneficial effects on gut barrier function and/or the development of regulatory T cell processes. Non-limiting examples of compounds that stimulate the pregnane X receptor include (i) desmolase, (ii) compounds or intermediates of hydroxysteroid dehydrogenase activity, or (iii) compounds or intermediates derived from flavonoid metabolism enzymes. Thus, bacteria that encode and express steroid desmolase and/or hydroxysteroid dehydrogenase enzymes are expected to produce compounds that stimulate the pregnane X receptor. Clostridium sardiniensis and Clostridium scindens are non-limiting examples of bacterial species that produce compounds capable of stimulating the pregnane X receptor.

In some embodiments, the microbial consortium comprises at least one bacterial species that produces compounds capable of stimulating the RAR-related orphan receptor gamma (RORgamma) pathways, for example, to stimulate development of regulatory T cell responses via direct stimulation of RORgamma-activated pathways in gut antigen presenting cells and/or epithelial cells that then stimulate regulatory T cell responses. In one embodiment, the viable, culturable, anaerobic gut bacterial strain that produces compounds endogenously or by metabolizing ingested precursors, that is capable of stimulating the RORgamma (RAR-related orphan receptor gamma) pathways to stimulate development of regulatory T cell responses is a strain that expresses at least one cholesterol reductase and other enzymes capable of metabolizing sterol compounds. Non-limiting examples of microbes that produce compounds that stimulate the RORgamma pathway include Clostridium scindens, Clostridia hiranonsis, and Clostridium sardiniensis. In one instance, those species express bile acid transforming enzymes that can also produce RORgamma pathway agonists.

In some embodiments, a microbial consortium described herein improves gut function, for example, by stimulating host mucins and complex glycoconjugates and improving colonization by protective commensal species. In one embodiment, the microbial consortium comprises at least one bacterial species, such as Bacteroides vulgatus, that stimulates production of mucins and complex glycoconjugates by the host.

Immunomodulation: Other exemplary compositions useful for treatment of food allergy contain bacterial species capable of altering the proportion of immune subpopulations, e.g., T cell subpopulations, e.g., Tregs in the subject.

For example, immunomodulatory bacteria can increase or decrease the proportion of Treg cells, Th17 cells, Th1 cells, or Th2 cells in a subject. The increase or decrease in the proportion of immune cell subpopulations can be systemic, or it can be localized to a site of action of the colonized consortium, e.g., in the gastrointestinal tract or at the site of a distal dysbiosis. In some embodiments, a microbial consortium comprising immunomodulatory bacteria is used for treatment of food allergy based on the desired effect of the probiotic composition on the differentiation and/or expansion of subpopulations of immune cells in the subject.

In one embodiment, the microbial consortium contains immunomodulatory bacteria that increase the proportion of Treg cells in a subject or in a particular location in a subject, e.g., the gut tissues. In one embodiment, a microbial consortium contains immunomodulatory bacteria that increase the proportion of Th17 cells in a subject. In another embodiment, a microbial consortium contains immunomodulatory bacteria that decrease the proportion of Th17 cells in a subject. In one embodiment, a microbial consortium contains immunomodulatory bacteria that increase the proportion of Th1 cells in a subject. In another embodiment, a microbial consortium contains immunomodulatory bacteria that decrease the proportion of Th1 cells in a subject. In one embodiment, a microbial consortium contains immunomodulatory bacteria that increase the proportion of Th2 cells in a subject. In another embodiment, a microbial consortium contains immunomodulatory bacteria that decrease the proportion of Th2 cells in a subject.

In one embodiment, a microbial consortium contains immunomodulatory bacteria capable of modulating the proportion of one or more of Treg cells, Th17 cells, Th1 cells, Th2 cells, and combinations thereof in a subject. Certain immune cell profiles can be particularly desirable to treat or prevent inflammatory disorders, such as food allergies. For example, in some embodiments, treatment or prevention of e.g., food allergy can be promoted by increasing numbers of Treg cells and Th2 cells, and decreasing numbers of Th17 cells and Th1 cells. Accordingly, a microbial consortium for the treatment or prevention of food allergy can contain a microbial consortium capable of promoting Treg cells and Th2 cells, and reducing Th17 and Th1 cells.

In one embodiment, the anaerobic gut bacterial strain in the methods and compositions described herein express agonists capable of binding to and modulating responses mediated by Toll-like receptors (TLR), CD14 and/or lipid binding proteins in antigen presenting cells, gut epithelial cells and/or T cells to promote the development of regulatory T cells. Non-limiting examples of TLR agonists include lipopolysaccharide (LPS), exopolysaccharides (PSA), peptidoglycan or CpG motifs produced by commensal members of Bacteroides, or lipoteichoic acids (LTA) produced by members of Clostridium. In one embodiment, an anaerobic gut bacterial strain that acts as a TLR agonist is selected from the following Table.

| Family | Genus | Species |
|---|---|---|
| Clostridieaceae | Clostridium, Hungatella | Hungatella hathawayi |
| Eubacteriaceae | Eubacterium | Eubacterium rectale |
| Erysipelotrichaceae | Erysipelatoclostridium | Erysipelatoclostridium |
| | (formerly species in | ramosum (Clostridium |
| | genus Clostridium) | ramosum) |
| Lachnospiraceae | Blautia, Butyrovibrio, | Butyrovibrio crossatus, |
| | Cellulosyliticum, | Roseburia intestinalis, |
| | Clostridium cluster | Clostridium scindens, |
| | XIVa species, | Clostridium hylemonae, |
| | Coprococcus, Dorea, | Clostridium symbiosum |
| | Lachnospira, | |
| | Robinsonella, | |
| | Roseburia, | |
| Ruminococcaceae | Faecalobacterium, | Faecalibacterium |
| | Ruminococcus, | prausnitzii, |
| | Subdoligranulum, | Subdoligranulum |
| | Clostridium cluster | variabile |
| | XIVa species | |
| Bacteroidaceae | Bacteroides | Bacteroides |
| | | thetaiotaomicron, |
| | | Bacteroides fragilis, |
| | | Bacteroides ovatus |
| Prophyromonadaceae | Parabacteroides, | Parabacteroides |
| | Porphyromonas, | goldsteinii, |
| | Tannerella | Parabacteroides |
| | | merdae, |
| | | Parabacteroides |
| | | distasonis |
| Prevotellaceae | Prevotella | Prevotella tannerae |

Bile Acid Transformation: Primary bile acids (e.g., cholic and chenodeoxycholic acids in humans) are generated in the liver of mammals, including humans, mainly by conjugation with the amino acids taurine or glycine, and are secreted in bile. In the intestinal tract, primary bile acids are metabolized by microbes that transform the primary bile acids to secondary bile acids. Intestinal microbial transformation of primary bile acids can include deconjugation, deglucuronidation, oxidation of hydroxyl groups, reduction of oxo groups to yield epimeric hydroxyl bile acids, esterification, and dehydroxylation. Non-limiting examples of bacteria that perform deconjugation of primary bile acids include Bacteroides, Bifidobacterium, Clostridium, and Lactobacillus. Non-limiting examples of bacteria that perform oxidation and epimerization of primary bile acids include Bacteroides, Clostridium, Egghertella, Eubacterium, Peptostreptococcus, and Ruminococcus. Non-limiting examples of bacteria that perform 7-dehydroxylation of primary bile acids include Clostridium, and Eubacterium. Non-limiting examples of bacteria that perform esterification of primary bile acids include Bacteroides, Eubacterium, and Lactobacillus.

In one embodiment, a microbial consortium as described herein comprises at least one bacterial constituent that transforms bile acids by deconjugation. In another embodiment, a microbial consortium as described herein comprises at least one bacterial constituent that transforms bile acids by 7-dehydroxylation. In another embodiment, a microbial consortium as described herein comprises at least one bacterial constituent that transforms bile acids by esterification.

In one embodiment, a microbial consortium as described herein comprises at least 2, at least 3, at least 4, at least 5, at least 6, at least 7, at least 8, at least 9, at least 10, at least 11 or more bacterial constituents that perform bile acid transformation.

In one embodiment, a microbial consortium as described herein comprises 11 or fewer, 10 or fewer, 9 or fewer, 8 or fewer, 7 or fewer, 6 or fewer, 5 or fewer, 4 or fewer, 3 or fewer, 2 or fewer 1 or fewer or zero bacterial constituents that perform bile acid transformation, such as deconjugation, esterification or 7-dehydroxylation.

In one embodiment, a microbial consortium comprises at least one anaerobic gut bacterial strain that alone, or in combination, performs the full complement of bile acid transformations.

Targets of GP-IIa consortium members: The microbial consortium as described herein has multiple targets within the host subject. Representative targets are summarized in the following Table.

Targets of GP-IIa consortium members

| | B. | | | | |
|---|---|---|---|---|---|
| Host Target | fragilis | B. ovatus | B. vulgatus | P. distasonis | P. melaninogenica |
| Bile salt | + | + | + | + | + |
| transformation | | | | | |
| Mucin layer and | + | + | + | Unknown | Unknown |
| glycoconjugate | | | | | |
| modulation | | | | | |
| Immunostimulatory | + | + | + | + | + |
| Treg induction | + | + | + | + | Unknown |

Engineered microbes: In some embodiments, one or more members of the microbial consortium comprises an engineered microbe(s). For example, engineered microbes include microbes harboring i) one or more introduced genetic changes, such change being an insertion, deletion, translocation, or substitution, or any combination thereof, of one or more nucleotides contained on the bacterial chromosome or on an endogenous plasmid, wherein the genetic change can result in the alteration, disruption, removal, or addition of one or more protein coding genes, non-protein-coding genes, gene regulatory regions, or any combination thereof, and wherein such change can be a fusion of two or more separate genomic regions or can be synthetically derived; ii) one or more foreign plasmids containing a mutant copy of an endogenous gene, such mutation being an insertion, deletion, or substitution, or any combination thereof, of one or more nucleotides; and iii) one or more foreign plasmids containing a mutant or non-mutant exogenous gene or a fusion of two or more endogenous, exogenous, or mixed genes. The engineered microbe(s) can be produced using techniques including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, or any combination thereof.

Excluded Bacteria: In one embodiment, a microbial consortium does not include an organism conventionally classified as a pathogenic or opportunistic organism. It is possible that a function shared by all members of a given taxonomic group could be beneficial, e.g., for providing particular metabolites, yet for other reasons the overall effect of one or more particular members of the group is not beneficial and is, for example, pathogenic. Clearly, members of a given taxonomic group that cause pathogenesis, e.g., acute gastrointestinal pathologies, are to be excluded from the therapeutic or preventive methods and compositions described herein.

In one embodiment, the bacterial composition does not comprise at least one of: Acidaminococcus intestinalis, Escherichia coli, Lactobacillus casei, Lactobacillus paracasei, Raoultella sp., and Streptococcus mitis.

In another embodiment, the bacterial composition does not comprise at least one of Bamesiella intestinihominis; Lactobacillus reuteri; Enterococcus hirae, Enterococus faecium, or Enterococcus durans; Anaerostipes caccae or Clostridium indolis; Staphylococcus wameri or Staphylococcus pasteuri; and Adlercreutzia equolifaciens.

In another embodiment, the bacterial composition does not comprise at least one of Clostridium botulinum, Clostridium cadaveris, Clostridium chauvoei, Clostridium clostridioforme, Clostridium cochlearium, Clostridium difficile, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium septicum, Clostridium sordellii, Clostridium sphenoides, and Clostridium tetani.

In another embodiment, the bacterial composition does not comprise at least one of Escherichia coli, and Lactobacillus johnsonii.

In another embodiment, the bacterial composition does not comprise at least one of Clostridium innocuum, Clostridium butyricum, Escherichia coli, and Blautia producta (previously known as Peptostreptococcus productus).

In another embodiment, the bacterial composition does not comprise at least one of Eubacteria, Fusobacteria, Propionibacteria, Escherichia coli, and Gemmiger.

In another embodiment, the compositions described herein do not comprise pathogenic bacteria such as e.g., Yersinia, Vibrio, Treponema, Streptococcus, Staphylococcus, Shigella, Salmonella, Rickettsia, Orientia, Pseudomonas, Neisseria, Mycoplasma, Mycobacterium, Listeria, Leptospira, Legionella, Klebsiella, Helicobacter, Haemophilus, Francisella, Escherichia, Ehrlichia, Enterococcus, Coxiella, Corynebacterium, Chlamydia, Chlamydophila, Campylobacter, Burkholderia, Brucella, Borrelia, Bordetella, Bacillus, multi-drug resistant bacteria, extended spectrum beta-lactam resistant Enterococci (ESBL), Carbapenem-resistant Enterobacteriaceae (CRE), and vancomycin-resistant Enterococci (VRE).

In other embodiments, the compositions described herein do not comprise pathogenic species or species, such as Aeromonas hydrophila, Campylobacter fetus, Plesiomonas shigelloides, Bacillus cereus, Campylobacter jejuni, Clostridium botulinum, Clostridium difficile, Clostridium perfringens, enteroaggregative Escherichia coli, enterohemorrhagic Escherichia coli, enteroinvasive Escherichia coli, enterotoxigenic Escherichia coli (such as, but not limited to, LT and/or ST), Escherichia coli O157:H7, Helicobacter pylori, Klebsiellia pneumonia, Lysteria monocytogenes, Plesiomonas shigelloides, Salmonella spp., Salmonella typhi, Salmonella paratyphi, Shigella spp., Staphylococcus spp., Staphylococcus aureus, vancomycin-resistant enterococcus spp., Vibrio spp., Vibrio cholerae, Vibrio parahaemolyticus, Vibrio vulnificus, and Yersinia enterocolitica.

In one embodiment, the microbial consortia and compositions thereof do not comprise Escherichia coli, Klebsiella pneumoniae, Proteus mirabilis, Enterobacter cloacae, and/or Bilophila wadsworthia.

Reduction of pathogenic organisms: In some embodiments, compositions comprising a microbial consortium as described herein offer a protective or therapeutic effect against dysbiosis or against infection by one or more GI pathogens of interest. In one embodiment, a microbial consortium as described herein reduces the biomass of one or more dysbiotic or pathogenic bacterial species or strains.

In one embodiment, a microbial consortium as described herein decreases the biomass of one or more dysbiotic or pathogenic bacterial species or strains by at least 10% compared to the biomass of the one or more dysbiotic or pathogenic bacterial species or strains in the absence of treatment with such microbial consortium. In other embodiments the biomass of one or more pathogenic bacterial species or strains is decreased by at least 20%, at least 30%, at least 40%, at least 50%, at least 60%, at least 70%, at least 80%, at least 90%, at least 95%, at least 99%, or even 100% (i.e., below detectable limits of the assay) as compared to the biomass of the dysbiotic or pathogenic bacterial species or strains in the gut of the subject prior to treatment with the microbial consortium or compositions thereof.

In some embodiments, a microbial consortium as described herein alters the gut environment such that the number, biomass, or activity of one or more dysbiotic or pathogenic organisms is decreased by at least 10% (e.g., at least 20%, at least 30%, at least 40%, at least 50%, at least 60%, at least 70%, at least 80%, at least 90%, at least 95%, at least 99%, or even 100% (i.e., below detectable limits of the assay)). As but one example, colonization of Bacteroides reduces the biomass of dysbiotic species in the Enterobacteriaceae or Desulfonovibriacaea Families.

In some embodiments, the pathogenic bacterium is selected from the group consisting of Yersinia, Vibrio, Treponema, Streptococcus, Staphylococcus, Escherichia/Shigella, Salmonella, Rickettsia, Orientia, Pseudomonas, Neisseria, Mycoplasma, Mycobacterium, Listeria, Leptospira, Legionella, Klebsiella, Helicobacter, Haemophilus, Francisella, Escherichia, Ehrlichia, Enterococcus, Coxiella, Corynebacterium, Clostridium, Chlamydia, Chlamydophila, Campylobacter, Burkholderia, Brucella, Borrelia, Bordetella, Bifidobacterium, Bacillus, Bilophila, Desulfovibrio, multi-drug resistant bacteria, extended spectrum beta-lactam resistant Enterococci (ESBL), Carbapenem-resistant Enterobacteriaceae (CRE), and vancomycin-resistant Enterococci (VRE).

In some embodiments, these pathogens include, but are not limited to, Aeromonas hydrophila, Campylobacter fetus, Plesiomonas shigelloides, Bacillus cereus, Campylobacter jejuni, Clostridium botulinum, Clostridium difficile, Clostridium perfringens, enteroaggregative Escherichia coli, entero hemorrhagic Escherichia coli, enteroinvasive Escherichia coli, enterotoxigenic Escherichia coli (such as, but not limited to, LT and/or ST), Escherichia coli 0157:H7, Helicobacter pylori, Klebsiellia pneumonia, Lysteria monocytogenes, Plesiomonas shigelloides, Salmonella spp., Salmonella typhi, Salmonella paratyphi, Shigella spp., Staphylococcus spp., Staphylococcus aureus, vancomycin-resistant enterococcus spp., Vibrio spp., Vibrio cholerae, Vibrio parahaemolyticus, Vibrio vulnificus, and Yersinia enterocolitica.

In one embodiment, the pathogen of interest is at least one pathogen chosen from Clostridium difficile, Salmonella spp., pathogenic Escherichia coli, vancomycin-resistant Enterococcus spp., and extended spectrum beta-lactam resistant Enterococci (ESBL).

Methods for testing the efficacy of the compositions comprising a microbial composition to reduce the number, biomass, or activity of one or more dysbiotic or pathogenic organisms are discussed in the following. While certain of the methods are described in the following in terms of assaying reduced number, biomass or activity of Clostridium difficile, one of skill in the art can readily adapt the methods to measure the number, biomass or activity of one or more further microbial species or strains.

In one embodiment, provided is an in Vitro Assay utilizing competition between the bacterial compositions or subsets thereof and Clostridium difficile or other dysbiotic or pathogenic strain. This test in known in the art and as such is not described in detail herein.

In another embodiment, provided is an In Vitro Assay utilizing 10% (wt/vol) Sterile-Filtered Feces. This assay tests for the protective effect of the bacterial compositions and screens in vitro for combinations of microbes that inhibit the growth of a given pathogenic or dysbiotic microbe. The assay can operate in automated high-throughput or manual modes. Under either system, human or animal feces can be re-suspended in an anaerobic buffer solution, such as pre-reduced PBS or other suitable buffer, the particulate removed by centrifugation, and filter sterilized. This 10% sterile-filtered feces material serves as the base media for the in vitro assay. To test a bacterial composition, an investigator can add it to the sterile-filtered feces material for a first incubation period and then can inoculate the incubated microbial solution with a pathogenic or dysbiotic microbe of interest for a second incubation period. The resulting titer of the pathogenic or dysbiotic microbe is quantified by any number of methods such as those described below, and the change in the amount of pathogen is compared to standard controls including the pathogenic or dysbiotic microbe cultivated in the absence of the bacterial composition. The assay is conducted using at least one control. Feces from a healthy subject can be used as a positive control. As a negative control, antibiotic-treated feces or heat-treated feces can be used. Various bacterial compositions can be tested in this material and the bacterial compositions optionally compared to the positive and/or negative controls. The ability to inhibit the growth of a pathogenic or dysbiotic microbe can be measured by plating the incubated material on selective media and counting colonies. After competition between the bacterial composition and the pathogenic or dysbiotic microbe, each well of the in vitro assay plate is serially diluted ten-fold six times, and plated on selective media. For Clostridium difficile this would include, for example, cycloserine cefoxitin mannitol agar (CCMA) or cycloserine cefoxitin fructose agar (CCFA), and incubated. Colonies of the pathogenic or dysbiotic microbes are then counted to calculate the concentration of viable cells in each well at the end of the competition.

Alternatively, the ability to inhibit the growth of a pathogenic or dysbiotic species can be measured by quantitative PCR (qPCR). Standard techniques can be followed to generate a standard curve for the pathogenic or dysbiotic strain of interest. Genomic DNA can be extracted from samples using commercially-available kits, such as the Mo Bio Powersoil®-htp 96 Well Soil DNA Isolation Kit (Mo Bio Laboratories, Carlsbad, Calif.), the Mo Bio Powersoil® DNA Isolation Kit (Mo Bio Laboratories, Carlsbad, Calif.), or the QIAamp DNA Stool Mini Kit (QIAGEN, Valencia, Calif.) according to the manufacturer's instructions. The qPCR can be conducted using HotMasterMix (5PRIME, Gaithersburg, Md.) and primers specific for the pathogenic or dysbiotic microbe of interest, and can be conducted on a MicroAmp® Fast Optical 96-well Reaction Plate with Barcode (0.1 mL) (Life Technologies, Grand Island, N.Y.) and performed on a BioRad C1000™ Thermal Cycler equipped with a CFX96™ Real-Time System (BioRad, Hercules, Calif.), with fluorescent readings of the FAM and ROX channels. The Cq value for each well on the FAM channel is determined by the CFX Manager™ software version 2.1. The log₁₀ (cfu/ml) of each experimental sample is calculated by inputting a given sample's Cq value into linear regression model generated from the standard curve comparing the Cq values of the standard curve wells to the known log₁₀ (cfu/ml) of those samples. The skilled artisan can employ alternative qPCR modes.

Also provided are In Vivo Assays establishing the protective effect of bacterial compositions. The assay is described in terms of protective effect against Clostridium difficile, but can be adapted by one of skill in the art for other pathogens or dysbiotic species. Provided is an in vivo mouse model to test for the protective effect of the bacterial compositions against Clostridium difficile. In this model (based on Chen, et al., Gastroenterology 135(6):1984-1992 (2008)), mice are made susceptible to Clostridium difficile by a 7 day treatment (days −12 to −5 of experiment) with 5 to 7 antibiotics (including kanamycin, colistin, gentamycin, metronidazole and vancomycin and optionally including ampicillin and ciprofloxacin) delivered via their drinking water, followed by a single dose with Clindamycin on day −3, then challenged three days later on day 0 with 10⁴ spores of Clostridium difficile via oral gavage (i.e., oro-gastric lavage). Bacterial compositions can be given either before (prophylactic treatment) or after (therapeutic treatment) Clostridium difficile gavage. Further, bacterial compositions can be given after (optional) vancomycin treatment to assess their ability to prevent recurrence and thus suppress the pathogen in vivo. The outcomes assessed each day from day −1 to day 6 (or beyond, for prevention of recurrence) are weight, clinical signs, mortality and shedding of Clostridium difficile in the feces. Weight loss, clinical signs of disease and Clostridium difficile shedding are typically observed without treatment. Vancomycin provided by oral gavage on days −1 to 4 protects against these outcomes and serves as a positive control. Clinical signs are subjective, and scored each day by the same experienced observer. Animals that lose greater than or equal to 25% of their body weight are euthanized and counted as infection-related mortalities. Feces are gathered from mouse cages (5 mice per cage) each day, and the shedding of Clostridium difficile spores is detected in the feces using a selective plating assay as described for the in vitro assay above, or via qPCR for the toxin gene. The effects of test materials including 10% suspension of human feces (as a positive control), bacterial compositions, or PBS (as a negative vehicle control), are determined by introducing the test article in a 0.2 mL volume into the mice via oral gavage on day −1, one day prior to Clostridium difficile challenge, on day 1, 2 and 3 as treatment or post-vancomycin treatment on days 5, 6, 7 and 8. Vancomycin, as discussed above, is given on days 1 to 4 as another positive control. Alternative dosing schedules and routes of administration (e.g. rectal) may be employed, including multiple doses of test article, and 10³ to 10¹³ of a given organism or composition may be delivered.

Enhancement of beneficial organisms: In some embodiments, compositions comprising a microbial consortium offer a therapeutic effect of enhancing beneficial organisms in the GI tract. In one embodiment, a microbial consortium as described herein increases the biomass of one or more beneficial bacterial species by at least 10%. In other embodiments the biomass of one or more beneficial bacterial species is increased by at least 20%, at least 30%, at least 40%, at least 50%, at least 60%, at least 70%, at least 80%, at least 90%, at least 1-fold, at least 2-fold, at least 5-fold, at least 10-fold, at least 50-fold, at least 100-fold, at least 500-fold, at least 1000-fold, at least 5000-fold, at least 10,000-fold, at least 15,000-fold or at least 20,000-fold over the biomass of the beneficial bacterial species in the gut of the subject prior to treatment with the microbial consortium or compositions thereof. In one embodiment, the beneficial organisms are commensal bacterial species that currently reside or exist in the gut. In another embodiment, the beneficial organisms are one or more of the bacterial species in the microbial consortium itself.

In some embodiments, a microbial consortium as described herein alters the gut environment such that the number, biomass, or activity of one or more beneficial organisms is increased by at least 10% (e.g., by at least 20%, at least 30%, at least 40%, at least 50%, at least 60%, at least 70%, at least 80%, at least 90%, at least 1-fold, at least 2-fold, at least 5-fold, at least 10-fold, at least 50-fold, at least 100-fold, at least 500-fold, at least 1000-fold, at least 5000-fold, at least 10,000-fold, at least 15,000-fold or at least 20,000-fold). For example, the microbial consortium stimulates the host's production of mucins and complex glycoconjugates to improve gut barrier function and colonization of beneficial organisms, additional probiotic compositions, or the microbial consortium itself. In some embodiments, the microbial composition for enhancing the biomass and/or activity of beneficial organisms comprises e.g., Bacteroides species, which enhances colonization by other Bacteroidetes and Clostridiales. In some embodiments, the microbial consortium influences gut pH, reduction of oxygen tension, secretion of glycosidases, and improving the reduction potential of the gut lumen to improve the colonization of beneficial organisms.

In another embodiment, the beneficial species comprises a Clostridium spp, such as Clostridium ramosum, Clostridium scindens, Clostridium hiranonsis, Clostridium bifermentans, Clostridium leptum, Clostridium sardiniensis, Clostridium hathewayi, Clostridium nexile, Clostridium hylemonae, Clostridium glycyrrhizinilyticum, Clostridium lavalense, Clostridium limetarium, Clostridium symbiosum, or Clostridium sporosphaeroides.

### Characterization of Bacteria and Bacterial Consortia

In certain embodiments, methods are provided for testing certain characteristics of compositions comprising a species of viable gut bacteria and/or microbial consortium. For example, the sensitivity of bacterial compositions to certain environmental variables is determined, e.g., in order to select for particular desirable characteristics in a given composition, formulation and/or use. For example, the bacterial constituents of the composition can be tested for pH resistance, bile acid resistance, and/or antibiotic sensitivity, either individually on a constituent-by-constituent basis or collectively as a bacterial composition comprised of multiple bacterial constituents (collectively referred to in this section as a microbial consortium).

pH Sensitivity Testing: If a pharmaceutical composition will be administered other than to the colon or rectum (i.e., for example, an oral route), optionally testing for pH resistance enhances the selection of microbes or therapeutic compositions that will survive at the highest yield possible through the varying pH environments of the distinct regions of the GI tract or genitourinary tracts. Understanding how the bacterial compositions react to the pH of the GI or genitourinary tracts also assists in formulation, so that the number of microbes in a dosage form can be increased if beneficial and/or so that the composition can be administered in an enteric-coated capsule or tablet or with a buffering or protective composition.

As the pH of the stomach can drop to a pH of 1 to 2 after a high-protein meal for a short time before physiological mechanisms adjust it to a pH of 3 to 4 and often resides at a resting pH of 4 to 5, and as the pH of the small intestine can range from a pH of 6 to 7.4, bacterial compositions can be prepared that survive these varying pH ranges (specifically wherein at least 1%, 5%, 10%, 15%, 20%, 25%, 30%, 40%, 50%, 60%, 70%, 80%, 90%, or as much as 100% of the bacteria can survive gut transit times through various pH ranges). This can be tested by exposing the bacterial composition to varying pH ranges for the expected gut transit times through those pH ranges. Therefore, as a non-limiting example only, 18-hour cultures of compositions comprising one or more bacterial species or species can be grown in standard media, such as gut microbiota medium (“GMM”, see Goodman et al., PNAS 108(15):6252-6257 (2011)) or another animal-products-free medium, with the addition of pH adjusting agents for a pH of 1 to 2 for 30 minutes, a pH of 3 to 4 for 1 hour, a pH of 4 to 5 for 1 to 2 hours, and a pH of 6 to 7.4 for 2.5 to 3 hours. An alternative method for testing stability to acid is described in e.g., U.S. Pat. No. 4,839,281. Survival of bacteria can be determined by culturing the bacteria and counting colonies on appropriate selective or non-selective media.

Bile Acid Sensitivity Testing: Additionally, in some embodiments, testing for bile-acid resistance enhances the selection of microbes or therapeutic compositions that will survive exposures to bile acid during transit through the GI tract. Bile acids are secreted into the small intestine and can, like pH, affect the survival of bacterial compositions. This can be tested by exposing the compositions to bile acids for the expected gut exposure time to bile acids. For example, bile acid solutions can be prepared at desired concentrations using 0.05 mM Tris at pH 9 as the solvent. After the bile acid is dissolved, the pH of the solution can be adjusted to 7.2 with 10% HCl. Bacterial components of the therapeutic compositions can be cultured in 2.2 ml of a bile acid composition mimicking the concentration and type of bile acids in the patient, 1.0 ml of 10% sterile-filtered feces media and 0.1 ml of an 18-hour culture of the given strain of bacteria. Incubations can be conducted for from 2.5 to 3 hours or longer. An alternative method for testing stability to bile acid is described in e.g., U.S. Pat. No. 4,839,281. Survival of bacteria can be determined by culturing the bacteria and counting colonies on appropriate selective or non-selective media.

Antibiotic Sensitivity Testing: As a further optional sensitivity test, the bacterial components of the microbial compositions can be tested for sensitivity to antibiotics. In one embodiment, the bacterial components can be chosen so that they are sensitive to antibiotics such that if necessary they can be eliminated or substantially reduced from the patient's gastrointestinal tract by at least one antibiotic targeting the bacterial composition.

Adherence to Gastrointestinal Cells: The compositions can optionally be tested for the ability to adhere to gastrointestinal cells. A method for testing adherence to gastrointestinal cells is described in e.g., U.S. Pat. No. 4,839,281.

Identification of Immunomodulatory Bacteria: In some embodiments, immunomodulatory bacteria are identified by the presence of nucleic acid sequences that modulate sporulation. In particular, signature sporulation genes are highly conserved across members of distantly related genera including Clostridium and Bacillus. Traditional approaches of forward genetics have identified many, if not all, genes that are essential for sporulation (spo). The developmental program of sporulation is governed in part by the successive action of four compartment-specific sigma factors (appearing in the order σF, σE, σG and σ), whose activities are confined to the forespore (σF and σG) or the mother cell (σE and σK). In other embodiments, immunomodulatory bacteria are identified by the biochemical activity of DPA producing enzymes or by analyzing DPA content of cultures. As part of the bacterial sporulation, large amounts of DPA are produced, and comprise 5-15% of the mass of a spore. Because not all viable spores germinate and grow under known media conditions, it is difficult to assess a total spore count in a population of bacteria. As such, a measurement of DPA content highly correlates with spore content and is an appropriate measure for characterizing total spore content in a bacterial population.

In other embodiments, immunomodulatory bacteria are identified by screening bacteria to determine whether the bacteria induce secretion of pro-inflammatory or anti-inflammatory cytokines by host cells. For example, human or mammalian cells capable of cytokine secretion, such as immune cells (e.g., PBMCs, macrophages, T cells, etc.) can be exposed to candidate immunomodulatory bacteria, or supernatants obtained from cultures of candidate immunomodulatory bacteria, and changes in cytokine expression or secretion can be measured using standard techniques, such as ELISA, immunoblot, Luminex™, antibody array, quantitative PCR, microarray, etc. Bacteria can be selected for inclusion (or exclusion) as a species viable gut bacterium or inclusion (or exclusion) in a microbial consortium based on the ability to induce a desired cytokine profile in human or mammalian cells. For example, anti-inflammatory bacteria can be selected for inclusion (or alternatively exclusion) as a species viable gut bacterium or inclusion (or exclusion) in a microbial consortium or composition thereof, based on the ability to induce secretion of one or more anti-inflammatory cytokines, and/or the ability to reduce secretion of one or more pro-inflammatory cytokines. Anti-inflammatory cytokines include, for example, IL-10, IL-13, IL-9, IL-4, IL-5, and combinations thereof. Other inflammatory cytokines include, for example, TGFβ. Pro-inflammatory cytokines include, for example, IFNγ, IL-12p70, IL-1α, IL-6, IL-8, MCP1, MIP1α, MIP1β, TNFα, and combinations thereof. In some embodiments, anti-inflammatory bacteria can be selected for inclusion (or exclusion) as a species viable gut bacterium or inclusion (or exclusion) in a microbial consortium based on the ability to modulate secretion of one or more anti-inflammatory cytokines and/or the ability to reduce secretion of one or more pro-inflammatory cytokines by a host cell induced by a bacterium of a different type (e.g., a bacterium from a different species or from a different strain of the same species).

In other embodiments, immunomodulatory bacteria are identified by screening bacteria to determine whether the bacteria impact the differentiation and/or expansion of particular subpopulations of immune cells. For example, candidate bacteria can be screened for the ability to promote differentiation and/or expansion of Treg cells, Th17 cells, Th1 cells and/or Th2 cells from precursor cells, e.g. naive T cells. By way of example, naïve T cells can be cultured in the presence of candidate bacteria or supernatants obtained from cultures of candidate bacteria, and numbers of Treg cells, Th17 cells, Th1 cells and/or Th2 cells can be determined using standard techniques, such as FACS analysis. Markers indicative of Treg cells include, for example, CD25⁺CD127^{lo}. Markers indicative of Th17 cells include, for example, CXCR3⁻CCR6⁺. Markers indicative of Th1 cells include, for example, CD4⁺, CXCR3⁺, and CCR6⁻. Markers indicative of Th2 cells include, for example, CD⁴⁺, CCR⁴⁺, and CXCR3⁻, CCR6⁻. Other markers indicative of particular T cells subpopulations are known in the art, and may be used in the assays described herein, e.g., to identify populations of immune cells impacted by candidate immunomodulatory bacteria. Bacteria can be selected for inclusion (or exclusion) as a species viable gut bacterium or inclusion (or exclusion) in a microbial consortium based on the ability to promote differentiation and/or expansion of a desired immune cell subpopulation.

In other embodiments, immunomodulatory bacteria are identified by screening bacteria to determine whether the bacteria secrete short chain fatty acids (SCFA), such as, for example, butyrate, acetate, propionate, or valerate, or combinations thereof. For example, secretion of short chain fatty acids into bacterial supernatants can be measured using standard techniques. In one embodiment, bacterial supernatants can be screened to measure the level of one or more short chain fatty acids using NMR, mass spectrometry (e.g., GC-MS, tandem mass spectrometry, matrix-assisted laser desorption/ionization, etc.), ELISA, or immunoblot. Expression of bacterial genes responsible for production of short chain fatty acids can also be determined by standard techniques, such as Northern blot, microarray, or quantitative PCR.

Exemplary minimal microbial consortia: Minimal microbial consortia are shown herein in the Examples section and can prevent and/or treat existing symptoms of a food allergy. These exemplary minimal microbial consortia should not be construed as limiting and are intended only for the better understanding of the methods and compositions described herein.

In one embodiment, a minimal microbial consortium consists essentially of: Clostridum ramosum, Clostridum scindens, Clostridum hiranonsis, Clostridum bifermentans, Clostridum leptum and Clostridum sardiniensis.

In one embodiment, a minimal microbial consortium consisting essentially of: Clostridum ramosum, Clostridum scindens, Clostridum hiranonsis, Clostridum bifermentans, Clostridum leptum and Clostridum sardiniensis is used in the prevention and/or treatment of existing allergic reactions to food.

In one embodiment, a minimal microbial consortium consists essentially of: Bacteroides fragilis, Bacteroides ovatus, Bacteroides vulgatus, Parabacteroides distasonis, and Prevotella melaninogenica.

In one embodiment, a minimal microbial consortium consisting essentially of: Bacteroides fragilis, Bacteroides ovatus, Bacteroides vulgatus, Parabacteroides distasonis, and Prevotella melaninogenica is used to treat existing allergic reactions to food.

By “consists essentially of” in this context is meant that if the addition of another microbe does not improve the treatment or prevention of allergy as described and defined herein, that microbe is not essential to the protective or therapeutic effect.

### Prebiotics

A prebiotic is a selectively fermented ingredient that allows specific changes, both in the composition and/or activity in the gastrointestinal microbiota, that confers neutral or positive benefits upon host well-being and health. Prebiotics can include complex carbohydrates, amino acids, peptides, or other nutritional components useful for the survival, colonization and persistence of the bacterial composition. Prebiotics include, but are not limited to, amino acids, biotin, fructooligosaccharide, galactooligosaccharides, inulin, lactulose, mannan oligosaccharides, oligofructose-enriched inulin, oligofructose, oligodextrose, tagatose, trans-galactooligosaccharide, and xylooligosaccharides.

Suitable prebiotics are usually plant-derived complex carbohydrates, oligosaccharides or polysaccharides. Generally, prebiotics are indigestible or poorly digested by humans and serve as a food source for bacteria. Prebiotics, which can be used in the pharmaceutical dosage forms, and pharmaceutical compositions provided herein include, without limitation, galactooligosaccharides (GOS), trans-galactooligosaccharides, fructooligosaccharides or oligofructose (FOS), inulin, oligofructose-enriched inulin, lactulose, arabinoxylan, xylooligosaccharides (XOS), mannooligosaccharides, gum guar, gum Arabic, tagatose, amylose, amylopectin, xylan, pectin, and the like and combinations of thereof. Prebiotics can be found in certain foods, e.g., chicory root, Jerusalem artichoke, Dandelion greens, garlic, leek, onion, asparagus, wheat bran, wheat flour, banana, milk, yogurt, sorghum, burdock, broccoli, Brussels sprouts, cabbage, cauliflower, collard greens, kale, radish and rutabaga, and miso. Alternatively, prebiotics can be purified or chemically or enzymatically synthesized.

In some embodiments, the composition comprises at least one prebiotic. In one embodiment, the prebiotic is a carbohydrate. In some embodiments, the composition comprises a prebiotic mixture, which comprises at least one carbohydrate. A “carbohydrate” refers to a sugar or polymer of sugars. The terms “saccharide,” “polysaccharide,” “carbohydrate,” and “oligosaccharide” can be used interchangeably. Most carbohydrates are aldehydes or ketones with many hydroxyl groups, usually one on each carbon atom of the molecule. Carbohydrates generally have the molecular formula (CH₂O)n. A carbohydrate can be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates can contain modified saccharide units, such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates can exist in many different forms, for example, conformers, cyclic forms, acyclic forms, stereoisomers, tautomers, anomers, and isomers. Carbohydrates can be purified from natural (e.g., plant or microbial) sources (i.e., they are enzymatically synthesized), or they can be chemically synthesized or modified.

Suitable prebiotic carbohydrates can include one or more of a carbohydrate, carbohydrate monomer, carbohydrate oligomer, or carbohydrate polymer. In certain embodiments, the pharmaceutical composition or dosage form comprises at least one type of microbe and at least one type of non-digestible saccharide, which includes non-digestible monosaccharides, non-digestible oligosaccharides, or non-digestible polysaccharides. In one embodiment, the sugar units of an oligosaccharide or polysaccharide can be linked in a single straight chain or can be a chain with one or more side branches. The length of the oligosaccharide or polysaccharide can vary from source to source. In one embodiment, small amounts of glucose can also be contained in the chain. In another embodiment, the prebiotic composition can be partially hydrolyzed or contain individual sugar moieties that are components of the primary oligosaccharide (see e.g., U.S. Pat. No. 8,486,668).

Prebiotic carbohydrates can include, but are not limited to monosaccharides (e.g., trioses, tetroses, pentoses, aldopentoses, ketopentoses, hexoses, cyclic hemiacetals, ketohexoses, heptoses) and multimers thereof, as well as epimers, cyclic isomers, stereoisomers, and anomers thereof. Non-limiting examples of monosaccharides include (in either the L- or D-conformation) glyceraldehyde, threose, ribose, altrose, glucose, mannose, talose, galactose, gulose, idose, lyxose, arabanose, xylose, allose, erythrose, erythrulose, tagalose, sorbose, ribulose, psicose, xylulose, fructose, dihydroxyacetone, and cyclic (alpha or beta) forms thereof. Multimers (disaccharides, trisaccharides, oligosaccharides, polysaccharides) thereof include, but are not limited to, sucrose, lactose, maltose, lactulose, trehalose, cellobiose, kojibiose, nigerose, isomaltose, sophorose, laminaribiose, gentioboise, turanose, maltulose, palatinose, gentiobiulose, mannobiose, melibiulose, rutinose, rutinulose, xylobiose, primeverose, amylose, amylopectin, starch (including resistant starch), chitin, cellulose, agar, agarose, xylan, glycogen, bacterial polysaccharides such as capsular polysaccharides, LPS, and peptidoglycan, and biofilm exopolysaccharide (e.g., alginate, EPS), N-linked glycans, and O-linked glycans. Prebiotic sugars can be modified and carbohydrate derivatives include amino sugars (e.g., sialic acid, N-acetylglucosamine, galactosamine), deoxy sugars (e.g., rhamnose, fucose, deoxyribose), sugar phosphates, glycosylamines, sugar alcohols, and acidic sugars (e.g., glucuronic acid, ascorbic acid).

In one embodiment, the prebiotic carbohydrate component of the pharmaceutical composition consists essentially of one or more non-digestible saccharides.

In one embodiment, the prebiotic carbohydrate component of the pharmaceutical composition allows the commensal colonic microbiota, comprising microorganisms associated with a healthy-state microbiome or presenting a low risk of a patient developing an autoimmune or inflammatory condition, to be regularly maintained. In one embodiment, the prebiotic carbohydrate allows the co-administered or co-formulated microbe or microbes to engraft, grow, and/or be regularly maintained in a mammalian subject. In some embodiments, the mammalian subject is a human subject, for example, a human subject having or suspected of having a food allergy.

In some embodiments, the prebiotic favors the growth of an administered microbe, wherein the growth of the administered microbe and/or the fermentation of the administered prebiotic by the administered microbe slows or reduces the growth of a pathogen or pathobiont. For example, FOS, neosugar, or inulin promotes the growth of acid-forming bacteria in the colon such as bacteria belonging to the genus Lactobacillus or Bifidobacterium and Lactobacillus acidophilus and Bifidobacterium bifidus can play a role in reducing the number of pathogenic bacteria in the colon (see e.g., U.S. Pat. No. 8,486,668). Other polymers, such as various galactans, lactulose, and carbohydrate based gums, such as psyllium, guar, carrageen, gellan, and konjac, are also known to improve gastrointestinal (GI) health.

In some embodiments, the prebiotic comprises one or more of GOS, lactulose, raffinose, stachyose, lactosucrose, FOS (i.e., oligofructose or oligofructan), inulin, isomalto-oligosaccharide, xylo-oligosaccharide, paratinose oligosaccharide, transgalactosylated oligosaccharides (i.e., transgalacto-oligosaccharides), transgalactosylate disaccharides, soybean oligosaccharides (i.e., soyoligosaccharides), gentiooligosaccharides, glucooligosaccharides, pecticoligosaccharides, palatinose polycondensates, difructose anhydride III, sorbitol, maltitol, lactitol, polyols, polydextrose, reduced paratinose, cellulose, β-glucose, β-galactose, β-fructose, verbascose, galactinol, and β-glucan, guar gum, pectin, high, sodium alginate, and lambda carrageenan, or mixtures thereof. The GOS may be a short-chain GOS, a long-chain GOS, or any combination thereof. The FOS can be a short-chain FOS, a long-chain FOS, or any combination thereof.

In some embodiments, the prebiotic composition comprises two carbohydrate species (non-limiting examples being a GOS and FOS) in a mixture of at least 1:1, at least 2:1, at least 5:1, at least 9:1, at least 10:1, about 20:1, or at least 20:1.

In some embodiments, the prebiotic comprises a mixture of one or more non-digestible oligosaccharides, non-digestible polysaccharides, free monosaccharides, non-digestible saccharides, starch, or non-starch polysaccharides.

Oligosaccharides are generally considered to have a reducing end and a non-reducing end, whether or not the saccharide at the reducing end is in fact a reducing sugar. Most oligosaccharides described herein are described with the name or abbreviation for the non-reducing saccharide (e.g., Gal or D-Gal), preceded or followed by the configuration of the glycosidic bond (α or β), the ring bond, the ring position of the reducing saccharide involved in the bond, and then the name or abbreviation of the reducing saccharide (e.g., Glc or D-Glc). The linkage (e.g., glycosidic linkage, galactosidic linkage, glucosidic linkage) between two sugar units can be expressed, for example, as 1,4, 1->4, or (1-4).

Both FOS and GOS are non-digestible saccharides. β glycosidic linkages of saccharides, such as those found in, but not limited to, FOS and GOS, make these prebiotics mainly non-digestible and unabsorbable in the stomach and small intestine α-linked GOS (α-GOS) is also not hydrolyzed by human salivary amylase, but can be used by Bifidobacterium bifidum and Clostridium butyricum (Yamashita A. et al., 2004. J. Appl. Glycosci. 51:115-122). FOS and GOS can pass through the small intestine and into the large intestine (colon) mostly intact, except where commensal microbes and microbes administered as part of a pharmaceutical composition are able to metabolize the oligosaccharides.

GOS (also known as galacto-oligosaccharides, galactooligosaccharides, trans-oligosaccharide (TOS), trans-galacto-oligosaccharide (TGOS), and trans-galactooligosaccharide) are oligomers or polymers of galactose molecules ending mainly with a glucose or sometimes ending with a galactose molecule and have varying degree of polymerization (generally the DP is between 2-20) and type of linkages. In one embodiment, GOS comprises galactose and glucose molecules. In another embodiment, GOS comprises only galactose molecules. In a further embodiment, GOS are galactose-containing oligosaccharides of the form of [β-D-Gal-(1-6)]ₙ-β-D-Gal-(1-4)-D-Glc wherein n is 2-20. In another embodiment, GOS are galactose-containing oligosaccharides of the form Glc α1-4-[β Gal 1-6)]ₙ where n=2-20. In another embodiment, GOS are in the form of α-D-Glc (1-4)-[β-D-Gal-(1-6)-]ₙ where n=2-20. Gal is a galactopyranose unit and Glc (or Glu) is a glucopyranose unit.

In one embodiment, a prebiotic composition comprises a GOS-related compound. A GOS-related compound can have the following properties: a) a “lactose” moiety; e.g., GOS with a gal-glu moiety and any polymerization value or type of linkage; orb) be stimulatory to “lactose fermenting” microbes in the human GI tract; for example, raffinose (gal-fru-glu) is a “related” GOS compound that is stimulatory to both lactobacilli and bifidobacteria.

Linkages between the individual sugar units found in GOS and other oligosaccharides include β-(1-6), β-(1-4), β-(1-3) and β-(1-2) linkages. In one embodiment, the administered oligosaccharides (e.g., GOS) are branched saccharides. In another embodiment, the administered oligosaccharides (e.g., GOS) are linear saccharides.

Alpha-GOS (also called alpha-bond GOS or alpha-linked GOS) are oligosaccharides having an alpha-galactopyranosyl group. Alpha-GOS comprises at least one alpha glycosidic linkage between the saccharide units. Alpha-GOS are generally represented by α-(Gal). (n usually represents an integer of 2 to 10) or α-(Gal). Glc (n usually represents an integer of 1 to 9). Examples include a mixture of α-galactosylglucose, α-galactobiose, α-galactotriose, α-galactotetraose, and higher oligosaccharides. Additional non-limiting examples include melibiose, manninootriose, raffinose, stachyose, and the like, which can be produced from beat, soybean oligosaccharide, and the like.

Commercially available and enzyme synthesized alpha-GOS products are also useful for the compositions described herein. Synthesis of alpha-GOS with an enzyme is conducted utilizing the dehydration condensation reaction of α-galactosidase with the use of galactose, galactose-containing substance, or glucose as a substrate. The galactose-containing substance includes hydrolysates of galactose-containing substances, for example, a mixture of galactose and glucose obtained by allowing beta-galactosidase to act on lactose, and the like. Glucose can be mixed separately with galactose and be used as a substrate with α-galactosidase (see e.g., WO 02/18614). Methods of preparing alpha-GOS have been described (see e.g., EP 514551 and EP2027863).

In one embodiment, a GOS composition comprises a mixture of saccharides that are alpha-GOS and saccharides that are produced by transgalactosylation using β-galactosidase. In another embodiment, GOS comprises alpha-GOS. In another embodiment, alpha-GOS comprises α-(Gal)₂ from 10% to 100% by weight. In one embodiment, GOS comprises only saccharides that are produced by transgalactosylation using β-galactosidase.

In one embodiment, the pharmaceutical composition comprises, in addition to one or more microbes, an oligosaccharide composition that is a mixture of oligosaccharides comprising 1-20% by weight of di-saccharides, 1-20% by weight tri-saccharides, 1-20% by weight tetra-saccharides, and 1-20% by weight penta-saccharides. In another embodiment, an oligosaccharide composition is a mixture of oligosaccharides consisting essentially of 1-20% by weight of di-saccharides, 1-20% by weight tri-saccharides, 1-20% by weight tetra-saccharides, and 1-20% by weight penta-saccharides.

In one embodiment, a prebiotic composition is a mixture of oligosaccharides comprising 1-20% by weight of saccharides with a degree of polymerization (DP) of 1-3, 1-20% by weight of saccharides with DP of 4-6, 1-20% by weight of saccharides with DP of 7-9, and 1-20% by weight of saccharides with DP of 10-12, 1-20% by weight of saccharides with DP of 13-15.

In another embodiment, a prebiotic composition comprises a mixture of oligosaccharides comprising 50-55% by weight of di-saccharides, 20-30% by weight tri-saccharides, 10-20% by weight tetra-saccharide, and 1-10% by weight penta-saccharides. In one embodiment, a GOS composition is a mixture of oligosaccharides comprising 52% by weight of di-saccharides, 26% by weight tri-saccharides, 14% by weight tetra-saccharide, and 5% by weight penta-saccharides. In another embodiment, a prebiotic composition comprises a mixture of oligosaccharides comprising 45-55% by weight tri-saccharides, 15-25% by weight tetra-saccharides, 1-10% by weight penta-saccharides.

In certain embodiments, the composition comprises a mixture of neutral and acid oligosaccharides as disclosed in e.g., WO 2005/039597 (N.V. Nutricia) and US Patent Application 20150004130. In one embodiment, the acid oligosaccharide has a degree of polymerization (DP) between 1 and 5000. In another embodiment, the DP is between 1 and 1000. In another embodiment, the DP is between 2 and 250. If a mixture of acid oligosaccharides with different degrees of polymerization is used, the average DP of the acid oligosaccharide mixture is preferably between 2 and 1000. The acid oligosaccharide can be a homogeneous or heterogeneous carbohydrate. The acid oligosaccharides can be prepared from pectin, pectate, alginate, chondroitine, hyaluronic acids, heparin, heparane, bacterial carbohydrates, sialoglycans, fucoidan, fucooligosaccharides or carrageenan, and are preferably prepared from pectin or alginate. The acid oligosaccharides can be prepared by the methods described in e.g., WO 01/60378, which is hereby incorporated by reference. The acid oligosaccharide is preferably prepared from high methoxylated pectin, which is characterized by a degree of methoxylation above 50%. As used herein, “degree of methoxylation” (also referred to as DE or “degree of esterification”) is intended to mean the extent to which free carboxylic acid groups contained in the polygalacturonic acid chain have been esterified (e.g. by methylation). In some embodiments, the acid oligosaccharides have a degree of methoxylation above about 10%, above about 20%, above about 50%, above about 70%. In some embodiments, the acid oligosaccharides have a degree of methylation above about 10%, above about 20%, above about 50%, above about 70%.

The term neutral oligosaccharides as used in the present invention refers to saccharides which have a degree of polymerization of monose units exceeding 2, exceeding 3, exceeding 4, or exceeding 10, which are not or only partially digested in the intestine by the action of acids or digestive enzymes present in the human upper digestive tract (small intestine and stomach) but which are fermented by the human intestinal flora and preferably lack acidic groups. The neutral oligosaccharide is structurally (chemically) different from the acid oligosaccharide. The term “neutral oligosaccharides”, as used herein, refers to saccharides which have a degree of polymerization of the oligosaccharide below 60 monose units. The term “monose units” refers to units having a closed ring structure e.g., the pyranose or furanose forms. In some embodiments, the neutral oligosaccharide comprises at least 90% or at least 95% monose units selected from the group consisting of mannose, arabinose, fructose, fucose, rhamnose, galactose, -D-galactopyranose, ribose, glucose, xylose and derivatives thereof, calculated on the total number of monose units contained therein. Suitable neutral oligosaccharides are preferably fermented by the gut flora. Non-limiting examples of suitable neutral oligosaccharides are cellobiose (4-O-β-D-glucopyranosyl-D-glucose), cellodextrins ((4-O-β-D-glucopyranosyl)n-D-glucose), β-cyclo-dextrins (Cyclic molecules of α-1-4-linked D-glucose; α-cyclodextrin-hexamer, β-cyclodextrin-heptamer and γ-cyclodextrin-octamer), indigestible dextrin, gentiooligosaccharides (mixture of β-1-6 linked glucose residues, some 1-4 linkages), glucooligosaccharides (mixture of α-D-glucose), isomaltooligosaccharides (linear α-1-6 linked glucose residues with some 1-4 linkages), isomaltose (6-O-α-D-glucopyranosyl-D-glucose); isomaltriose (6-O-α-D-glucopyranosyl-(1-6)-α-D-glucopyranosyl-D-glucose), panose (6-O-α-D-glucopyranosyl-(1-6)-α-D-glucopyranosyl-(1-4)-D-glucose), leucrose (5-O-α-D-glucopyranosyl-D-fructopyranoside), palatinose or isomaltulose (6-O-α-D-glucopyranosyl-D-fructose), theanderose (O-α-D-glucopyranosyl-(1-6)-O-α-D-glucopyranosyl-(1-2)-B-D-fructo furanoside), D-agatose, D-lyxo-hexylose, lactosucrose (O-β-D-galactopyranosyl-(1-4)-O-α-D-glucopyranosyl-(1-2)-β-D-fructofuranoside), α-galactooligosaccharides including raffinose, stachyose and other soy oligosaccharides (O-α-D-galactopyranosyl-(1-6)-α-D-glucopyranosyl-β-D-fructofuranoside), (3-galactooligosaccharides or transgalacto-oligosaccharides (β-D-galactopyranosyl-(1-6)-[β-D-glucopyranosyl]n-(1-4) α-D glucose), lactulose (4-O-β-D-galactopyranosyl-D-fructose), 4′-galatosyllactose (β-D-galactopyranosyl-(1-4)-O-β-D-glucopyranosyl-(1-4)-D-glucopyranose), synthetic galactooligosaccharide (neogalactobiose, isogalactobiose, galsucrose, isolactose I, II and III), fructans-Levan-type (β-D-(2→6)-fructofuranosyl)n α-D-glucopyranoside), fructans-Inulin-type (β-D-((2→1)-fructofuranosyl)n α-D-glucopyranoside), 1 f-β-fructofuranosylnystose (β-D-((2→1)-fructofuranosyl)n B-D-fructofuranoside), xylooligo-saccharides (B-D-((1→4)-xylose)n, lafinose, lactosucrose and arabinooligosaccharides.

In some embodiments, the neutral oligosaccharide is selected from the group consisting of fructans, fructooligosaccharides, indigestible dextrins galactooligo-saccharides (including transgalactooligosaccharides), xylooligosaccharides, arabinooligo-saccharides, glucooligosaccharides, mannooligosaccharides, fucooligosaccharides and mixtures thereof.

Suitable oligosaccharides and their production methods are further described in Laere K. J. M. (Laere, K. J. M., Degradation of structurally different non-digestible oligosaccharides by intestinal bacteria: glycosylhydrolases of Bi. adolescentis. PhD-thesis (2000), Wageningen Agricultural University, Wageningen, The Netherlands), the entire content of which is hereby incorporated by reference. Transgalactooligosaccharides (TOS) are for example sold under the trademark Vivinal™ (Borculo Domo Ingredients, Netherlands). Indigestible dextrin, which can be produced by pyrolysis of corn starch, comprises α(1→4) and α(1→6) glucosidic bonds, as are present in the native starch, and contains 1→2 and 1→3 linkages and levoglucosan. Due to these structural characteristics, indigestible dextrin contains well-developed, branched particles that are partially hydrolyzed by human digestive enzymes. Numerous other commercial sources of indigestible oligosaccharides are readily available and known to skilled persons in the art. For example, transgalactooligosaccharide is available from Yakult Honsha Co., Tokyo, Japan. Soybean oligosaccharide is available from Calpis Corporation distributed by Ajinomoto U.S.A. Inc., Teaneck, N.J.

In a further embodiment, the prebiotic mixture of the pharmaceutical composition described herein comprises an acid oligosaccharide with a DP between 1 and 5000, prepared from pectin, alginate, and mixtures thereof; and a neutral oligosaccharide, selected from the group of fructans, fructooligosaccharides, indigestible dextrins, galactooligosaccharides including transgalacto-oligosaccharides, xylooligosaccharides, arabinooligosaccharides, glucooligosaccharides, mannooligosaccharides, fucooligosaccharides, and mixtures thereof.

In certain embodiments, the prebiotic mixture comprises xylose. In other embodiments, the prebiotic mixture comprises a xylose polymer (i.e. xylan). In some embodiments, the prebiotic comprises xylose derivatives, such as xylitol, a sugar alcohol generated by reduction of xylose by catalytic hydrogenation of xylose, and also xylose oligomers (e.g., xylooligosaccharide). While xylose can be digested by humans, via xylosyltransferase activity, most xylose ingested by humans is excreted in urine. In contrast, some microorganisms are efficient at xylose metabolism or can be selected for enhanced xylose metabolism. Microbial xylose metabolism can occur by at least four pathways, including the isomerase pathway, the Weimburg pathway, the Dahms pathway, and, for eukaryotic microorganisms, the oxido-reductase pathway.

The xylose isomerase pathway involves the direct conversion of D-xylose into D-xylulose by xylose isomerase, after which D-xylulose is phosphorylated by xylulose kinase to yield D-xylolose-5-phosphate, an intermediate of the pentose phosphate pathway.

In the Weimberg pathway, D-xylose is oxidized to D-xylono-lactone by a D-xylose dehydrogenase. Then D-xylose dehydrogenase is hydrolyzed by a lactonase to yield D-xylonic acid, and xylonate dehydratase activity then yields 2-keto-3-deoxy-xylonate. The final steps of the Weimberg pathway are a dehydratase reaction to form 2-keto glutarate semialdehyde and an oxidizing reaction to form 2-ketoglutarate, an intermediate of the Krebs cycle.

The Dahms pathway follows the same mechanism as the Weimberg pathway but diverges once it has yielded 2-keto-3-deoxy-xylonate. In the Dahms pathway, an aldolase splits 2-keto-3-deoxy-xylonate into pyruvate and glycolaldehyde.

The xylose oxido-reductase pathway, also known as the xylose reductase-xylitol dehydrogenase pathway, begins by the reduction of D-xylose to xylitol by xylose reductase followed by the oxidation of xylitol to D-xylulose by xylitol dehydrogenase. As in the isomerase pathway, the next step in the oxido-reductase pathway is the phosphorylation of D-xylulose by xylulose kinase to yield D-xylolose-5-phosphate.

Xylose is present in foods like fruits and vegetables and other plants such as trees for wood and pulp production. Thus, xylose can be obtained in the extracts of such plants. Xylose can be obtained from various plant sources using known processes including acid hydrolysis followed by various types of chromatography. Examples of such methods to produce xylose include those described in Maurelli, L. et al. (2013), Appl. Biochem. Biotechnol. 170:1104-1118; Hooi H. T et al. (2013), Appl. Biochem. Biotechnol. 170:1602-1613; Zhang H-J. et al. (2014), Bioprocess Biosyst. Eng. 37:2425-2436.

Culture and Storage of Monotherapy or Consortium constituents

For banking, the species included in the bacterial composition can be (1) isolated directly from a specimen or taken from a banked stock, (2) optionally cultured on a nutrient agar or broth that supports growth to generate viable biomass, and (3) the biomass optionally preserved in multiple aliquots in long-term storage.

In embodiments using a culturing step, the agar or broth contains nutrients that provide essential elements and specific factors that enable growth. An example would be a medium composed of 20 g/L glucose, 10 g/L yeast extract, 10 g/L soy peptone, 2 g/L citric acid, 1.5 g/L sodium phosphate monobasic, 100 mg/L ferric ammonium citrate, 80 mg/L magnesium sulfate, 10 mg/L hemin chloride, 2 mg/L calcium chloride, and 1 mg/L menadione. A variety of microbiological media and variations are well known in the art (e.g. R. M. Atlas, Handbook of Microbiological Media (2010) CRC Press). Medium can be added to the culture at the start, can be added during the culture, or can be intermittently/continuously flowed through the culture. The species in the bacterial composition can be cultivated alone, as a subset of the bacterial composition, or as an entire collection comprising the bacterial composition. As an example, a first strain can be cultivated together with a second strain in a mixed continuous culture, at a dilution rate lower than the maximum growth rate of either cell to prevent the culture from washing out of the cultivation.

The inoculated culture is incubated under favorable conditions for a time sufficient to build biomass. For bacterial compositions for human use this is often at normal body temperature (37° C.), pH, and other parameter with values similar to the normal human niche. The environment can be actively controlled, passively controlled (e.g., via buffers), or allowed to drift. For example, for anaerobic bacterial compositions (e.g., gut microbiota), an anoxic/reducing environment can be employed. This can be accomplished by addition of reducing agents/factors such as cysteine to the broth, and/or stripping it of oxygen. As an example, a culture of a bacterial composition can be grown at 37° C., pH 7, in the medium above, pre-reduced with 1 g/L cysteine⋅HCI.

When the culture has generated sufficient biomass, it can be preserved for banking or storage. The organisms can be placed into a chemical milieu that protects from freezing (adding ‘cryoprotectants’), drying (‘lyoprotectants’), and/or osmotic shock (‘osmoprotectants’), dispensing into multiple (optionally identical) containers to create a uniform bank, and then treating the culture for preservation. Containers are generally impermeable and have closures that assure isolation from the environment. Cryopreservation treatment is accomplished by freezing a liquid at ultra-low temperatures (e.g., at or below −80° C.). Dried preservation removes water from the culture by evaporation (in the case of spray drying or ‘cool drying’) or by sublimation (e.g., for freeze drying, spray freeze drying). Removal of water improves long-term bacterial composition storage stability at temperatures elevated above cryogenic. If the bacterial composition comprises spore forming species and results in the production of spores, the final composition can be purified by additional means such as density gradient centrifugation preserved using the techniques described above. Bacterial composition banking can be done by culturing and preserving the species individually, or by mixing the species together to create a combined bank. As an example of cryopreservation, a bacterial composition culture can be harvested by centrifugation to pellet the cells from the culture medium, the supernatant decanted and replaced with fresh culture broth containing 15% glycerol. The culture can then be aliquoted into 1 mL cryotubes, sealed, and placed at −80° C. for long-term viability retention. This procedure achieves acceptable viability upon recovery from frozen storage.

Organism production can be conducted using similar culture steps to banking, including medium composition and culture conditions. It can be conducted at larger scales of operation, especially for clinical development or commercial production. At larger scales, there can be several subcultivations of the bacterial composition prior to the final cultivation. At the end of cultivation, the culture is harvested to enable further formulation into a dosage form for administration. This can involve concentration, removal of undesirable medium components, and/or introduction into a chemical milieu that preserves the bacterial composition and renders it acceptable for administration via the chosen route. For example, a bacterial composition can be cultivated to a concentration of 10¹⁰ CFU/mL, then concentrated 20-fold by tangential flow microfiltration; the spent medium may be exchanged by diafiltering with a preservative medium consisting of 2% gelatin, 100 mM trehalose, and 10 mM sodium phosphate buffer. The suspension can then be freeze-dried to a powder and titrated.

After drying, the powder can be blended to an appropriate potency, and mixed with other cultures and/or a filler such as microcrystalline cellulose for consistency and ease of handling, and the bacterial composition formulated as provided herein.

In one embodiment, a composition comprising a species of viable gut bacteria and/or a microbial consortium as described herein, is not a fecal transplant. In some embodiments all or essentially all of the bacterial entities present in a purified population are originally obtained from a fecal material and subsequently, e.g., for production of pharmaceutical compositions, are grown in culture as described herein or otherwise known in the art. In one embodiment, the bacterial cells are cultured from a bacterial stock and purified as described herein. In one embodiment, each of the populations of bacterial cells are independently cultured and purified, e.g., each population is cultured separately and subsequently mixed together. In one embodiment, one or more of the populations of bacterial cells in the composition are co-cultured.

### Dosage, Administration and Formulations

In some embodiments, cells over a range of, for example, 2-5×10⁵, or more, e.g., 1×10⁶, 1×10⁷, 1×10⁸, 5×10⁸, 1×10⁹, 5×10⁹, 1×10¹⁰, 5×10¹⁰ or more can be administered in a composition comprising a species of viable gut bacteria and/or a microbial consortium. The dosage range for the bacteria depends upon the potency, and include amounts large enough to produce the desired effect, e.g., reduction in at least one symptom of a food allergy in a treated subject. The dosage should not be so large as to cause unacceptable adverse side effects. Generally, the dosage will vary with the type of illness, and with the age, condition, and sex of the patient. The dosage can be determined by one of skill in the art and can also be adjusted by the individual physician in the event of any complication.

For use in the various aspects described herein, an effective amount of cells in a composition as described herein comprises at least 10² bacterial cells, at least 1×10² bacterial cells, at least 1×10⁴ bacterial cells, at least 1×10⁵ bacterial cells, at least 1×10⁶ bacterial cells, at least 1×10⁷ bacterial cells, at least 1×10⁸ bacterial cells, at least 1×10⁹ bacterial cells, at least 1×10¹⁰ bacterial cells, at least 1×10¹¹ bacterial cells, at least 1×10¹² bacterial cells or more. Where a viable gut bacteria and/or microbial consortium is isolated and/or purified from a subject that is tolerant to a selected food allergen, the bacterial cells can be derived from one or more donors, or can be obtained from an autologous source. In some embodiments of the aspects described herein, the cells of the viable gut bacteria and/or microbial consortium are expanded or maintained in culture prior to administration to a subject in need thereof. In one embodiment, the viable gut bacteria and/or microbial consortium is obtained from a microbe bank. Members of a therapeutic or preventive/prophylactic consortium are generally administered together, e.g., in a single admixture. However, it is specifically contemplated herein that members of a given consortium can be administered as separate dosage forms or sub-mixtures or sub-combinations of the consortium members. Thus, for a consortium of e.g., six members, the consortium can be administered, for example, as a single preparation including all six members (in one or more dosage units, e.g., one or more capsules) or as two or more separate preparations that, in sum, include all members of the given consortium. While administration as a single admixture is preferred, a potential advantage of the use of e.g., individual units for each member of a consortium, is that the actual species administered to any given subject can be tailored, if necessary, by selecting the appropriate combination of, for example, single species dosage units that together comprise the desired consortium.

Biomass of administered species, per dose, vs. known in vivo biomass: It is contemplated herein that the viable gut bacteria composition and/or consortium composition is formulated to deliver a larger biomass than the normal biomass of the commensal organisms in a “healthy” individual. For example, the range of biomasses contemplated for delivery and colonization can be found in Table 1, column 2, as compared to the normal biomass in a healthy individual as shown in Table 1, columns 3 & 4. The table below shows the range of administered biomasses of organisms relative to published data at specific locations. Note, in many cases the bacterial quantitation in Gustafsson, 1982 was to general categories of organisms, such as Clostridia, and incorporated multiple species under those headers. Individual species in the consortia would thus likely be less than the actual highest reported biomass at the specific locations; the small and large intestinal biomass data should thus be considered an upper-bound for what might occur in vivo in normal individuals.

| | | Small | | |
|---|---|---|---|---|
| | Consortia | Intestinal | Large Intestinal | |
| Species | Biomass | Biomass | Biomass | Reference |
| Bacteroides fragilis | 1 × 10⁷-5 × 10⁸ | <10³ CFU/g in | 10⁸-10¹¹ CFU/g | Gustafsson, 1982. |
| | CFU/mL | duodenum- | | [1] |
| | | jejunum | | |
| | | 10³-10⁸ CFU/g | | |
| | | in ileum | | |
| B. thetaiotaomicron | 1 × 10⁷-5 × 10⁸ | <10³ CFU/g in | 10⁸-10¹¹ CFU/g | Gustafsson, 1982. |
| | CFU/mL | duodenum- | | Bry, 1996. [2] |
| | | jejunum | | |
| | | 10³-10⁸ CFU/g | | |
| | | in ileum | | |
| B. ovatus | 1 × 10⁷-5 × 10⁸ | <10³ CFU/g in | 10⁸-10¹¹ CFU/g | Gustafsson, 1982. |
| | CFU/mL | duodenum- | | |
| | | jejunum | | |
| | | 10³-10⁸ CFU/g | | |
| | | in ileum | | |
| B. vulgatus | 1 × 10⁷-5 × 10⁸ | <10³ CFU/g in | 10⁸-10¹¹ CFU/g | Gustafsson, 1982. |
| | CFU/mL | duodenum- | | Pinto 2017, |
| | | jejunum | | Medina 2017 |
| | | 10³-10⁸ CFU/g | | |
| | | in ileum | | |
| P. distasonis | 1 × 10⁷-5 × 10⁸ | <10³ CFU/g in | 0-1 × 10⁸ | Gustafsson, 1982. |
| | CFU/mL | duodenum- | CU/mL | |
| | | jejunum | | |
| | | 10³-10⁸ CFU/g | | |
| | | in ileum | | |
| P. melaninogenica | 1 × 10⁷-5 × 10⁸ | <10³ CFU/g in | 0-1 × 10⁶ | Finegold, 1977 |
| | CFU/mL | oral cavity | CFU/mL | |
| C. bifermentans | 1 × 10⁷-5 × 10⁸ | <10³ CFU/g in | 0-10⁶ CFU/g | Gustafsson, 1982. |
| | CFU/mL | duodenum- | | |
| | | jejunum | | |
| | | 10²-10⁴ CFU/g | | |
| | | in ileum | | |
| C. hiranonsis | 1 × 10⁷-5 × 10⁸ | <10³ CFU/g in | 0-10⁶ CFU/g | Gustafsson, 1982. |
| | CFU/mL | duodenum- | | |
| | | jejunum | | |
| | | 10²-10⁴ CFU/g | | |
| | | in ileum | | |
| C. leptum | 1 × 10⁷-5 × 10⁸ | <10³ CFU/g in | 0-10⁶ CFU/g | Gustafsson, 1982. |
| | CFU/mL | duodenum- | | |
| | | jejunum | | |
| | | 10²-10⁴ CFU/g | | |
| | | in ileum | | |
| Clostridium | 1 × 10⁷-5 × 10⁸ | <10³ CFU/g in | 0-10⁶ CFU/g | Gustafsson, 1982. |
| ramosum | CFU/mL | duodenum- | | |
| | | jejunum | | |
| | | 10²-10⁴ CFU/g | | |
| | | in ileum | | |
| C. sardiniensis | 1 × 10⁷-5 × 10⁸ | <10³ CFU/g in | 0-10⁶ CFU/g | Gustafsson, 1982. |
| | CFU/mL | duodenum- | | |
| | | jejunum | | |
| | | 10²-10⁴ CFU/g | | |
| | | in ileum | | |
| C. scindens | 1 × 10⁷-5 × 10⁸ | <10³ CFU/g in | 0-10⁶ CFU/g | Gustafsson, 1982. |
| | CFU/mL | duodenum- | | |
| | | jejunum | | |
| | | 10²-10⁴ CFU/g | | |
| | | in ileum | | |
| Parabacteroides | 1 × 10⁷-5 × 10⁸ | <10³ CFU/g in | 0-10⁶ CFU/g | Gustafsson, 1982. |
| goldsteinii | CFU/mL | duodenum- | | |
| | | jejunum | | |
| | | 10³-10⁸ CFU/g | | |
| | | in ileum | | |
| Prevotella tannerae | 1 × 10⁷-5 × 10⁸ | <10³ CFU/g in | 0-10⁶ CFU/g | Gustafsson, 1982. |
| | CFU/mL | duodenum- | | |
| | | jejunum | | |
| | | <10⁴ CFU/g in | | |
| | | ileum | | |
| Subdoligranulum | 5x10⁶-2x10⁷ | | | |
| variabile | CFU/mL | | | |

A pharmaceutical composition comprising a viable gut bacteria and/or microbial consortium can be administered by any method suitable for depositing in the gastrointestinal tract, preferably the colon, of a subject (e.g., human, mammal, animal, etc.). Examples of routes of administration include rectal administration by colonoscopy, suppository, enema, upper endoscopy, or upper push enteroscopy. Additionally, intubation through the nose or the mouth by nasogastric tube, nasoenteric tube, or nasal jejunal tube can be utilized. Oral administration by a solid such as a pill, tablet, a suspension, a gel, a geltab, a semisolid, a tablet, a sachet, a lozenge or a capsule or microcapsule, or as an enteral formulation, or re-formulated for final delivery as a liquid, a suspension, a gel, a geltab, a semisolid, a tablet, a sachet, a lozenge or a capsule, or as an enteral formulation can be utilized as well. Also contemplated herein are food items that are inoculated with a microbial consortium as described herein. Compositions can also be treated or untreated fecal flora, entire (or substantially entire) microbiota, or partially, substantially or completely isolated or purified fecal flora, and can be lyophilized, freeze-dried or frozen, or processed into a powder.

In some embodiments, the compositions described herein can be administered in a form containing one or more pharmaceutically acceptable carriers. Suitable carriers are well known in the art and vary with the desired form and mode of administration of the composition. For example, pharmaceutically acceptable carriers can include diluents or excipients such as fillers, binders, wetting agents, disintegrators, surface-active agents, glidants, lubricants, and the like. Typically, the carrier may be a solid (including powder), liquid, or combinations thereof. Each carrier is preferably “acceptable” in the sense of being compatible with the other ingredients in the composition and not injurious to the subject. The carrier may be biologically acceptable and inert (e.g., it permits the composition to maintain viability of the biological material until delivered to the appropriate site).

Oral compositions can include an inert diluent or an edible carrier. For the purpose of oral therapeutic administration, the active compound can be incorporated with excipients and used in the form of tablets, lozenges, pastilles, troches, or capsules, e.g., gelatin capsules. Oral compositions can also be prepared by combining a composition of the present disclosure with a food. In one embodiment a food used for administration is chilled, for instance, iced flavored water. In certain embodiments, the food item is not a potentially allergenic food item (e.g., not soy, wheat, peanut, tree nuts, dairy, eggs, shellfish or fish). Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, orange flavoring, or other suitable flavorings. These are for purposes of example only and are not intended to be limiting.

The compositions comprising a viable gut bacteria and/or microbial consortium can also be prepared in the form of suppositories (e.g., with conventional suppository bases such as cocoa butter and other glycerides) or retention enemas for rectal delivery. The compositions can be prepared with carriers that will protect the viable gut bacteria and/or consortium against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from, for instance, Alza Corporation and Nova Pharmaceuticals, Inc. Liposomal suspensions can also be used as pharmaceutically acceptable carriers. These can be prepared according to methods known to those skilled in the art.

In some embodiments, a composition can be encapsulated (e.g., enteric-coated formulations). For instance, when the composition is to be administered orally, the dosage form is formulated so the composition is not exposed to conditions prevalent in the gastrointestinal tract before the small intestine, e.g., high acidity and digestive enzymes present in the stomach. The encapsulation of compositions for therapeutic use is routine in the art. Encapsulation can include hard-shelled capsules, which can be used for dry, powdered ingredients soft-shelled capsules. Capsules can be made from aqueous solutions of gelling agents such as animal protein (e.g., gelatin), plant polysaccharides or derivatives like carrageenans and modified forms of starch and cellulose. Other ingredients can be added to a gelling agent solution such as plasticizers (e.g., glycerin and or sorbitol), coloring agents, preservatives, disintegrants, lubricants and surface treatment.

In one embodiment, a viable gut bacteria and/or a microbial consortium as described herein is formulated with an enteric coating. An enteric coating can control the location of where a microbial consortium is released in the digestive system. Thus, an enteric coating can be used such that a viable gut bacteria-containing and/or microbial consortium-containing composition does not dissolve and release the microbes in the stomach, which can be a toxic environment for many microbes, but rather travels to the small intestine, where it dissolves and releases the microbes in an environment where they can survive. An enteric coating can be stable at low pH (such as in the stomach) and can dissolve at higher pH (for example, in the small intestine). Material that can be used in enteric coatings includes, for example, alginic acid, cellulose acetate phthalate, plastics, waxes, shellac, and fatty acids (e.g., stearic acid, palmitic acid). Enteric coatings are described, for example, in U.S. Pat. Nos. 5,225,202, 5,733,575, 6,139,875, 6,420,473, 6,455,052, and 6,569,457, all of which are herein incorporated by reference in their entirety. The enteric coating can be an aqueous enteric coating. Examples of polymers that can be used in enteric coatings include, for example, shellac (trade name EmCoat 120 N, Marcoat 125); cellulose acetate phthalate (trade names AQUACOAT™, AQUACOAT ECD, SEPIFILM™, KLUCEL™, and METOLOSE™); polyvinylacetate phthalate (trade name SURETERIC™); and methacrylic acid (trade name EUDRAGIT™).

In one embodiment, an enteric coated probiotic composition comprising members of a viable gut bacteria and/or microbial consortium as described herein is administered to a subject. In another embodiment, an enteric coated probiotic and prebiotic composition is administered to a subject.

Formulations suitable for rectal administration include gels, creams, lotions, aqueous or oily suspensions, dispersible powders or granules, emulsions, dissolvable solid materials, douches, and the like. The formulations are preferably provided as unit-dose suppositories comprising the active ingredient in one or more solid carriers forming the suppository base, for example, cocoa butter. Suitable carriers for such formulations include petroleum jelly, lanolin, polyethyleneglycols, alcohols, and combinations thereof. Alternatively, colonic washes with the rapid recolonization deployment agent of the present disclosure can be formulated for colonic or rectal administration.

Formulations suitable for oral administration may be provided as discrete units, such as tablets, capsules, cachets, syrups, elixirs, prepared food items, microemulsions, solutions, suspensions, lozenges, or gel-coated ampules, each containing a predetermined amount of the active compound; as powders or granules; as solutions or suspensions in aqueous or non-aqueous liquids; or as oil-in-water or water-in-oil emulsions.

In some embodiments, the viable gut bacteria and/or microbial consortium can be formulated in a food item. Some non-limiting examples of food items to be used with the methods and compositions described herein include: popsicles, cheeses, creams, chocolates, milk, meat, drinks, yogurt, pickled vegetables, kefir, miso, sauerkraut, etc. In other embodiments, the food items can be juices, refreshing beverages, tea beverages, drink preparations, jelly beverages, and functional beverages; alcoholic beverages such as beers; carbohydrate-containing foods such as rice food products, noodles, breads, and pastas; paste products such as fish, hams, sausages, paste products of seafood; retort pouch products such as curries, food dressed with a thick starchy sauce, and Chinese soups; soups; dairy products such as milk, dairy beverages, ice creams, cheeses, and yogurts; fermented products such as fermented soybean pastes, fermented beverages, and pickles; bean products; various confectionery products including biscuits, cookies, and the like, candies, chewing gums, gummies, cold desserts including jellies, cream caramels, and frozen desserts; instant foods such as instant soups and instant soy-bean soups; and the like. It is preferred that food preparations not require cooking after admixture with the microbial consortium to avoid killing the microbes.

Formulations of a viable gut bacteria and/or microbial consortium can be prepared by any suitable method, typically by uniformly and intimately admixing the consortium with liquids or finely divided solid carriers or both, in the required proportions and then, if necessary, shaping the resulting in mixture into the desired shape. In addition, the viable gut bacteria and/or microbial consortium can be treated to prolong shelf-life, preferably the shelf-life of the pre-determined gut flora will be extended via freeze drying.

In some embodiments, the viable gut bacteria and/or microbial consortium as described herein is combined with one or more additional probiotic organisms prior to treatment of a subject. As used herein, the term “probiotic” refers to microorganisms that form at least a part of the transient or endogenous flora or microbial consortium and thereby exhibit a beneficial prophylactic and/or therapeutic effect on the host organism. Probiotics are generally known to be clinically safe (i.e., non-pathogenic) by those individuals skilled in the art. Typical lactic acid-producing bacteria useful as a probiotic of this invention are efficient lactic acid producers which include non-pathogenic members of the Bacillus genus which produce bacteriocins or other compounds which inhibit the growth of pathogenic organisms.

Exemplary lactic acid-producing, non-pathogenic Bacillus species include, but are not limited to: Bacillus coagulans; Bacillus coagulans Hammer; and Bacillus brevis subspecies coagulans.

Exemplary lactic acid-producing Lactobacillus species include, but are not limited to: Lactobacillus acidophilus, Lactobacillus casei, Lactobacillus DDS-1, Lactobacillus GG, Lactobacillus rhamnosus, Lactobacillus plantarum, Lactobacillus reuteri, Lactobacillus gasserii, Lactobacillus jensenii, Lactobacillus delbruekii, Lactobacillus, bulgaricus, Lactobacillus salivarius and Lactobacillus sporogenes (also designated as Bacillus coagulans). Exemplary lactic acid-producing Sporolactobacillus species include all Sporolactobacillus species, for example, Sporolactobacillus P44.

Exemplary lactic acid-producing Bifidiobacterium species include, but are not limited to: Bifidiobacterium adolescentis, Bifidiobacterium animalis, Bifidiobacterium bifidum, Bifidiobacterium bifidus, Bifidiobacterium breve, Bifidiobacterium infant's, Bifidiobacterium infantus, Bifidiobacterium longum, and any genetic variants thereof.

Examples of suitable non-lactic acid-producing Bacillus include, but are not limited to: Bacillus subtilis, Bacillus uniflagellatus, Bacillus lateropsorus, Bacillus laterosporus BOD, Bacillus megaterium, Bacillus polymyxa, Bacillus licheniformis, Bacillus pumilus, and Bacillus sterothermophilus. Other species that could be employed due to probiotic activity include members of the Streptococcus (Enterococcus) genus. For example, Enterococcus faecium, is commonly used as a livestock probiotic and, thus, could be utilized as a co-administration agent. Furthermore, it is also intended that any of the acid-producing species of probiotic or nutritional bacteria known in the art can be used in the compositions comprising a microbial consortium as described herein.

A nutrient supplement comprising the viable gut bacteria and/or microbial consortium as described herein can include any of a variety of nutritional agents, including vitamins, minerals, essential and nonessential amino acids, carbohydrates, lipids, foodstuffs, dietary supplements, short chain fatty acids and the like. Preferred compositions comprise vitamins and/or minerals in any combination. Vitamins for use in a composition as described herein can include vitamins B, C, D, E, folic acid, K, niacin, and like vitamins. The composition can contain any or a variety of vitamins as may be deemed useful for a particularly application, and therefore, the vitamin content is not to be construed as limiting. Typical vitamins are those, for example, recommended for daily consumption and in the recommended daily amount (RDA), although precise amounts can vary. The composition can preferably include a complex of the RDA vitamins, minerals and trace minerals as well as those nutrients that have no established RDA, but have a beneficial role in healthy human or mammal physiology. The preferred mineral format would include those that are in either the gluconate or citrate form because these forms are more readily metabolized by lactic acid bacteria. In a related embodiment, the compositions described herein are contemplated to comprise a microbial consortium in combination with a viable lactic acid bacteria in combination with any material to be adsorbed, including but not limited to nutrient supplements, foodstuffs, vitamins, minerals, medicines, therapeutic compositions, antibiotics, hormones, steroids, and the like compounds where it is desirable to insure efficient and healthy absorption of materials from the gastrointestinal tract into the blood. The amount of material included in the composition can vary widely depending upon the material and the intended purpose for its absorption, such that the composition is not to be considered as limiting.

In some embodiments, the compositions described herein can further include a prebiotic and/or a fiber. Many forms of “fiber” exhibit some level of prebiotic effect. Thus, there is considerable overlap between substances that can be classified as “prebiotics” and those that can be classified as “fibers”. Non-limiting examples of prebiotics suitable for use in the compositions and methods include psyllium, fructo-oligosaccharides, inulin, oligofructose, galacto-oligosaccharides, isomalto-oligosaccharides xylo-oligosaccharides, soy-oligosaccharides, gluco-oligosaccharides, mannan-oligosaccharides, arabinogalactan, arabinxylan, lacto sucrose, gluconannan, lactulose, polydextrose, oligodextran, gentioligosaccharide, pectic oligosaccharide, xanthan gum, gum arabic, hemicellulose, resistant starch and its derivatives, and mixtures and/or combinations thereof. The compositions can comprise from about 100 mg to about 100 g, alternatively from about 500 mg to about 50 g, and alternatively from about 1 g to about 40 g, of prebiotic, per day or on a less than daily schedule.

Aspects of the technology described herein also include short chain fatty acids (SCFAs) and medium chain triglycerides (MCTs). Short chain fatty acids can have immunomodulatory (i.e., immunosuppressive) effects and therefore their production (i.e., biosynthesis or conversion by fermentation) is advantageous for the prevention, control, mitigation, and treatment of autoimmune and/or inflammatory disorders (Lara-Villoslada F. et al., 2006. Eur J Nutr. 45(7): 418-425). In germ-free mice and vancomycin-treated conventional mice, administration of SCFA (acetate, propionate, or butyrate) restored normal numbers of Tregs in the large intestine (Smith P M, et al. Science. 2013; 569-573). Short-chain fatty acids (SCFA) are produced by some bacteria as a byproduct of xylose fermentation. SCFA are one of the most abundant metabolites produced by the gut microbiome, particularly the family Clostridiaceae, including members of the genus Clostridium, Ruminococcus, or Blautia. In some aspects, the pharmaceutical composition, dosage form, or kit comprises at least one type of microbe (e.g., one or more microbial species, such as a bacterial species, or more than one strain of a particular microbial species) and at least one type of prebiotic such that the composition, dosage form, or kit is capable of increasing the level of one or more immunomodulatory SCFA (e.g., acetate, propionate, butyrate, or valerate) in a mammalian subject. Optionally, the pharmaceutical composition, dosage form, or kit further comprises one or more substrates of one or more SCFA-producing fermentation and/or biosynthesis pathways. In certain embodiments, the administration of the composition, dosage form, or kit to a mammalian subject results in the increase of one or more SCFAs in the mammalian subject by approximately 1.5-fold, 2-fold, 5-fold, 10-fold, 20-fold, 50-fold, 100-fold, or greater than 100-fold. In some embodiments, the dysbiosis is caused by a deficiency in microbes that produce short chain fatty acids. Accordingly, in some embodiments, the probiotic composition can contain a species of bacteria that produce short chain fatty acids.

MCTs passively diffuse from the GI tract to the portal system (longer fatty acids are absorbed into the lymphatic system) without requirement for modification like long-chain fatty acids or very-long-chain fatty acids. In addition, MCTs do not require bile salts for digestion. Patients who have malnutrition or malabsorption syndromes are treated with MCTs because they do not require energy for absorption, use, or storage. Medium-chain triglycerides are generally considered a good biologically inert source of energy that the human body finds reasonably easy to metabolize. They have potentially beneficial attributes in protein metabolism, but may be contraindicated in some situations due to their tendency to induce ketogenesis and metabolic acidosis. Due to their ability to be absorbed rapidly by the body, medium-chain triglycerides have found use in the treatment of a variety of malabsorption ailments. MCT supplementation with a low-fat diet has been described as the cornerstone of treatment for primary intestinal lymphangiectasia (Waldmann's disease). MCTs are an ingredient in parenteral nutritional emulsions.

Also contemplated herein are kits comprising, at a minimum, a viable gut bacteria and/or a microbial consortium prep or formulations comprising all of the members of the consortium in an admixture or comprising all of the members of the consortium in sub-combinations or sub-mixtures. In some embodiments, the kit further comprises empty capsules to be filled by the practitioner and/or one or more reagents for enteric coating such capsules. It is also contemplated herein that the microbe preparation is provided in a dried, lyophilized or powdered form.

In one embodiment, a kit comprises at least two species selected from the group consisting of: Bacteroides fragilis, Bacteroides ovatus, Bacteroides vulgatus, Parabacteroides distasonis and Prevotella melaninogenica. In another embodiment a kit comprises at least three, at least four, at least five, or all six of the species form the group of: Clostridium ramosum, Clostridium scindens, Clostridium hiranonsis, Clostridium bifermentans, Clostridium leptum, and Clostridium sardiniensis.

In another embodiment, the kit comprises Subdoligranulum variabile. In another embodiment, the kit comprises Subdoligranulum variabile and at least one other species of viable gut bacteria and/or microbial consortium described herein.

In another embodiment, the kits comprise at least two, at least three, at least four, or all five species selected from the group consisting of: Bacteroides fragilis, Bacteroides ovatus, Bacteroides vulgatus, Parabacteroides distasonis, Prevotella melaninogenica, and at least one, at least two, at least three, at least four, at least five, or all six species selected from the group consisting of: Clostridium ramosum, Clostridium scindens, Clostridium hiranonsis, Clostridium bifermentans, Clostridium leptum, and Clostridium sardiniensis. In another embodiment, the kit comprises at least one reducing agent such as N-acetylcysteine, cysteine, or methylene blue for growing, maintaining and/or encapsulating the microbes under anaerobic conditions. The kits described herein are also contemplated to include cell growth media and supplements necessary for expanding the microbial preparation. The kits described herein are also contemplated to include one or more prebiotics as described herein.

Prior to administration of the bacterial composition, the patient may optionally have a pretreatment protocol to prepare the gastrointestinal tract to receive the bacterial composition. In certain embodiments, the pretreatment protocol is advisable, such as when a patient has an acute infection with a highly resilient pathogen. In other embodiments, the pretreatment protocol is entirely optional, such as when the pathogen causing the infection is not resilient, or the patient has had an acute infection that has been successfully treated but where the physician is concerned that the infection may recur. In these instances, the pretreatment protocol can enhance the ability of the bacterial composition to affect the patient's microbiome. In an alternative embodiment, the subject is not pre-treated with an antibiotic.

As one way of preparing the patient for administration of the microbial ecosystem, at least one antibiotic can be administered to alter the bacteria in the patient. As another way of preparing the patient for administration of the microbial ecosystem, a standard colon-cleansing preparation can be administered to the patient to substantially empty the contents of the colon, such as used to prepare a patient for a colonoscopy. By “substantially emptying the contents of the colon,” this application means removing at least 75%, at least 80%, at least 90%, at least 95%, or about 100% of the contents of the ordinary volume of colon contents. Antibiotic treatment can precede the colon-cleansing protocol.

If a patient has received an antibiotic for treatment of an infection, or if a patient has received an antibiotic as part of a specific pretreatment protocol, in one embodiment the antibiotic should be stopped in sufficient time to allow the antibiotic to be substantially reduced in concentration in the gut before the bacterial composition is administered. In one embodiment, the antibiotic may be discontinued 1, 2, or 3 days before the administration of the bacterial composition. In one embodiment, the antibiotic can be discontinued 3, 4, 5, 6, or 7 antibiotic half-lives before administration of the bacterial composition. If the pretreatment protocol is part of treatment of an acute infection, the antibiotic may be chosen so that the infection is sensitive to the antibiotic, but the constituents in the bacterial composition are not sensitive to the antibiotic.

Any of the preparations described herein can be administered once on a single occasion or on multiple occasions, such as once a day for several days or more than once a day on the day of administration (including twice daily, three times daily, or up to five times daily). Or the preparation can be administered intermittently according to a set schedule, e.g., once weekly, once monthly, or when the patient relapses from the primary illness. In another embodiment, the preparation can be administered on a long-term basis to assure the maintenance of a protective or therapeutic effect.

In one embodiment, a first species of viable gut bacteria and/or microbial consortium comprising at least two bacterial species known to enhance colonization of beneficial organisms (e.g., Bacteroides vulgatus and Bacteroides ovatus) is administered to a subject prior to administration of a second species of viable gut bacteria and/or microbial consortium.

Another aspect described herein relates to a method for enhancing the colonization and/or persistence of a species of viable gut bacteria and/or microbial consortium, the method comprising administering a first species of viable gut bacteria and/or microbial consortium comprising at least two bacterial species selected from the group consisting of: Bacteroides fragilis, Bacteroides ovatus, Bacteroides vulgatus, Parabacteroides distasonis, Prevotella melaninogenica, to a subject prior to administering a second species of viable gut bacteria and/or microbial consortium comprising at least 4 bacterial species selected from the group consisting of: Clostridium ramosum, C. scindens, C. hiranonsis, C. bifermentans, C. leptum and C. sardiniensis, wherein the first microbial consortium enhances the colonization and/or persistence of the second microbial consortium.

It is also contemplated herein that a first species of viable gut bacteria and/or microbial consortium comprising at least two bacterial species selected from the group consisting of: Clostridium ramosum, C. scindens, C. hiranonsis, C. bifermentans, C. leptum and C. sardiniensis, is administered to a subject prior to administering a second species of viable gut bacteria and/or microbial consortium comprising at least two bacterial species selected from the group consisting of: Bacteroides fragilis, Bacteroides ovatus, Bacteroides vulgatus, Parabacteroides distasonis, Prevotella melaninogenica.

It is also contemplated herein that a species of viable gut bacteria and/or first microbial consortium comprising at least two bacterial species selected from the group consisting of: Clostridium ramosum, C. scindens, C. hiranonsis, C. bifermentans, C. leptum and C. sardiniensis, is administered to a subject in combination with (e.g., simultaneously) a second species of viable gut bacteria and/or microbial consortium comprising at least two bacterial species selected from the group consisting of: Bacteroides fragilis, Bacteroides ovatus, Bacteroides vulgatus, Parabacteroides distasonis, and Prevotella melaninogenica.

### Efficacy

Typically, a food allergy response can manifest with one of more of the following symptoms or indicators: (i) a marked drop in core body temperature, (ii) an increase in total IgE, (iii) an increase in allergen-specific IgE, (iv) mast cell expansion, (v) release of mast cell granule protease 1 (MMCP-1) and (vi) increase in Th2 cell skewing. Thus, efficacious treatment and/or prevention of food allergy using the methods and compositions described herein can reduce or eliminate at least one of the symptoms or indicators associated with food allergy, as described above. “Reduced” symptoms or indicators mean at least 20% reduced, at least 30% reduced, at least 40% reduced, at least 50% reduced, at least 60% reduced, at least 70% reduced, at least 80% reduced, at least 90% reduced, at least 95% reduced, at least 98% reduced or even at least 99% or further reduction. Methods for the measurement of each of these parameters are known to those of ordinary skill in the art.

The methods and compositions described herein provide treatment or prevention of food allergy involving or provoking anaphylaxis—i.e., IgE-mediated histamine release or direct allergen-mediated degranulation of mast cells and basophils and resulting pathology. Non-limiting examples include allergy or anaphylactic reaction to peanut, tree nuts, and shellfish, among others noted elsewhere herein. Food sensitivity, e.g., lactose intolerance or gluten intolerance involves different mechanisms. While it is contemplated that a microbial consortium as described herein can benefit those with food sensitivities (e.g., by reducing or eliminating a dysbiotic state and thereby reducing gut inflammation), the distinction between food sensitivities and food allergies should be specifically noted. First and foremost, sensitivities do not provoke an anaphylactic response.

Effective prevention of food allergy can be assessed using an accepted animal model, such as that described herein or others known to those of ordinary skill in the art, wherein a regimen that sensitizes the animals to a given food allergen in the absence of microbial consortium treatment fails to provoke a substantial allergic response in animals administered a protective microbial consortium as described herein. As used herein, the term “fails to provoke a substantial allergic response” means that there is less than 20% of the allergic response (as measured by one or more of the criteria (i)-(vi) described above) seen in animals sensitized to the allergen but without administration of a protective or therapeutic microbial consortium as described herein. In human clinical practice, prevention or cure can be evaluated by administration of the given microbial consortium followed by administration of an allergen under controlled circumstances in a doctor's office or hospital setting. For prevention, the microbial consortium can be administered prior to a patient's initial exposure to or consumption of a given food allergen. For therapy for established food allergy, the microbial consortium can be administered as described herein, followed by consumption of the food allergen in a controlled clinical setting. A lack of allergic reaction, or even a reduced allergic reaction relative to the patient's previous allergic responses to the allergen (i.e., at least 20% reduced, at least 30% reduced, at least 40% reduced, at least 50% reduced, at least 60% reduced, at least 70% reduced, at least 80% reduced, at least 90% reduced, at least 95% reduced, at least 98% reduced or even at least 99% or further reduction) is evidence of effective treatment.

Repeated administration of the species of viable gut bacteria and/or microbial consortium may be beneficial to maintain a protective or curative effect.

In addition, efficacy of a particular formulation can be determined in vitro or in an in vivo or in situ mouse model as described in herein or as known in the art (e.g., Noval Rivas et al. J Allergy Clin Immunol (2013) 131(1):201-212 or Noval Rivas et al., Immunity (2015) 42:512-523, the contents of which are each incorporated herein in their entirety).

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A pharmaceutical composition comprising:
   (i) a preparation comprising a species of viable gut bacteria, in an amount sufficient to treat or prevent a food allergy when administered to an individual in need thereof, and
   (ii) a pharmaceutically acceptable carrier.
2. The pharmaceutical composition of paragraph 1, wherein the species of viable gut bacteria is Subdoligranulum variabile.
3. The pharmaceutical composition of paragraph 1 or paragraph 2, formulated to deliver the viable bacteria to the small intestine.
4. The pharmaceutical composition of any one of paragraphs 1-3, wherein the pharmaceutically acceptable carrier comprises an enteric coating composition that encapsulates the species of viable gut bacteria.
5. The composition of paragraph 4, wherein the enteric-coating composition is in the form of a capsule, gel, pastille, tablet or pill.
6. The composition of any one of paragraphs 1-5, wherein the composition is formulated to deliver a dose of at least 5×10⁶ colony forming units per mL (CFU/m)-2×10⁷ CFU/m.
7. The composition of any one of paragraphs 1-6, wherein the composition is formulated to deliver at least 5×10⁶ CFU/m-2×10⁷ CFU/m in less than 30 capsules per one time dose.
8. The composition of any one of paragraphs 1-7, wherein the composition is frozen for storage.
9. The composition of any one of paragraphs 1-8, wherein the species of viable gut bacteria are encapsulated under anaerobic conditions.
10. The composition of paragraph 9, wherein anaerobic conditions comprise one or more of the following:
   (i) oxygen impermeable capsules,
   (ii) addition of a reducing agent including N-acetylcysteine, cysteine, or methylene blue to the composition, or
   (iii) use of spores for organisms that sporulate.
11. The composition of any one of paragraphs 1-10, wherein the composition comprises a 16S rDNA sequence at least 97% identical to a 16S rDNA sequence present in a reference strain operational taxonomic unit for Subdoligranulum variabile.
12. The composition of any one of paragraphs 1-11, wherein the enteric-coating comprises a polymer, nanoparticle, fatty acid, shellac, or a plant fiber.
13. The composition of any one of paragraphs 1-12, wherein the species of viable gut bacteria is encapsulated, lyophilized, formulated in a food item, or is formulated as a liquid, gel, fluid-gel, or nanoparticles in a liquid.
14. The composition of any one of paragraphs 1-13, further comprising a pre-biotic composition.
15. A pharmaceutical composition comprising:
   (i) a preparation comprising a microbial consortium of isolated bacteria that comprises two to twenty species of viable gut bacteria, at least two of which are selected from the group consisting of Bacteroides fragilis, Bacteroides ovatus, Bacteroides vulgatus, Parabacteroides distasonis, and Prevotella melaninogenica, in an amount sufficient to treat or prevent a food allergy when administered to an individual in need thereof, and
   (ii) a pharmaceutically acceptable carrier.
16. A pharmaceutical composition comprising:

The pharmaceutical composition of paragraph 15, wherein the viable gut bacteria are anaerobic gut bacteria.
17. The pharmaceutical composition of paragraph 15 or paragraph 16, formulated to deliver the viable bacteria to the small intestine.
18. The pharmaceutical composition of any one of paragraphs 15-17, wherein the pharmaceutically acceptable carrier comprises an enteric coating composition that encapsulates the microbial consortium.
19. The composition of paragraph 18, wherein the enteric-coating composition is in the form of a capsule, gel, pastille, tablet or pill.
20. The composition of any one of paragraphs 15-19, wherein the viable gut bacteria are human gut bacteria.
21. The composition of any one of paragraphs 15-20, wherein the consortium comprises at least three species selected from the group consisting of Bacteroides fragilis, Bacteroides ovatus, Bacteroides vulgatus, Parabacteroides distasonis, and Prevotella melaninogenica.
22. The composition of any one of paragraphs 15-21, wherein the consortium comprises at least four species selected from the group consisting of Bacteroides fragilis, Bacteroides ovatus, Bacteroides vulgatus, Parabacteroides distasonis, and Prevotella melaninogenica.
23. The composition of any one of paragraphs 15-22, wherein the consortium comprises each of the species Bacteroides fragilis, Bacteroides ovatus, Bacteroides vulgatus, Parabacteroides distasonis, and Prevotella melaninogenica.
24. The composition of any one of paragraphs 15-23, wherein the consortium further comprises at least one species selected from the group consisting of Clostridium ramosum, Clostridium scindens, Clostridium hiranonsis, Clostridium bifermentans, Clostridium leptum, and Clostridium sardiniensis.
25. The composition of any one of paragraphs 15-24, wherein the consortium further comprises at least two species selected from the group consisting of Clostridium ramosum, Clostridium scindens, Clostridium hiranonsis, Clostridium bifermentans, Clostridium leptum, and Clostridium sardiniensis.
26. The composition of any one of paragraphs 15-25, wherein the consortium further comprises at least three species selected from the group consisting of Clostridium ramosum, Clostridium scindens, Clostridium hiranonsis, Clostridium bifermentans, Clostridium leptum, and Clostridium sardiniensis.
27. The composition of any one of paragraphs 15-26, wherein the consortium further comprises at least four species selected from the group consisting of Clostridium ramosum, Clostridium scindens, Clostridium hiranonsis, Clostridium bifermentans, Clostridium leptum, and Clostridium sardiniensis.
28. The composition of any one of paragraphs 15-27, wherein the consortium further comprises at least five species selected from the group consisting of Clostridium ramosum, Clostridium scindens, Clostridium hiranonsis, Clostridium bifermentans, Clostridium leptum, and Clostridium sardiniensis.
29. The composition of any one of paragraphs 15-28, wherein the consortium further comprises each of the species Clostridium ramosum, Clostridium scindens, Clostridium hiranonsis, Clostridium bifermentans, Clostridium leptum, and Clostridium sardiniensis.
30. The composition of any one of paragraphs 15-29, wherein the species of viable gut bacteria are present in substantially equal biomass.
31. The composition of any one of paragraphs 15-30, wherein the composition is formulated to deliver a dose of at least 1×10⁹ colony forming units (CFUs).
32. The composition of any one of paragraphs 15-31, wherein the composition is formulated to deliver at least 1×10⁹ CFUs in less than 30 capsules per one time dose.
33. The composition of any one of paragraphs 15-32, wherein the composition is frozen for storage.
34. The composition of any one of paragraphs 15-33, wherein the species of viable gut bacteria are encapsulated under anaerobic conditions.
35. The composition of paragraph 34, wherein anaerobic conditions comprise one or more of the following:
   (i) oxygen impermeable capsules,
   (ii) addition of a reducing agent including N-acetylcysteine, cysteine, or methylene blue to the composition, or
   (iii) use of spores for organisms that sporulate.
36. The composition of any one of paragraphs 15-35, wherein the composition comprises at least two bacterial species, each comprising a 16S rDNA sequence at least 97% identical to a 16S rDNA sequence present in a reference strain operational taxonomic unit, the reference strain selected from the species Bacteroides fragilis, Bacteroides ovatus, Bacteroides vulgatus, Parabacteroides distasonis and Prevotella melaninogenica.
37. The composition of any one of paragraphs 15-36, wherein the composition comprises at least three bacterial species, each comprising a 16S rDNA sequence at least 97% identical to a 16S rDNA sequence present in a reference strain operational taxonomic unit, the reference strain selected from the species Bacteroides fragilis, Bacteroides ovatus, Bacteroides vulgatus, Parabacteroides distasonis and Prevotella melaninogenica.
38. The composition of any one of paragraphs 15-37, wherein the composition comprises at least four bacterial species, each comprising a 16S rDNA sequence at least 97% identical to a 16S rDNA sequence present in a reference strain operational taxonomic unit, the reference strain selected from the species Bacteroides fragilis, Bacteroides ovatus, Bacteroides vulgatus, Parabacteroides distasonis and Prevotella melaninogenica.
39. The composition of any one of paragraphs 15-38, wherein the composition comprises at least five bacterial species, each comprising a 16S rDNA sequence at least 97% identical to a 16S rDNA sequence present in a reference strain operational taxonomic unit, the reference strains including each of the species Bacteroides fragilis, Bacteroides ovatus, Bacteroides vulgatus, Parabacteroides distasonis and Prevotella melaninogenica.
40. The composition of any one of paragraphs 15-39, wherein the composition does not comprise any of the Species Escherichia coli, Klebsiella pneumoniae, Proteus mirabilis, Enterobacter cloacae, Bilophila wadsworthia, Alistipes onderdonkii, Desulfovibrio species, Lactobacillus johnsonii, or Parasutterella excrementihominis.
41. The composition of any one of paragraphs 15-40, wherein the composition does not comprise bacteria of the Genera Bilophila, Enterobacter, Escherichia, Klebsiella, Proteus, Alistipes, Blautia, Desulfovibrio, and Parasutterella.
42. The composition of any one of paragraphs 15-41, wherein the composition does not comprise bacteria of the Families Desulfovibrionaceae, Enterobacteriaceae, Rikenellaceae, and Sutterellaceae.
43. The composition of any one of paragraphs 15-42, wherein the composition does not comprise bacteria of the Families Lactobacillaceae, or Enterbacteriaceae.
44. The composition of any one of paragraphs 15-43, wherein the composition does not comprise bacteria of the Order Burkholdales, Desulfovibrionales, or Enterobacteriales.
45. The composition of paragraph 15, which comprises at least four species of viable non-pathogenic gut bacteria.
46. The composition of paragraph 15, which comprises at least two and up to eleven species of viable non-pathogenic gut bacteria.
47. The composition of paragraph 15, wherein the consortium comprises Bacteroides fragilis, Bacteroides ovatus, Bacteroides vulgatus, Parabacteroides distasonis, and Prevotella melaninogenica.
48. The composition of paragraph 15, wherein the consortium comprises: Bacteroides fragilis, Bacteroides ovatus, Bacteroides vulgatus, Parabacteroides distasonis, Prevotella melaninogenica, Clostridium ramosum, Clostridium scindens, Clostridium rhiranonsis, Clostridium bifermentans, Clostridium leptum, and Clostridium sardiniensis
49. The composition of paragraph 15, wherein the consortium consists essentially of Bacteroides fragilis, Bacteroides ovatus, Bacteroides vulgatus, Parabacteroides distasonis and Prevotella melaninogenica.
50. The composition of paragraph 15, wherein the consortium consists essentially of: Clostridium ramosum, Clostridium scindens, Clostridium hiranonsis, Clostridium bifermentans, Clostridium leptum, Clostridium sardiniensis, Bacteroides fragilis, Bacteroides ovatus, Bacteroides vulgatus, Parabacteroides distasonis, and Prevotella melaninogenica.
51. The composition of any one of paragraphs 15-50, wherein the enteric-coating comprises a polymer, nanoparticle, fatty acid, shellac, or a plant fiber.
52. The composition of any one of paragraphs 15-51, wherein the consortium is encapsulated, lyophilized, formulated in a food item, or is formulated as a liquid, gel, fluid-gel, or nanoparticles in a liquid.
53. The composition of any one of paragraphs 15-52, further comprising a pre-biotic composition.
54. A pharmaceutical composition comprising:
   (i) a preparation comprising at least two species of viable, anaerobic gut bacteria selected from the group consisting of: Bacteroides fragilis, Bacteroides ovatus, Bacteroides vulgatus, Parabacteroides distasonis, and Prevotella melaninogenica, in an amount sufficient to treat or prevent a food allergy when administered to an individual in need thereof, and
   (ii) a pharmaceutically acceptable carrier.
55. A pharmaceutical composition comprising:
   (i) a preparation comprising at least three species of viable, anaerobic gut bacteria selected from the group consisting of: Bacteroides fragilis, Bacteroides ovatus, Bacteroides vulgatus, Parabacteroides distasonis, and Prevotella melaninogenica, in an amount sufficient to treat or prevent a food allergy when administered to an individual in need thereof, and
   (ii) a pharmaceutically acceptable carrier.
56. A pharmaceutical composition comprising:
   (i) a preparation comprising at least four species of viable, anaerobic gut bacteria selected from the group consisting of: Bacteroides fragilis, Bacteroides ovatus, Bacteroides vulgatus, Parabacteroides distasonis, and Prevotella melaninogenica, in an amount sufficient to treat or prevent a food allergy when administered to an individual in need thereof, and
   (ii) a pharmaceutically acceptable carrier.
57. A pharmaceutical composition comprising:
   (i) a preparation comprising viable, anaerobic gut bacteria including each of Bacteroides fragilis, Bacteroides ovatus, Bacteroides vulgatus, Parabacteroides distasonis, and Prevotella melaninogenica, in an amount sufficient to treat or prevent a food allergy when administered to an individual in need thereof, and
   (ii) a pharmaceutically acceptable carrier.
58. The pharmaceutical composition of any one of paragraphs 54-57, which comprises not more than forty species of viable, anaerobic gut bacteria.
59. The pharmaceutical composition of any one of paragraphs 54-57, which comprises not more than thirty species of viable, anaerobic gut bacteria.
60. The pharmaceutical composition of any one of paragraphs 54-57, which comprises not more than twenty species of viable, anaerobic gut bacteria.
61. The pharmaceutical composition of any one of paragraphs 54-57, which comprises not more than fifteen species of viable, anaerobic gut bacteria.
62. The pharmaceutical composition of any one of paragraphs 54-57, which comprises not more than eleven species of viable, anaerobic gut bacteria.
63. The composition of any one of paragraphs 54-57, which further comprises at least one species of bacteria selected from the group consisting of: Clostridium ramosum, Clostridium scindens, Clostridium hiranonsis, Clostridium bifermentans, Clostridium leptum, and Clostridium sardiniensis.
64. The composition of any one of paragraphs 54-57, which further comprises at least two species of bacteria selected from the group consisting of: Clostridium ramosum, Clostridium scindens, Clostridium hiranonsis, Clostridium bifermentans, Clostridium leptum, and Clostridium sardiniensis.
65. The composition of any one of paragraphs 54-57, which further comprises at least three species of bacteria selected from the group consisting of: Clostridium ramosum, Clostridium scindens, Clostridium hiranonsis, Clostridium bifermentans, Clostridium leptum, and Clostridium sardiniensis.
66. The composition of any one of paragraphs 54-57, which further comprises at least four species of bacteria selected from the group consisting of: Clostridium ramosum, Clostridium scindens, Clostridium hiranonsis, Clostridium bifermentans, Clostridium leptum, and Clostridium sardiniensis
67. The composition of any one of paragraphs 54-57, which further comprises at least five species of bacteria selected from the group consisting of: Clostridium ramosum, Clostridium scindens, Clostridium hiranonsis, Clostridium bifermentans, Clostridium leptum, and Clostridium sardiniensis.
68. The composition of any one of paragraphs 54-57, which further comprises Clostridium ramosum, Clostridium scindens, Clostridium hiranonsis, Clostridium bifermentans, Clostridium leptum, and Clostridium sardiniensis.
69. The pharmaceutical composition of any one of paragraphs 45-68, wherein the microbial species do not comprise any of the Species Escherichia coli, Klebsiella pneumoniae, Proteus mirabilis, Enterobacter cloacae, Bilophila wadsworthia, Alistipes onderdonkii, Desulfovibrio species, Lactobacillus johnsoni, and Parasutterella excrementihominis.
70. The pharmaceutical composition of any one of paragraphs 45-68, wherein the microbial species do not comprise bacteria of the Genera Bilophila, Enterobacter, Escherichia, Klebsiella, Proteus, Alistipes, Blautia, Desulfovibrio, and Parasutterella.
71. The pharmaceutical composition of any one of paragraphs 45-68, wherein the microbial species do not comprise bacteria of the Families Desulfovibrionaceae, Enterobacteriaceae, Rikenellaceae, and Sutterellaceae.
72. The pharmaceutical composition of any one of paragraphs 45-68, wherein the microbial species do not comprise bacteria of the Families Lactobacillaceae, or Enterbacteriaceae
73. The pharmaceutical composition of any one of paragraphs 45-68, wherein the microbial species do not comprise bacteria of the Order Burkholdales, Desulfovibrionales, or Enterobacteriales.
74. The pharmaceutical composition of any one of paragraphs 45-73, wherein the pharmaceutically acceptable carrier comprises an enteric coating composition that encapsulates the microbial consortium.
75. The pharmaceutical composition of any one of paragraphs 45-74, formulated to deliver the viable bacteria to the small intestine.
76. The composition of any one of paragraphs 45-75, wherein the pharmaceutically acceptable carrier comprises a capsule, gel, pastille, tablet or pill.
77. The composition of any one of paragraphs 45-76, wherein the consortium of viable gut bacteria is formulated with an enteric coating.
78. The composition of any one of paragraphs 54-77, wherein the species of viable gut bacteria are human gut bacteria.
79. The composition of any one of paragraphs 54-78, wherein the species of viable gut bacteria are present in substantially equal biomass.
80. The composition of any one of paragraphs 54-79, wherein the composition is formulated to deliver a dose of at least 1×10⁹ colony forming units (CFUs).
81. The composition of any of paragraphs 54-80, wherein the composition is formulated to deliver at least 1×10⁹ CFUs in less than 30 capsules per one time dose.
82. The composition of any of paragraphs 54-81, wherein the composition is frozen for storage.
83. The composition of any of paragraphs 54-82, wherein the species of viable gut bacteria are encapsulated under anaerobic conditions.
84. The composition of paragraph 83, wherein anaerobic conditions comprise one or more of the following:
   (i) oxygen impermeable capsules,
   (ii) addition of a reducing agent including N-acetylcysteine, cysteine, or methylene blue to the composition, or
   (iii) use of spores for organisms that sporulate.
85. The composition of paragraph 77, wherein the enteric-coating comprises a polymer, nanoparticle, fatty acid, shellac, or a plant fiber.
86. The composition of any one of paragraphs 54-85, further comprising a pre-biotic composition.
87. The composition of any one of paragraphs 54-86, wherein the composition is encapsulated, a lyophilisate, formulated in a food item, or is formulated as a liquid, gel, fluid-gel, or nanoparticles in a liquid.
88. A method for preventing the onset of a food allergy in a subject, the method comprising: administering to a subject a composition of any one of paragraphs 1-87, thereby preventing the onset of a food allergy in the subject.
89. The method of paragraph 88, wherein the composition is administered by oral administration, enema, suppository, or orogastric tube.
90. The method of either of paragraphs 88 or 89 wherein the species of viable gut bacteria are isolated and/or purified from a subject known to be tolerant to a selected food allergen.
91. The method of any one of paragraphs 88-90, wherein the species of viable gut bacteria are prepared by culture under anaerobic conditions.
92. The method of any one of paragraphs 88-91, wherein the species of viable gut bacteria are formulated to maintain anaerobic conditions.
93. The method of paragraph 92, wherein anaerobic conditions are maintained by one or more of the following:
   (i) oxygen impermeable capsules,
   (ii) addition of a reducing agent including N-acetylcysteine, cysteine, or methylene blue to the composition, or
   (iii) use of spores for organisms that sporulate.
94. The method of any one of paragraphs 88-93, wherein the composition administered further comprises a pre-biotic composition.
95. The method of any one of paragraphs 88-94, wherein the composition is enteric-coated.
96. The method of any one of paragraphs 88-95, wherein the treatment administered prevents and/or reverses T_{H}2 programming of Tregs and other mucosal T cell populations.
97. The method of any one of paragraphs 88-96, wherein the subject is a human subject.
98. The method of any one of paragraphs 88-97, further comprising a step of diagnosing the subject as likely to develop a food allergy.
99. The method of any one of paragraphs 88-98, further comprising a step of testing a fecal sample from the subject for the presence and/or levels of the bacteria in the minimal microbial consortium.
100. The method of any one of paragraphs 88-99, wherein the food allergy comprises allergy to soy, wheat, eggs, dairy, peanuts, tree nuts, shellfish, fish, mushrooms, stone fruits and other fruits.
101. The method of any one of paragraphs 88-100, wherein the composition is administered before the first exposure to a potential food allergen.
102. The method of any one of paragraphs 88-101, wherein the composition is administered upon clinical signs of atopic symptoms.
103. The method of any one of paragraphs 88-102, wherein the composition is administered to an individual with diagnosed food allergy.
104. The method of any one of paragraphs 88-103, wherein the subject is pretreated with an antibiotic.
105. A method for reducing or eliminating a subject's immune reaction to a food allergen, the method comprising: administering to a subject a composition of any of paragraphs 1-87, thereby reducing or eliminating a subject's immune reaction to a food allergen.
106. The method of paragraph 105, wherein the composition is administered by oral administration, enema, suppository, or orogastric tube.
107. The method of paragraph 105 or 106, wherein the treatment prevents and/or reverses T_{H}2 programming of Tregs.
108. The method of any one of paragraphs 105-107, wherein the subject is a human subject.
109. The method of any one of paragraphs 105-108, further comprising a step of diagnosing the subject as having an IgE-mediated food allergy.
110. The method of any one of paragraphs 105-109, further comprising a step of testing a fecal sample from the subject for the presence and/or levels of the bacteria in the minimal microbial consortium.
111. The method of any one of paragraphs 105-110, wherein the food allergy comprises allergy to soy, wheat, eggs, dairy, peanuts, tree nuts, shellfish, fish, mushrooms, stone fruits or other fruits.
112. The method of any one of paragraphs 105-111, wherein the composition is administered after an initial exposure and/or reaction to a potential food allergen.
113. The method of any one of paragraphs 105-112, wherein the biomass of each of the microbes in the administered compositions is greater than the biomass of each of the microbes relative to a reference.
114. The method of any one of paragraphs 105-113, wherein the subject is pretreated with an antibiotic.
115. The method of any one of paragraphs 105-114, wherein the subject is pretreated with a fasting period not longer than 24 hours.
116. A method of monitoring a subject's microbiome, the method comprising: determining the presence and/or biomass in a biological sample obtained from a subject, and wherein if at two or more species selected from the group consisting of Bacteroides fragilis, Bacteroides ovatus, Bacteroides vulgatus, Parabacteroides distasonis, and Prevotella melaninogenica, are absent or low relative to a reference, the subject is treated with the composition of any one of paragraphs 1-87.
117. The method of paragraph 116, wherein the method further comprises predicting that a subject will have an immune response to a food allergen when the at least two members are absent, the biomass of the at least two members is low relative to a reference, or at least one member of a dysbiotic species is present or elevated relative to a reference.
118. The method of paragraph 116, wherein the method is repeated at least one additional time.
119. The method of paragraph 116, wherein the biological sample is a fecal sample.
120. A method of treating atopic disease in an individual in need thereof, the method comprising administering a composition of any one of paragraphs 1-87 to the individual.
121. The method of paragraph 120, wherein the administration shifts the balance of Th1/Th2 cells towards Th1 T cells.
122. The method of paragraph 120, wherein the administration reduces the number or activity of Th2 T cells.
123. A method of reducing the number or activity of Th2 cells in a tissue of an individual in need thereof, the method comprising administering a composition of any one of paragraphs 1-87 to the individual.
124. The method of paragraph 123, wherein the tissue is a gut tissue.
125. A synergistic microbial composition comprising:
   (a) a first microbial consortium consisting essentially of two to five species of viable gut bacteria selected from the group consisting of: Bacteroides fragilis, Bacteroides ovatus, Bacteroides vulgatus, Parabacteroides distasonis, and Prevotella melaninogenica; and
   (b) a second microbial consortium consisting essentially of one to six species of viable gut bacteria selected from the group consisting of: Clostridium ramosum, Clostridium scindens, Clostridium hiranonsis, Clostridium bifermentans, Clostridium leptum, and Clostridium sardiniensis, wherein one or more members of the second microbial consortium increases the colonization and/or persistence of one or more members of the first microbial consortium in a mammalian host.
126. A synergistic microbial composition comprising:
   (a) a first microbial consortium consisting essentially of one to six species of viable gut bacteria selected from the group consisting of: Clostridium ramosum, Clostridium scindens, Clostridium hiranonsis, Clostridium bifermentans, Clostridium leptum, and Clostridium sardiniensis; and
   (b) a second microbial consortium consisting essentially of two to five species of viable gut bacteria, wherein the species of viable gut bacteria are selected from the group consisting of: Bacteroides fragilis, Bacteroides ovatus, Bacteroides vulgatus, Parabacteroides distasonis, and Prevotella melaninogenica, wherein one or more members of the second microbial consortium increases the colonization and/or persistence of one or more members of the first microbial consortium in a mammalian host.

Some embodiments of the technology described herein can be defined according to any of the following numbered paragraphs:
1. A pharmaceutical composition comprising:
   (i) a preparation comprising a species of viable gut bacteria, in an amount sufficient to treat or prevent a dysbiosis when administered to an individual in need thereof, and
   (ii) a pharmaceutically acceptable carrier.
2. The pharmaceutical composition of paragraph 1, wherein the species of viable gut bacteria is Subdoligranulum variabile.
3. The pharmaceutical composition of paragraph 1 or paragraph 2, formulated to deliver the viable bacteria to the small intestine.
4. The pharmaceutical composition of any one of paragraphs 1-3, wherein the pharmaceutically acceptable carrier comprises an enteric coating composition that encapsulates the species of viable gut bacteria.
5. The composition of paragraph 4, wherein the enteric-coating composition is in the form of a capsule, gel, pastille, tablet or pill.
6. The composition of any one of paragraphs 1-5, wherein the composition is formulated to deliver a dose of at least 5×10⁶ colony forming units per mL (CFU/mL)-2×10⁷ CFU/mL.
7. The composition of any one of paragraphs 1-6, wherein the composition is formulated to deliver at least 5×10⁶ CFU/mL-2×10⁷ CFU/mL in less than 30 capsules per one time dose.
8. The composition of any one of paragraphs 1-7, wherein the composition is frozen for storage.
9. The composition of any one of paragraphs 1-8, wherein the species of viable gut bacteria are encapsulated under anaerobic conditions.
10. The composition of paragraph 9, wherein anaerobic conditions comprise one or more of the following:
   (i) oxygen impermeable capsules,
   (ii) addition of a reducing agent including N-acetylcysteine, cysteine, or methylene blue to the composition, or
   (iii) use of spores for organisms that sporulate.
11. The composition of any one of paragraphs 1-10, wherein the composition comprises a 16S rDNA sequence at least 97% identical to a 16S rDNA sequence present in a reference strain operational taxonomic unit for Subdoligranulum variabile.
12. The composition of any one of paragraphs 1-11, wherein the enteric-coating comprises a polymer, nanoparticle, fatty acid, shellac, or a plant fiber.
13. The composition of any one of paragraphs 1-12, wherein the species of viable gut bacteria is encapsulated, lyophilized, formulated in a food item, or is formulated as a liquid, gel, fluid-gel, or nanoparticles in a liquid.
14. The composition of any one of paragraphs 1-13, further comprising a pre-biotic composition.
15. The pharmaceutical composition of any one of paragraphs 1-14, wherein the dysbiosis is associated with an inflammatory disease or a metabolic disorder.
16. The pharmaceutical composition of any one of paragraphs 1-15, wherein the dysbiosis is associated with an atopic disease or disorder.
17. The pharmaceutical composition of paragraph 16, wherein the atopic disease or disorder is selected from the group consisting of: food allergy, eczema, asthma, and rhinoconjunctivitis.
18. A method for treating or preventing the onset of a dysbiosis in a subject, the method comprising: administering to a subject a pharmaceutical composition of any one of paragraphs 1-17, thereby treating or preventing dysbiosis in the subject.
19. A method for the treatment, or prevention of gut inflammation or a metabolic disease or disorder, the method comprising: administering to a subject a pharmaceutical composition of any one of paragraphs 1-17, thereby treating, or preventing the gut inflammation or metabolic disease or disorder in the subject.
20. A method for the treatment, or prevention of an atopic disease or disorder, the method comprising: administering to a subject a pharmaceutical composition of any one of paragraphs 1-17, thereby treating, or preventing the atopic disease or disorder in the subject.
21. The method of paragraph 20, wherein the atopic disease or disorder is selected from the group consisting of: food allergy, eczema, asthma, and rhinoconjunctivitis.
22. The method of any one of paragraphs 18-21, wherein the pharmaceutical composition is administered by oral administration, enema, suppository, or orogastric tube.
23. The method of any one of paragraphs 18-22, wherein the species of viable gut bacteria are isolated and/or purified from a subject known to be tolerant to a selected allergen.
24. The method of any one of paragraphs 18-23, wherein the species of viable gut bacteria are prepared by culture under anaerobic conditions.
25. The method of any one of paragraphs 18-24, wherein the species of viable gut bacteria are formulated to maintain anaerobic conditions.
26. The method of paragraph 25, wherein anaerobic conditions are maintained by one or more of the following:
   (i) oxygen impermeable capsules,
   (ii) addition of a reducing agent including N-acetylcysteine, cysteine, or methylene blue to the composition, or
   (iii) use of spores for organisms that sporulate.
27. The method of any one of paragraphs 18-26, further comprising administering a pre-biotic composition.
28. The method of any one of paragraphs 18-27, wherein the pharmaceutical composition is enteric-coated.
29. The method of any one of paragraphs 18-28, wherein the treatment administered prevents and/or reverses T_{H}2 programming.
30. The method of any one of paragraphs 18-29, wherein the subject is a human subject.
31. The method of any one of paragraphs 18-30, wherein the subject is under the age of 2 years old.
32. The method of any one of paragraphs 18-30, wherein the subject is age 2 to under 5 years old.
33. The method of any one of paragraphs 18-30, wherein the subject is age 5 to under 12 years old
34. The method of any one of paragraphs 18-30, wherein the subject is age 12 to under 18 years old.
35. The method of any one of paragraphs 18-30, wherein the subject is age 18 to under 65 years old.
36. The method of any one of paragraphs 18-30, wherein the subject is over age 65 years old.
37. The method of any one of paragraphs 18-36, further comprising a step of diagnosing the subject as having or likely to develop an inflammatory disease or an atopic disease or disorder.
38. The method of any one of paragraphs 18-37, further comprising a step of testing a fecal sample from the subject for the presence and/or levels of one or more of the bacteria in the pharmaceutical composition.
39. The method of any one of paragraphs 18-38, wherein the atopic disease is a food allergy, and wherein the food allergy comprises allergy to soy, wheat, eggs, dairy, peanuts, tree nuts, shellfish, fish, mushrooms, stone fruits and/or other fruits.
40. The method of any one of paragraphs 18-39, wherein the pharmaceutical composition is administered before the first exposure to a potential food allergen.
41. The method of any one of paragraphs 18-40, wherein the pharmaceutical composition is administered upon clinical signs of atopic symptoms.
42. The method of any one of paragraphs 18-41, wherein the pharmaceutical composition is administered to an individual with diagnosed with a food allergy.
43. The method of any one of paragraphs 18-42, wherein the subject is pretreated with an antibiotic.
44. A method for reducing or eliminating a subject's immune reaction to an allergen, the method comprising: administering to a subject a pharmaceutical composition of any of paragraphs 1-17, thereby reducing or eliminating a subject's immune reaction to the allergen.
45. The method of paragraph 44, wherein the pharmaceutical composition is administered by oral administration, enema, suppository, or orogastric tube.
46. The method of paragraph 44 or 45, wherein the treatment prevents and/or reverses T_{H}2 programming.
47. The method of any one of paragraphs 44-46, wherein the subject is a human subject.
48. The method of any one of paragraphs 44-47, wherein the subject is under the age of 2 years old.
49. The method of any one of paragraphs 44-47, wherein the subject is age 2 to under 5 years old.
50. The method of any one of paragraphs 44-47, wherein the subject is age 5 to under 12 years old
51. The method of any one of paragraphs 44-47, wherein the subject is age 12 to under 18 years old.
52. The method of any one of paragraphs 44-47, wherein the subject is age 18 to under 65 years old.
53. The method of any one of paragraphs 44-47, wherein the subject is over age 65 years old.
54. The method of any one of paragraphs 44-53, further comprising a step of diagnosing the subject as having an IgE-mediated allergy.
55. The method of any one of paragraphs 44-54, further comprising a step of testing a fecal sample from the subject for the presence and/or levels of one or more of the bacteria in the pharmaceutical composition.
56. The method of any one of paragraphs 44-55, wherein the IgE-mediated allergy is a food allergy selected from the group consisting of: allergy to soy, wheat, eggs, dairy, peanuts, tree nuts, shellfish, fish, mushrooms, stone fruits or other fruits.
57. The method of any one of paragraphs 44-56, wherein the pharmaceutical composition is administered after an initial exposure and/or reaction to a potential allergen.
58. The method of any one of paragraphs 44-57, wherein the biomass of each of the microbes in the administered compositions is greater than the biomass of each of the microbes relative to a reference.
59. The method of any one of paragraphs 44-58, wherein the subject is pretreated with an antibiotic.
60. The method of any one of paragraphs 44-59, wherein the subject is pretreated with a fasting period not longer than 24 hours.
61. A method of monitoring a subject's microbiome, the method comprising: determining the presence and/or biomass in a biological sample obtained from a subject, and wherein if at least one or more species selected from the group consisting of Subdoligranulum variabile, Bacteroides fragilis, Bacteroides ovatus, Bacteroides vulgatus, Parabacteroides distasonis, and Prevotella melaninogenica, are absent or low relative to a reference, the subject is treated with the pharmaceutical composition of any one of paragraphs 1-17.
62. The method of paragraph 61, wherein the method further comprises predicting that a subject will have an immune response to an allergen when the at least one member is absent, the biomass of the at least one member is low relative to a reference, or at least one member of a dysbiotic species is present or elevated relative to a reference.
63. The method of paragraph 61 or 62, wherein the method is repeated at least one additional time.
64. The method of any one of paragraphs 61-63, wherein the biological sample is a fecal sample.
65. A method of treating atopic disease or disorder in an individual in need thereof, the method comprising administering a pharmaceutical composition of any one of paragraphs 1-17 to the individual.
66. The method of paragraph 65, wherein the administration shifts the balance of Tₕ1/Tₕ2 cells towards Th1 T cells.
67. The method of paragraph 65 or 66, wherein the administration reduces the number or activity of Th2 T cells.
68. A method of reducing the number or activity of Th2 cells in a tissue of an individual in need thereof, the method comprising administering a pharmaceutical composition of any one of paragraphs 1-17 to the individual.
69. The method of paragraph 68, wherein the tissue is a gut tissue.
70. The pharmaceutical composition of any one of paragraphs 1-17, for use in treating or preventing gut inflammation.
71. The pharmaceutical composition of any one of paragraphs 1-17, for use in treating or preventing a metabolic disease or disorder.
72. The pharmaceutical composition of any one of paragraphs 1-17, for use in treating or preventing an atopic disease or disorder
73. The pharmaceutical composition of any one of paragraphs 1-17, for use in treating or preventing a food allergy.
74. The pharmaceutical composition of any one of paragraphs 1-17, for use in treating or preventing eczema.
75. The pharmaceutical composition of any one of paragraphs 1-17, for use in treating or preventing asthma.
76. The pharmaceutical composition of any one of paragraphs 1-17, for use in treating or preventing rhinoconjunctivitis.
77. Use of the pharmaceutical composition of any one of paragraphs 1-17, for treating or preventing gut inflammation.
78. Use of the pharmaceutical composition of any one of paragraphs 1-17, for treating or preventing a metabolic disease or disorder.
79. Use of the pharmaceutical composition of any one of paragraphs 1-17, for treating or preventing an atopic disease or disorder
80. Use of the pharmaceutical composition of any one of paragraphs 1-17, for treating or preventing a food allergy.
81. Use of the pharmaceutical composition of any one of paragraphs 1-17, for treating or preventing eczema.
82. Use of the pharmaceutical composition of any one of paragraphs 1-17, for treating or preventing asthma.
83. Use of the pharmaceutical composition of any one of paragraphs 1-17, for treating or preventing rhinoconjunctivitis.

### EXAMPLES

The data provided herein, e.g., in the figures and elsewhere, show that a pharmaceutical composition comprising Subdoligranulum variabile can protect against developing food allergy in a mouse model. Treatment with such a composition can reverse T_{H}2 programming of Tregs. Treatment and/or prevention of food allergy using a similar composition in humans is specifically indicated.

Further methods for testing or measuring the efficacy of a microbial therapy in a mouse model of food allergy are known in the art and/or can be found in e.g., Noval Rivas et al. J Allergy Clin Immunol (2013) 131(1):201-212 or Noval Rivas et al., Immunity (2015) 42:512-523, the contents of which are each incorporated herein in their entirety.

### Example 1: Therapeutic Microbiota to Treat Food Allergy

### Summary

Food allergy is a growing national problem, affecting 6% of children, and 3% of US teens and adults. Unfortunately for these children and their families, the standard of care remains to avoid offending foods and manage symptoms as they occur. Therapies using oral desensitization, alone or with anti-IgE (Omalizumab™), remain experimental with limited success. Needed are therapies that target the aberrant immune responses. As such, this study shows the use of gut microbiota as a therapeutic intervention to promote tolerizing responses that can prevent or mitigate effects of Th2/allergic responses.

Food allergies occur with development of Th2-allergic responses to foodstuffs, in contrast to tolerizing T-regulatory responses that mitigate such responses mucosally. The Th2 responses promote food antigen-specific IgE antibody and recruitment of mucosal mast cells, in contrast to regulatory responses, which inhibit these effects. Once sensitized to one or more food antigens, re-exposure can induce life-threatening anaphylactic responses. Capacity to promote tolerizing responses supports a broad-based therapeutic approach that can act at the earliest stages of exposure as well as in the already-sensitized patient to prevent aberrant allergic responses across a spectrum of foodstuffs.

Leveraging the genetically susceptible IL4RA F709 mouse model of food allergy defined human commensal communities that can both prevent and cure food allergies in preclinical models have been developed. These communities leverage a new therapeutic pathway for patients—immunomodulation from the luminal side of the gut, the space in which the gut microbiota resides. Human gut microbiota consists of many hundreds of species that provide critical functions in normal human development and health, from maturing of the immune system, providing essential nutrients such as B vitamins and vitamin K, and assisting in digestion and metabolism of dietary and exogenous compounds, including drugs and ingested foodstuffs.

### Consortia Development

For pre-clinical studies in mice the component members are grown individually in nutrient-rich media under appropriate anaerobic conditions, quantitated for biomass, and then the consortium is mixed under anaerobic conditions with approximately equal biomass of each component organism to a final concentration ˜5.0×10⁸ colony forming units (CFU)/mL. Input culture volumes for each species have been in the range of 100 mL-1 L. As needed, cultures with a stationary phase biomass <5×10⁸ CFU/mL are concentrated by centrifugation with re-suspension handled under anaerobic conditions.

When mixed, the total biomass remains approximately 5×10⁸ CFU/mL. 2 mL aliquots are placed in cryovials with an anaerobic/pre-reduced atmosphere, snap frozen on liquid nitrogen and stored at −80° C. until use. Rapid freezing has shown to have <½ log effects on the biomass of the component organisms and no effect on efficacy in animal models. For studies, tubes are thawed and mice administered 200 uL of this solution weekly to twice weekly by oral gavage, resulting in a total introduced biomass of 1×10⁸ CFU/mouse. The measurements of gut contents in adult mice (stomach through anus) range from 4-8 mL of material. The gavaged consortium is thus 2.5-5% of the total volume of contents in the mouse gut and >10% of the volume of contents in the small bowel.

In terms of pre-existing microbial biomass from the conventional microbiota—the mouse small intestine on average has ˜10⁴ CFU/mL in proximal duodenum with increase to 10⁸ CFU/mL in the ileum. Biomass increases to 10⁹-10¹⁰ CFU/mL in the cecum and colon. From the standpoint of microbial biomass at locations in the mouse small bowel, the primary site of action of the consortia to promote regulatory T cell responses, the consortium is 10,000× the biomass of the duodenal microbiota, and 1-2× the biomass of the jejunal and ileal microbiota.

In comparison, the adult human gut may contain 4.5 L of material, of which 1 L relates to ingested foodstuffs with 3.5 L of secretions including saliva, bile, and other fluids from the pancreas and intestines. These fluids and electrolytes are largely resorbed in the right colon, subsequent to fecal compaction and passage. Within the intestines, the biomass of organisms also varies, with the highest concentration in the cecum and right side of the colon (10¹⁰-10¹² CFU/mL). In contrast, in the small intestine—the believed site of action, the biomass also ranges from 10⁴ CFU/mL in the duodenum to 10⁸ CFU/mL in the ileum.

### Human Dosing

The CFU/dose for humans is based on the following parameters:

(1) Treatment of Clostridium difficile with oral capsule formulations of human stool: Data from OpenBiome and other groups have shown successful treatment of Clostridium difficile colitis with capsule formulation that administers 3-5×10⁹ CFU in a range of 12-30 capsules taken per one-time dose. A standard 12-capsule regimen is expected to deliver approximately 4.2×10⁹ CFU per dose.

(2) Alter the small intestinal microbiota to promote immunomodulation. An encapsulated formulation releasing contents in the proximal small bowel will deliver a dose of 3-5×10⁹ CFU, exceeding the duodenal biomass by a factor of 10,000, and approaching a 1:1 ratio with communities in the jejunum and ileum.

Other formulations including nanoparticles in liquid, with optional pre-biotic compounds to enhance colonization and viability, or a reconstituted lyophilisate are contemplated, however given the need to prevent exposure to oxygen and for ease of storage and administration, the first formulation uses encapsulated material.

### Administration

Given the obligately anaerobic nature of the component species, phase I studies will use encapsulated formulations with the following properties:

Stage I:
Can be swallowed by an adult or child >8 years of age.
Excludes oxygen
Holds a volume so that a person needs to take 15 or less capsules per dose
Can be stored frozen (−20° C. or −80° C.) and thawed prior to administration
Releases contents after passage through the stomach

In one embodiment, the capsules used by OpenBiome™ for oral FMT therapy are used to encapsulate the GP-IIa mixtures. Other options are also available commercially and contemplated herein. In some embodiments, the capsules consist of frozen material (in order to ensure an adequate product) that is thawed prior to administration and is encapsulated, free of oxygen, with material that survives intact into the small intestine.

### Scale of Culture

The animal studies used pilot cultures in the range of 100-1000 mL. To generate human doses, the culture is scaled by at least a factor of 10. The following steps are contemplated herein.
(1) Perform growth curves in different media conditions—to optimize growth conditions and correlate an OD600 with plated biomass.
(2) Grow the component members anaerobically in liquid media. Media is pre-reduced and incubated at 37° C. with some level of agitation (e.g., 150 rpm or with stirring/fermenter baffles) to insure a maximal culture density. Depending upon the fermenter system, nitrogen or anaerobic gas mixtures can be sparged to maintain anaerobic conditions. However, none of the component species require H₂ or CO₂ for growth, beyond maintaining appropriate acid/base balance.
(3) Concentrate select members as needed to obtain desired input density: commonly done by centrifugation at 5-10K RPM with pull-off of supernatant under anaerobic conditions and resuspension in a lesser volume of new culture media or appropriate suspension buffer. The new culture density is confirmed by OD600 reading and viability by plating to solid media.
(4) Aggregate the cultured into the combined consortium: Estimated biomass from the OD600 readings are used to estimate the volume and prepare the aggregate.
(5) Prepare capsules: done under anaerobic conditions to preserve viability.
(6) Store capsules: optimal to store at conditions available clinically, e.g. −20° C.
(7) Quality Control: In addition to QC for prior steps and media, the final community will be evaluated to insure the appropriate species are present and in desired viable biomass. Analyses on materials for pre-clinical studies used 16S rRNA gene phylotyping with culture a qPCR-based methods. Metagenomic approaches may also be used to rule-out contamination with nonbacterial species or viruses.

It is further contemplated herein that growth conditions are optimized for the scaled cultures.

In some embodiments, media formulations are developed such that they lack animal products and/or substrates that might be associated with sensitizing antigens in foodstuffs. In addition, one of skill in the art can assess if additives to the inoculum enhance viability in capsules and once released in vivo. Materials can include preservatives and prebiotic compounds.

Stage II: It is further contemplated herein that the consortia described herein are formulated as a liquid formulation that can be administered to infants and young children. It is contemplated herein that such formulations comprise mixtures of spores from sporulating species, leveraging non-sporulating obligate anaerobes with limited aerotolerance, and including reducing factors in a liquid formula to buffer against short-term exposure to oxygen in ambient air and upon entry into the digestive tract. Compounds such as the amino acid cysteine or n-acetylcysteine, which have been used therapeutically in infants and have a robust safety profile are contemplated.

Additional pre-clinical animal models for use in testing formulations include e.g., neonatal swine models of food allergy, including ones for foodstuffs common in the diet of both humans and pigs.

### Example 2: OTU Clustering Method for Data from Human and Animal Studies

DNA extraction and sequencing for 16S rRNA gene phylotyping. A multiplexed amplicon library covering the V4 region of the 16S rDNA gene was generated from DNA extracted from human stool, mouse fecal pellets or segments of snap-frozen gut tissues using MO BIO™ Power-Fecal™ DNA Isolation Kits (MO BIO™ Laboratories) with custom modification to enhance lysis of Gram positive commensals with thick cell walls. The rest of library preparation followed the protocol of with dual-index barcodes. Aggregated libraries are sequenced with paired-end 250 bp reads on the Illumina™ MiSeq platform. The aggregate library pool was size selected from 300-500 bp on a Pippin™ prep 1.5% agarose cassette (Sage Sciences™) according to the manufacturer's instructions. Concentration of the pool is measured by qPCR (Kapa Biosystems™) and loaded onto the MiSeg™ (Illumina™) at 6-9 pM with 20% phiX spike-in to compensate for low base diversity according to Illumina™'s standard loading protocol.

16S rRNA Data preprocessing. Sequencing aims to obtain 10-50K usable reads per sample after quality filtering. Raw sequencing reads were processed using the mothur software package (v.1.35.1) and custom Python and R scripts, which perform de-noising, quality filtering, alignment against the ARB Silva reference database of 16S rDNA gene sequences, and clustering into Operational Taxonomic Units (OTUs) at 97% identity.

16S rRNA Data Analysis. To statistically test for differences between control and food allergic subjects in abundances of microbial taxa (OTUs), the DESeq2 software package was employed to support of analyses relative to host co-variates such as age, food allergy status, diet and antibiotic use in human cohort, OTUs showing significant differences were defined by: (1) adjusted p-value<=0.1; (2) relative abundance>=0.01 in either control or food allergic groups; (3) absolute value of log 2 fold changes>=2.

To improve the resolution of taxonomic calls and show phylogenetic relationships, a separate method used the pplacer software package to perform phylogenetic placement of individual OTU. Pplacer uses a likelihood-based methodology to place short sequencing reads of 16S rRNA amplicons on a reference tree, and also generates taxonomic classifications of the short sequencing reads using a least common ancestor-based algorithm. The reference tree required for phylogenetic placement is generated using full-length or near full-length (>1,200 nt) 16S rDNA sequences of type species from the Ribosomal Database Project (RDP).

For all statistical testing for 16S rDNA data analysis, p-values were adjusted for multiple hypothesis testing using the method of Benjamini and Hochberg (BH). Heat map plots are generated using custom R scripts.

Alpha diversity values (richness of a sample in terms of the diversity of the OTUs observed in it) were calculated using Shannon entropy to measure diversity in each sample. Beta-diversity values (distance between samples based on differences in OTUs present in each sample) were calculated using the unweighted/weighted Unifrac dissimilarity measure, to assess differences in overall microbial community structure.

(3) OTU Mappings of the Defined Species

The following operational taxonomic units map to the defined species, as identified in gnotobiotic mice colonized with these consortia. Fecal pellets were subjected to the above described 16S rRNA gene phylotyping over the V4 variable region.

TABLE 2
Mapping of the defined therapeutic species to OTU based on the 16S rRNA V4 region.

| Species | OTU taxonomic mappings, V4 region- |
|---|---|
| Bacteroides | Bacteria: Bacteroidetes: Bacteroidia: Bacteroidales |
| fragilis | Bacteria: Bacteroidetes: Bacteroidia: Bacteroidales: Bacteroidaceae |
| | Bacteria: Bacteroidetes: Bacteroidia: Bacteroidales: Bacteroidaceae: Bacteroides |
| Bacteroides | Bacteria: Bacteroidetes: Bacteroidia: Bacteroidales |
| thetaiotaomicron | Bacteria: Bacteroidetes: Bacteroidia: Bacteroidales: Bacteroidaceae |
| | Bacteria: Bacteroidetes: Bacteroidia: Bacteroidales: Bacteroidaceae: Bacteroides |
| Bacteroides | Bacteria: Bacteroidetes: Bacteroidia: Bacteroidales |
| ovatus | Bacteria: Bacteroidetes: Bacteroidia: Bacteroidales: Bacteroidaceae |
| | Bacteria: Bacteroidetes: Bacteroidia: Bacteroidales: Bacteroidaceae: Bacteroides |
| Clostridium | Bacteria: Firmicutes: Clostridia: Clostridiales: Peptostreptococcaceae |
| bifermentans | Bacteria: Firmicutes: Clostridia: Clostridiales: Peptostreptococcaceae: Clostridium cluster |
| | XI |
| Clostridium | Bacteria: Firmicutes: Clostridia: Clostridiales: Peptostreptococcaceae |
| hiranonsis | Bacteria: Firmicutes: Clostridia: Clostridiales: Peptostreptococcaceae: Clostridium cluster |
| | XI |
| Clostridium | Bacteria: Firmicutes: Clostridia: Clostridiales: Ruminococcaceae |
| leptum | Bacteria: Firmicutes: Clostridia: Clostridiales: Ruminococcaceae: Clostridium cluster IV |
| Clostridium | Bacteria: Firmicutes: Erysipelotrichia: Erysipelotrichales: Erysipelotrichaceae |
| ramosum | Erysipelotrichales: Erysipelotrichaceae: Clostridium cluster XVIII |
| Clostridium | Bacteria: Firmicutes: Clostridia: Clostridiales: |
| sardiniensis | Bacteria: Firmicutes: Clostridia: Clostridiales: Clostridiaceae I |
| (absonum) | |
| Clostridium | Bacteria: Firmicutes: Clostridia: Clostridiales: Lachnospiraceae |
| scindens | Bacteria: Firmicutes: Clostridia: Clostridiales: Lachnospiraceae: Clostridium cluster XIVa |
| Parabacteroides | Bacteria: Bacteroidetes: Bacteroidia: Bacteroidales: Porphyoromondaceae |
| goldsteinii | Bacteria: Bacteroidetes: Bacteroidia: Bacteroidales: Porphyoromondaceae: Parabacteroides |
| Prevotella | Bacteria: Bacteroidetes: Bacteroidia: Bacteroidales: |
| tannerae | Bacteria: Bacteroidetes: Bacteroidia: Bacteroidales: Prevotellaceae |
| | Bacteria: Bacteroidetes: Bacteroidia: Bacteroidales: Prevotellaceae: Prevotella |

TABLE 3
Mapping of the dysbiotic consortium species to OTU based on the 16S rRNA V4 region

| Species | OTU mapping |
|---|---|
| Bilophila | Bacteria: Proteobacteria: Deltaproteobacteria: Desulfovibrionales |
| wadsworthia | Bacteria: Proteobacteria: Deltaproteobacteria: Desulfovibrionales: Desolfovibrionaceae |
| | Bacteria: Proteobacteria: Deltaproteobacteria: Desulfovibrionales: Desolfovibrionaceae: |
| | Bilophila |
| Enterohacter | Bacteria: Proteobacteria: Gammaproteobacteria: Enterobacteriales |
| cloacae | Bacteria: Proteobacteria: Gammaproteobacteria: Enterobacteriales: Enterobacteriaceae |
| | Bacteria: Proteobacteria: Gammaproteobacteria: Enterobacteriales: Enterobacteriaceae: |
| | Enterbacter |
| Escherichia | Bacteria: Proteobacteria: Gammaproteobacteria: Enterobacteriales |
| coli | Bacteria: Proteobacteria: Gammaproteobacteria: Enterobacteriales: Enterobacteriaceae |
| | Bacteria: Proteobacteria: Gammaproteobacteria: Enterobacteriales: Enterobacteriaceae: |
| | Escherichia |
| Klebsiella | Bacteria: Proteobacteria: Gammaproteobacteria: Enterobacteriales |
| pneumonia | Bacteria: Proteobacteria: Gammaproteobacteria: Enterobacteriales: Enterobacteriaceae |
| | Bacteria: Proteobacteria: Gammaproteobacteria: Enterobacteriales: Enterobacteriaceae: |
| | Klebsiella |
| Proteus | Bacteria: Proteobacteria: Gammaproteobacteria: Enterobacteriales |
| mirabilis | Bacteria: Proteobacteria: Gammaproteobacteria: Enterobacteriales: Enterobacteriaceae |
| | Bacteria: Proteobacteria: Gammaproteobacteria: Enterobacteriales: Enterobacteriaceae: |
| | Proteus |

Species of microorganisms associated with protection from the development of food allergy were identified in a longitudinal study of pediatric human subjects (TABLE 4).

TABLE 4
Additional “beneficial” OTU identified in the longitudinal pediatric human cohort as associated with protection
from development of food allergy.

| | Nearest species |
|---|---|
| | mapping(s) with |
| OTU taxonomic mappings with sequencing of the V4 region- | pplacer |
| Bacteria: Firmicutes: Clostridia: Clostridiales: Lachnospiraceae | Clostridium hathewayi |
| Bacteria: Firmicutes: Clostridia: Clostridiales: Lachnospiraceae: Clostridium cluster | |
| XIVa | |
| Bacteria: Firmicutes: Clostridia: Clostridiales: Lachnospiraceae: Hungatella | |
| Bacteria: Firmicutes: Clostridia: Clostridiales: Lachnospiraceae | Clostridium nexile, |
| Bacteria: Firmicutes: Clostridia: Clostridiales: Lachnospiraceae: Clostridium cluster | Clostridium |
| XIVa | hylemonae, |
| | Clostridium |
| | glycyrrhizinilyticum, |
| | Clostridium |
| | scindens, |
| | Clostridium |
| | lavalense, |
| | Clostridium |
| | fimetarium, |
| | Clostridium |
| | symbiosum |
| Bacteria: Firmicutes: Clostridia: Clostridiales: Ruminococcaceae | Clostridium |
| Bacteria: Firmicutes: Clostridia: Clostridiales: Ruminococcaceae: Clostridium cluster | sporosphaeroides |
| IV | |
| Bacteria: Firmicutes: Negativicutes: Selenomonadales: Veillonellaceae | Dialister |
| Bacteria: Firmicutes: Negativicutes: Selenomonadales: Veillonellaceae: Dialister | proprionicifaciens, |
| | Dialister |
| | succinatiphilus |
| Bacteria: Bacteroidetes: Bacteroidia: Bacteroidales: Porphyoromondaceae | Parabacteroides |
| Bacteria: Bacteroidetes: Bacteroidia: Bacteroidales: Porphyoromondaceae: | distasonis, |
| Parabacteroides | Parabacteroides |
| | goldsteinii, |
| | Parabacteroides |
| | merdae |
| Bacteria: Firmicutes: Clostridia: Clostridiales: Peptostreptococcaceae | Peptostreptococcus |
| Bacteria: Firmicutes: Clostridia: Clostridiales: Peptostreptococcaceae: | anaerobius |
| Peptostreptococcus | |
| Bacteria: Firmicutes: Clostridia: Clostridiales: Ruminococcaceae | Subdoligranulum |
| Bacteria: Firmicutes: Clostridia: Clostridiales: Ruminococcaceae: Subdoligranulum | variabile |
| Bacteria: Firmicutes: Negativicutes: Selenomonadales: Veillonellaceae | Veilonella ratti |
| Bacteria: Firmicutes: Negativicutes: Selenomonadales: Veillonellaceae: Veillonella | |

Microorganisms that are associated with the development of food allergy were identified in a longitudinal study of pediatric human subjects (TABLE 5).

TABLE 5
Additional “dysbiotic” OTU identified in the longitudinal pediatric human cohort as associated with development
of food allergy.

| | Nearest |
|---|---|
| | species |
| | mapping |
| OTU taxonomic mappings with sequencing of the V4 region | with pplacer |
| Bacteroidetes: Bacteroidia: Bacteroidales: Rikenellaceae: | Alistipes |
| Bacteroidetes: Bacteroidia: Bacteroidales: Rikenellaceae: Alistipes | onderdonkii |
| Firmicutes: Clostridia: Clostridiales: Lachnospiraceae | Blautia |
| Firmicutes: Clostridia: Clostridiales: Lachnospiraceae: Blautia | wexlerae, |
| | Blautia |
| | henselae |
| Bacteria: Proteobacteria: Deltaproteobacteria: Desulfovibrionales | Bilophila |
| Bacteria: Proteobacteria: Deltaproteobacteria: Desulfovibrionales: Desolfovibrionaceae | wadsworthia, |
| Bacteria: Proteobacteria: Deltaproteobacteria: Desulfovibrionales: Desolfovibrionaceae: | Desulfovibrio |
| Bilophila | species |
| Bacteria: Proteobacteria: Deltaproteobacteria: Desulfovibrionales: Desolfovibrionaceae: | |
| Bilophila: Desulfovibrio | |
| Firmicutes: Bacilli: Lactobacillales: Lactobacillaceae: Lactobacillus | Lactobacillus |
| | johnsoni |
| Bacteria: Proteobacteria: Betaproteobacteria: Burkholderales: | Parasutterella |
| Bacteria: Proteobacteria: Betaproteobacteria: Burkholderales: Sutterellaceae | excrementihominis |
| Bacteria: Proteobacteria: Betaproteobacteria: Burkholderales: Sutterellaceae: | |
| Parasutterella | |
| Firmicutes: Clostridia: Clostridiales: Lachnospiraceae | Roseburia |
| Firmicutes: Clostridia: Clostridiales: Lachnospiraceae: Roseburia | inulinivorans |

### Example 3: Microbiology-Level Activities Used in Selection of Defined Species

The species in the defined consortia were selected per known biochemical, immunologic and microbiologic functions with capacity to affect beneficial immunomodulatory responses in the host. Without wishing to be bound by theory, microbiologic mechanisms of action can include the following.

### Adjuvant Effects of Microbial Products:

Described herein are embodiments of microbial products to stimulate the development, proliferation, and activity of regulatory T cells (T_{regs}) and other immune cell pathways. The production of key microbial antigens from commensal anaerobes, including their lipoteichoic acid (LTA), exo-polysaccharides (PSA), LPS, bacterial flagellin, and bacterial DNA can act through stimulating toll-like-receptor pathways (e.g., TLR→MyD88 and other immune cell pathways) to skew mucosal T cells to a regulatory vs. allergic phenotype. In contrast, published data have shown that bacterial cell wall fractions from members of the negative control consortium can promote aberrant stimulation of both allergic (Tₕ2) and pro-inflammatory (Tₕ1) responses. The distinct portions of these molecules that skew towards tolerance vs. allergy or inflammation highlight the interplay between mammalian hosts and colonizing microbiota, including the microbial products that signal the host to maintain a healthy homeostasis versus elicit pathogenic immune responses.

Mucosal and immunoprotective functions of microbial end-products of metabolism: Short chain fatty acids (SCFA) are natural end-products of microbial anaerobic fermentation as are additional small molecule metabolites from anaerobic fermentation of different carbon sources. End-products such as butyrate have been shown to provide a primary energy source to the gut epithelium and to contribute to the development of tolerizing responses in mucosal locations. The consortia selected produce a dominance of butyrate and propionate from the fermentation of simple and complex carbohydrates that may be in the gut lumen, per the diet and secretion of host factors. These factors would likely act in combination with other microbial activities to mediate the desired immunomodulatory effects.

Biochemical activities: The species selected perform the full complement of bile acid transformations and also transform a variety of other molecules including other cholesterol-derivatives, biogenic amines, lipids and production of aryl hydrocarbons which may serve as microbial siderophores, quorum sensing molecules and other metabolic intermediates within the microbial cell. Such metabolites are potentially capable of stimulating host aryl-hydrocarbon receptor (AHR) pathways which have also been demonstrated to promote tolerizing responses in the gut mucosa.

Gut conditioning: Microbiologically, select members of the consortia are known to aid the subsequent colonization, biochemical and further immunoprotective roles of other species. Both Bacteroides fragilis and Bacteroides thetaiotaomicron, when included in defined flora, assist the growth of more fastidious members of the Bacteroidetes, Firmicutes and Actinobacteria. Clostridium ramosum has demonstrated comparable effects in defined colonizations of germ-free mice with other commensals. Effects are multi-factorial, and include maturing of gut epithelial responses, altered host secretion of glycoconjugates which can serve as carbon sources for the commensal flora, enhancing gut peristalsis and digestion, reducing lumen gut oxygen tension so more obligately anaerobic species can flourish, and releasing metabolites, and/or extracellular products of microbial digestion which support the growth of additional species by providing carbon and/or nitrogen sources, vitamins, and other essential micronutrients.

Reducing the biomass of dysbiotic or pathogenic species: Animal models conducted by our group have also shown that the species in the gut protect communities can reduce the biomass of the Proteobacterial species in the negative control consortium. Without wishing to be bound by theory, mechanistically these biomass reductions also reduce the antigen burden of products from these species that preferentially skew towards allergic responses.

### Example 4: Gut-Protect (GP-II) Consortium-GPIIa

Composition of GP-IIa: Bacteroides fragilis, Bacteroides ovatus, Bacteroides vulgatus, Parabacteroides distasonis, Prevotella melaninogenica

GP-IIa can be used to prevent, treat, and cure food allergy in a mouse IL4raF709 food allergy model.

### Introduction:

The role of pathogenic dysbiosis in food allergy (FA) remains unclear. It was observed that FA infants exhibited dysbiotic fecal microbiota that evolved compositionally over time. Both infants and mice with FA had decreased secretory IgA and increased IgE binding to fecal bacteria, indicative of a broader breakdown of oral tolerance in FA than hitherto appreciated. A consortium of commensal human Clostridiales species, reflective of taxa impacted by dysbiosis, suppressed FA in mice and normalized the gut mucosal immune responses, as did a separate immunomodulatory consortium of human origin Bacteroidales species. The two consortia induced distinct subsets of regulatory T (Treg) cells that were deficient in FA subjects and mice. Thus, different commensals act to stimulate specific Treg cell populations to protect against FA, while dysbiosis impairs this regulatory response to promote disease.

Food allergy (FA) is a major public health concern, whose prevalence has grown dramatically over the past decade. FA now affects 6% of children under 5 years, and 3% of teens and adults. Most FA is acquired in the first or second year of life, indicating that early childhood exposures have profound long-term health consequences. The hygiene hypothesis stipulates that microbial exposures play a critical role in the development of protection against allergic diseases, and that alterations in those exposures, including changes in the host microbial flora, may underlie the rise in allergic diseases. In that regard, several studies have shown that factors impacting gut microbial colonization and composition early in life, including method of delivery (i.e., cesarean section), antibiotic use, and breastfeeding influence the development of atopic disease. Less information is available on the role of gut microbiota in human FA. Reduced gut microbiota diversity and an elevated ratio of the abundance of Enterobacteriaceae to Bacteroidaceae species in early infancy have been associated with subsequent food sensitization, suggesting that the initial stages of gut colonization with particular microbial communities may contribute to the development of atopic disease, including FA.

Prior studies have shown that the presence and composition of the gut microbiota influences the host's susceptibility to FA. Mice raised in a sterile environment cannot be tolerized to antigens given orally, have reduced IgA levels and IL-10 producing regulatory T (Treg) cells. In contrast, colonization with Segmented Filamentous Bacteria (SFB) and Clostridia species promotes the development of IL-17 producing T cells and Treg cells, respectively.

The results provided herein show that in a FA-prone genetic mouse model (Il4ra^{F709} mice), the acquisition of FA is associated with a gut microbiota signature that is distinct from that of FA-tolerant mice. Furthermore, transfer of fecal microbiota from FA but not tolerant mice to germ-free (GF) recipients transmitted susceptibility to FA. More recently, Stefka et al found that sensitization to a food allergen was increased in mice that have been treated with antibiotics or were devoid of commensal microbiota. By selectively colonizing gnotobiotic mice, allergy-protective capacity was conferred by a Clostridia-predominant microbiota.

These findings suggest that unfavorable alterations in the development of the gut microbiota early in life favor the emergence of dysbiotic communities with concomitant reductions in beneficial species. In combination, such changes may result in a failure to promote tolerant immune responses, thus raising the host's susceptibility to allergic and inflammatory responses. Mechanisms by which the commensal microbiota may promote oral tolerance to food allergens include their enhancement of epithelial cell barrier integrity and elicitation of protective mucosal Treg cell responses. The production of short-chain fatty acids, such as acetate, propionate and butyrate, by commensals such as Clostridia species, reinforce mucosal tolerance by recruiting and stabilizing Treg cells in the gut. Colonization with commensal bacteria also expands populations of induced Treg (iTreg) cells in the gut.

Here, it is demonstrated that FA infants manifest an evolving dysbiosis that impacts beneficial gut commensals. Furthermore, administration of defined bacterial consortia of human-origin commensals, one composed of culturable species from the order Clostridiales and the other of species from order Bacteroidales, successfully prevented FA and suppressed established disease in FA-prone Il4ra^{F709} mice. Both consortia conferred protection by inducing protective Treg cell populations, which are deficient in FA subjects and FA-prone mice. Thus, these results identify a common mechanism by which commensals prevent FA, and they underscore the potential for employing defined microbial consortia as oral microbial therapy in promoting disease prevention and remission.

### Results:

Promotion of Oral Tolerance in FA by Immunomodulatory Human Bacteroidales Species.

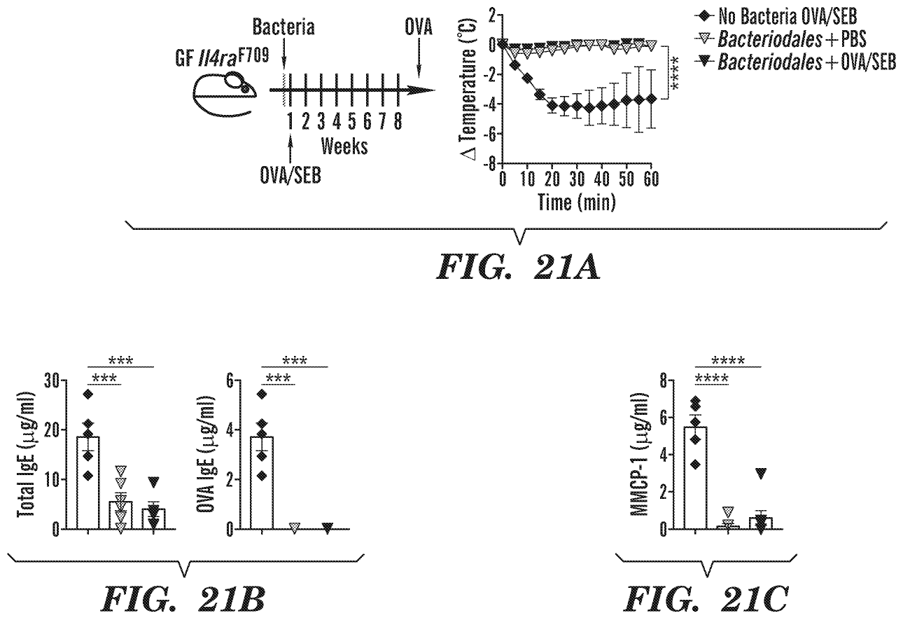

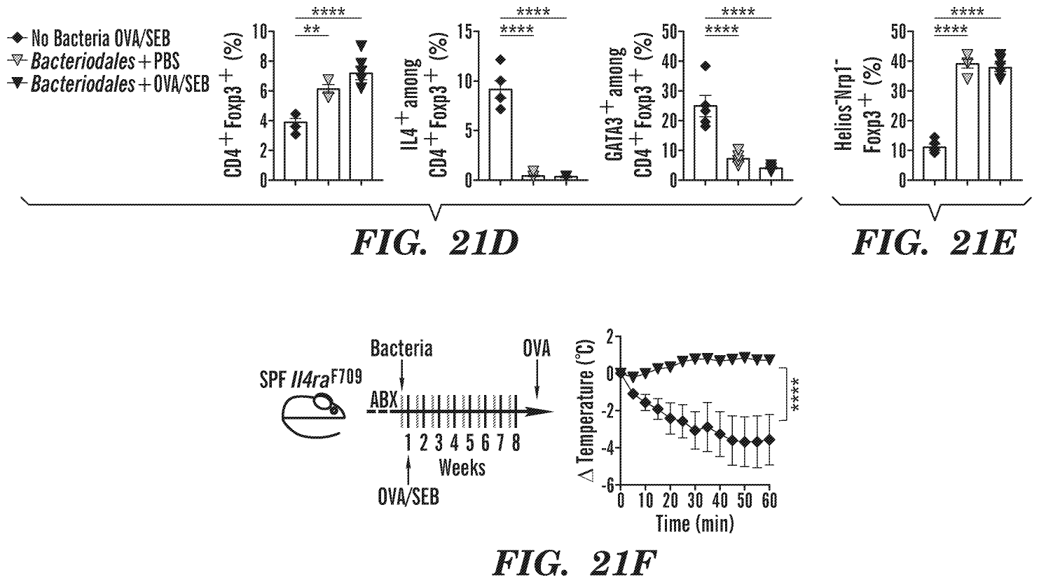

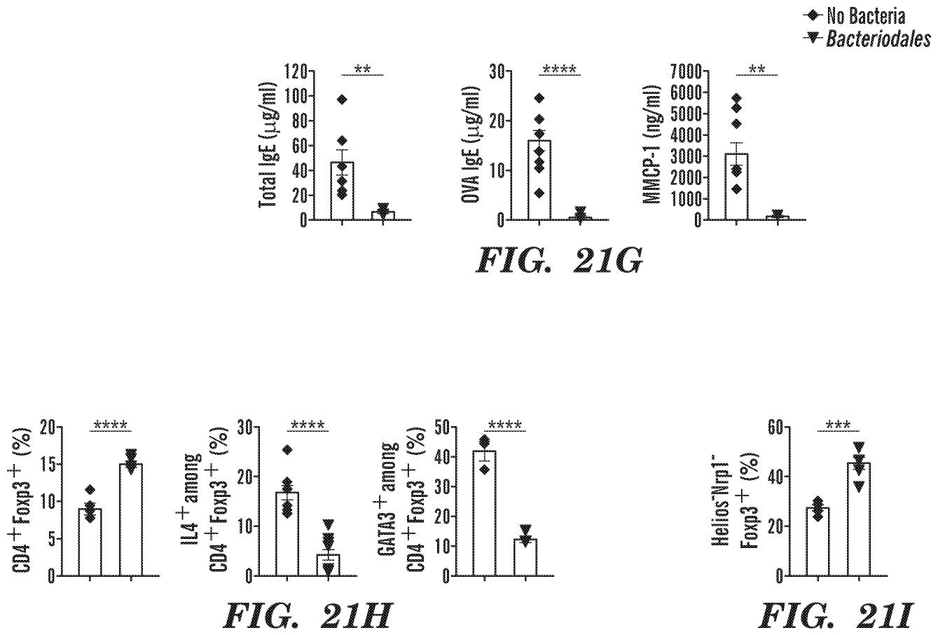

To determine if the capacity to promote oral tolerance in FA was restricted to Clostridiales species or was shared by other immunomodulatory bacteria, a consortium of five human-origin Bacteroidales species were tested, including B. fragilis, B. ovatus, B. vulgatus, P. melaninogenica, and P. distasonis (OTU24, CRS P. distasonis). Similar to the case of the Clostridiales consortium, the choice of these species reflected their availability as type species, their well-characterized genomic and metabolic profiles, ease of culturability and their previous implication in promoting Treg cells in the gut. Results revealed that colonization with the Bacteroidales consortium completely protected against the induction of FA in GF Il4ra^{F709} mice upon their sensitization with OVA/SEB (FIG. 21A-21E). Furthermore, the Bacteroidales consortium protected conventional SPF Il4ra^{F709} mice from developing FA when it was given in tandem with OVA/SEB during the sensitization protocol, as per the Clostridiales mix (FIG. 21F-21I). These results established that protection against FA is not a unique attribute of Clostridia species but could be affected by other immunomodulatory bacteria.

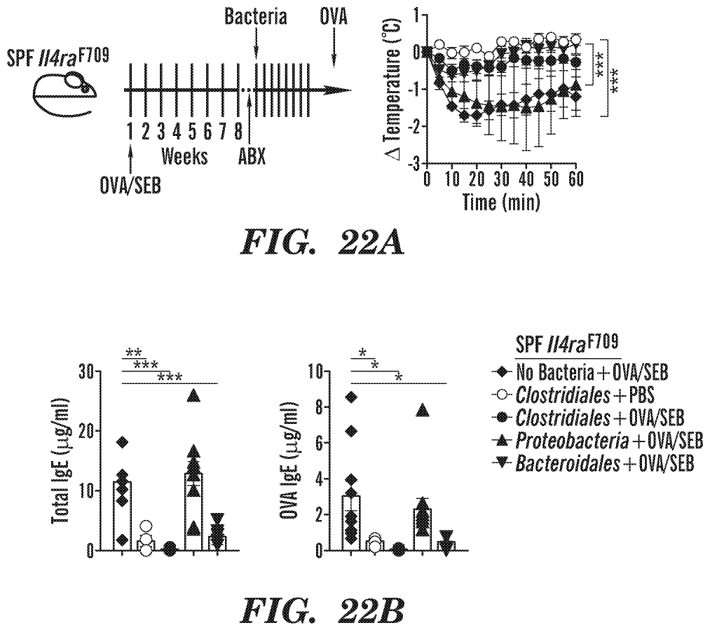

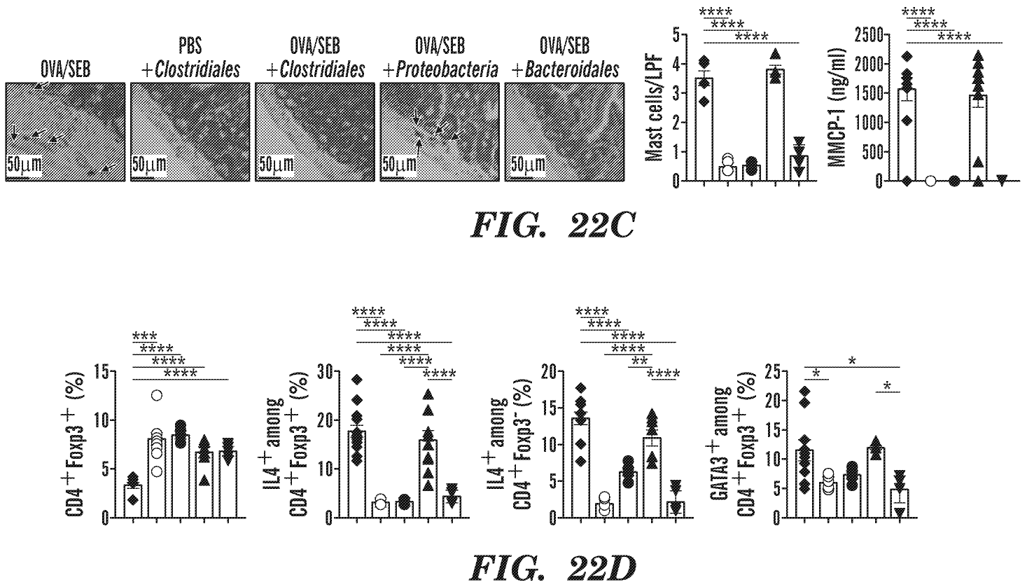

To determine whether bacteriotherapy with the Clostridiales and Bacteroidales consortia could suppress FA once the disease was established, conventional R4raF709 mice were sensitized with OVA/SEB once weekly for eight weeks to establish disease. The mice were then treated with a short course of antibiotics and further sensitized with OVA/SEB for an additional 4 weeks with or without bacterial therapy with either the Clostridiales, Bacteroidales or Proteobacteria consortium. The mice were then challenged orally with OVA and analyzed. Results showed that therapy with either the Clostridiales or Bacteroidales but not the Proteobacteria consortium prevented the OVA/SEB-sensitized Il4ra^{F709} mice from reacting to the OVA challenge (FIG. 22A). The Clostridiales and Bacteroidales but not the Proteobacteria consortium suppressed the total and OVA-specific serum IgE responses, the rise in serum MMCP-1 post OVA challenge, and the mast cell expansion (FIG. 22B, 22C). While all bacterial consortia increased the frequencies of MLN Treg cells in this disease curative model (FIG. 22D), only the Clostridiales and Bacteroidales consortia but not the Proteobacteria consortium suppressed the food allergy-associated Treg cell Th2 cell-like reprogramming (FIG. 22D).

### Bacteroidales Consortium Demonstrates Extended Persistence In Vivo in Mice After a Single Dose.

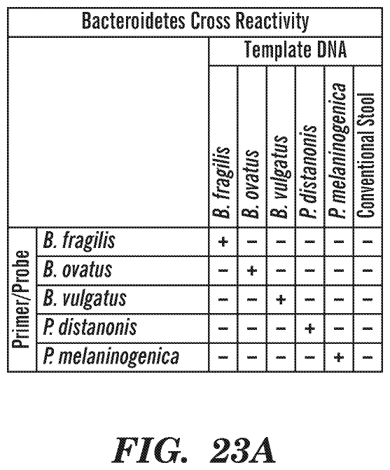

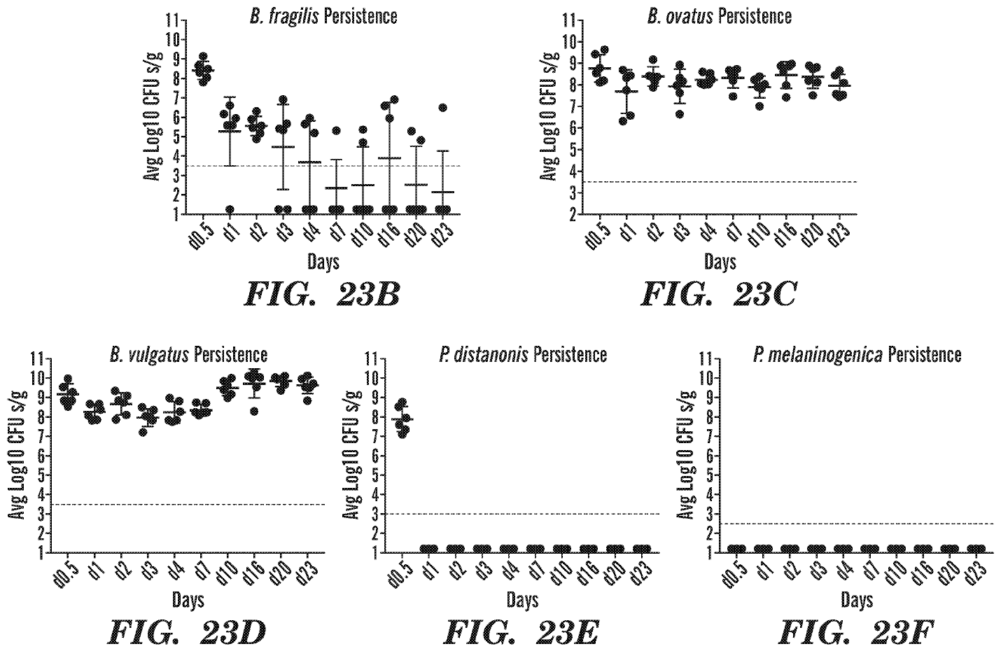

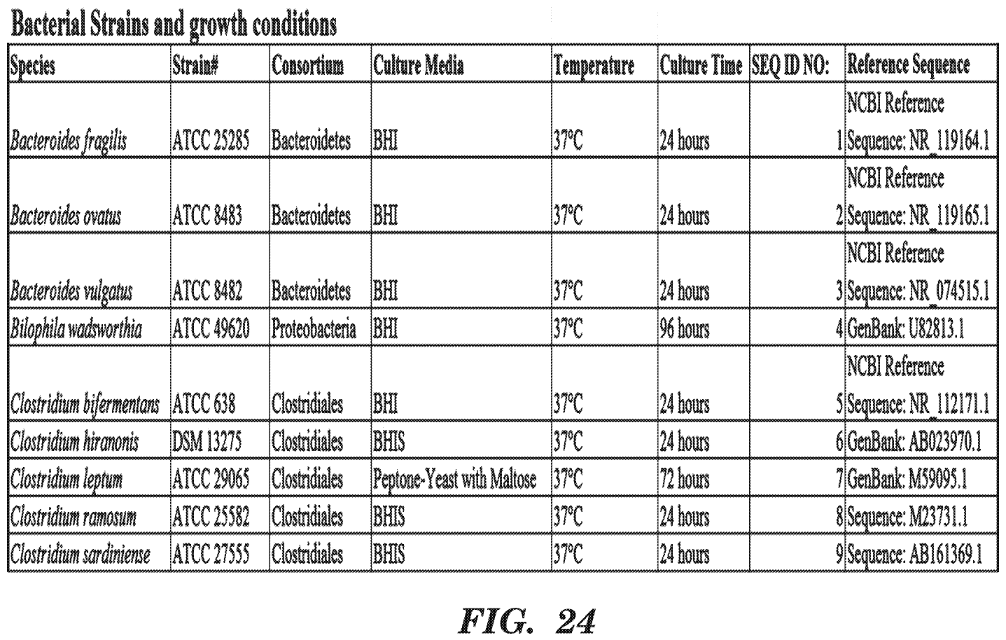

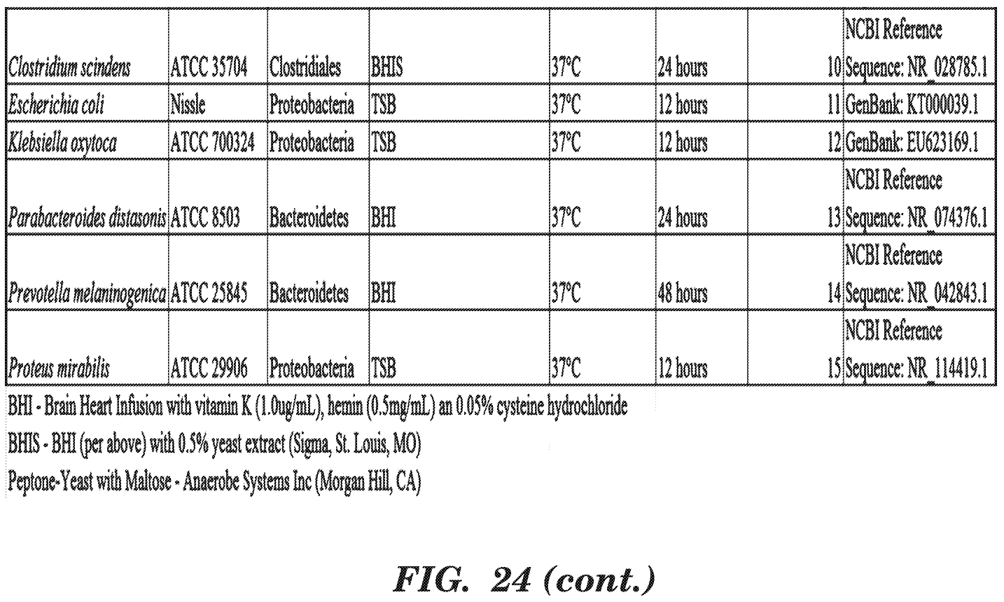

To determine persistence of the consortium members, conventional IL4raF709 mice receiving a single dose of the consortium were followed for 3 weeks with serial collection of fecal samples prior to and after dosing. The dose contained approximately 1×10⁷ CFU/g of each organism. Specific quantitative qPCR probes for each species, and that did not cross-react with conventional microbiota (FIG. 23A), were used to assess molecular biomass of the organisms administered in the consortium dose. As shown in FIG. 23B-F B. vulgatus and B. ovatus persisted in all mice during the sampling period. In more than half of mice, B. fragilis was detectable. P. distasonis was detectable at 12 hours but not thereafter. P. melaninogenica was not detectable at 12 hours after administration (gut transit time in adult mice is 6-7 hours).

| SEQUENCES | |
|---|---|
| (Bacteroides fragilis strain ATCC 25285 16S ribosomal RNA, partial | |
| sequence); NCBI Reference Sequence: NR_119164.1 | |
| SEQ ID NO: 1 | |
| 1 | ATGAACGCTA GCTACAGGCT TAACACATGC AAGTCGAGGG GCATCAGGAA GAAAGCTTGC |
| 61 | TTTCTTTGCT GGCGACCGGC GCACGGGTGA GTAACACGTA TCCAACCTGC CCTTTACTCG |
| 121 | GGGATAGCCT TTCGAAAGAA AGATTAATAC CCGATAGCAT AATGATTCCG CATGGTTTCA |
| 181 | TTATTAAAGG ATTCCGGTAA AGGATGGGGA TGCGTTCCAT TAGGTTGTTG GTGAGGTAAC |
| 241 | GGCTCACCAA GCCTTCGATG GATAGGGGTT CTGAGAGGAA GGTCCCCCAC ATTGGAACTG |
| 301 | AGACACGGTC CAAACTCCTA CGGGAGGCAG CAGTGAGGAA TATTGGTCAA TGGGCGCTAG |
| 361 | CCTGAACCAG CCAAGTAGCG TGAAGGATGA AGGCTCTATG GGTCGTAAAC TTCTTTTATA |
| 421 | TAAGAATAAA GTGCAGTATG TATACTGTTT TGTATGTATT ATATGAATAA GGATCGGCTA |
| 481 | ACTCCGTGCC AGCAGCCGCG GTAATACGGA GGATCCGAGC GTTATCCGGA TTTATTGGGT |
| 541 | TTAAAGGGAG CGTAGGTGGA CTGGTAAGTC AGTTGTGAAA GTTTGCGGCT CAACCGTAAA |
| 601 | ATTGCAGTTG ATACTGTCAG TCTTGAGTAC AGTAGAGGTG GGCGGAATTC GTGGTGTAGC |
| 661 | GGTGAAATGC TTAGATATCA CGAAGAACTC CGATTGCGAA GGCAGCTCAC TGGACTGCAA |
| 721 | CTGACACTGA TGCTCGAAAG TGTGGGTATC AAACAGGATT AGATACCCTG GTAGTCCACA |
| 781 | CAGTAAACGA TGAATACTCG CTGTTTGCGA TATACAGTAA GCGGCCAAGC GAAAGCATTA |
| 841 | AGTATTCCAC CTGGGGAGTA CGCCGGCAAC GGTGAAACTC AAAGGAATTG ACGGGGGCCC |
| 901 | GCACAAGCGG AGGAACATGT GGTTTAATTC GATGATACGC GAGGAACCTT ACCCGGGCTT |
| 961 | AAATTGCAGT GGAATGATGT GGAAACATGT CAGTGAGCAA TCACCGCTGT GAAGGTGCTG |
| 1021 | CATGGTTGTC GTCAGCTCGT GCCGTGAGGT GTCGGCTTAA GTGCCATAAC GAGCGCAACC |
| 1081 | CTTATCTTTA GTTACTAACA GGTTATGCTG AGGACTCTAG AGAGACTGCC GTCGTAAGAT |
| 1141 | GTGAGGAAGG TGGGGATGAC GTCAAATCAG CACGGCCCTT ACGTCCGGGG CTACACACGT |
| 1201 | GTTACAATGG GGGGTACAGA AGGCAGCTAG CGGGTGACCG TATGCTAATC CCAAAATCCT |
| 1261 | CTCTCAGTTC GGATCGAAGT CTGCAACCCG ACTTCGTGAA GCTGGATTCG CTAGTAATCG |
| 1321 | CGCATCAGCC ACGGCGCGGT GAATACGTTC CCGGGCCTTG TACACACCGC CCGTCAAGCC |
| 1381 | ATGGGAGCCG GGGGTACCTG AAGTACGTAA CCGCAAGGAT CGTCCTAGGG TAAAAC |
| (Bacteroides ovatus strain ATCC 8483 16S ribosomal RNA, partial | |
| sequence); NCBI Reference Sequence: NR_119165.1 | |
| SEQ ID NO: 2 | |
| 1 | ATGAACGCTA GCTACAGGCT TAACACATGC AAGTCGAGGG GCAGCATTTT NGTTTGCTTG |
| 61 | CAAACTGAAG ATGGCGACCG GCGCACGGGT GAGTAACACG TATCCAACCT GCCGATAACT |
| 121 | CCGGNATAGC CTTTCGAAAG AAAGATTAAT ACCNGATAGC ATACGAANAN CGCATGNTAN |
| 181 | TTTTATTAAA GAATTTCGGT TATCGATGGG GATGCGTTCC ATTAGTTTGT TGGCGGGGTA |
| 241 | ACGGCCCACC AAGACTACGA TGGATAGGGG TTCTGAGAGG AAGGTCCCCC ACATTGGAAC |
| 301 | TGAGACACGG TCCAAACTCC TACGGGAGGC AGCAGTGAGG AATATTGGTC AATGGGCGAG |
| 361 | AGCCTGAACC AGCCAAGTAG CGTGAAGGAT GANGGCCCTA TGGGTCGTAA ACTTCTTTTA |
| 421 | TATGGGAATA AAGTNTTCCA CGTGTGGAAT TTTGTATGTA CCATATGAAT AAGGATCGGC |
| 481 | TAACTCCGTG CCAGCAGCCG CGGTAATACG GAGGATCCGA GCGTTATCCG GATTTATTGG |
| 541 | GTTTAAAGGG AGCGTAGGTG GATTGTTAAG TCAGTTGTGA AAGTTTGCGG CTCAACCGTA |
| 601 | AAATTGCAGT TGAAACTGGC AGTCTTGAGT ACAGTAGAGG TGGGCGGAAT TCGTGGTGTA |
| 661 | GCGGTGAAAT GCTTAGATAT CACGAAGAAC TCCGATTGCG AAGGCAGCTC ACTAGACTGN |
| 721 | NACTGACACT GATGCTCGAA AGTGTGGGTA TCAAACAGGA TTAGATACCC TGGTAGTCCA |
| 781 | CACAGTAAAC GATGAATACT CGCTGTTTGC GATATACAGT AAGCGGCCAA GCGAAAGCAT |
| 841 | TAAGTATTCC ACCTGGGGAG TACGCCGGCA ACGGTGAAAC TCAAAGGAAT TGACGGGGGC |
| 901 | CNGCACAAGC GGAGGAACAT GTGGTTTAAT TCGATGATAC GCGAGGAACC TTACCCGGGC |
| 961 | TTAAATTGCA ACNGAATATA TTGGAAACAG TATAGCCGNA AGGCTGTTGT GAAGGTGCTG |
| 1021 | CATGGTTGTC GTCAGCTCGT GCCGTGAGGT GTCGGCTTAA GTGCCATAAC GAGCGCAACC |
| 1081 | CNTATCTTTA GTTACTAACA GGTTATGCTG AGGACTCTAG AGAGACTGCC GTCGTAAGAT |
| 1141 | GTGAGGAAGG TGGGGATGAC GTCAAATCAG CACGGCCCTT ACGTCCGGGG CTACACACGT |
| 1201 | GTTACAATGG GGGGTACAGA AGGCAGCTAC CNGGNGACAG GATGCTAATC CCAAAAACCT |
| 1261 | CTCTCAGTTC GGATCGAAGT CTGCAACCCG ACTTCGTGAA GCTGGATTCG CTAGTAATCG |
| 1321 | CGCATCAGCC ATGGCGCGGT GAATACGTTC CCGGGCCTTG TACACACCGC CCGTCAAGCC |
| 1381 | ATGAAAGCCG GGGGT |
| (Bacteroides vulgatus strain ATCC 8482 16S ribosomal RNA, partial | |
| sequence); NCBI Reference Sequence: NR_074515.1 | |
| SEQ ID NO: 3 | |
| 1 | TATTACAATG AAGAGTTTGA TCCTGGCTCA GGATGAACGC TAGCTACAGG CTTAACACAT |
| 61 | GCAAGTCGAG GGGCAGCATG GTCTTAGCTT GCTAAGGCCG ATGGCGACCG GCGCACGGGT |
| 121 | GAGTAACACG TATCCAACCT GCCGTCTACT CTTGGACAGC CTTCTGAAAG GAAGATTAAT |
| 181 | ACAAGATGGC ATCATGAGTC CGCATGTTCA CATGATTAAA GGTATTCCGG TAGACGATGG |
| 241 | GGATGCGTTC CATTAGATAG TAGGCGGGGT AACGGCCCAC CTAGTCTTCG ATGGATAGGG |
| 301 | GTTCTGAGAG GAAGGTCCCC CACATTGGAA CTGAGACACG GTCCAAACTC CTACGGGAGG |
| 361 | CAGCAGTGAG GAATATTGGT CAATGGGCGA GAGCCTGAAC CAGCCAAGTA GCGTGAAGGA |
| 421 | TGACTGCCCT ATGGGTTGTA AACTTCTTTT ATAAAGGAAT AAAGTCGGGT ATGGATACCC |
| 481 | GTTTGCATGT ACTTTATGAA TAAGGATCGG CTAACTCCGT GCCAGCAGCC GCGGTAATAC |
| 541 | GGAGGATCCG AGCGTTATCC GGATTTATTG GGTTTAAAGG GAGCGTAGAT GGATGTTTAA |
| 601 | GTCAGTTGTG AAAGTTTGCG GCTCAACCGT AAAATTGCAG TTGATACTGG ATATCTTGAG |
| 661 | TGCAGTTGAG GCAGGCGGAA TTCGTGGTGT AGCGGTGAAA TGCTTAGATA TCACGAAGAA |
| 721 | CTCCGATTGC GAAGGCAGCC TGCTAAGCTG CAACTGACAT TGAGGCTCGA AAGTGTGGGT |
| 781 | ATCAAACAGG ATTAGATACC CTGGTAGTCC ACACGGTAAA CGATGAATAC TCGCTGTTTG |
| 841 | CGATATACTG CAAGCGGCCA AGCGAAAGCG TTAAGTATTC CACCTGGGGA GTACGCCGGC |
| 901 | AACGGTGAAA CTCAAAGGAA TTGACGGGGG CCCGCACAAG CGGAGGAACA TGTGGTTTAA |
| 961 | TTCGATGATA CGCGAGGAAC CTTACCCGGG CTTAAATTGC AGATGAATTA CGGTGAAAGC |
| 1021 | CGTAAGCCGC AAGGCATCTG TGAAGGTGCT GCATGGTTGT CGTCAGCTCG TGCCGTGAGG |
| 1081 | TGTCGGCTTA AGTGCCATAA CGAGCGCAAC CCTTGTTGTC AGTTACTAAC AGGTTCCGCT |
| 1141 | GAGGACTCTG ACAAGACTGC CATCGTAAGA TGTGAGGAAG GTGGGGATGA CGTCAAATCA |
| 1201 | GCACGGCCCT TACGTCCGGG GCTACACACG TGTTACAATG GGGGGTACAG AGGGCCGCTA |
| 1261 | CCACGCGAGT GGATGCCAAT CCCCAAAACC TCTCTCAGTT CGGACTGGAG TCTGCAACCC |
| 1321 | GACTCCACGA AGCTGGATTC GCTAGTAATC GCGCATCAGC CACGGCGCGG TGAATACGTT |
| 1381 | CCCGGGCCTT GTACACACCG CCCGTCAAGC CATGGGAGCC GGGGGTACCT GAAGTGCGTA |
| 1441 | ACCGCGAGGA GCGCCCTAGG GTAAAACTGG TGACTGGGGC TAAGTCGTAA CAAGGTAGCC |
| 1501 | GTACCGGAAG |
| (Bilophila wadsworthia 16S ribosomal RNA gene, partial sequence); | |
| GenBank: U82813.1 | |
| SEQ ID NO: 4 | |
| 1 | CTTAACACAT GCAAGTCGAA CGTGAAAGTC CTTCGGGATG AGTAAAAGTG GCGCACGGGT |
| 61 | GAGTAACGCG TGGATAATCT ACCCTTAAGA TGGGGATAAC GGCTGGAAAC GGTCGCTAAT |
| 121 | ACCGAATACG CTCCCGATTT TATCATTGGG GGGAAAGATG GCCTCTGCTT GCAAGCTATC |
| 181 | GCTTAAGGAT GAGTCCGCGT CCCATTAGCT AGTTGGCGGG GTAACGGCCC ACCAAGGCAA |
| 241 | CGATGGGTAG CCGGTCTGAG AGGATGACCG GCCACACTGG AACTGGAACA CGGTCCAGAC |
| 301 | TCCTACGGGA GGCAGCAGTG GGGAATATTG CGCAATGGGC GAAAGCCTGA CGCAGCGACG |
| 361 | CCGCGTGAGG GATGAAGGTT CTCGGATCGT AAACCTCTGT CAGGGGGGAA GAAACCCCCT |
| 421 | CGTGTGAATA ATGCGAGGGC TTGACGGTAC CCCCAAAGGA AGCACCGGCT AACTCCGTGC |
| 481 | CAGCAGCCGC GGTAATACGG AGGGTGCAAG CGTTAATCGG AATCACTGGG CGTAAAGCGC |
| 541 | ACGTACGCGG CTTGGTAAGT CAGGGGTGAA ATCCCACAGC CCAACTGTGG AACTGCCTTT |
| 601 | GATACTGCCA CGCTTGAGTA CCGGAGAGGG TGGCGGAATT CCAGGTGTAG GAGTGAAATC |
| 661 | CGTAGATATC TGGAGGAACA CCGGTGGCGA AGGCGGCCAC CTGGACGGTA ACTGACGCTG |
| 721 | AGGTGCGAAA GCGTGGGTAG CAAACAGGAT TAGATACCCT GGTAGTCCAC GCTGTAAACG |
| 781 | ATGGGTGCNG GGTGCTGGGA TGTATGTCTC GGTGCCGTAG CTAACGCGAT AAGCACCCCG |
| 841 | CCTGGGGAGT ACGGTCGCAA GGCTGAAACT CAAAGAAATT GACGGGGGCC CGCACAAGCG |
| 901 | GTGGAGTATG TGGTTTAATT CGATGCAACG CGAAGAACCT TACCCAGGCT TGACATCTAG |
| 961 | GGAACCCTTC GGAAATGAAG GGGTGCCCTT CGGGGAGCCC TAAGACAGGT GCTGCATGGC |
| 1021 | TGTCGTCAGC TCGTGCCGTG AGGTGTTGGG TTAAGTCCCG CAACGAGCGC AACCCCTATC |
| 1081 | TTCAGTTGCC AGCAGGTAAG GCTGGGCACT CTGGAGAGAC CGCCCCGGTC AACGGGGAGG |
| 1141 | AAGGTGGGGA CGACGTCAAG TCATCATGGC CCTTACGCCT GGGGCTACAC ACGTACTACA |
| 1201 | ATGGCGCGCA CAAAGGGTAG CGAGACCGCG AGGTGGAGCC AATCCCAAAA AACGCGTCCC |
| 1261 | AGTCCGGATT GGAGTCTGCA ACTCGACTCC ATGAAGTCGG AATCGCTAGT AATTCGAGAT |
| 1321 | CAGCATGCTC GGGTGAATGC GTTCCCGGGC CTTGTACACA CCGCCCGTCA CACCACGAAA |
| 1381 | GTCGGTTTTA CCCGAAGCCG GTGAGCTAAC TCGCAAGAGG AGCAGCCGTC TACGGTAGGG |
| 1441 | CCGATGATTG GGGTGAAGTC GTAACAA |
| (Clostridium bifermentans strain ATCC 638 16S ribosomal RNA | |
| gene, partial sequence); NCBI Reference Sequence: NR_112171.1 | |
| SEQ ID NO: 5 | |
| 1 | CATRGCTCAG GATGAACGCT GGCGGCGTGC CTAACACATG CAAGTCGAGC GATCTCTTCG |
| 61 | GAGAGAGCGG CGGACGGGTG AGTAACGCGT GGGTAACCTG CCCTGTACAC ACGGATAACA |
| 121 | TACCGAAAGG TATACTAATA CGGGATAACA TATGAAAGTC GCATGGCTTT TGTATCAAAG |
| 181 | CTCCGGCGGT ACAGGATGGA CCCGCGTCTG ATTAGCTAGT TGGTAAGGTA ATGGCTTACC |
| 241 | AAGGCAACGA TCAGTAGCCG ACCTGAGAGG GTGATCGGCC ACACTGGAAC TGAGACACGG |
| 301 | TCCAGACTCC TACGGGAGGC AGCAGTGGGG AATATTGCAC AATGGGCGAA AGCCTGATGC |
| 361 | AGCAACGCCG CGTGAGCGAT GAAGGCCTTC GGGTCGTAAA GCTCTGTCCT CAAGGAAGAT |
| 421 | AATGACGGTA CTTGAGGAGG AAGCCCCGGC TAACTACGTG CCAGCAGCCG CGGTAATATG |
| 481 | TAGGGGGCTA GCGTTATCCG GAATTACTGG GCGTAAAGGG TGCGTAGGTG GTTTTTTAAG |
| 541 | TCAGAAGTGA AAGGCTACGG CTCAACCGTA GTAAGCTTTT GAAACTAGAG AACTTGAGTG |
| 601 | CAGGAGAGGA GAGTAGAATT CCTAGTGTAG CGGTGAAATG CGTAGATATT AGGAGGAATA |
| 661 | CCAGTAGCGA AGGCGGCTCT CTGGACTGTA ACTGACACTG AGGCACGAAA GCGTGGGGAG |
| 721 | CAAACAGGAT TAGATACCCT GGTAGTCCAC GCCGTAAACG ATGAGTACTA GGTGTCGGGG |
| 781 | GTTACCCCCC TCGGTGCCGC ACTAACGCAT TAAGTACTCC GCCTGGGAAG TACGCTCGCA |
| 841 | AGAGTGAAAC TCAAAGGAAT TTDCGGGGAC CCGCACAAGT AGCGGAGCAT GTGGTTTAAT |
| 901 | TCGAAGCAAC GCGAAGAACC TTACCTAAGC TTGACATCCC ACTGACCTCT CCCTAATCGG |
| 961 | AGATTTCCCT TCGGGGACAG TGGTGACAGG TGGTGCATGG TTGTCGTCAG CTCGTGTCGT |
| 1021 | GAGATGTTGG GTTAAGTCCC GCAACGAGCG CAACCCTTGC CTTTAGTTGC CAGCATTAAG |
| 1081 | TTGGGCACTC TAGAGGGACT GCCGAGGATA ACTCGGAGGA AGGTGGGGAT GACGTCAAAT |
| 1141 | CATCATGCCC CTTATGCTTA GGGCTACACA CGTGCTACAA TGGGTGGTAC AGAGGGTTGC |
| 1201 | CAAGCCGCGA GGTGGAGCTA ATCCCTTAAA GCCATTCTCA GTTCGGATTG TAGGCTGAAA |
| 1261 | CTCGCCTACA TGAAGCTGGA GTTACTAGTA ATCGCAGATC AGAATGCTGC GGTGAATGCG |
| 1321 | TTCCCGGGTC TTGTACACAC CGCCCGTCAC ACCATGGAAG TTGGGGGCGC CCGAAGCCGG |
| 1381 | TTAGCTAACC TTTTAGGAAG CGGCCGTCGA AGGTGAACAA ATGACTGGGG TGAAGTCGTA |
| 1441 | ACAAGGTANC CGTATCGGAA GGTGCGGCBG GATCAA |
| (Clostridium hiranonis gene for 16S rRNA, partial sequence, | |
| strain: TO-931); GenBank: AB023970.1 | |
| SEQ ID NO: 6 | |
| 1 | ACATGCAAGT CGAGCGATTC TCTTCGGAGA AGAGCGGCGG ACGGGTGAGT AACGCGTGGG |
| 61 | TAACCTGCCC TGTACACACG GATAACATAC CGAAAGGTAT GCTAATACGG GATAATATAT |
| 121 | AAGAGTCGCA TGACTTTTAT ATCAAAGATT TTTCGGTACA GGATGGACCC GCGTCTGATT |
| 181 | AGCTTGTTGG CGGGGTAACG GCCCACCAAG GCGACGATCA GTAGCCGACC TGAGAGGGTG |
| 241 | ATCGGCCACA TTGGAACTGA GACACGGTCC AAACTCCTAC GGGAGGCAGC AGTGGGGAAT |
| 301 | ATTGCACAAT GGGCGCAAGC CTGATGCAGC AACGCCGCGT GAGCGATGAA GGCCTTCGGG |
| 361 | TCGTAAAGCT CTGTCCTCAA GGAAGATAAT GACGGTACTT GAGGAGGAAG CCCCGGCTAA |
| 421 | CTACGTGCCA GCAGCCGCGG TAATACGTAG GGGGCTAGCG TTATCCGGAT TTACTGGGCG |
| 481 | TAAAGGGTGC GTAGGCGGTC TTTCAAGTCA GGAGTTAAAG GCTACGGCTC AACCGTAGTA |
| 541 | AGCTCCTGAT ACTGTCTGAC TTGAGTGCAG GAGAGGAAAG CGGAATTCCC AGTGTAGCGG |
| 601 | TGAAATGCGT AGATATTGGG AGGAACACCA GTAGCGAAGG CGGCTTTCTG GACTGTAACT |
| 661 | GACGCTGAGG CACGAAAGCG TGGGGAGCAA ACAGGATTAG ATACCCTGGT AGTCCACGCT |
| 721 | GTAAACGATG AGTACTAGTT GTCGGAGGTT ACCCCTTCGG TGCCGCAGCT AACGCATTAA |
| 781 | GTACTCCGCC TGGGGAGTAC GCACGCAAGT GTGAAACTCA AAGGAATTGA CGGGGACCCG |
| 841 | CACAAGTAGC GGAGCATGTG GTTTAATTCG AAGCAACGCG AAGAACCTTA CCTAGGCTTG |
| 901 | ACATCCTTCT GACCGAGGAC TAATCTCCTC TTTCCCTCCG GGGACAGAAG TGACAGGTGG |
| 961 | TGCATGGTTG TCGTCAGCTC GTGTCGTGAG ATGTTGGGTT AAGTCCCGCA ACGAGCGCAA |
| 1021 | CCCTTGTCTT TAGTTGCCAT CATTAAGTTG GGCACTCTAG AGAGACTGCC AGGGATAACC |
| 1081 | TGGAGGAAGG TGGGGATGAC GTCAAATCAT CATGCCCCTT ATGCCTAGGG CTACACACGT |
| 1141 | GCTACAATGG GTGGTACAGA GGGCAGCCAA GCCGTGAGGT GGAGCAAATC CCTTAAAGCC |
| 1201 | ATTCTCAGTT CGGATTGTAG GCTGAAACTC GCCTACATGA AGCTGGAGTT ACTAGTAATC |
| 1261 | GCAGATCAGA ATGCTGCGGT GAATGCGTTC CCGGGTCTTG TACACACCGC CCGTCACACC |
| 1321 | ATGGGAGTTG GAGACACCCG AAGCCGACTA TCTAACCTTT TGGGAGAAGT CGTCCCCCTC |
| 1381 | GAATCAATAC CCC |
| (Clostridium leptum 16S ribosomal RNA); GenBank: M59095.1 | |
| SEQ ID NO: 7 | |
| 1 | NNNNNNNNNN NNNNNNNNNN NNNNNNNNNN NNNNNNNNNN NNNNNNNNNN NNNNNNNNNN |
| 61 | NNNNNNNNNN NNNNNNNNNN NNNNTTGGAT TTAACTTAGT GGCGGACGGG TGAGTAACGC |
| 121 | GTGAGTAACC TGCCTTTCAG AGGGGGATAA CGTTCTGAAA AGAACGCTAA TACCGCATAA |
| 181 | CATCAATTTA TCGCATGATA GGTTGATCAA AGGAGCAATC CGCTGGAAGA TGNACTCGCG |
| 241 | TCCGATTAGC CAGTTGGCGG GGTAACGGCC NACCAAAGCG ACGATCGGTA GCCGGACTGA |
| 301 | GAGGTTGAAC GGCCACATTG GGACTGAGAC ACGGCCNNGA CTCCTACGGG AGGCAGCAGT |
| 361 | GGGGGATATT GCACAATGGG GGAAACCCNG ATGCAGCAAC GCCGCGTGAG GGAAGAAGGT |
| 421 | TTTCGGATTG TAAACCTCTG TTCTTAGTGA CGATAATGAC GGTAGCTAAG GAGAAAGCTC |
| 481 | CNNNNAACTA CGTGCCAGCA GCCGCGGTAA TACGTAGGGA GCNAGCGTTG TCCGGATTTA |
| 541 | CTGGGTGTAA AGGGTGCGTA GGCGGCGAGG CAAGTCAGGC GTGAAATCTA TGGGCTTAAC |
| 601 | CCATAAACTG CGCTTGAAAC TGTCTTGCTT GAGTGAAGTA GAGGTAGGCG GAATTCCCNG |
| 661 | TGTAGCGGTN AAATGCGTAG AGATCGGGAG GAACACCAGT GGCGAAGGCG GCCTACTGGG |
| 721 | CTTTAACTGA CGCTGAAGCA CGAAAGCATG GGTAGCAAAC AGGATTAGAT ACCCTGGTAG |
| 781 | TCCATGCCGT AAACGATGAT TACTAGGTGT GNNGGGGGTC TNACCCNNTC CGTGCCGCAG |
| 841 | TTAACACAAT AAGTAATCCA CCTGGGGAGT ACGGCCGCAA GGTTGAAACT CAAAGGAATT |
| 901 | GACGGNNNCC CGCACAAGCA GTGGAGTATG TNGTTTAATT CGAANNAACG CGAAGAACCT |
| 961 | TACCAGGNCT TGACATCCGT CTAACGAAGC AGAGATGCAT TAGGTGCCCT TCGGGGNAAG |
| 1021 | GCGAGACAGG TGGTGCATGG TTGTCGTCAG CTCGTGTCGT GAGATGTTGG GTTAAGTCNN |
| 1081 | GCAACGAGCG CAACNCTTGT TTCTAGTTGC TACGCAAGAG CACTCTAGAG AGACTGCCGT |
| 1141 | TGACAAAACG GAGGAAGGTG GGGACGACGT CAAATCATCA TGCCCNNTAT GACCTGGGCC |
| 1201 | ACACACGTAC TACAATGGCT GTANACAGAG GGAAGCAAAG CCGCGAGGTG GAGCAAAACC |
| 1261 | CTAAAAGCAG TCCCAGTTCG GATCGCAGGC TGCAACCCGC CTGCGTGAAG TCGGAATTGC |
| 1321 | TAGTAATCGC GGATCAGCAT GCCGCGGTGA ATACGTTCCC GGGCNNTGTA CACACCGCCC |
| 1381 | GTCACACCAT GGGAGCCGGT AATACCCGAA GCCAGTAGTT CAACCGCAAG GAGAGCGCTG |
| 1441 | TCGAAGGTAG GATTGGCGAC NNGGG |
| (C. ramosum 16S ribosomal RNA small subunit.); Accesion: M23731.1 | |
| SEQ ID NO: 8 | |
| 1 | ACAATGGAGA GTTTGATCCT GGCTCAGGAT GAACGCTGGC GGCGTGCCTA ATACATGCAA |
| 60 | GTCGAACGCN AGCACTTGTG CTTCGAGTGG CGAACGGGTG AGTAATACAT AAGTAACCTG |
| 120 | CCCTAGACAG GGGGATAACT ATTGGAAACG ATAGCTAAGA CCGCATAGGT ACGGACACTG |
| 180 | CATGGTGACC GTATTAAAAG TGCCTCAAAG CACTGGTAGA GGATGGACTT ATGGCGCATT |
| 240 | AGCTAGTTGG CGGGGTAACG GCCCACCAAG GCGACGATGC GTAGCCGACC TGAGAGGGTG |
| 300 | ACCGGCCACA CTGGGACTGA GACACGGCCC AGACTCCTAC GGGAGGCAGC AGTAGGGAAT |
| 360 | TTTCGGCAAT GGGGGAAACC CTGACCGAGC AACGCCGCGT GAAGGAAGAA GGTTTTCGGA |
| 420 | TTGTAAACTT CTGTTATAAA GGAAGAACGG CGGCTACAGG AAATGGTAGC CGAGTGACGG |
| 480 | TACTTTATTA GAAAGCCACG GCTAACTACG TGCCAGCAGC CGCGGTAATA CGTAGGTGGC |
| 540 | NAGCGTTATC CGGAATTATT GGGCGTAAAG AGGGAGCAGG CGGCAGCAAG GGTCTGTGGT |
| 600 | GAAAGCCTGA AGCTTAACTT CAGTAAGCCA TAGAAACCAG GCAGCTAGAG TGCAGGAGAG |
| 660 | GATCGTGGAA TTCCATGTGT AGCGGTGAAA TGCGTAGATA TATGGAGGAA CACCAGTGGC |
| 720 | GAAGGCGACG ATCTGGCCTG CAACTGACGC TCAGTCCCGA AAGCGTGGGG AGCAAATAGG |
| 780 | ATTAGATACC CTAGTAGTCC ACGCCGTAAA CGATGAGTAC TAAGTGTTGG ATGTCAAAGT |
| 840 | TCAGTGCTGC AGTTAACGCA ATAAGTACTC CGCCTGAGTA GTACGTTCGC AAGAATGAAA |
| 900 | CTCAAAGGAA TTGACGGGGG CCCGCACAAG CGGTGGAGCA TGTGGTTTAA TTCGAAGCAA |
| 960 | CGCGAAGAAC CTTACCAGGT CTTGACATAC TCATAAAGGC TCCAGAGATG GAGAGATAGC |
| 1020 | TATATGAGAT ACAGGTGGTG CATGGTTGTC GTCAGCTCGT GTCGTGAGAT GTTGGGTTAA |
| 1080 | GTCCCGCAAC GAGCGCAACC CTTATCGTTA GTTACCATCA TTAAGTTGGG GACTCTAGCG |
| 1140 | AGACTGCCAG TGACAAGCTG GAGGAAGGCG GGGATGACGT CAAATCATCA TGCCCCTTAT |
| 1200 | GACCTGGGCT ACACACGTGC TACAATGGAT GGTGCAGAGG GAAGCGAAGC CGCGAGGTGA |
| 1260 | AGCAAAACCC ATAAAACCAT TCTCAGTTCG GATTGTAGTC TGCAACTCGA CTACATGAAG |
| 1320 | TTGGAATCGC TAGTAATCGC GAATCAGCAT GTCGCGGTGA ATACGTTCTC GNGCCTTGTA |
| 1380 | CACACCGCCC GTCACACCAC GAGAGTTGAT AACACCCGAA GCNGGTGGCC TAACCGCAAG |
| 1440 | GAAGGAGCTG TCTAAGGTGG GATTGATGAT NGGGGNNNNN NNGTAACAAG GTATCCCTAC |
| 1500 | GNGAACGNNN NNNNNGATCA CCTCCTTTCN |
| (Clostridium sardiniense gene for 16S rRNA, strain: DSM 599, | |
| sub_clone: c1.); Sequence: AB161369.1 | |
| SEQ ID NO: 9 | |
| TTTAAATTGAGAGTTTGATCCTGGCTCAGGACGAACGCTGGCGGCGTGCCTAACACATGC | |
| AAGTCGAGCGATGAAGTTTCCTTCGGGAAACGGATTAGCGGCGGACGGGTGAGTAACACG | |
| TGGGTAACCTGCCTCATAGAGGGGAATAGCCTTCCGAAAGGAAGATTAATACCGCATAAC | |
| ATTGCAGTTTCGCATGAAACAGCAATTAAAGGAGCAATCCGCTATGAGATGGACCCGCGG | |
| CGCATTAGCTAGTTGGTAAGGTAATGGCTTACCAAGGCGACGATGCGTAGCCGACCTGAG | |
| AGGGTGATCGGCCACATTGGGACTGAGACACGGCCCAGACTCCTACGGGAGGCAGCAGTG | |
| GGGAATATTGCACAATGGGGGAAACCCTGATGCAGCAACGCCGCGTGAGTGATGACGGTC | |
| TTCGGATTGTAAAGCTCTGTCTTTGGGGACGATAATGACGGTACCCAAGGAGGAAGCCAC | |
| GGCTAACTACGTGCCAGCAGCCGCGGTAATACGTAGGTGGCAAGCGTTGTCCGGATTTAC | |
| TGGGCGTAAAGGGAGCGTAGGCGGATTTTTAAGTGGGATGTGAAATACCCGGGCTCAACC | |
| TGGGTGCTGCATTCCAAACTGGGAATCTAGAGTGCAGGAGGGGAGAGTGGAATTCCTAGT | |
| GTAGCGGTGAAATGCGTAGAGATTAGGAAGAACACCAGTGGCGAAGGCGACTCTCTGGAC | |
| TGTAACTGACGCTGAGGCTCGAAAGCGTGGGGAGCAAACAGGATTAGATACCCTGGTAGT | |
| CCACGCCGTAAACGATGAATACTAGGTGTAGGGGTTTCGATACCTCTGTGCCGCCGCTAA | |
| CGCATTAAGTATTCCGCCTGGGGAGTACGGTCGCAAGATTAAAACTCAAAGGAATTGACG | |
| GGGGCCCGCACAAGTAGCGGAGCATGTGGTTTAATTCGAAGCAACGCGAAGAACCTTACC | |
| TAGACTTGACATCTTCTGCATTACCCTTAATCGGGGAAGTCCTTTCGGGGACAGAATGAC | |
| AGGTGGTGCATGGTTGTCGTCAGCTCGTGTCGTGAGATGTTGGGTTAAGTCCCGCAACGA | |
| GCGCAACCCCTATTGTTAGTTGCTACCATTAAGTTGAGCACTCTAGCGAGACTGCCCGGG | |
| TTAACCGGGAGGAAGGTGGGGATGACGTCAAATCATCATGCCCCTTATGTCTAGGGCTAC | |
| ACACGTGCTACAATGGCAAGTACAGAGAGATGCAATACCGTGAGGTGGAGCTAAACTTCA | |
| AAACTTGTCTCAGTTCGGATTGTAGGCTGAAACTCGCCTACATGAAGCTGGAGTTACTAG | |
| TAATCGCGAATCAGCATGTCGCGGTGAATACGTTCCCGGGCCTTGTACACACCGCCCGTC | |
| ACACCATGAGAGTTGGCAATACCCAAAGTTCGTGAGCTAACGCGTAAGCGAGGCAGCGAC | |
| CTAAGGTAGGGTCAGCGATTGGGGTGAAGTCGTAACAAGGTAGCCGTAGGAGAACCTGCG | |
| GCTGGATCACCTCCTTTCT | |
| (Clostridium scindens strain ATCC 35704 16S ribosomal RNA, | |
| partial sequence; NCBI Reference Sequence: NR_028785.1 | |
| SEQ ID NO: 10 | |
| 1 | GAGAGTTTGA TCCTGGCTCA GGATGAACGC TGGCGGCGTG CCTAACACAT GCAAGTCGAA |
| 61 | CGAAGCGCCT GGCCCCGACT TCTTCGGAAC GAGGAGCCTT GCGACTGAGT GGCGGACGGG |
| 121 | TGAGTAACGC GTGGGCAACC TGCCTTGCAC TGGGGGATAA CAGCCAGAAA TGGCTGCTAA |
| 181 | TACCGCATAA GACCGAAGCG CCGCATGGCG CGGCGGCCAA AGCCCCGGCG GTGCAAGATG |
| 241 | GGCCCGCGTC TGATTAGGTA GTTGGCGGGG TAACGGCCCA CCAAGCCGAC GATCAGTAGC |
| 301 | CGACCTGAGA GGGTGACCGG CCACATTGGG ACTGAGACAC GGCCCAGACT CCTACGGGAG |
| 361 | GCAGCAGTGG GGAATATTGC ACAATGGGGG AAACCCTGAT GCAGCGACGC CGCGTGAAGG |
| 421 | ATGAAGTATT TCGGTATGTA AACTTCTATC AGCAGGGAAG AAGATGACGG TACCTGACTA |
| 481 | AGAAGCCCCG GCTAACTACG TGCCAGCAGC CGCGGTAATA CGTAGGGGGC AAGCGTTATC |
| 541 | CGGATTTACT GGGTGTAAAG GGAGCGTAGA CGGCGATGCA AGCCAGATGT GAAAGCCCGG |
| 601 | GGCTCAACCC CGGGACTGCA TTTGGAACTG CGTGGCTGGA GTGTCGGAGA GGCAGGCGGA |
| 661 | ATTCCTAGTG TAGCGGTGAA ATGCGTAGAT ATTAGGAGGA ACACCAGTGG CGAAGGCGGC |
| 721 | CTGCTGGACG ATGACTGACG TTGAGGCTCG AAAGCGTGGG GAGCAAACAG GATTAGATAC |
| 781 | CCTGGTAGTC CACGCCGTAA ACGATGACTA CTAGGTGTCG GGTGGCAAGG CCATTCGGTG |
| 841 | CCGCAGCAAA CGCAATAAGT AGTCCACCTG GGGAGTACGT TCGCAAGAAT GAAACTCAAA |
| 901 | GGAATTGACG GGGACCCGCA CAAGCGGTGG AGCATGTGGT TTAATTCGAA GCAACGCGAA |
| 961 | GAACCTTACC TGATCTTGAC ATCCCGATGC CAAAGCGCGT AACGCGCTCT TTCTTCGGAA |
| 1021 | CATCGGTGAC AGGTGGTGCA TGGTTGTCGT CAGCTCGTGT CGTGAGATGT TGGGTTAAGT |
| 1081 | CCCGCAACGA GCGCAACCCC TATCTTCAGT AGCCAGCATT TTGGATGGGC ACTCTGGAGA |
| 1141 | GACTGCCAGG GAGAACCTGG AGGAAGGTGG GGATGACGTC AAATCATCAT GCCCCTTATG |
| 1201 | ACCAGGGCTA CACACGTGCT ACAATGGCGT AAACAAAGGG AGGCGAACCC GCGAGGGTGG |
| 1261 | GCAAATCCCA AAAATAACGT CTCAGTTCGG ATTGTAGTCT GCAACTCGAC TACATGAAGT |
| 1321 | TGGAATCGCT AGTAATCGCG AATCAGAATG TCGCGGTGAA TACGTTCCCG GGTCTTGTAC |
| 1381 | ACACCGCCCG TCACACCATG GGAGTCAGTA ACGCCCGAAG CCGGTGACCC AACCCGTAAG |
| 1441 | GGAGGGAGCC GTCGAAGGTG GGACCGATAA CTGGGGTGAA GTCGTAACAA GGTAGCCGTA |
| 1501 | TCGGAAGGTG CGGCTGGATC ACCTCCTTC |
| (Escherichia coli Nissle 1917 strain URCS8 16S ribosomal RNA | |
| gene, partial sequence); GenBank: KT000039.1 | |
| SEQ ID NO: 11 | |
| 1 | GCTTGCTCCA CCGGAAAAAG AAGAGTGGCG AACGGGTGAG TAACACGTGG GTAACCTGCC |
| 61 | CATCAGAAGG GGATAACACT TGGAAACAGG TGCTAATACC GTATAACAAT CGAAACCGCA |
| 121 | TGGTTTTGAT TTGAAAGGCG CTTTCGGGTG TCGCTGATGG ATGGACCCGC GGTGCATTAG |
| 181 | CTAGTTGGTG AGGTAACGGC TCACCAAGGC CACGATGCAT AGCCGACCTG AGAGGGTGAT |
| 241 | CGGCCACATT GGGACTGAGA CACGGCCCAA ACTCCTACGG GAGGCAGCAG TAGGGAATCT |
| 301 | TCGGCAATGG ACGAAAGTCT GACCGAGCAA CGCCGCGTGA GTGAAGAAGG TTTTCGGATC |
| 361 | GTAAAACTCT GTTGTTAGAG AAGAACAAGG ATGAGAGTAA CTGTTCATCC CTTGACGGTA |
| 421 | TCTAACCAGA AAGCCACGGC TAACTACGTG CCAGCAGCCG CGGTAATACG TAGGTGGCAA |
| 481 | GCGTTGTCCG GATTTATTGG GCGTAAAGCG AGCGCAGGCG GTTTCTTAAG TCTGATGTGA |
| 541 | AAGCCCCCGG CTCAACCGGG GAGGGTCATT GGAAACTGGG AGACTTGAGT GCAGAAGAGG |
| 601 | AGAGTGGAAT TCCATGTGTA GCGGTGAAAT GCGTAGATAT ATGGAGGAAC ACCAGTGGCG |
| 661 | AAGGCGGCTC TCTGGTCTGT AACTGACGCT GAGGCTCGAA AGCGTGGGGA GCAAACAGGA |
| 721 | TTAGATACCC TGGTAGTCCA CGCCGTAAAC GATGAGTGCT AAGTGTTGGA GGGTTTCCGC |
| 781 | CCTTCAGTGC TGCAGCTAAC GCATTAAGCA CTCCGCCTGG GGAGTACGAC CGCAAGGTTG |
| 841 | AAAC |
| (Klebsiella oxytoca culture-collection ATCC: 700324 clone d08 16S- | |
| 23S ribosomal RNA intergenic spacer, partial sequence; and tRNA-Ile and | |
| tRNA-Ala genes, complete sequence); GenBank: EU623169.1 | |
| SEQ ID NO: 12 | |
| 1 | CCTGAAAGAA CCTGCCTTTG TAGTGCTCAC ACAGATTGTC TGATGAAAAA TAAGCAGTAA |
| 61 | GAAAATCTCT GCAGGCTTGT AGCTCAGGTG GTTAGAGCGC ACCCCTGATA AGGGTGAGGT |
| 121 | CGGTGGTTCA AGTCCACTCA GGCCTACCAA ATTTCTGCTG ATGCTGCGTT GCGGCGACAC |
| 181 | TCACATACTT TAAGTATGTT TCGTGTCACC ACGCCTTGCC TCAACAGAAA TTAAGGTTGA |
| 241 | TGAGATTTTA ACTACGATGG GGCTATAGCT CAGCTGGGAG AGCGCCTGCT TTGCACGCAG |
| 301 | GAGGTCTGCG GTTCGATCCC GCATAGCTCC ACCATCATTA CTGCCAAAAA CAAGAAAACT |
| 361 | TCAGAGTGTA CCTGAAAAGG TTCACTGCGA AGTTTTGCTC TTTAAAAATC TGGATCAAGC |
| 421 | TGAAAATTGA AACGACACAC AGCTAATGTG TGTTCGAGTC TCTCAAATTT TCGCGACACG |
| 481 | ATGATGTTTC ACGAAACATC TTCGGGTTGT GA |
| (Parabacteroides distasonis strain ATCC 8503 16S ribosomal RNA, | |
| partial sequence); NCBI Reference Sequence: NR_074376.1 | |
| SEQ ID NO: 13 | |
| 1 | CAATTTAAAC AACGAAGAGT TTGATCCTGG CTCAGGATGA ACGCTAGCGA CAGGCTTAAC |
| 61 | ACATGCAAGT CGAGGGGCAG CGGGGTGTAG CAATACACCG CCGGCGACCG GCGCACGGGT |
| 121 | GAGTAACGCG TATGCAACTT GCCTATCAGA GGGGGATAAC CCGGCGAAAG TCGGACTAAT |
| 181 | ACCGCATGAA GCAGGGATCC CGCATGGGAA TATTTGCTAA AGATTCATCG CTGATAGATA |
| 241 | GGCATGCGTT CCATTAGGCA GTTGGCGGGG TAACGGCCCA CCAAACCGAC GATGGATAGG |
| 301 | GGTTCTGAGA GGAAGGTCCC CCACATTGGT ACTGAGACAC GGACCAAACT CCTACGGGAG |
| 361 | GCAGCAGTGA GGAATATTGG TCAATGGCCG AGAGGCTGAA CCAGCCAAGT CGCGTGAGGG |
| 421 | ATGAAGGTTC TATGGATCGT AAACCTCTTT TATAAGGGAA TAAAGTGCGG GACGTGTCCC |
| 481 | GTTTTGTATG TACCTTATGA ATAAGGATCG GCTAACTCCG TGCCAGCAGC CGCGGTAATA |
| 541 | CGGAGGATCC GAGCGTTATC CGGATTTATT GGGTTTAAAG GGTGCGTAGG CGGCCTTTTA |
| 601 | AGTCAGCGGT GAAAGTCTGT GGCTCAACCA TAGAATTGCC GTTGAAACTG GGGGGCTTGA |
| 661 | GTATGTTTGA GGCAGGCGGA ATGCGTGGTG TAGCGGTGAA ATGCATAGAT ATCACGCAGA |
| 721 | ACCCCGATTG CGAAGGCAGC CTGCCAAGCC ATTACTGACG CTGATGCACG AAAGCGTGGG |
| 781 | GATCAAACAG GATTAGATAC CCTGGTAGTC CACGCAGTAA ACGATGATCA CTAGCTGTTT |
| 841 | GCGATACACT GTAAGCGGCA CAGCGAAAGC GTTAAGTGAT CCACCTGGGG AGTACGCCGG |
| 901 | CAACGGTGAA ACTCAAAGGA ATTGACGGGG GCCCGCACAA GCGGAGGAAC ATGTGGTTTA |
| 961 | ATTCGATGAT ACGCGAGGAA CCTTACCCGG GTTTGAACGC ATTCGGACCG AGGTGGAAAC |
| 1021 | ACCTTTTCTA GCAATAGCCG TTTGCGAGGT GCTGCATGGT TGTCGTCAGC TCGTGCCGTG |
| 1081 | AGGTGTCGGC TTAAGTGCCA TAACGAGCGC AACCCTTGCC ACTAGTTACT AACAGGTTAG |
| 1141 | GCTGAGGACT CTGGTGGGAC TGCCAGCGTA AGCTGCGAGG AAGGCGGGGA TGACGTCAAA |
| 1201 | TCAGCACGGC CCTTACATCC GGGGCGACAC ACGTGTTACA ATGGCGTGGA CAAAGGGAGG |
| 1261 | CCACCTGGCG ACAGGGAGCG AATCCCCAAA CCACGTCTCA GTTCGGATCG GAGTCTGCAA |
| 1321 | CCCGACTCCG TGAAGCTGGA TTCGCTAGTA ATCGCGCATC AGCCATGGCG CGGTGAATAC |
| 1381 | GTTCCCGGGC CTTGTACACA CCGCCCGTCA AGCCATGGGA GCCGGGGGTA CCTGAAGTCC |
| 1441 | GTAACCGAAA GGATCGGCCT AGGGTAAAAC TGGTGACTGG GGCTAAGTCG TAACAAG |
| (Prevotella melaninogenica strain ATCC 25845 16S ribosomal RNA | |
| gene, partial sequence); NCBI Reference Sequence: NR_042843.1 | |
| SEQ ID NO: 14 | |
| 1 | GATGAACGCT AGCTACAGGC TTAACACATG CAAGTNGAGG GGAAACGGCA TTGAGTGCTT |
| 61 | GCACTCTTTG GACGTCGACC GGCGCACGGG TGAGTAACGC GTATCCAACC TTCCCATTAC |
| 121 | TGTGGGATAA CCTGCCGAAA GGCAGACTAA TACCGCATAG TCTTCGATGA CGGCATCAGA |
| 181 | TTTGAAGTAA AGATTTATCG GTAATGGATG GGGATGCGTC TGATTAGCTT GTTGGCGGGG |
| 241 | TAACGGCCCA CCAAGGCAAC GATCAGTAGG GGTTCTGAGA GGAAGGTCCC CCACATTGGA |
| 301 | ACTGAGACAC GGTCCAAACT CCTACGGGAG GCAGCAGTGA GGAATATTGG TCAATGGACG |
| 361 | GAAGTCTGAA CCAGCCAAGT AGCGTGCAGG ATGACGGCCC TATGGGTTGT AAACTGCTTT |
| 421 | TGTATGGGGA TAAAGTTAGG GACGTGTCCC TATTTGCAGG TACCATACGA ATAAGGACCG |
| 481 | GCTAATTCCG TGCCAGCAGC CGCGGTAATA CGGAAGGTCC AGGCGTTATC CGGATTTATT |
| 541 | GGGTTTAAAG GGAGCGTAGG CTGGAGATTA AGTGTGTTGT GAAATGTAGA CGCTCAACGT |
| 601 | CTGAATTGCA GCGCATACTG GTTTCCTTGA GTACGCACAA CGTTGGCGGA ATTCGTCGTG |
| 661 | TAGCGGTGAA ATGCTTAGAT ATGACGAAGA ACTCCGATTG CGAAGGCAGC TGACGGGAGC |
| 721 | GCAACTGACG CTTAAGCTCG AAGGTGCGGG TATCAAACAG GATTAGATAC CCTGGTAGTC |
| 781 | CGCACAGTAA ACGATGGATG CCCGCTGTTG GTACCTGGTA TCAGCGGCTA AGCGAAAGCA |
| 841 | TTAAGCATCC CACCTGGGGA GTACGCCGGC AACGGTGAAA CTCAAAGGAA TTGACGGGGG |
| 901 | CCCGCACAAG CGGAGGAACA TGTGGTTTAA TTCGATGATA CGCGAGGAAC CTTACCCGGG |
| 961 | CTTGAATTGC AGAGGAAGGA TTTAGAGATA ATGACGCCCT TCGGGGTCTC TGTGAAGGTG |
| 1021 | CTGCATGGTT GTCGTCAGCT CGTGCCGTGA GGTGTCGGCT TAAGTGCCAT AACGAGCGCA |
| 1081 | ACCCCTCTCT TCAGTTGCCA TCAGGTTAAG CTGGGCACTC TGGAGACACT GCCACCGTAA |
| 1141 | GGTGTGAGGA AGGTGGGGAT GACGTCAAAT CAGCACGGCC CTTACGTCCG GGGCTACACA |
| 1201 | CGTGTTACAA TGGCCGGTAC AGAGGGACGG TGTAATGCAA ATTGCATCCA ATCTTGAAAG |
| 1261 | CCGGTCCCAG TTCGGACTGG GGTCTGCAAC CCGACCCCAC GAAGCTGGAT TCGCTAGTAA |
| 1321 | TCGCGCATCA GCCATGGCGC GGTGAATACG TTCCCGGGCC TTGTACACAC CGCCCGTCAA |
| 1381 | GCCATGAAAG CCGGGGGTGC CTGAAGTCCG TGACCGCAAG GATCGGCCTA GGGCAAAACT |
| 1441 | GGTGATTGGG GCTAAGTCGT AACAAGGTAG CCGTACCGGA AGGTGCGGCT GGAACACCTC |
| 1501 | CTTTCT |
| (Proteus mirabilis strain ATCC 29906 16S ribosomal RNA gene, | |
| partial sequence); NCBI Reference Sequence: NR_114419.1 | |
| SEQ ID NO: 15 | |
| 1 | TGATCCTGGC TCAGATTGAA CGCTGGCGGC AGGCCTAACA CATGCAAGTC GAGCGGTAAC |
| 61 | AGGAGAAAGC TTGCTTTCTT GCTGACGAGC GGCGGACGGG TGAGTAATGT ATGGGGATCT |
| 121 | GCCCGATAGA GGGGGATAAC TACTGGAAAC GGTGGCTAAT ACCGCATAAT GTCTACGGAC |
| 181 | CAAAGCAGGG GCTCTTCGGA CCTTGCACTA TCGGATGAAC CCATATGGGA TTAGCTAGTA |
| 241 | GGTGGGGTAA AGGCTCACCT AGGCGACGAT CTCTAGCTGG TCTGAGAGGA TGATCAGCCA |
| 301 | CACTGGGACT GAGACACGGC CCAGACTCCT ACGGGAGGCA GCAGTGGGGA ATATTGCACA |
| 361 | ATGGGCGCAA GCCTGATGCA GCCATGCCGC GTGTATGAAG AAGGCCTTAG GGTTGTAAAG |
| 421 | TACTTTCAGC GGGGAGGAAG GTGATAAGGT TAATACCCTT ATCAATTGAC GTTACCCGCA |
| 481 | GAAGAAGCAC CGGCTAACTC CGTGCCAGCA GCCGCGGTAA TACGGAGGGT GCAAGCGTTA |
| 541 | ATCGGAATTA CTGGGCGTAA AGCGCACGCA GGCGGTCAAT TAAGTCAGAT GTGAAAGCCC |
| 601 | CGAGCTTAAC TTGGGAATTG CATCTGAAAC TGGTTGGCTA GAGTCTTGTA GAGGGGGGTA |
| 661 | GAATTCCATG TGTAGCGGTG AAATGCGTAG AGATGTGGAG GAATACCGGT GGCGAAGGCG |
| 721 | GCCCCCTGGA CAAAGACTGA CGCTCAGGTG CGAAAGCGTG GGGAGCAAAC AGGATTAGAT |
| 781 | ACCCTGGTAG TCCACGCTGT AAACGATGTC GATTTAGAGG TTGTGGTCTT GAACCGTGGC |
| 841 | TTCTGGAGCT AACGCGTTAA ATCGACCGCC TGGGGAGTAC GGCCGCAAGG TTAAAACTCA |
| 901 | AATGAATTGA CGGGGGCCCG CACAAGCGGT GGAGCATGTG GTTTAATTCG ATGCAACGCG |
| 961 | AAGAACCTTA CCTACTCTTG ACATCCAGCG AATCCTTTAG AGATAGAGGA GTGCCTTCGG |
| 1021 | GAACGCTGAG ACAGGTGCTG CATGGCTGTC GTCAGCTCGT GTTGTGAAAT GTTGGGTTAA |
| 1081 | GTCCCGCAAC GAGCGCAACC CTTATCCTTT GTTGCCAGCA CGTAATGGTG GGAACTCAAA |
| 1141 | GGAGACTGCC GGTGATAAAC CGGAGGAAGG TGGGGATGAC GTCAAGTCAT CATGGCCCTT |
| 1201 | ACGAGTAGGG CTACACACGT GCTACAATGG CAGATACAAA GAGAAGCGAC CTCGCGAGAG |
| 1261 | CAAGCGGAAC TCATAAAGTC TGTCGTAGTC CGGATTGGAG TCTGCAACTC GACTCCATGA |
| 1321 | AGTCGGAATC GCTAGTAATC GTAGATCAGA ATGCTACGGT GAATACGTTC CCGGGCCTTG |
| 1381 | TACACACCGC CCGTCACACC ATGGGAGTGG GTTGCAAAAG AAGTAGGTAG CTTAACCTTC |
| 1441 | GGGAGGGCGC TTACCACTTT GTGATTCATG ACTGGGGTGA AGTCGTAACA AGGTAGC |
| (Subdoligranulum variabile 16S rRNA gene, type strain BI 114T; strain | |
| also referred to as CCUG 47106 and/or DSM 15176); NCBI Reference | |
| Sequence: AJ518869.1 | |
| SEQ ID NO: 16 | |
| 1 | tgcaagtcga acggagttat ttcggttgaa gttttcggat ggatactggt ttaacttagt |
| 61 | ggcgaacggg tgagtaacgc gtgagtaacc tgccctggag tgggggacaa cagttggaaa |
| 121 | cgactgctaa taccgcataa gcccacgatc cggcatcgga ttgagggaaa aggatttatt |
| 181 | cgcttcagga tggactcgcg tccaattagc tagttggtga ggtaacggcc caccaaggcg |
| 241 | acgattggta gccggactga gaggttgaac ggccacattg ggactgagac acggcccaga |
| 301 | ctcctacggg aggcagcagt gggggatatt gcacaatggg ggaaaccctg atgcagcgac |
| 361 | gccgcgtgga ggaagaaggt tttcggattg taaactcctg tcgttaggga cgaatcttga |
| 421 | cggtacctaa caagaaagca ccggctaact acgtgccagc agccgcggta aaacgtaggg |
| 481 | tgcaagcgtt gtccggaatt actgggtgta aagggagcgc aggcggaccg gcaagttgga |
| 541 | agtgaaatct atgggctcaa cccataaatt gctttcaaaa ctgctggcct tgagtagtgc |
| 601 | agaggtaggt ggaattcccg gtgtagcggt ggaatgcgta gatatcggga ggaacaccag |
| 661 | tggcgaaggc gacctactgg gcaccaactg acgctgaggc tcgaaagcat gggtagcaaa |
| 721 | caggattaga taccctggta gtccatgccg taaacgatga ttactaggtg ttggaggatt |
| 781 | gaccccttca gtgccgcagt taacacaata agtaatccac ctggggagta cgaccgcaag |
| 841 | gttgaaactc aaaggaattg acgggggccc gcacaagcag tggagtatgt ggtttaattc |
| 901 | gaagcaacgc gaagaacctt accaggtctt gacatccgat gcatagtgca gagatgcatg |
| 961 | aagtccttcg ggacatcgag acaggtggtg catggttgtc gtcagctcgt gtcgtgagat |
| 1021 | gttgggttaa gtcccgcaac gagcgcaacc cttattgcca gttactacgc aagaggactc |
| 1081 | tggcgagact gccgttgaca aaacggagga aggtggggat gacgtcaaat catcatgccc |
| 1141 | tttatgacct gggctacaca cgtactacaa tggcgtttaa caaagagang caagaccgcg |
| 1201 | aggtggagca aaactcaaaa acaacgtctc agttcagatt gcaggctgca actcgcctgc |
| 1261 | atgaagtcgg aattgctagt aatcgcggat cagcatgccg cggtgaatac gttcccgggc |
| 1321 | cttgtacaca ccgcccgtca caccatgaga gccggggggg acccgaagtc ggtaagtaag |
| 1381 | tctaaccgca aggaggacgc cgccgaaggt aaaactggtg attgggtg |

### Example 5: Microbiota Therapy Acts Via a Regulatory T Cell MyD88/ROR-γt Pathway to Suppress Food Allergy

The role of dysbiosis in food allergy (FA) remains unclear. It was observed that dysbiotic fecal microbiota in FA infants evolved compositionally over time, and failed to protect against FA in mice. Both infants and mice with FA had decreased secretory IgA and increased IgE binding to fecal bacteria, indicative of a broader breakdown of oral tolerance in FA than hitherto appreciated. Therapy with Clostridiales species reflective of taxa impacted by dysbiosis, either as a small consortium or as monotherapy with Subdoligranulum variabile, suppressed FA in mice, as did a separate immunomodulatory consortium of Bacteroidales species. Bacteriotherapy induced regulatory T (Treg) cells expressing the transcription factor ROR-γt in a MyD88-dependent manner, which were deficient in FA subjects and mice and ineffectively induced by their microbiota. Deletion of Myd88 or Rorc in Treg cells abrogated protection by bacteriotherapy. Thus, commensals activate a microbial sensing MyD88/ROR-γt pathway in nascent Treg cells to protect against FA, while dysbiotic communities impair this regulatory response to promote disease.

### Introduction:

Food allergy (FA) is a major public health concern, whose prevalence has grown dramatically over the past decade. FA now affects 6% of children under 5 years, and 3% of teens and adults. Most FA is acquired in the first or second year of life, indicating that early childhood exposures have profound long-term health consequences. Several studies have shown that factors impacting gut microbial colonization and composition early in life, including method of delivery (i.e., cesarean section), antibiotic use, and breastfeeding influence the development of atopic disease. Less information is available on the role of gut microbiota in human FA. Reduced gut microbiota diversity and an elevated ratio of the abundance of Enterobacteriaceae to Bacteroidaceae species in early infancy have been associated with subsequent food sensitization, suggesting that the initial stages of gut colonization with particular microbial communities may contribute to the development of atopic disease, including FA.

Evidence points to a key role for the gut microbiota in FA. Mice raised in a sterile environment cannot be orally tolerized to antigens, have a reduced gut mucosal IgA levels and decreased IL-10 producing regulatory T (Treg) cells. In contrast, colonization with Clostridia species promotes the development of Treg cells. Herein, it is observed that in a FA-prone genetic mouse model (Il4ra^{F709} mice), the acquisition of FA is associated with a gut microbiota signature that is distinct from that of FA-tolerant mice. Transfer of fecal microbiota from FA but not tolerant mice to wild-type (WIT) germ-free (GF) recipients transmitted susceptibility to FA.

These findings suggest that unfavorable alterations in the gut microbiota early in life lead to dysbiosis with the loss of beneficial species. Such changes may derail oral immune tolerance and increase susceptibility to allergic and inflammatory responses. Mechanisms by which the commensal microbiota may promote oral tolerance to food allergens include elicitation of protective mucosal Treg cell responses and enhancement of epithelial cell barrier integrity. The production of short-chain fatty acids (SCFA), such as acetate, propionate and butyrate, by commensals such as Clostridia species, helps recruit and stabilize Treg cells in the gut. Colonization with commensal bacteria also expands a population of induced Treg (iTreg) cells in the gut that expresses the transcription factor retinoic acid orphan receptor-gamma (ROR-γt), which, without wishing to be bound by theory, have been proposed to regulate allergic inflammation by inhibiting Th2 cell responses.

Herein is demonstrated that FA infants manifest an evolving dysbiosis that impacts beneficial gut commensals. Furthermore, administration of defined bacterial consortia of human-origin commensals, one composed of culturable species from the order Clostridiales and the other from order Bacteroidales, prevented FA and cured established disease in Il4ra^{F709} mice. Both consortia activated a MyD88-dependent microbial sensing pathway in nascent gut Treg cells that induced their differentiation into disease-suppressing ROR-γt⁺ Treg cells. This population was found deficient in FA subjects and mice due to dysbiosis. These results identify a shared regulatory mechanism by which different commensals enforce oral tolerance and suppress FA, and underscore the potential for microbial therapies in treating this disorder.

### Results

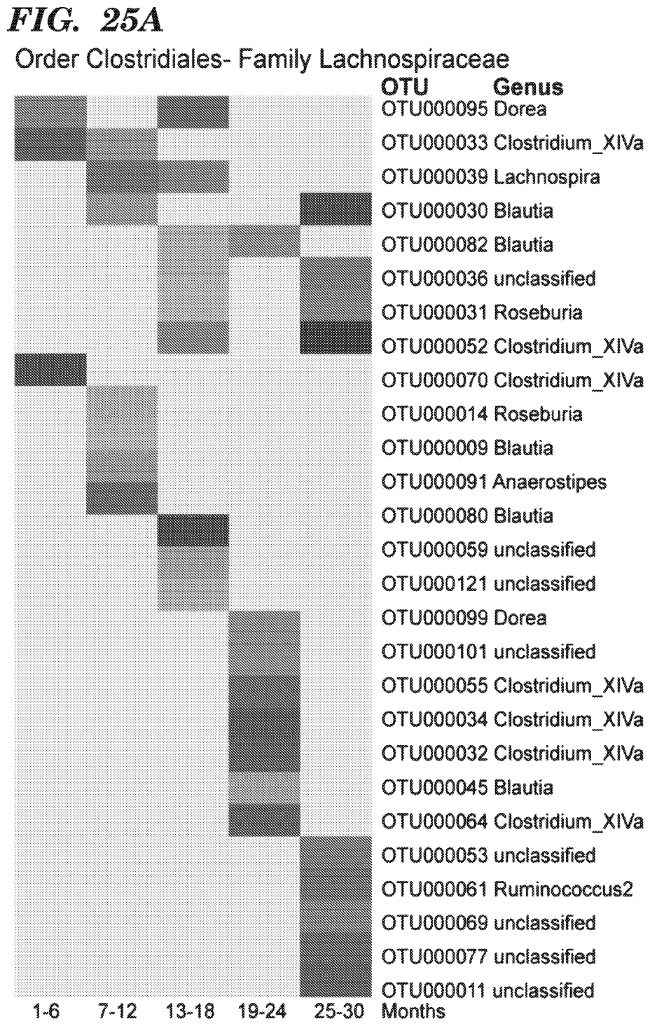

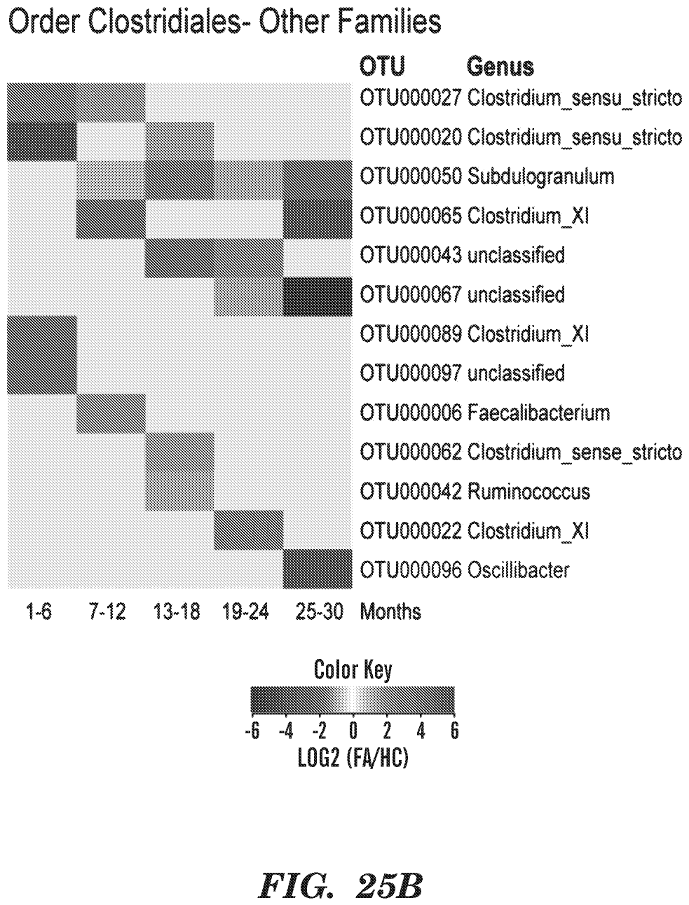

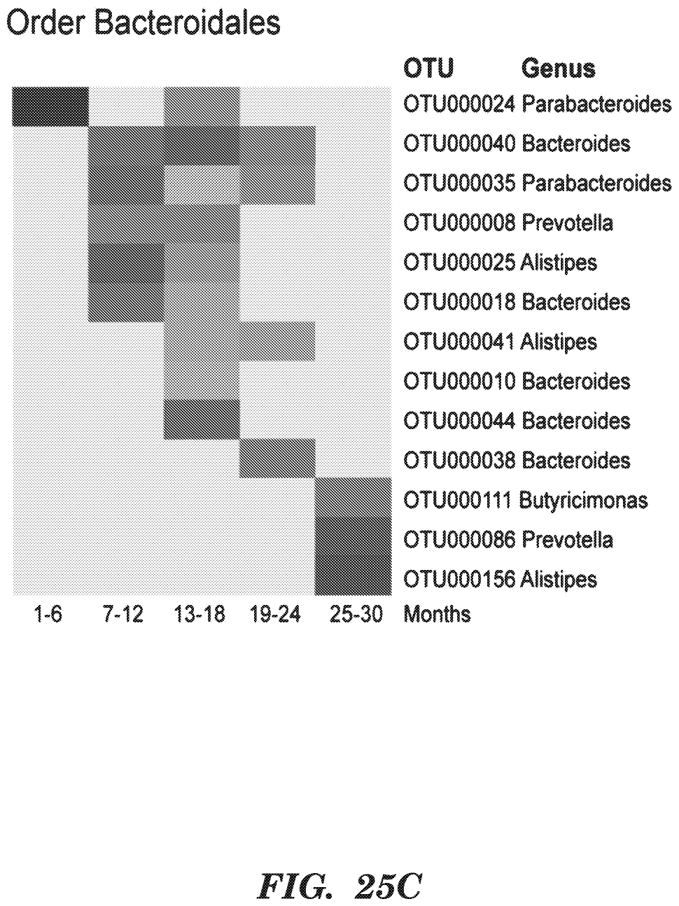

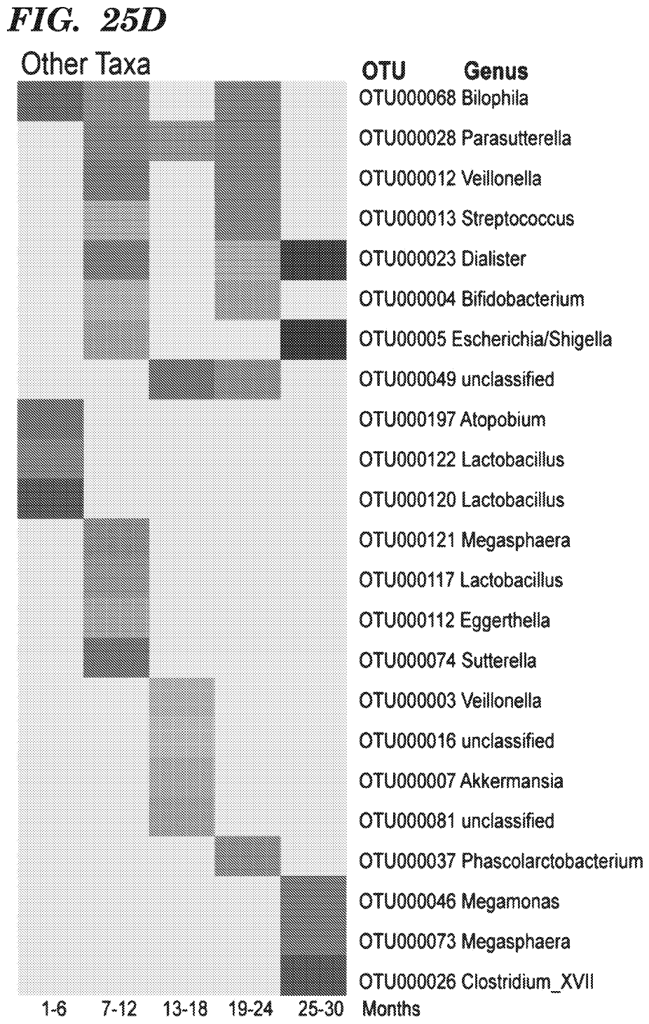

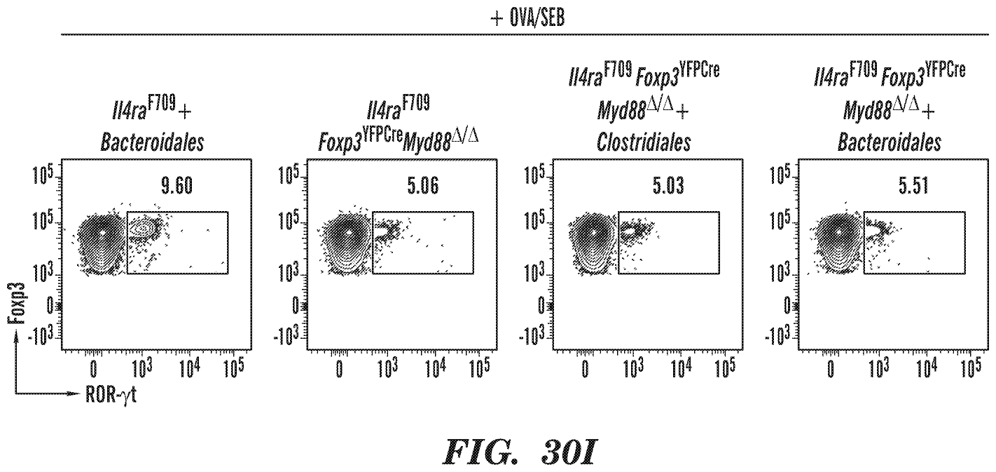

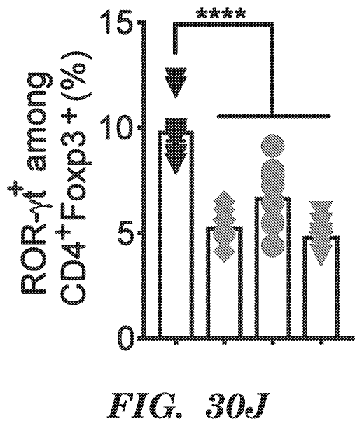

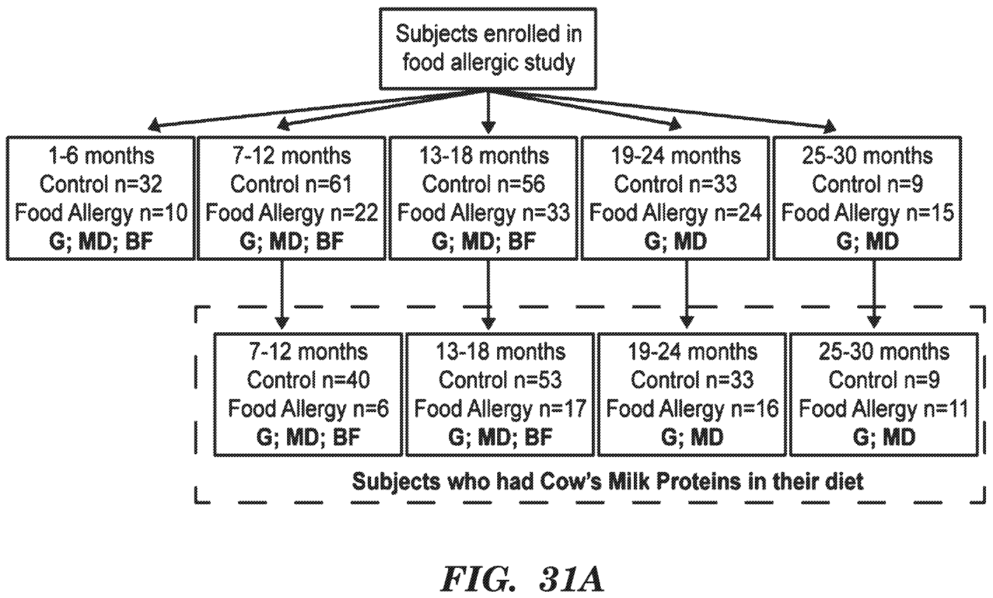

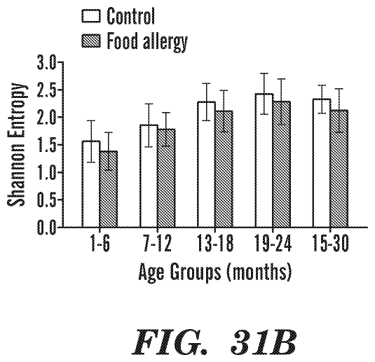

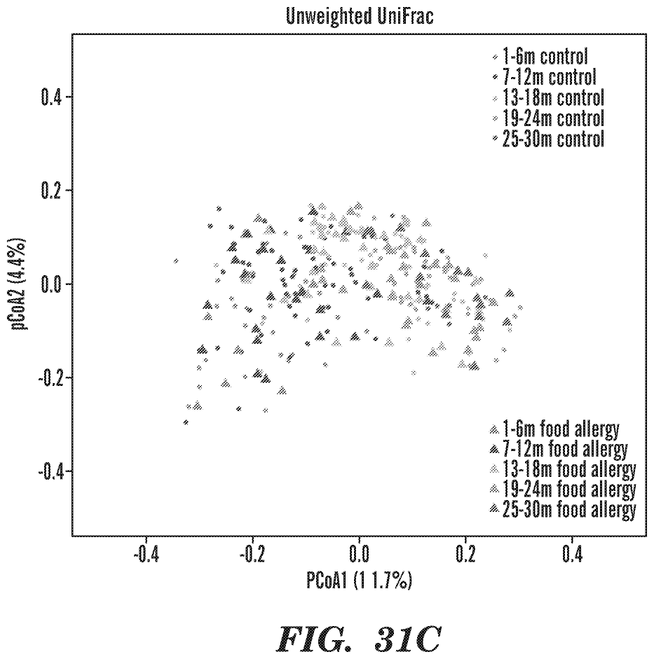

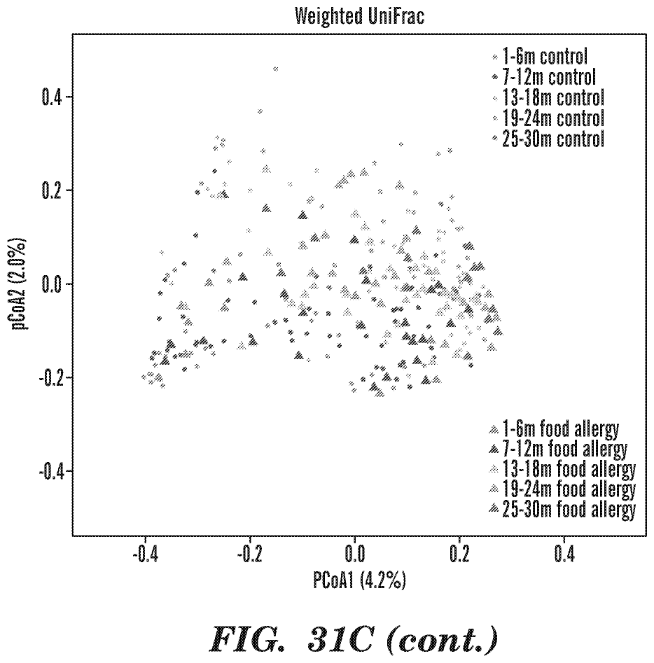

Patients with FA manifest early onset dynamic gut dysbiosis. Without wishing to be bound by theory, it is proposed that food allergy early in life is associated with gut microbial dysbiosis. To test this hypothesis, analysis was conducted of the fecal microbiota of 56 food allergic (FA) and 98 age matched control infants recruited at 1-15 months of age and periodically sampled every 4-6 months for up to 30 months of age. TABLE 6 summarizes subject demographics and FIG. 25A summarizes the distribution of samples collected by subject age. FA versus healthy control (HC) subjects demonstrated no significant differences in the overall ecological diversity of the fecal microbiota as assessed by measures of alpha and beta diversity (see e.g., FIG. 31B-33C). However, compositional differences in relative abundance among 77 Operational Taxonomic Units (OTUs) were observed in the fecal microbiome between age-stratified food allergic subjects and controls [False discovery rate (FDR)-adjusted p-value <0.1] (see e.g., FIG. 25 and TABLE 7). Within this subgroup of 77 taxa, differences for some taxa occurred across more than one age group in FA versus control patients, while other taxa showed significant differences primarily in specific age groups. These associations in FA patients occurred even when controlling for factors including gender, mode of delivery for all age groups, and breastfeeding until 18 months of age, using multivariate statistical models.

Among taxa associated with the development of FA, the earliest alterations showed increased relative abundance of taxa with closest reference species (CRS) Bilophila wadsworthia, Clostridium butyricum and Clostridium disporicum. The latter two species are members of the genus Clostridium sensu stricto (Clostridium Cluster I), and have been previously associated with allergen specific IgE in FA. Alterations in CRS including Parasutterella excrementihominis, Veillonella ratti, Bacteroides stercoris, Alistipes onderdonkii and Prevotella copri, and Roseburia inulinivorans followed in later age groups (see e.g., FIG. 25 and TABLE 7). In contrast, CRS Subdoligranulum variabile (OTU 50) was persistently decreased across several age groups in FA subjects one year and older relative to controls.

In addition to the aforementioned changes that were noted across several age groups, age-specific differences emerged in the microbiota of FA versus control subjects. Some of the earliest changes involved increases of several taxa in FA infants, including CRS Clostridium aldenense, Clostridium (Cellulosoyticum) lentocellulum, Peptostreptococcus anaerobius/stomatis and Lactobacillus johnsonii (1-6 months of age), and CRS Faecalibacterium prausnitzii, Blautia wexlerae, Anaerostipes caccae and Lactobacillus rossiae (7-12 months of age). Older FA subjects showed decreases in several Clostridiales taxa from cluster XIVa, including CRS Clostridium hathewayi, Clostridium symbiosum, Clostridium lavalense and Clostridium scindens.

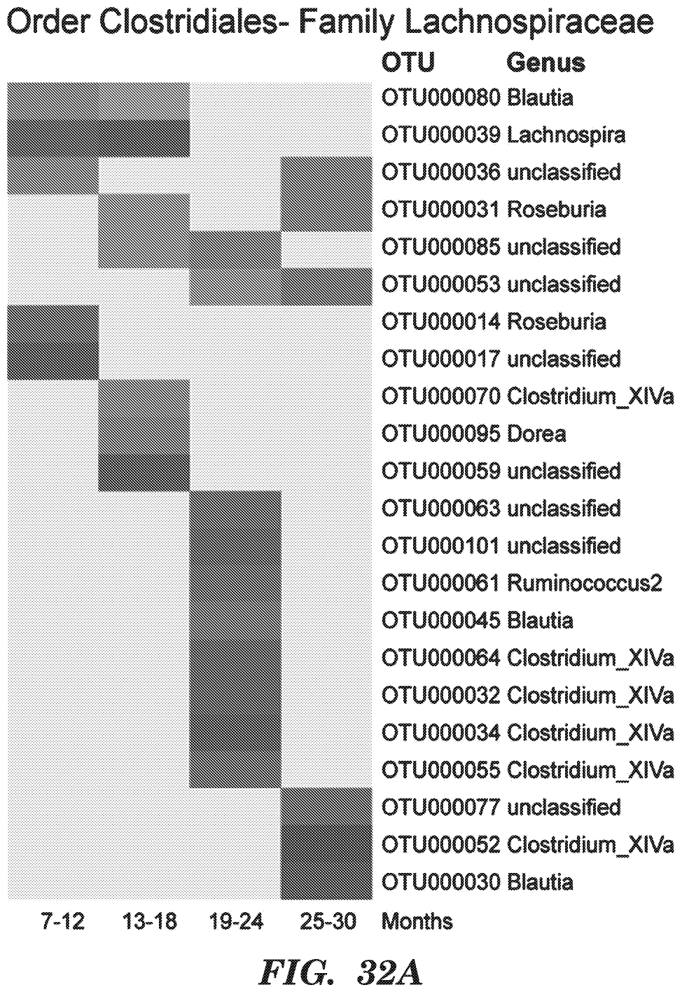

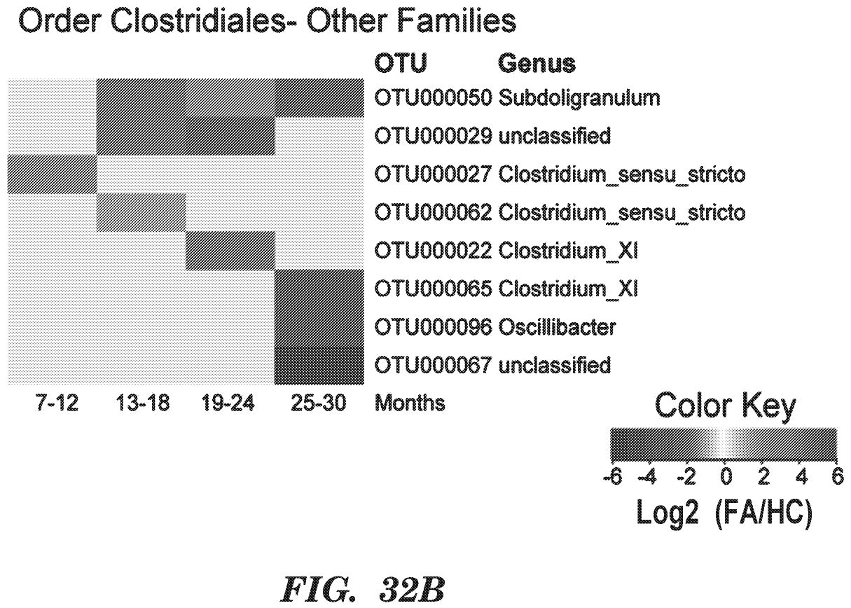

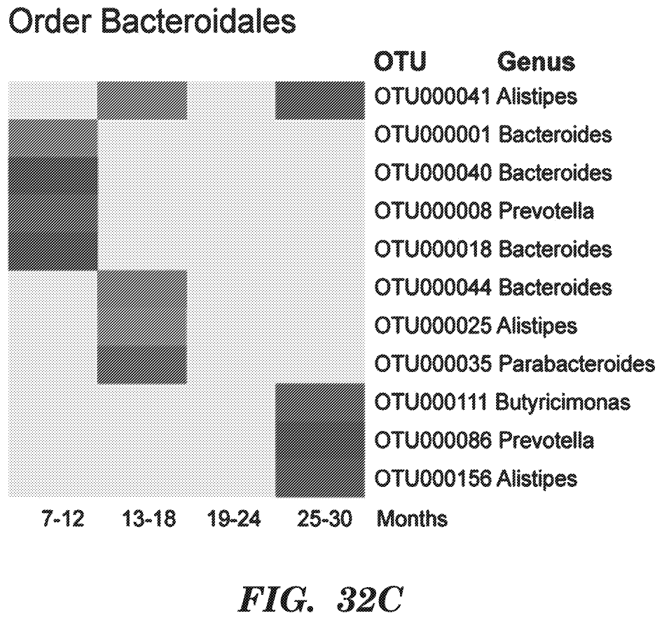

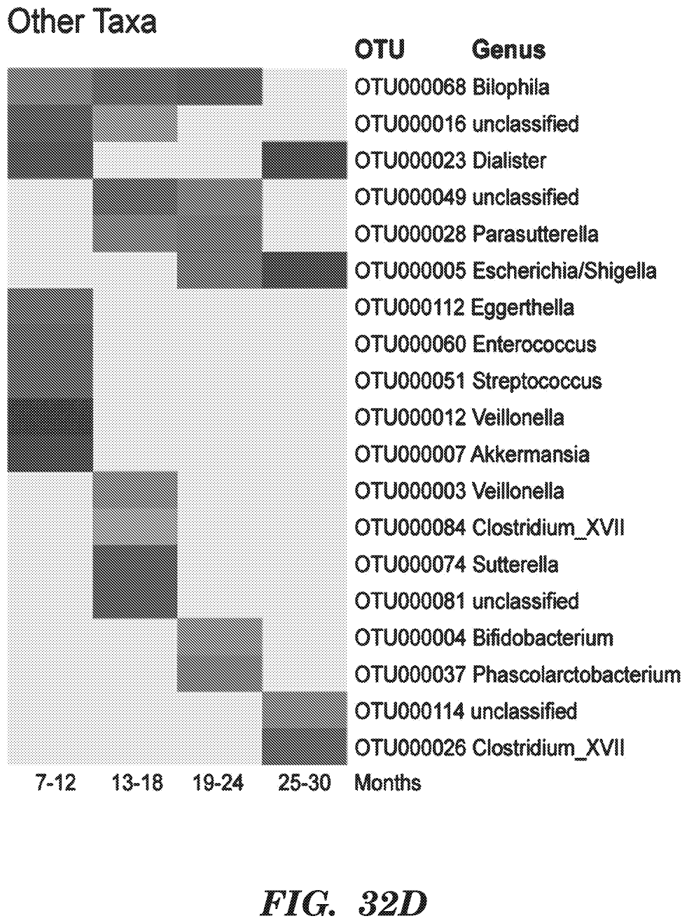

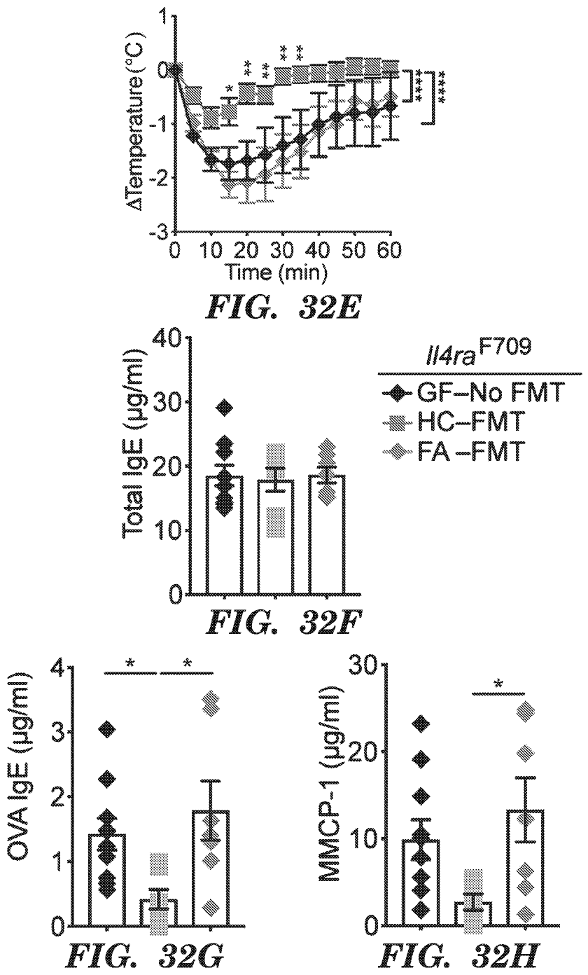

As cow's milk is an important food staple in childhood, and because milk avoidance due to allergy could represent a confounder that influences the composition of the microbiota, we compared the gut microbiota of control subjects who were consuming milk products to that of FA patients who were tolerant and consuming milk but were allergic to other food(s). When controlled for milk avoidance, 61 differentially abundant OTUs strongly overlapped with those identified in FA subjects not segregated for milk allergy (FDR-adjusted p-value <0.1), with 16 OTUs detected across more than one age group, including CRS Subdoligranulum variabile which was also persistently decreased in milk-tolerant FA subjects 1 year and older (see e.g., FIG. 32 and TABLE 8). These results indicated that dysbiosis was a general attribute of FA early in life.

The microbiota of FA subjects fail to protect against FA in a mouse disease model. To assess the functional significance of dysbiosis in FA, Il4ra^{F709} mice were employed, which are genetically prone to develop FA upon oral sensitization with food allergens. Adult GF Il4ra^{F709} mice that remained germfree or that received fecal microbiota transplants (FMT) from HC or FA infants, were sensitized with chicken egg ovalbumin (OVA) in the presence of the mucosal adjuvant staphylococcal enterotoxin B (SEB) and subsequently challenged with OVA. GF Il4ra^{F709} mice or those that received FMT from FA subjects exhibited a rapid and sustained drop in their core body temperature, consistent with anaphylaxis, whereas those that received donor microbiota from HC had a mild drop that rapidly reversed (see e.g., FIG. 32E). While the total serum IgE concentrations across the three OVA-sensitized mouse groups were similar, induction of OVA-specific IgE was markedly decreased in mice receiving donor microbiota from HC subjects as compared to those receiving donor microbiota from FA subjects or that remained GF (see e.g., FIG. 32F). Also, the increase in serum mouse mast cell protease 1 (MMCP1) concentrations post anaphylaxis was notably higher in those Il4ra^{F709} mice that were GF or that have received FMT from FA subjects as compared to mice that received FMT from healthy subjects (see e.g., FIG. 32G). These results indicated that the capacity of the gut commensal flora to impart protection against FA was profoundly impaired in FA as compared to HC subjects.

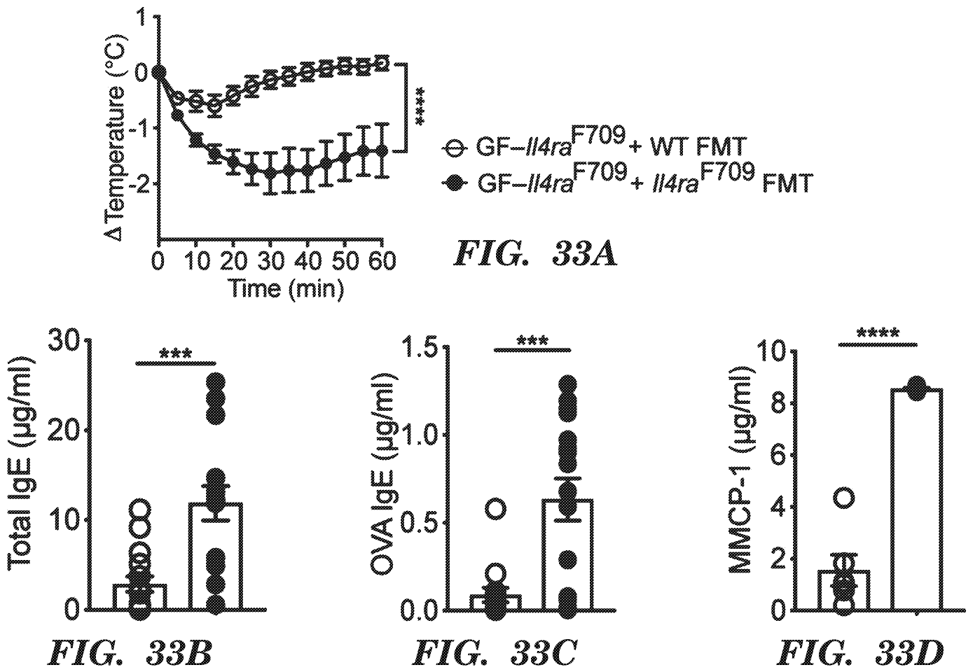

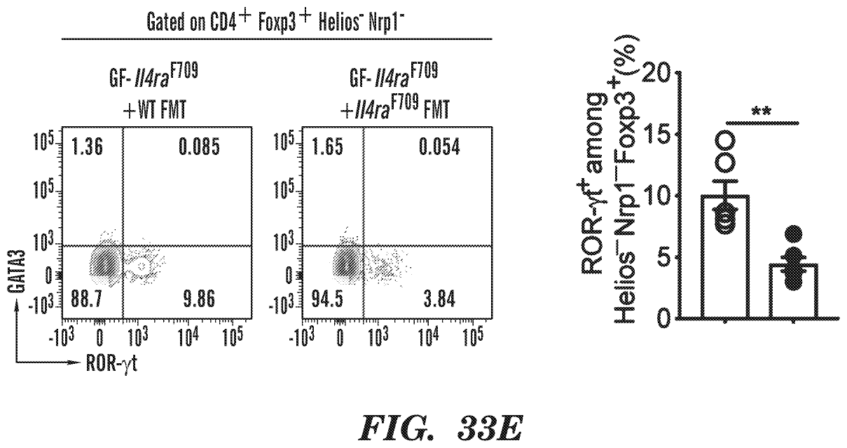

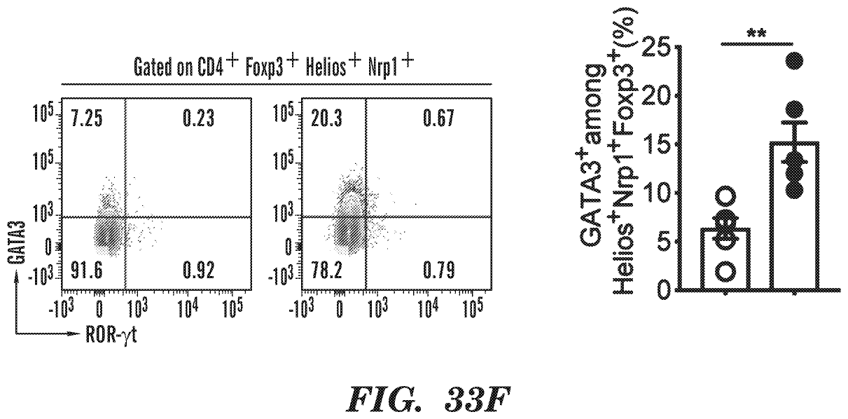

Similar to FA human subjects, Il4ra^{F709} mice also exhibit dysbiotic microbiota which, upon transfer into GF WT BALB/c mice, heightens their susceptibility to FA as compared to microbiota from control BLAB/c mice. Herein the capacity was analyzed of microbiota derived from specific pathogen-free (SPF) WT BALB/c mice, which are relatively resistant to FA induction, to rescue the FA phenotype of Il4ra^{F709} mice. GF Il4ra^{F709} mice that were reconstituted by FMT from WT BALB/c mice then sensitized with OVA/SEB and challenged with OVA were protected from FA. In contrast, those reconstituted with SPF Il4ra^{F709} mouse microbiota developed robust disease, as evidenced by a precipitous drop in core body temperature and increased serum MMCP1 concentrations upon oral challenge with OVA, with increased total and OVA-specific serum IgE concentrations (see e.g., FIG. 33). These results indicated that dysbiosis is also an essential pathogenic attribute of FA in the Il4ra^{F709} mice, and showed that dysbiosis promotes FA in both the human subjects and mice via common mechanisms.

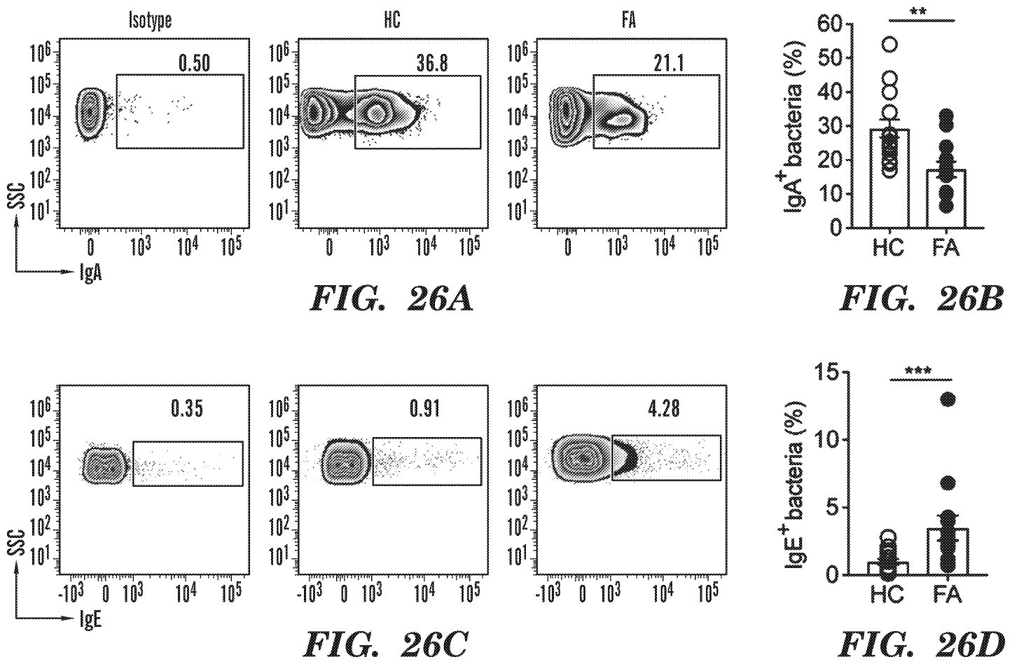

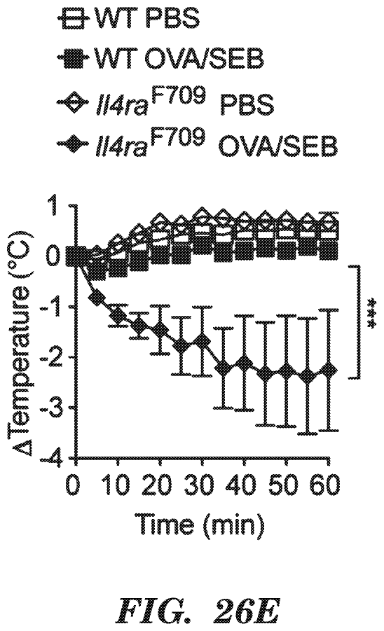

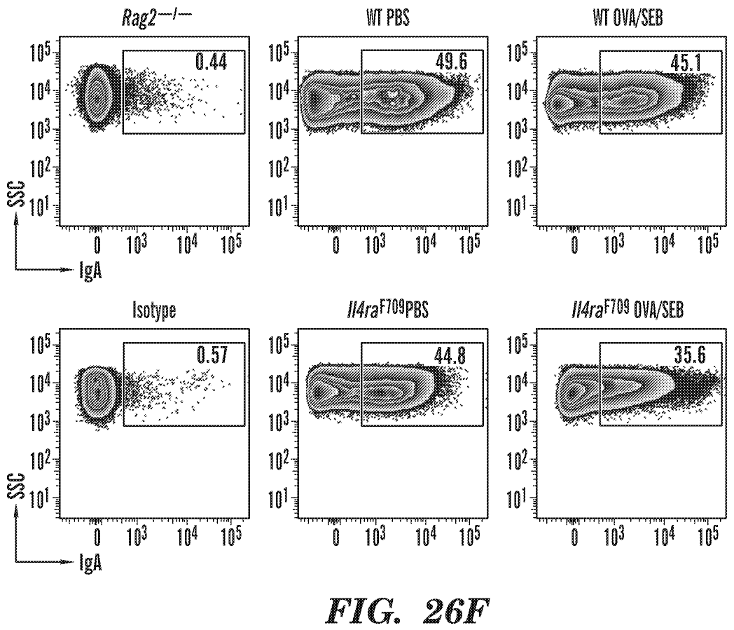

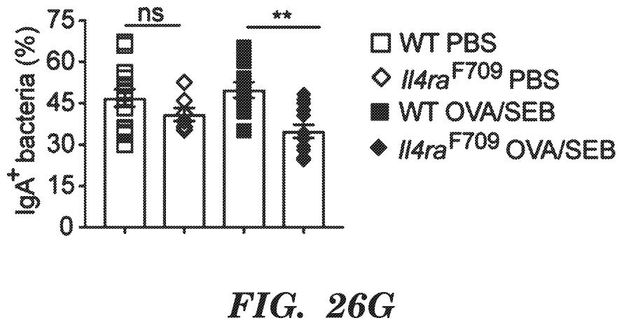

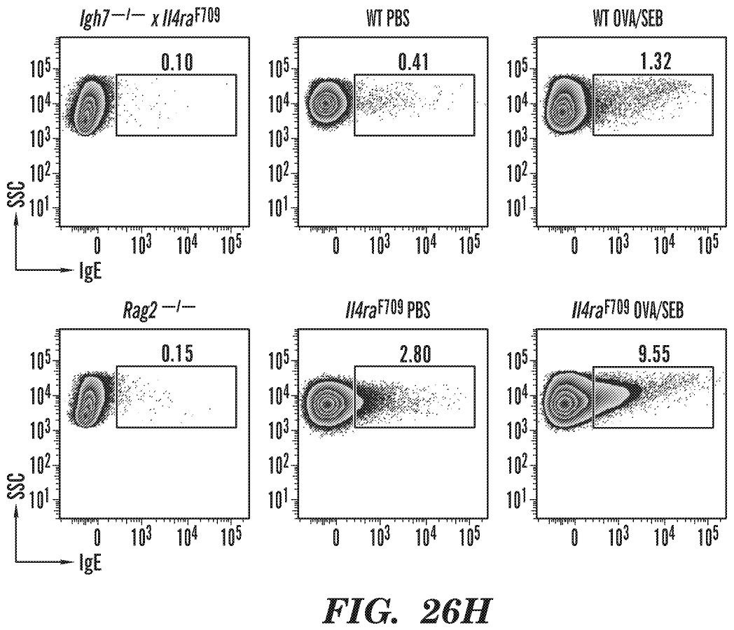

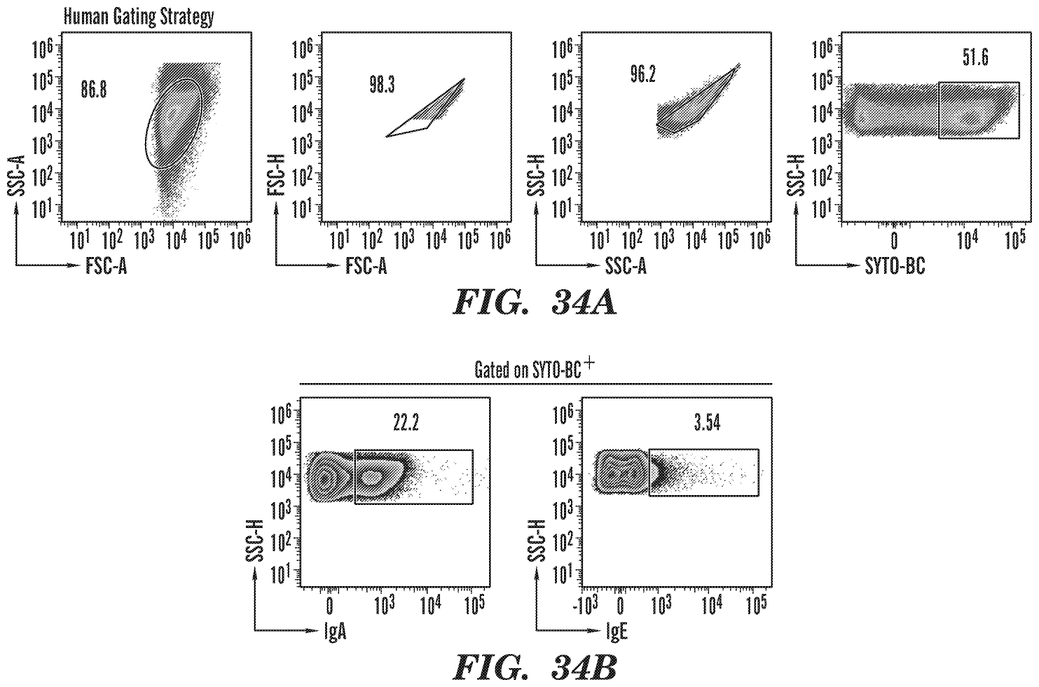

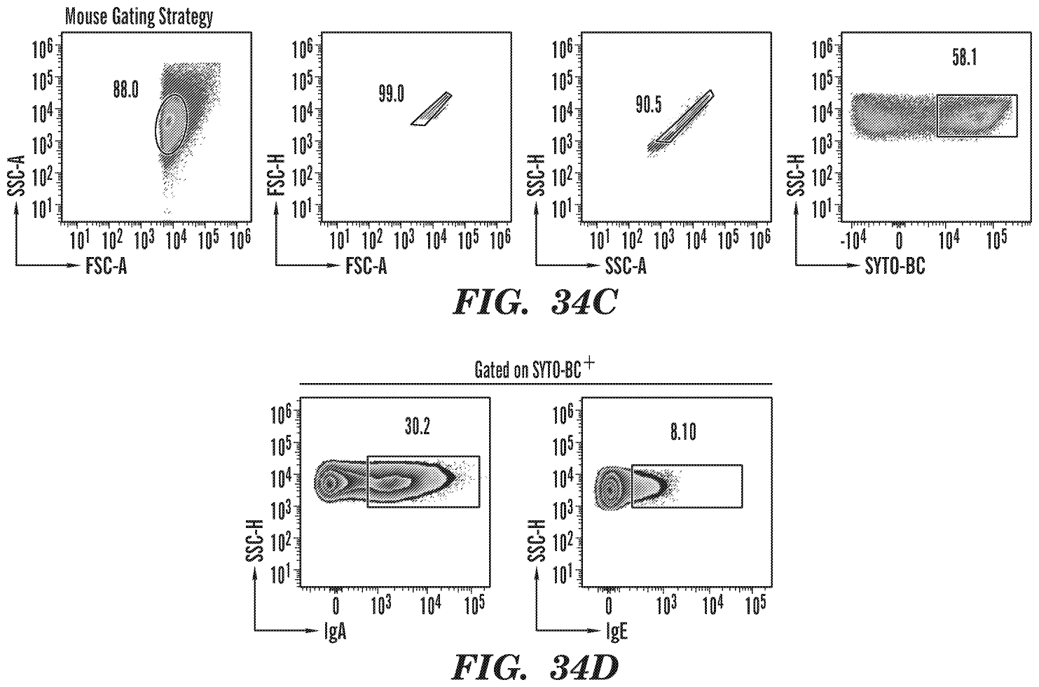

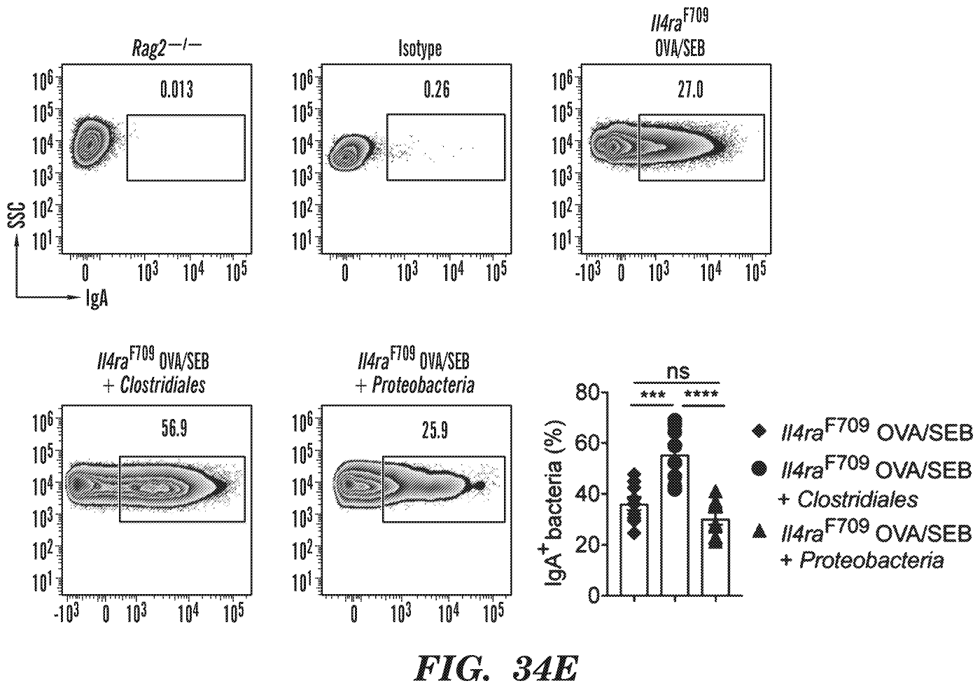

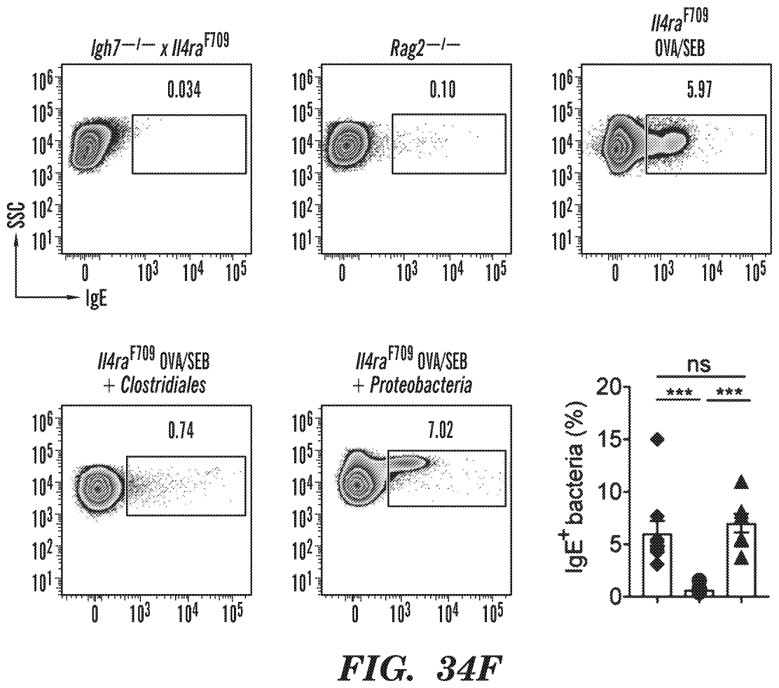

Dysbiosis in FA is associated with an altered immune response to the gut microbiota. The gut secretory IgA (sIgA) response shapes the composition of the microbial flora and helps maintain commensalism. Accordingly, by flow cytometry was used to analyze the binding of sIgA to the fecal flora of FA and control subjects. The gating strategy for immunoglobulin staining of human fecal flora is demonstrated in FIGS. 34A-34B. FA subjects displayed decreased sIgA binding of their fecal bacteria as compared to control subjects (see e.g., FIGS. 26A-26B). To explore whether FA is involved in dysregulated allergic responses to both food and bacteria, the binding of IgE to fecal bacteria was analyzed in FA and healthy subjects. Notably, FA subjects exhibited increased IgE binding to fecal bacteria, consistent with an allergic response against commensal species (see e.g., FIGS. 26C-D).

To more thoroughly investigate the mucosal antibody responses in FA, the binding of sIgA and IgE to the fecal bacteria of Il4ra^{F709} mice was analyzed, following gating strategies shown in FIGS. 34C-34D. The Il4ra^{F709} mice also exhibit dysbiotic microbiota which, upon transfer to germ free BALB/c mice heightens their susceptibility to food allergy induction. Accordingly, Il4ra^{F709} mice and control WT BALB/c mice were either sham sensitized with PBS or orally sensitized with chicken egg ovalbumin (OVA) in the presence of the mucosal adjuvant staphylococcal enterotoxin B (SEB) and subsequently challenged with OVA. OVA sensitized Il4ra^{F709} but not WT control mice challenged with OVA exhibited a rapid drop in their core body temperature, consistent with anaphylaxis (see e.g., FIG. 26E). Similar to FA subjects, the fecal bacteria of OVA-sensitized Il4ra^{F709} mice exhibited decreased sIgA binding as compared to similarly sensitized WT mice or sham sensitized Il4ra^{F709} and WT mice (see e.g., FIGS. 26F-26G). In addition, sensitization with OVA also resulted in an increased IgE binding to fecal bacteria of Il4ra^{F709} mice, but not WT controls (see e.g., FIGS. 26H-26I). The specificity of these results was further confirmed by the lack of sIgA or IgE binding to fecal bacteria of Rag2-deficient mice, which do not express immunoglobulins, and the lack of IgE binding to the fecal bacteria of double mutant Igh7^{-/-}Il4ra^{F709} mice, which carry a targeted deletion of the IgE heavy chain gene (see e.g., FIGS. 26F-26I). Overall, these results established that dysbiosis in FA human subjects and Il4ra^{F709} mice is associated with decreased sIgA responses and heightened T helper cell type 2 (Th2)/IgE responses to the commensal flora.

A defined consortium of human Clostridiales species promotes tolerance in experimental FA. Bacterial species of the order Clostridiales have been implicated in imparting oral tolerance by virtue of their immunomodulatory effects, including their production of SCFA, which stabilize iTreg cells and promote their retention in the gut. To test whether dysbiotic alterations in Clostridiales taxa affected by the dysbiosis contribute to oral tolerance breakdown in FA, the capacity was examined of a defined consortium of six Clostridiales type strains, chosen as representative of Clostridiales clusters impacted by the dysbiosis in the human study described herein, to suppress the induction of FA in Il4ra^{F709} mice (see e.g., TABLE 9). Parameters driving selection of the consortium members included well-characterized genomic and metabolic profiles, prior data of in vivo effects on gut epithelium and/or immune maturation from human or animal model systems, and ease of culturability for developing into a human therapeutic. Strains were also confirmed to be non-toxigenic against human fibroblasts or polarized gut epithelial cells. The consortium included C. sardiniense (cluster I, e.g. OTU 20), C. leptum (cluster IV, e.g. OTU 29, 50), C. hiranonis and C. bifermentans (cluster XI, e.g. OTU 22), and C. scindens (cluster XIVa, e.g. OTU 11). We also included in the consortium C. ramosum (Erysipelatoclostridium ramosum) (Clostridium cluster XVIII, e.g. OTU 26), per its defined immunomodulatory and metabolic effects on other microbes and the host. As a negative control, a consortium of species was employed from gamma and delta Proteobacteria classes, including E. coli, P. mirabilis, K oxytoca (Gammaproteobacteria; family Enterobacteriaceae), and B. wadsworthia (Deltaproteobacteria; family Desulfovibronaceae) (see e.g., TABLE 9). B. wadsworthia was shown to be increased early in life in FA subjects before declining sharply, and E. Coli was decreased across multiple time windows (see e.g., FIG. 25D and FIG. 31D), The two other members of the Proteobacteria consortium have been more broadly implicated in gut dysbiosis associated with bowel inflammation.

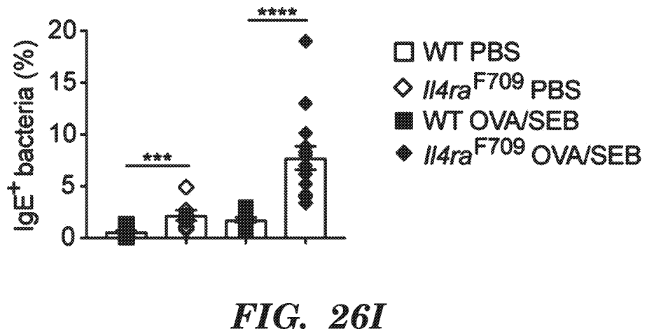

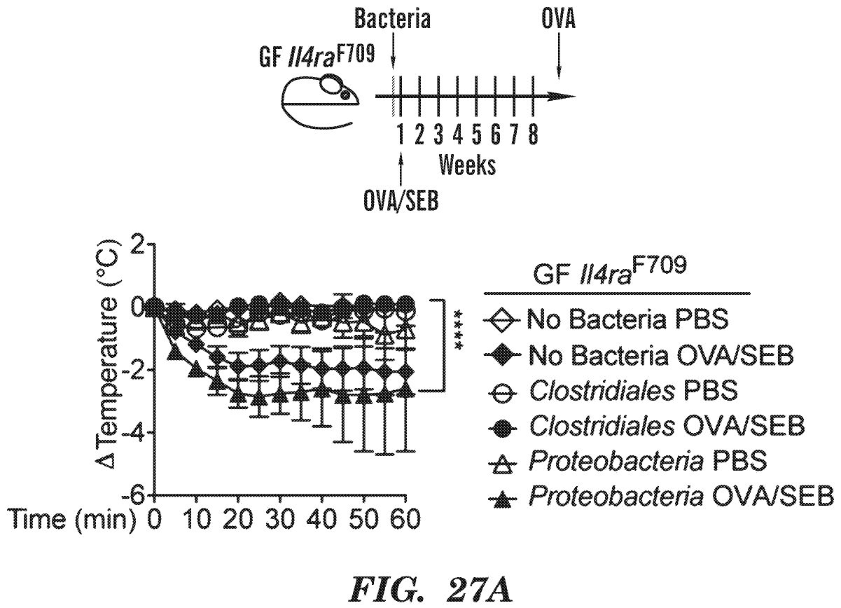

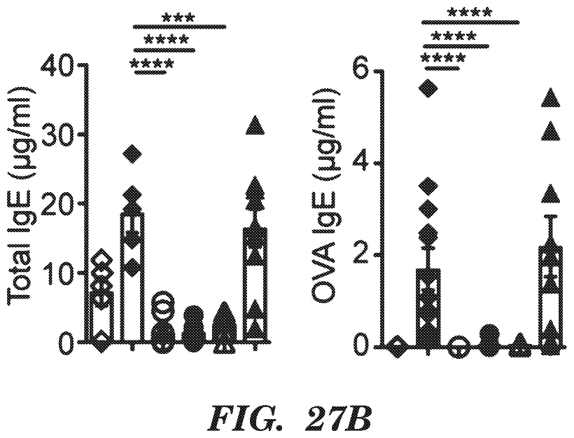

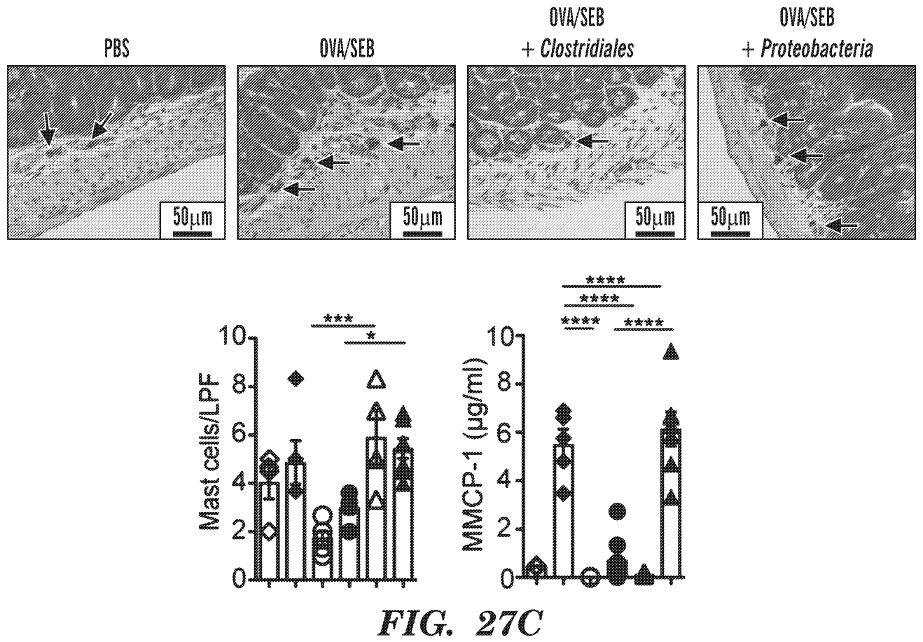

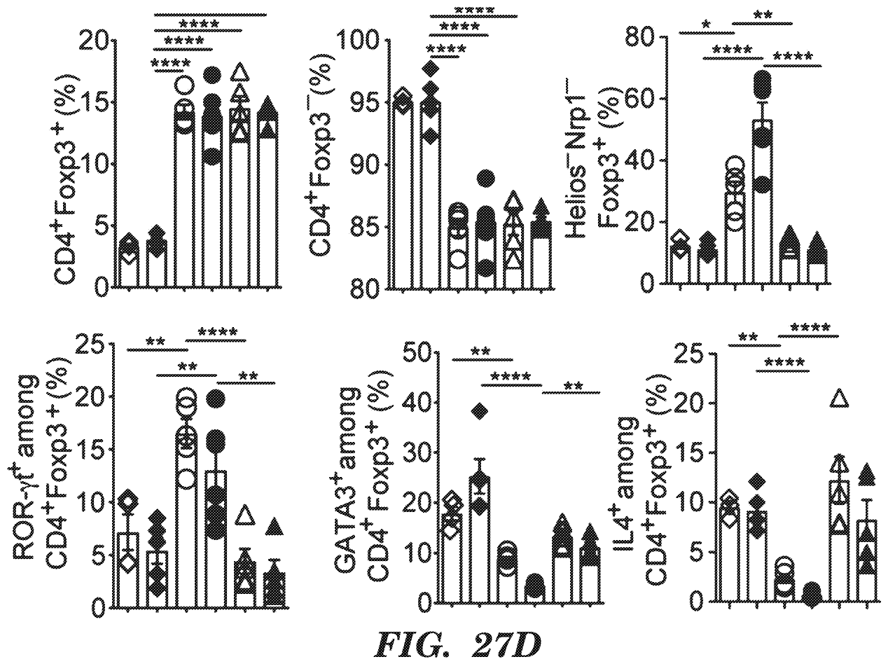

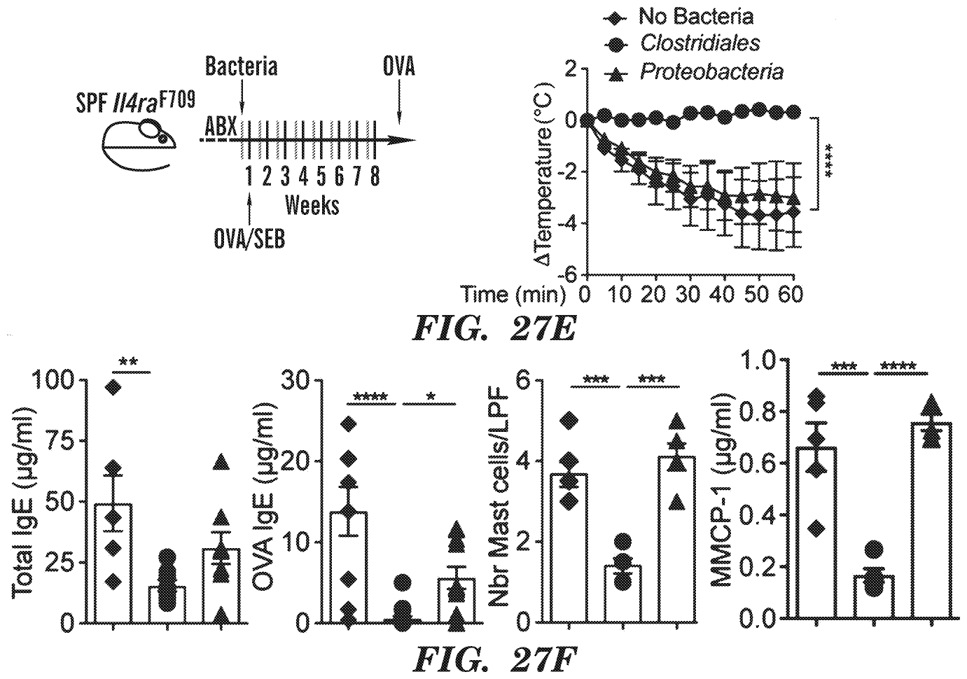

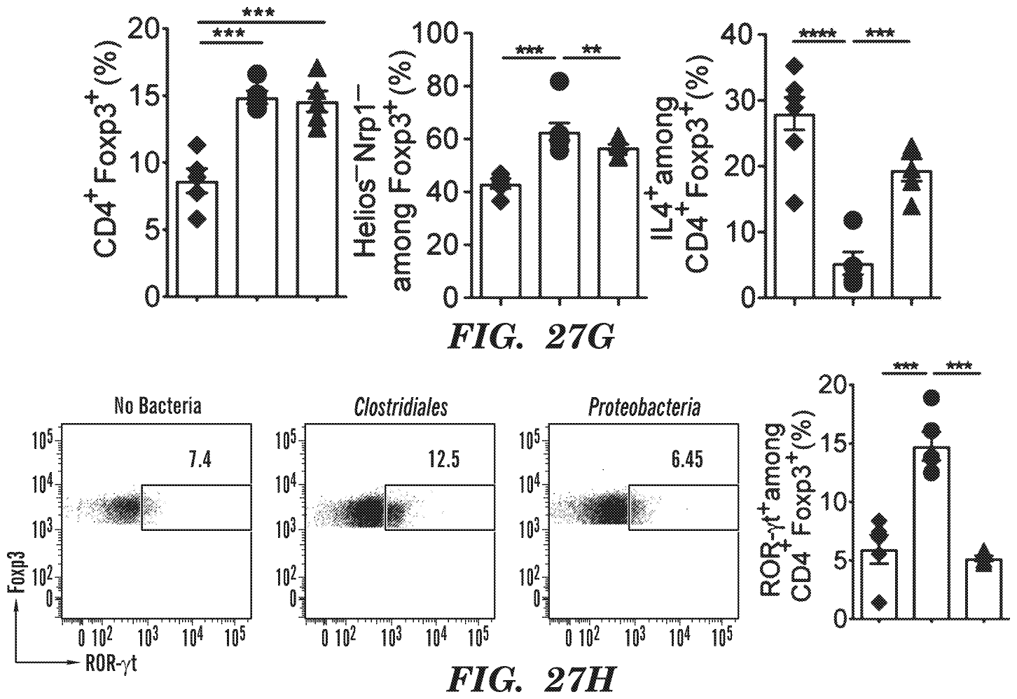

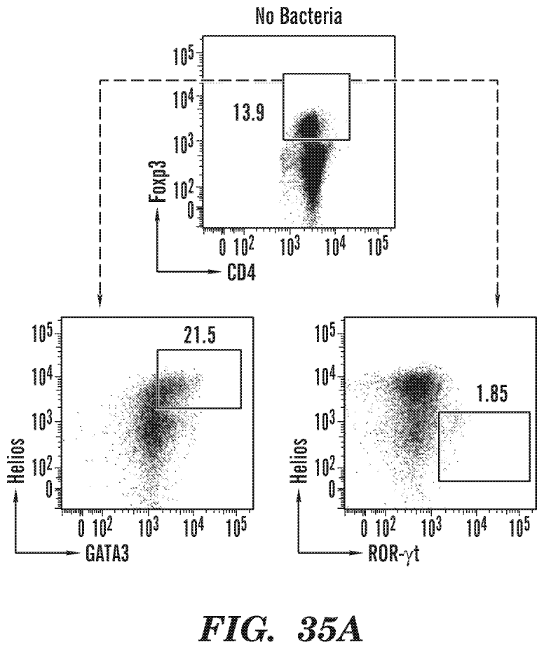

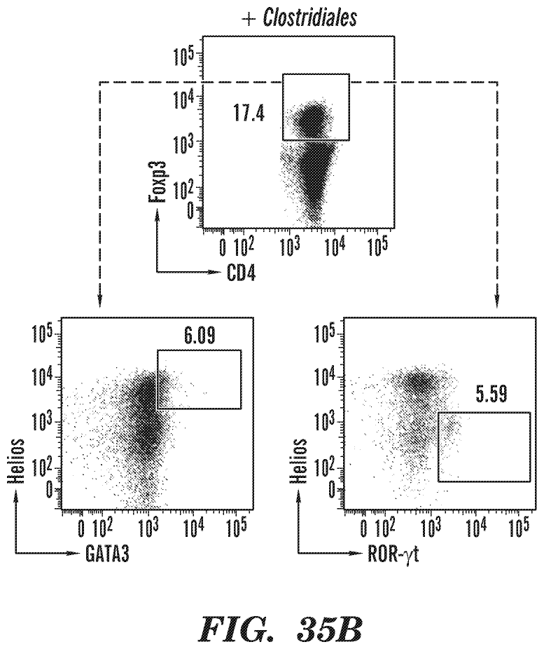

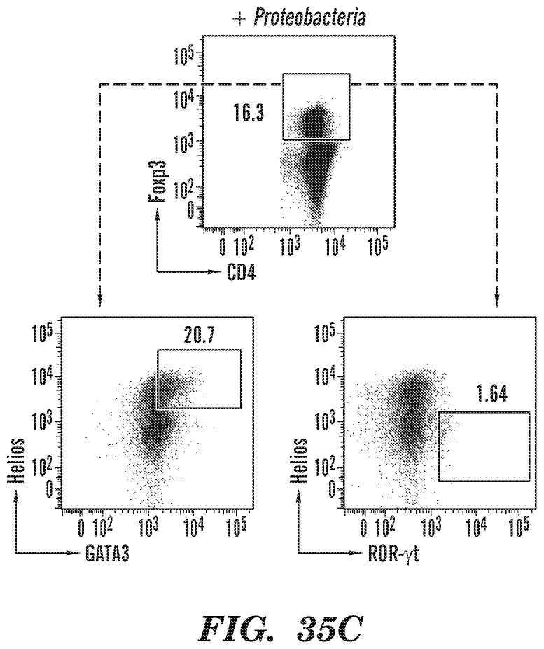

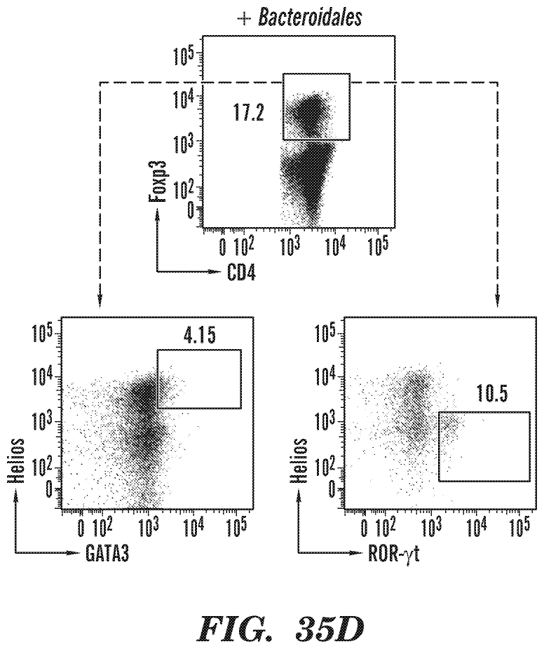

To determine whether the Clostridiales consortium directly promotes tolerance in FA, the induction of FA was analyzed in GF Il4ra^{F709} mice that were either reconstituted with the Clostridiales or Proteobacteria consortia or left in a GF state. OVA-sensitized GF control mice and those colonized with the Proteobacteria consortium exhibited robust anaphylaxis upon oral challenge with OVA, as evidenced by an acute and sustained drop in core body temperature. In contrast, those reconstituted with the Clostridiales consortium were fully protected and remained resistant to OVA-induced challenge, demonstrating no clinical symptoms of acute anaphylaxis (see e.g., FIG. 27A). Measures of allergic sensitization and anaphylaxis, including the rise in serum concentrations of total and OVA-specific IgE, small intestinal tissue mastocytosis and the increase in serum mouse mast cell protease 1 (MMCP1) concentrations post anaphylaxis, all of which were elevated in GF and Proteobacteria-supplemented mice, were inhibited by the Clostridiales consortium (see e.g., FIGS. 27B-27C).

Whereas treatment of GF Il4ra^{F709} mice with either the Clostridiales or Proteobacteria consortia increased the frequencies of CD4⁺Foxp3⁺ Treg cells in the mesenteric lymph nodes (MLN), only the Clostridiales consortium boosted the frequencies of iTreg cells, distinguished by their low expression of the markers Helios and neuropilin1 (Helios⁻Nrp1⁻), whose specificity is biased towards recognizing gut luminal antigens originating from foodstuffs or bacteria (see e.g., FIG. 27D). Treatment also increased the frequency of ROR-ye Treg cells, which have been implicated in the control of different gut Th cell-mediated immune responses, including Th1, Th2 and Th17 cell responses (see e.g., FIG. 27D). The ROR-γt⁺ Treg cells were predominantly iTreg cells as reflected by their low expression of the Helios marker (see e.g., FIG. 35). FA induction has been associated with the reprogramming of Treg cells into Th2 cell-like cells that play a direct role in disease pathogenesis. Consistent with these findings, a subset of MLN Treg cells of sham and OVA-sensitized GF mice exhibited increased expression of the Th2 master transcription factor GATA3 and the Th2 cytokine IL-4. These Th2 cell-like Treg cells are thymus-derived as reflected by their Helios^{high} phenotype (see e.g., FIG. 35). A similar increase in Treg cell IL-4 expression was found in mice colonized with the Proteobacteria consortium (see e.g., FIG. 27D). In contrast, the Clostridiales consortium suppressed the Th2 cell-like reprogramming of Treg cells and instead promoted the expression of ROR-γt in Treg cells independent of OVA sensitization, a gut Treg cell phenotype associated with decreased Th2 cell responses (see e.g., FIG. 27D). Overall, these results established that the Clostridiales consortium confers protection against FA in a genetically prone mouse model independent of other bacterial species.

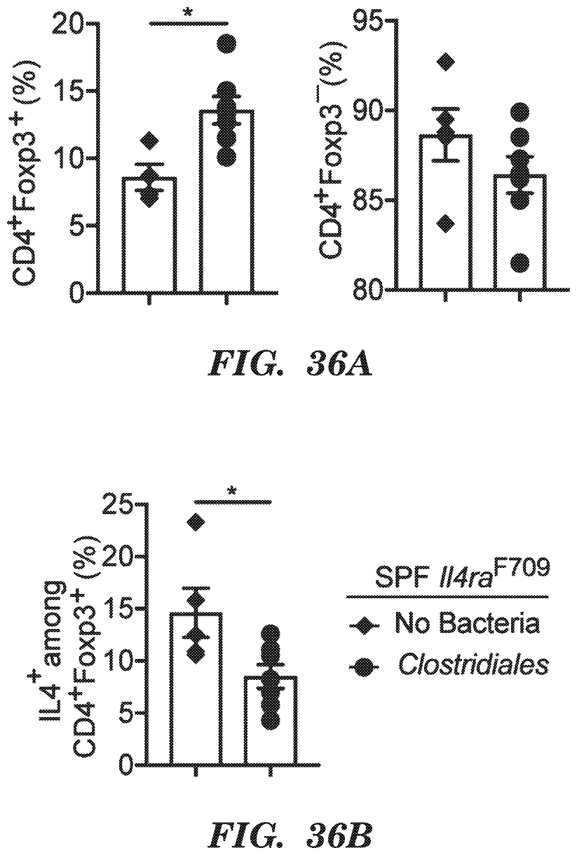

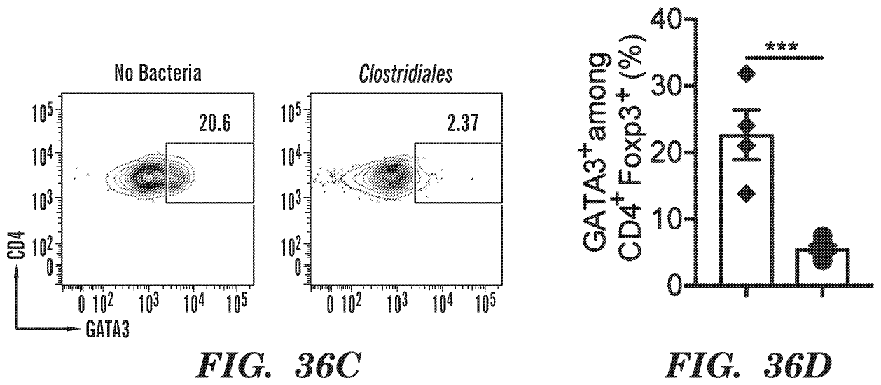

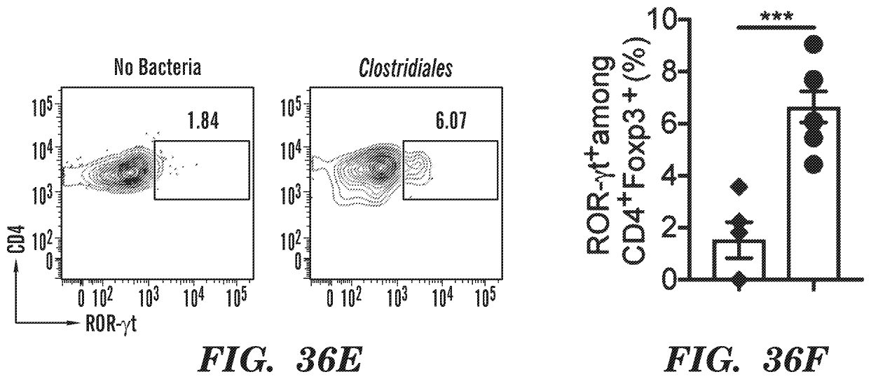

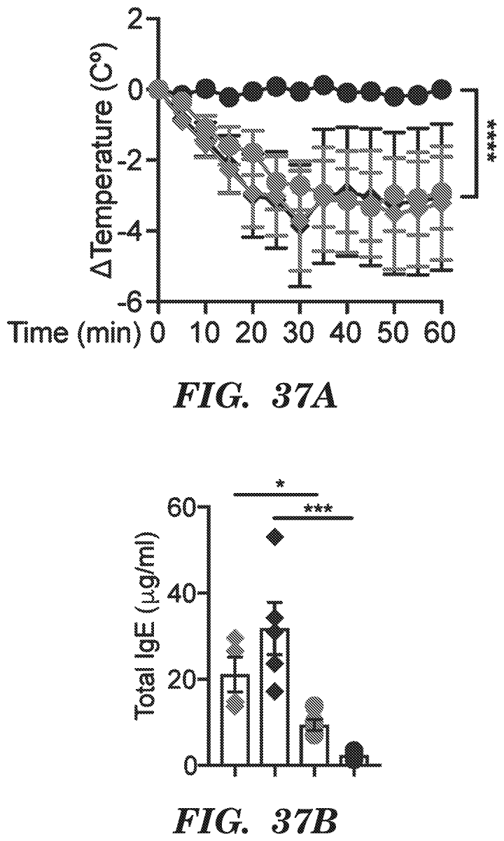

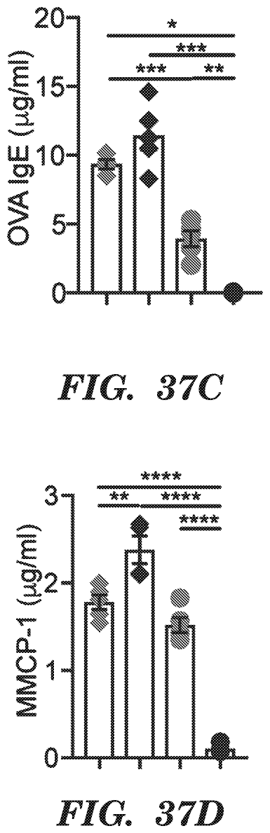

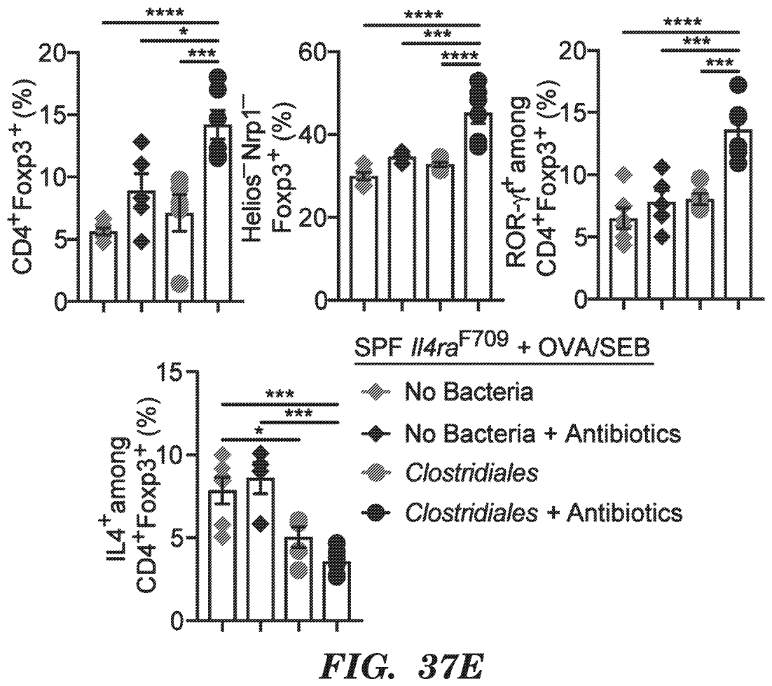

To determine whether the protection by the Clostridiales consortium against FA extended to mice colonized with complex conventional microbiota, SPF Il4ra^{F709} mice were treated for one week with antibiotics to create a niche for the therapeutic bacterial consortia, and were then given the respective Clostridiales or Proteobacteria consortia by gavage. Thereafter the mice underwent sensitization with OVA/SEB by gavage once weekly for 8 weeks with weekly bacterial therapy administered 3 days after each sensitization (see e.g., FIG. 27E). The mice were then orally challenged with OVA and analyzed for their FA response. The Clostridiales consortium completely protected OVA/SEB sensitized Il4ra^{F709} mice from developing anaphylaxis upon oral challenge with OVA (see e.g., FIG. 27E). Total and OVA-specific serum IgE responses, gut tissue mast cell expansion and serum MMCP1 concentrations post challenge were also sharply curtailed (see e.g., FIG. 27F). In contrast, mice treated with Proteobacteria were not protected. Conventional Il4ra^{F709} mice treated with the Clostridiales but not the Proteobacteria consortium exhibited increased Treg cells in the MLN, reflective of increased frequencies of Helios⁻Nrp1⁻ cells (see e.g., FIG. 27G). The Clostridiales but not the Proteobacteria consortium suppressed the Th2 cell-like reprogramming of gut Treg cells in the FA Il4ra^{F709} mice, as evidenced by their decreased IL-4 expression, consistent with improved Treg cell function, while increasing the frequencies of the ROR-γt⁺ iTreg cells (see e.g., FIGS. 27G-27H, FIG. 35). Analysis of small intestinal lamina propria lymphocytes (LPL) of FA Il4ra^{F709} mice also revealed increased ROR-γt⁺ iTreg cells and suppression of the Th2 cell response (see e.g., FIG. 36). Of note, prior antibiotic treatment of the mice markedly improved the therapeutic efficacy of the Clostridiales consortium, suggesting that it can serve to enhance the immunomodulatory functions of the consortium by reducing the abundance of interfering bacteria (see e.g., FIG. 37).

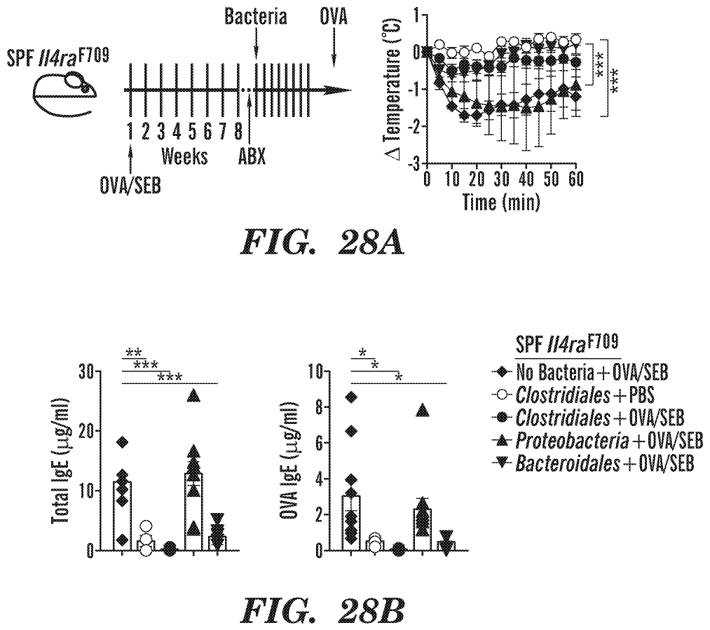

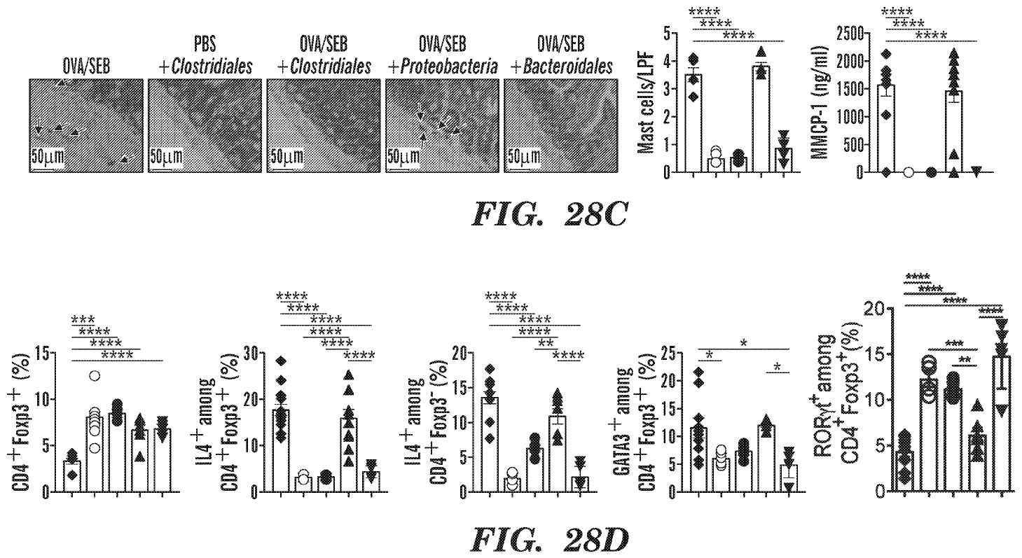

It was further examined whether treatment with the Clostridiales consortium corrected the altered mucosal immune response to the microbiota in FA Il4ra^{F709} mice. Treatment with the Clostridiales but not Proteobacteria consortium resulted in an increased sIgA response to the microbiota, as evidenced by increased sIgA staining of fecal bacteria (see e.g., FIG. 28E). Reciprocally, the Clostridiales but not Proteobacteria consortium suppressed the IgE anti-bacterial response (see e.g., FIG. 28F). These findings were indicative of the normalization by the Clostridiales consortium of the aberrant mucosal immune response to the gut microbiota in FA Il4ra^{F709} mice.

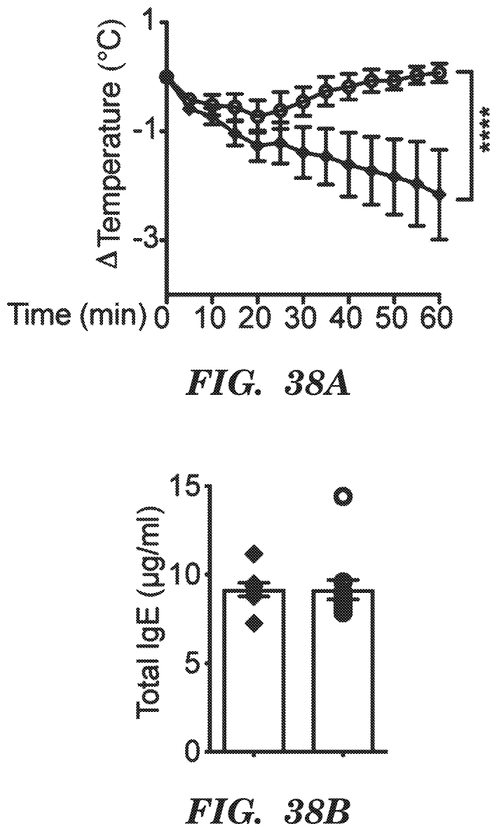

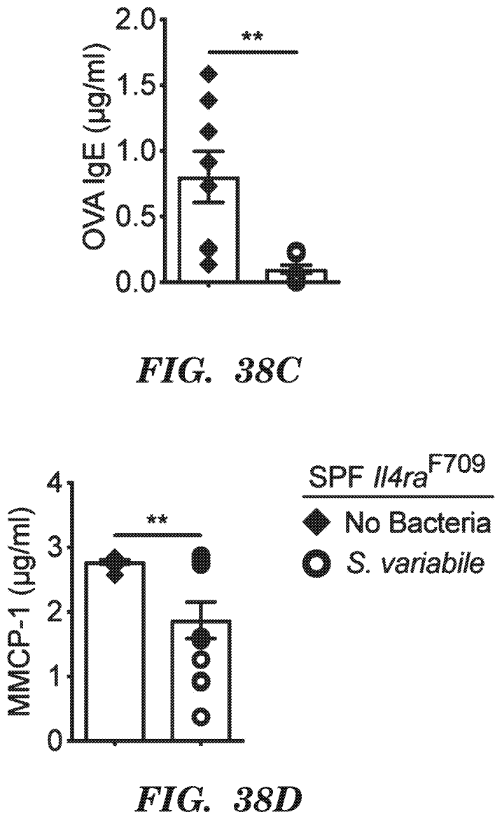

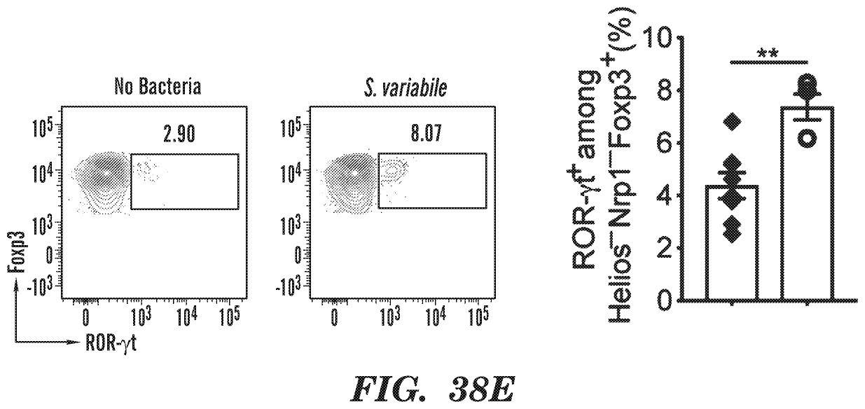

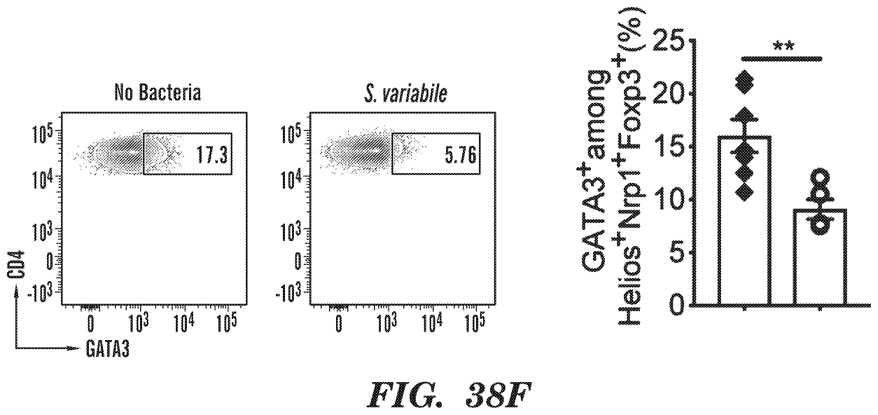

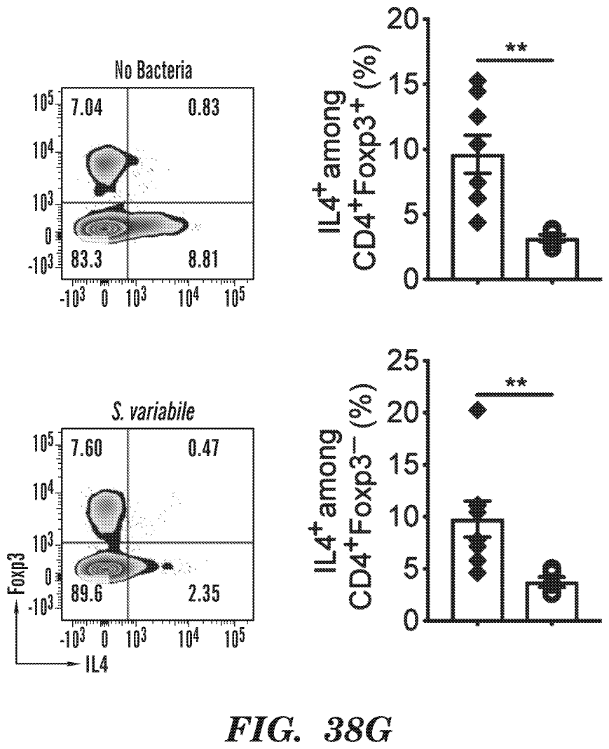

Of the Clostridial taxa affected by dysbiosis in FA, OTU 50, which mapped to Subdoligranulum variabile, stood out as being decreased across several time windows in FA subjects age 1 year and older, including those that were milk tolerant, suggesting that its deficiency may act as a switch to initiate or sustain FA in those subjects. Accordingly, the capacity was examined of monobacterial therapy with Subdoligranulum variabile to protect against FA in SPF Il4ra^{F709} mice. The mice were sensitized with OVA/SEB without or with added bacterial therapy following the protocol shown in FIG. 27E. Monotherapy with Subdoligranulum variabile protected the Il4ra^{F709} mice from developing FA, albeit less stringently than the Clostridiales consortium, in association with the induction of ROR-γt⁺ iTreg cells (see e.g., FIG. 38). These results reinforce the functional significance of the dysbiotic changes observed in FA subjects and are consistent with the loss of key immunomodulatory bacteria in FA subjects as critical to the pathogenesis of FA.

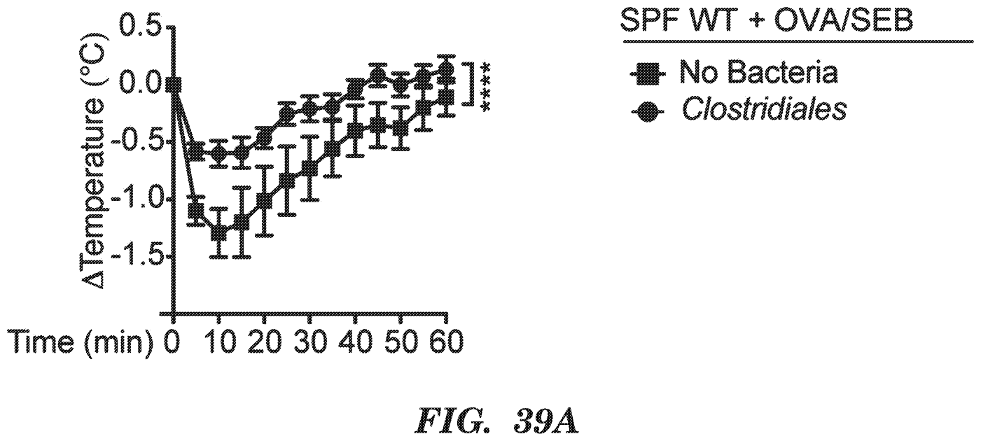

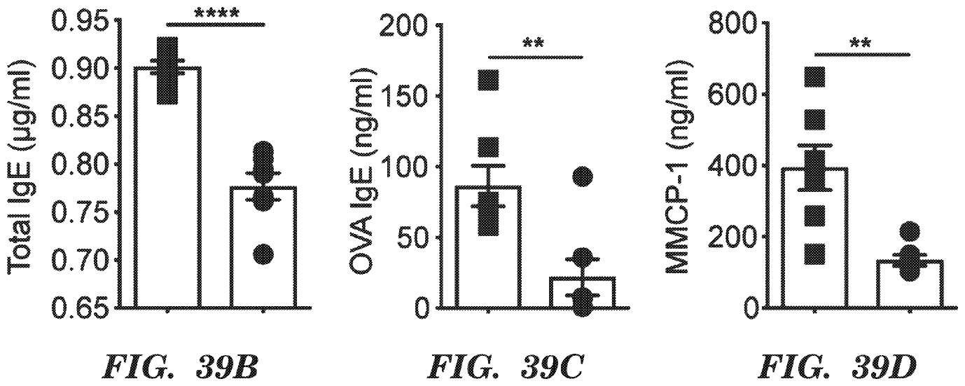

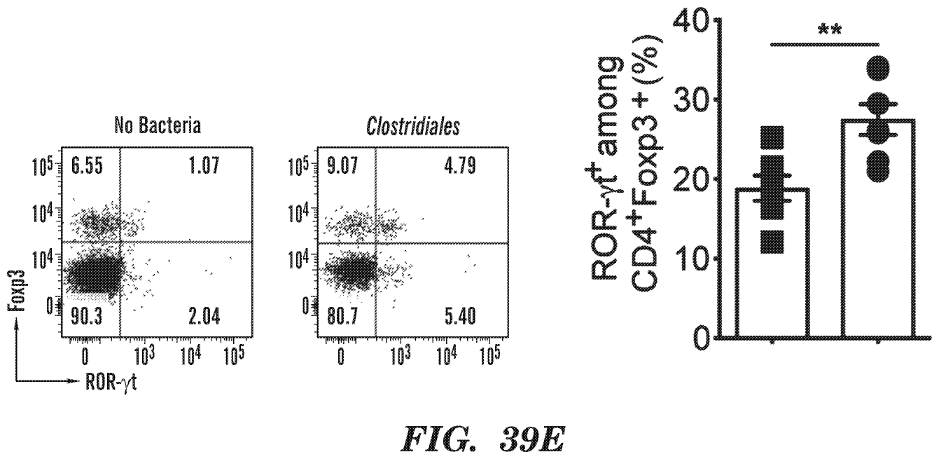

To determine whether the protection by the Clostridiales species extended to other models of FA, the capacity was examined of the Clostridiales consortium to protect against the induction of FA upon epicutaneous sensitization of WT mice with OVA, a model of food sensitization in human subjects with eczema. Treatment with the Clostridiales consortium greatly attenuated the induction of FA via the epicutaneous allergen sensitization route in association with the induction of ROR-γt⁺ iTreg cells. These results indicated that the Clostridiales consortium was protective against FA induced by different routes of allergen sensitization and on different mouse genetic backgrounds, intimating that it targeted fundamental immunological mechanisms involved in disease pathogenesis (see e.g., FIG. 39).

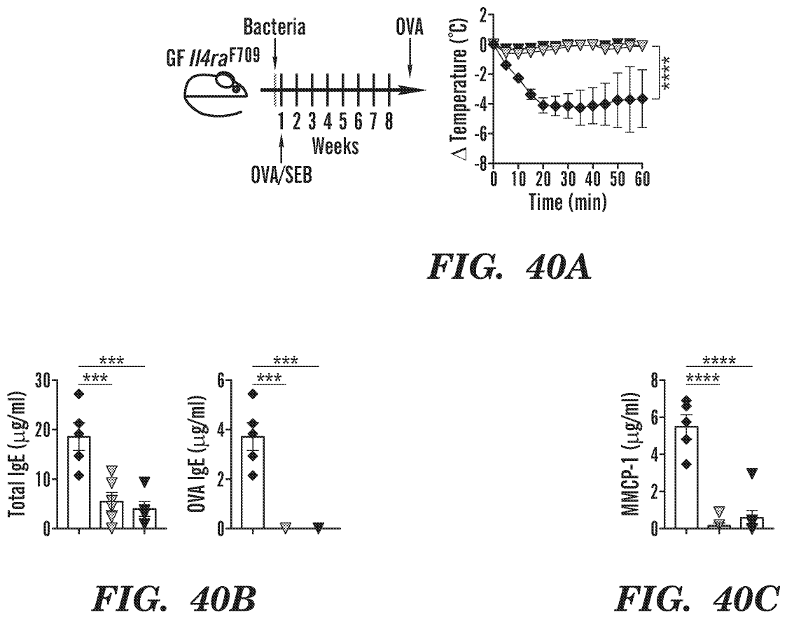

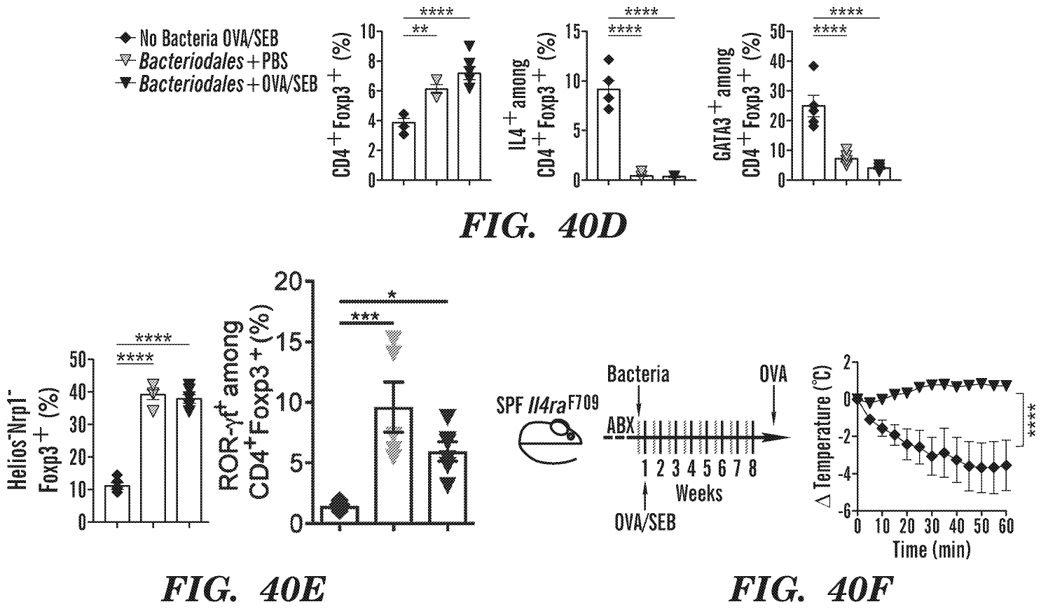

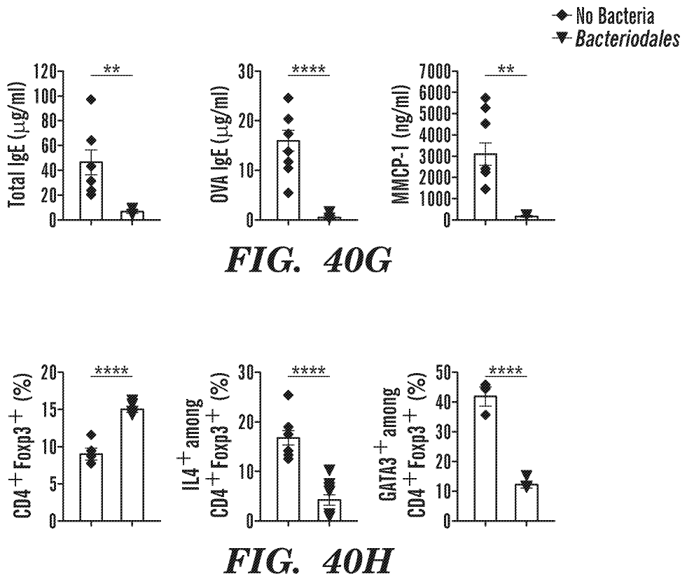

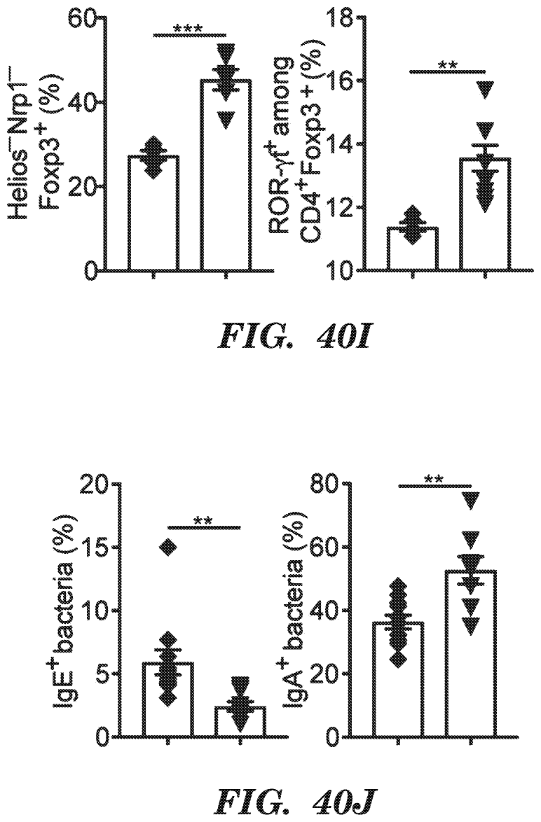

Promotion of oral tolerance in FA by immunomodulatory human Bacteroidales species. To determine if the capacity to promote oral tolerance in FA was restricted to Clostridiales species or was shared by other immunomodulatory bacteria, another defined consortium was tested comprised of five human-origin Bacteroidales species, including B. fragilis, B. ovatus, B. vulgatus, P. melaninogenica, and P. distasonis (OTU24, CRS P. distasonis). Similar to the case of the Clostridiales consortium, the choice of these species reflected their availability as type strains, their well-characterized genomic and metabolic profiles, ease of culturability, lack of in vitro toxicity with human cells, and the previous demonstration of their immunomodulatory functions. B. fragilis, B. ovatus and B. vulgatus are particularly potent in promoting of Treg cell formation in the gut. P. distasonis ameliorates experimental colitis, while P. melaninogenica promotes IL-10 production. Results revealed that treatment with the Bacteroidales consortium completely protected against the induction of FA in GF Il4ra^{F709} mice upon their sensitization with OVA/SEB (see e.g., FIGS. 40A-40E). Furthermore, the Bacteroidales consortium protected conventional SPF Il4ra^{F709} mice from developing FA when it was given in tandem with OVA/SEB during the sensitization protocol, as per the Clostridiales mix (see e.g., FIGS. 40E-40I). Similar to the Clostridiales consortium, the Bacteroidales consortium increased sIgA binding and suppressed IgE binding to the fecal bacteria of treated FA Il4ra^{F709} mice (see e.g., FIG. 40J). These results established that protection against FA is not a unique attribute of Clostridia species but could be affected by other immunomodulatory bacteria.

To determine whether bacteriotherapy with the Clostridiales and Bacteroidales consortia could suppress FA once the disease was established, conventional Il4ra^{F709} mice were sensitized with OVA/SEB once weekly for eight weeks to establish disease. The mice were then treated with a short course of antibiotics and further sensitized with OVA/SEB for an additional 4 weeks with or without bacterial therapy with either the Clostridiales, Bacteroidales or Proteobacteria consortium. The mice were then challenged orally with OVA and analyzed. Results showed that therapy with either the Clostridiales or Bacteroidales but not the Proteobacteria consortium prevented the OVA/SEB-sensitized Il4ra^{F709} mice from reacting to the OVA challenge (see e.g., FIG. 28A). The Clostridiales and Bacteroidales but not the Proteobacteria consortium suppressed the total and OVA-specific serum IgE responses, the rise in serum MMCP-1 post OVA challenge, and the mast cell expansion (see e.g., FIGS. 28B-28C). While all bacterial consortia increased the frequencies of MLN Treg cells in this disease curative model (see e.g., FIG. 28D), only the Clostridiales and Bacteroidales consortia but not the Proteobacteria consortium suppressed the food allergy-associated Treg cell Th2 cell-like reprogramming and increased the frequency of ROR-γt⁺ Treg cells (see e.g., FIG. 28D and FIG. 35).

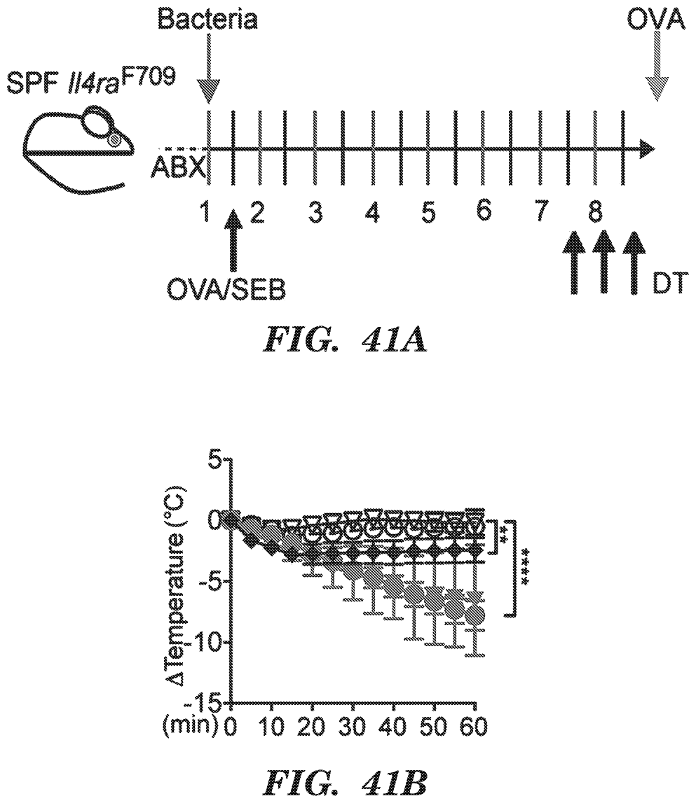

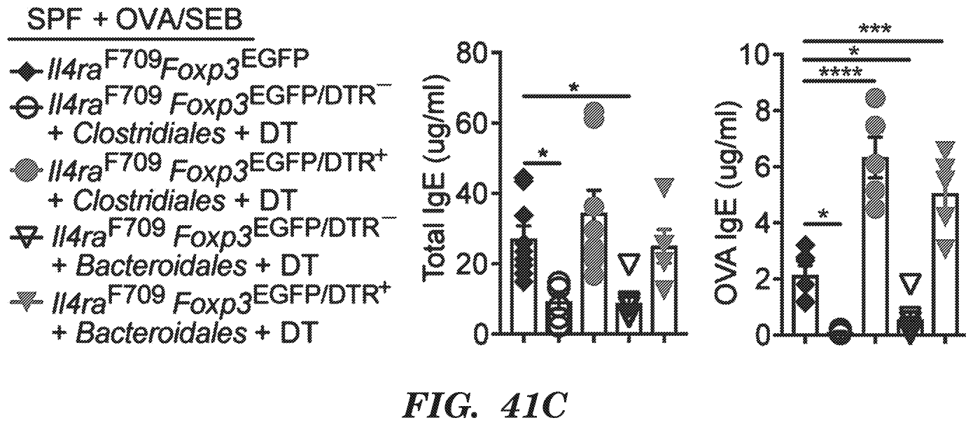

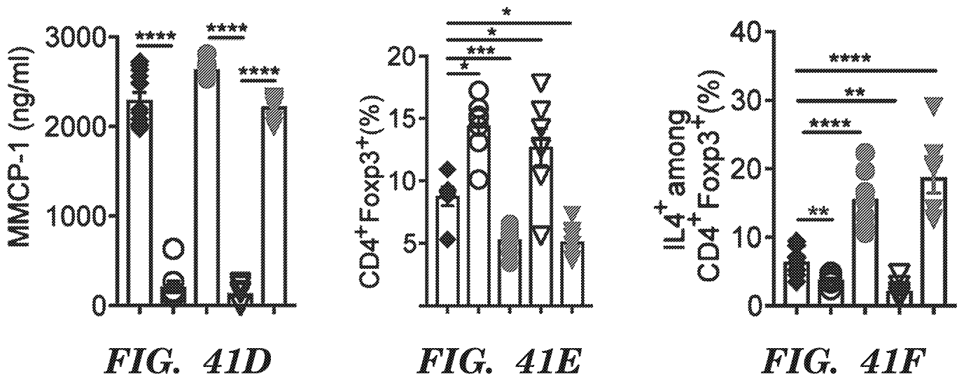

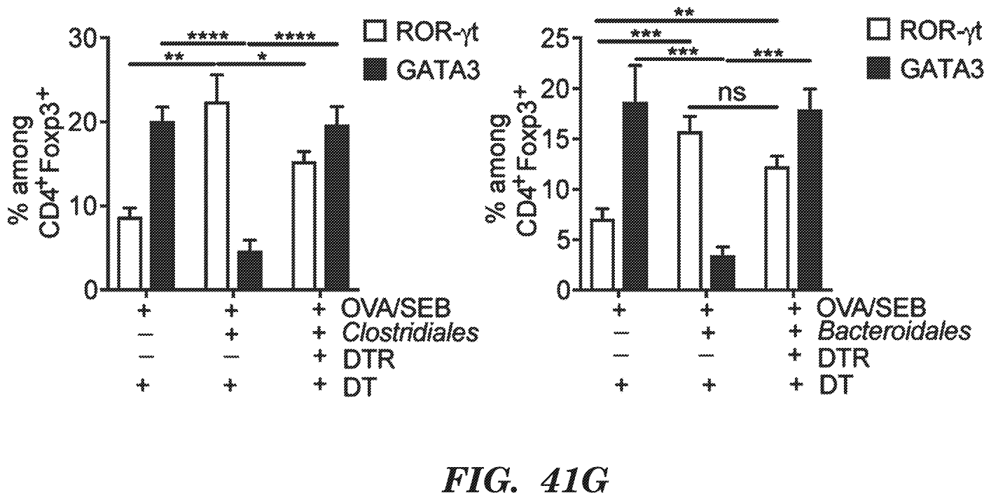

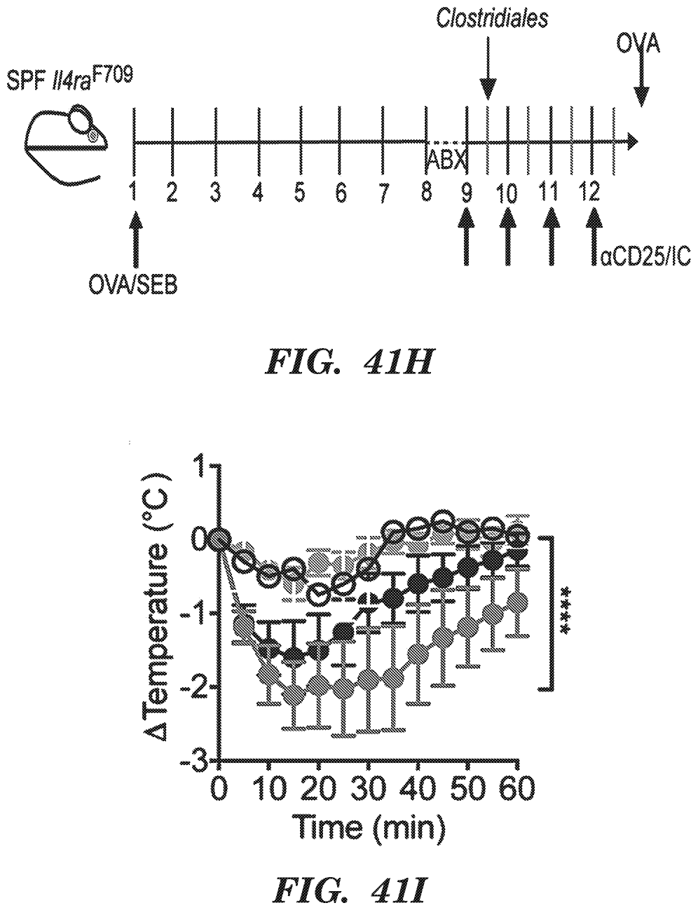

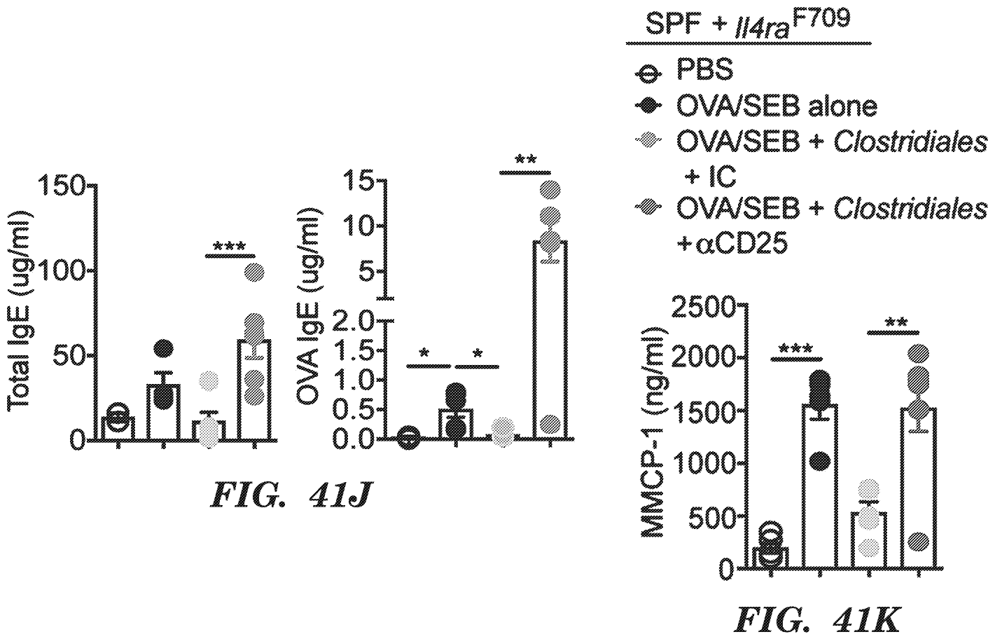

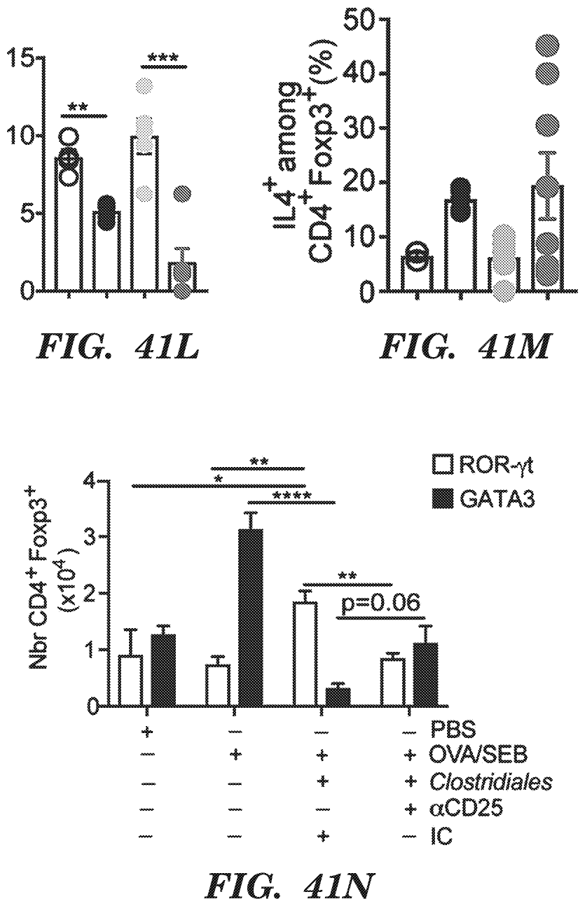

In vivo Treg cell depletion ablates the protective effects of bacterial therapy in FA. The role of Foxp3⁺ Treg cells was next examined in the protection against FA conferred by both the Clostridiales and Bacteroidales consortia using Il4raF709Foxp3^{EGFP/DTR+} mice, which specifically express the diphtheria toxin (DT) receptor on their Foxp3⁺ Treg cells, allowing for their depletion upon treatment of the mice with DT. In mice sensitized with OVA/SEB for 8 weeks, injection of DT thrice over the last two weeks prior to challenge with OVA abrogated the protection against anaphylaxis imparted by both consortia with increased total and OVA-specific IgE responses and heightened MMCP-1 release (see e.g., FIGS. 41A-41D). In the OVA/SEB-sensitized Il4ra^{F709}Foxp3^{EGFP/DTR+} mice treated with either the Clostridiales or Bacteroidales consortium, co-treatment with DT reduced the Treg cell frequencies in the gut and skewed them towards a Th2-cell like phenotype with increased IL-4 production (see e.g., FIGS. 41E-41F), while reducing the expansion of ROR-γt⁺ Treg cells normally induced by the FA-protective bacteria mixes in favor of GATA3⁺ Treg cells (see e.g., FIG. 41G). All these findings were recapitulated by using another model of in vivo Treg depletion involving the administration of an anti-CD25 monoclonal antibody (mAb). In Il4ra^{F709} mice in which FA was established by sensitization with OVA/SEB, co-treatment with the anti-CD25 mAb but not an isotype control mAb abrogated the capacity of the Clostridiales consortium to rescue established disease, with exaggerated IgE and mast cell responses (see e.g., FIGS. 41H-41K). Anti-CD25 mAb treatment similarly decreased the Treg cells in the gut and suppressed the Clostridiales mix-induced ROR-γt⁺ iTreg cells skewing in favor of IL-4 and GATA3 expression (see e.g., FIGS. 41L-41N).

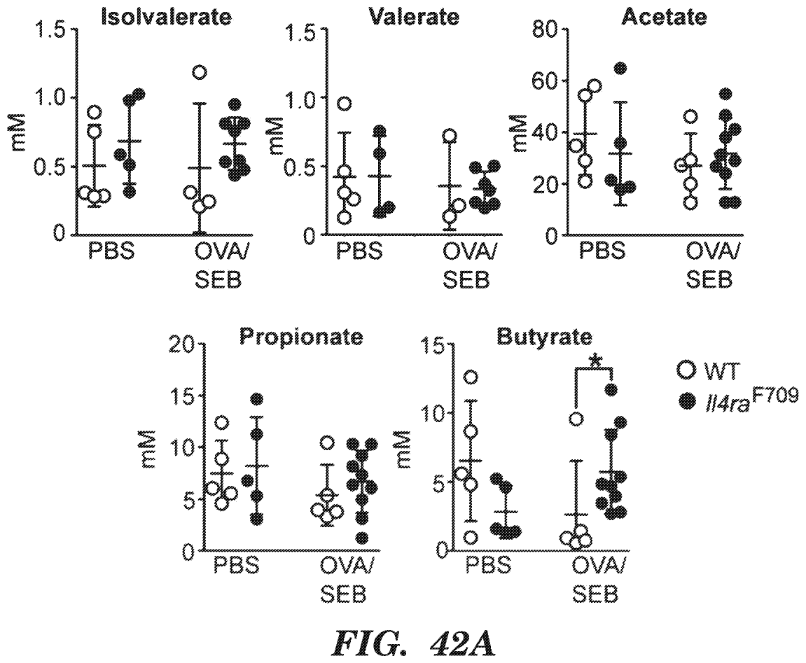

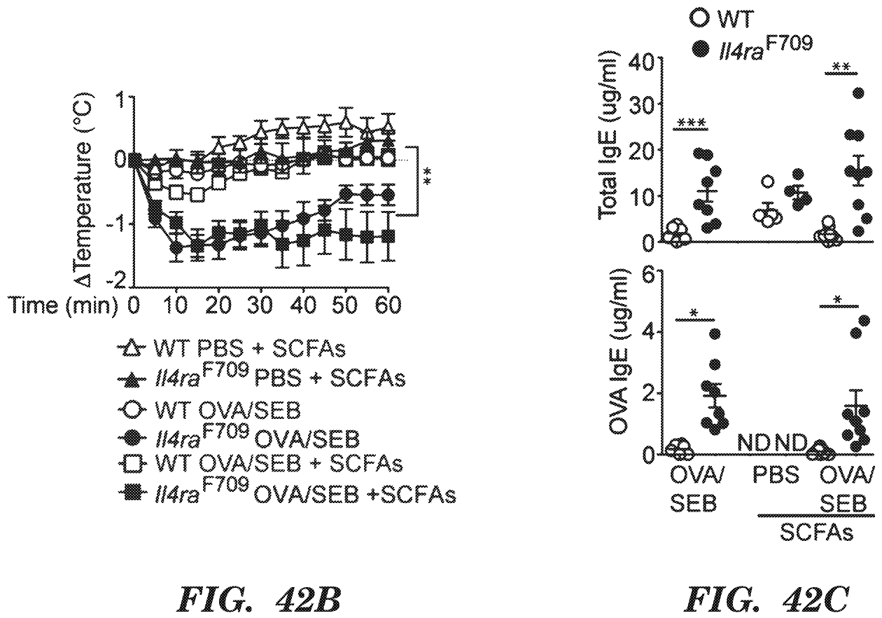

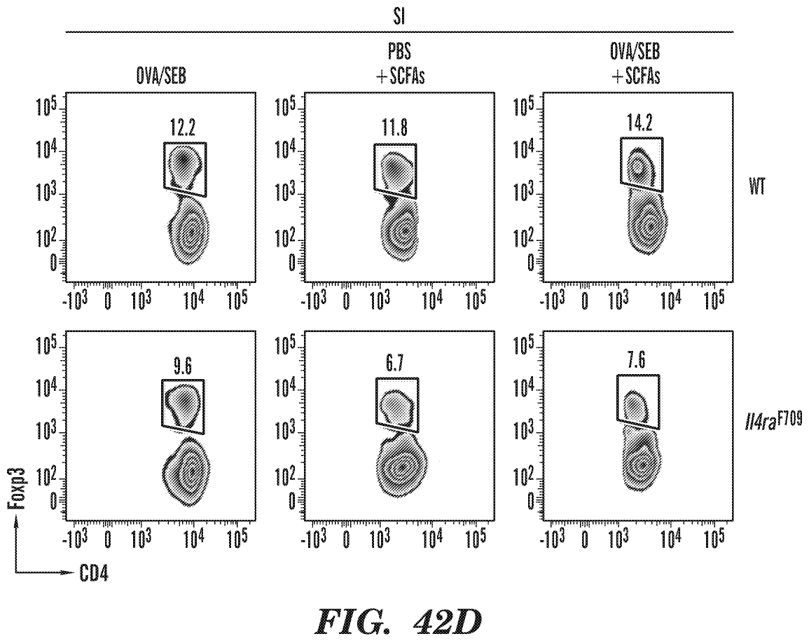

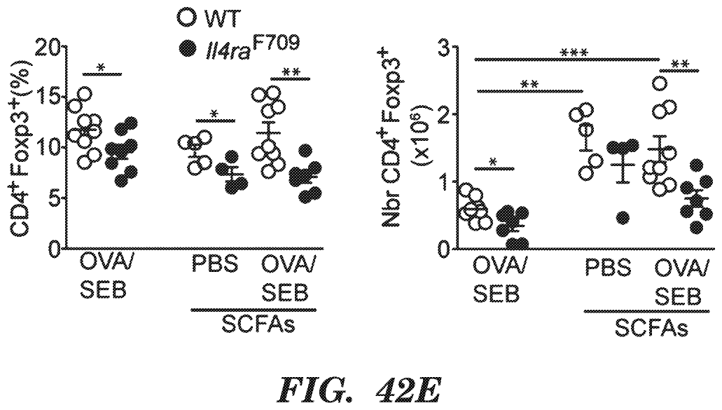

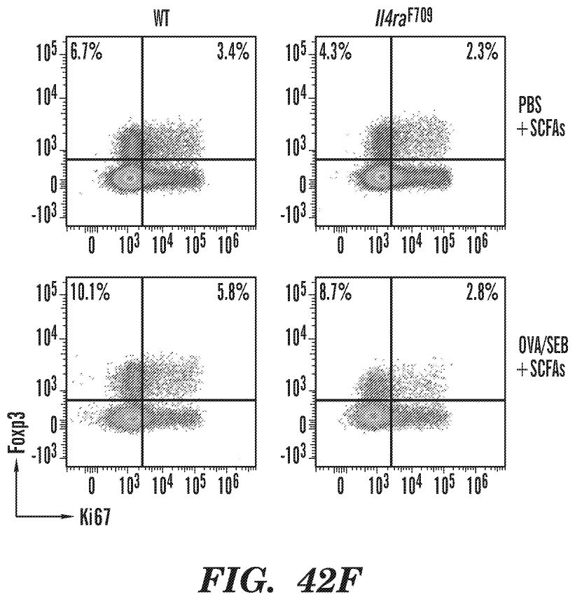

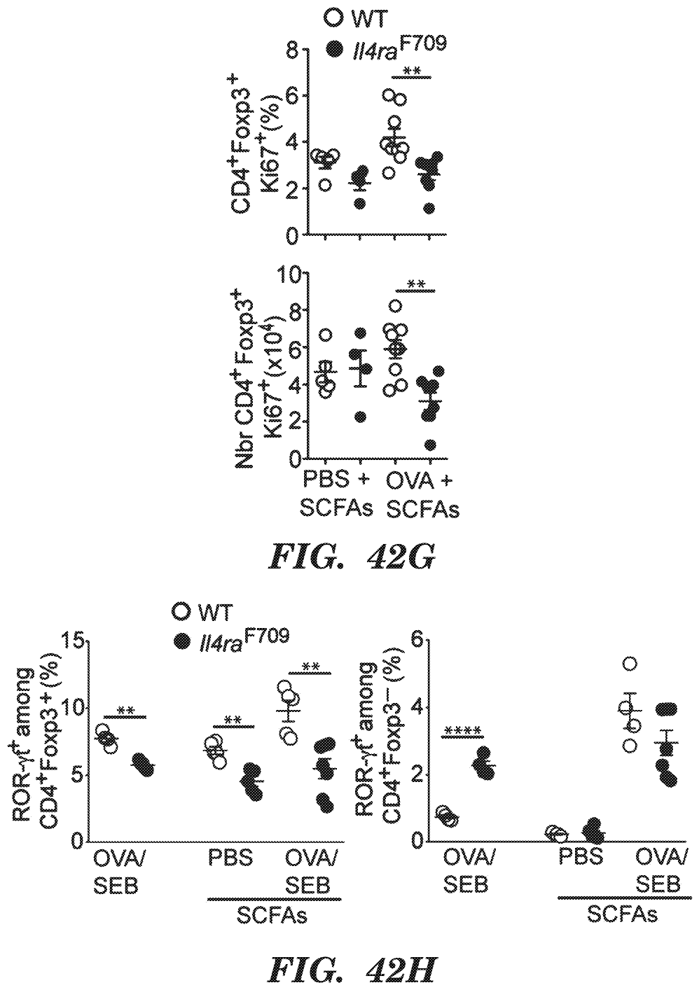

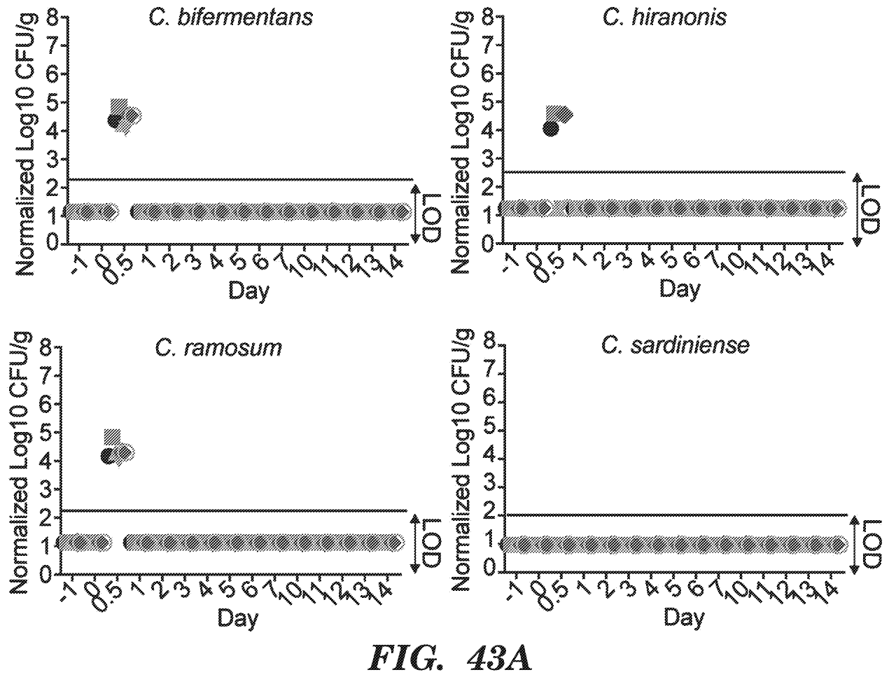

Oral Supplementation with SCFA does not protect against FA. To determine the mechanisms by which the Clostridiales and Bacteroidales consortia rescued FA in Il4ra^{F709} mice, the role of SCFA in this protection was examined given that they have been implicated in mediating tolerance induction in the gut by commensals. Analysis of SCFA in the fecal pellets of sham (PBS) and OVA/SEB-sensitized WT and Il4ra^{F709} mice revealed no differences among the respective groups in the concentrations of acetate, propionate, valerate and isovalerate, while butyrate was increased in the Il4ra^{F709} mice (see e.g., FIG. 42A). The capacity of the Clostridiales, Bacteroidales and Proteobacteria consortia to produce SCFA when introduced into GF Il4ra^{F709} mice was further examined. All three consortia produced similar amounts of acetate, as detected in the fecal samples of reconstituted mice. The Bacteroidales consortium selectively produced propionate but not butyrate, consistent with known capacity of Bacteroidales species to preferentially produce this SCFA. In contrast, butyrate production was low to absent in the fecal samples of mice reconstituted with the Clostridiales consortium, consistent with the poor production of butyrate by consortium members, including C. scindens and C. ramosum (see e.g., TABLE 10). These results indicated no clear correlation between the consortia-produced SCFA and protection against FA in Il4ra^{F709} mice.

The capacity of therapy with SCFA to protect against FA in Il4ra^{F709} mice was then examined by supplementing the drinking water with a mixture of acetate, propionate, and butyrate, given at 150 mM each, for the entire oral sensitization period. However, SCFA therapy failed to protect the mice against FA, as reflected by anaphylaxis following oral challenge with OVA and by the induction of total and OVA-specific serum IgE responses (see e.g., FIGS. 42B-42C). SCFA treatment induced increased numbers of gut Treg cells in OVA/SEB sensitized WT but not Il4ra^{F709} mice, reflecting increased frequencies and numbers of proliferating Treg cells in WT mice as measured by their expression of the proliferating cell marker Ki67 (see e.g., FIGS. 42D-42G). Similarly, SCFA treatment failed to increase the frequency of MLN ROR-γt⁺ Treg cells in sham or OVA/SEB-sensitized Il4ra^{F709} mice (see e.g., FIG. 42H). These results indicated that unlike the case with the Clostridiales or Bacteroidales consortia, therapy with SCFA failed to rescue FA or induce ROR-γt Treg cells in Il4ra^{F709} mice.

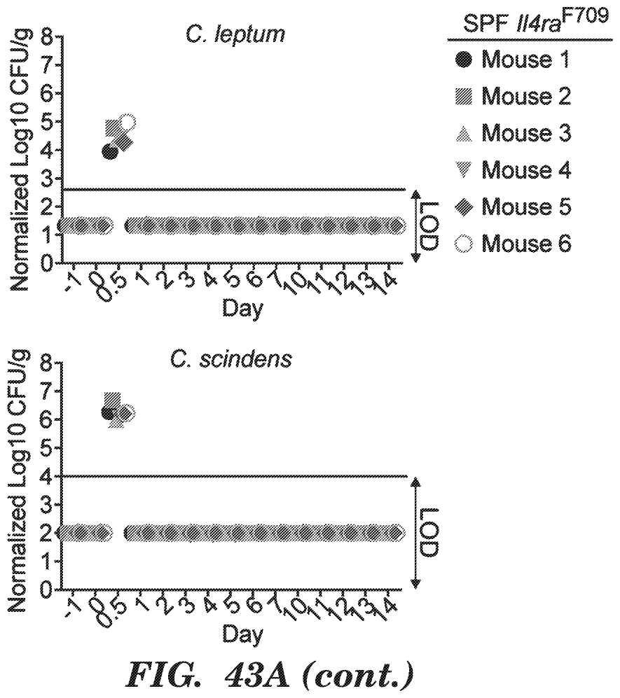

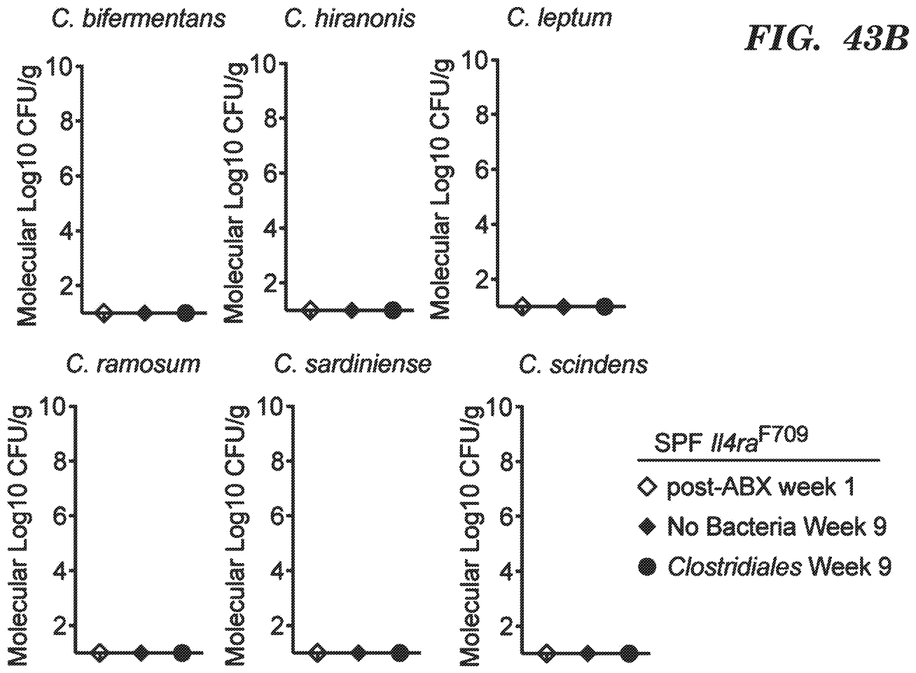

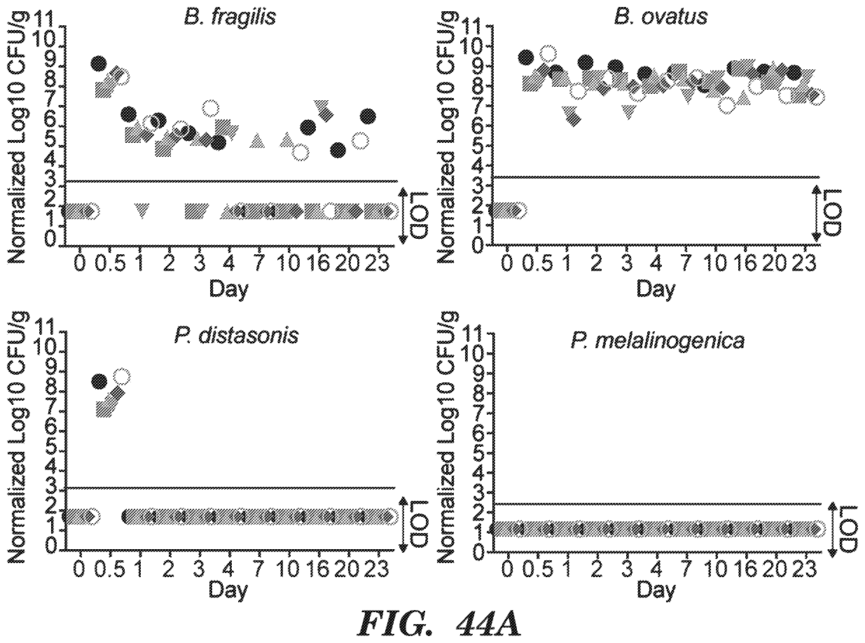

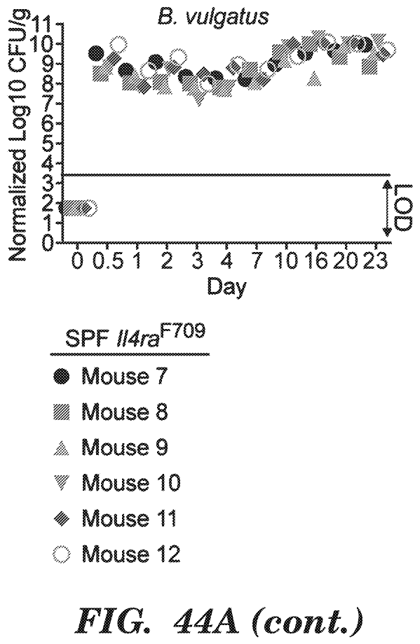

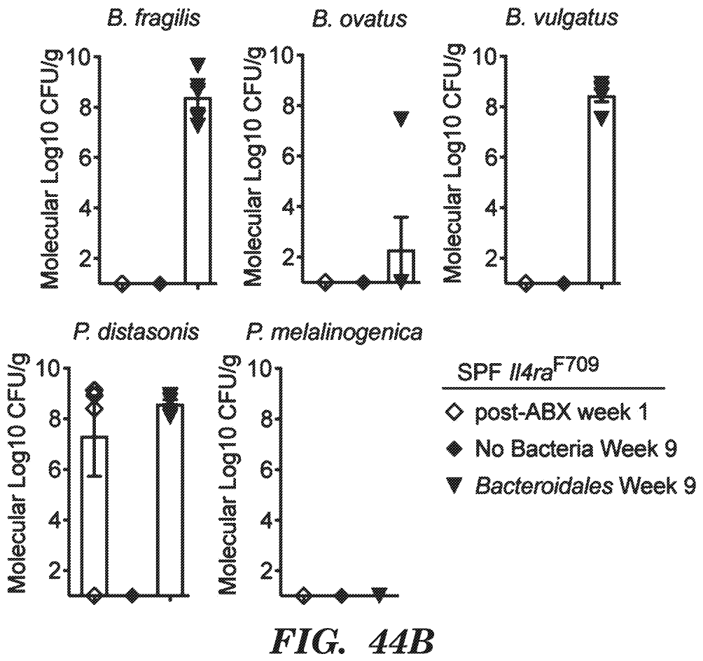

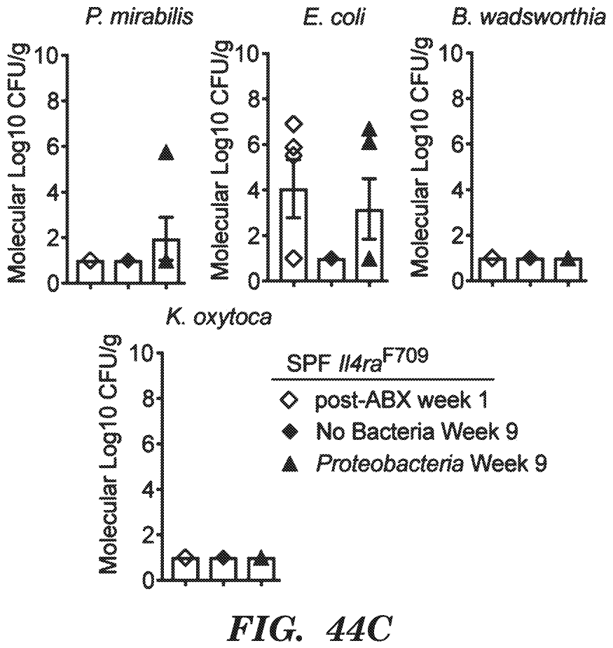

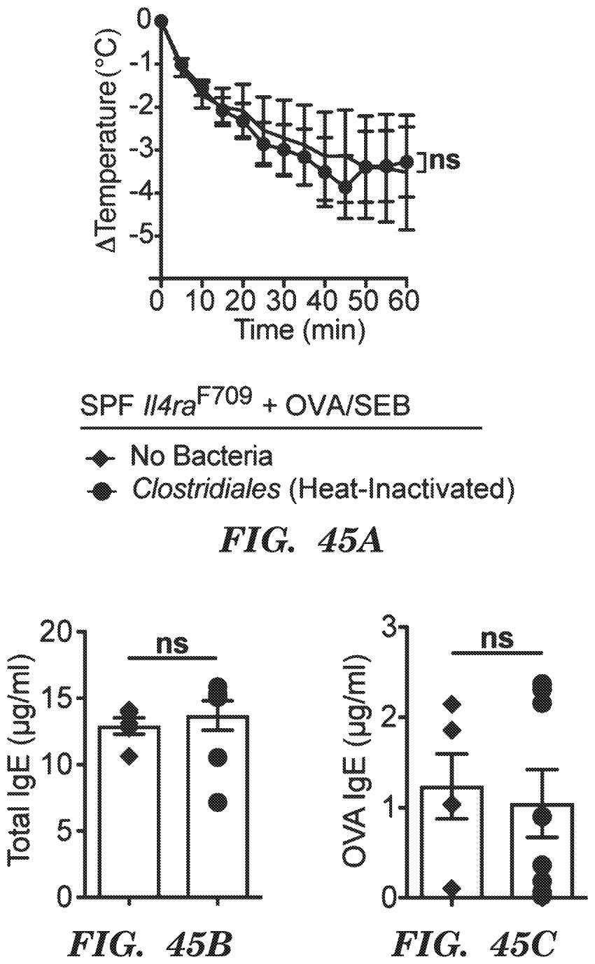

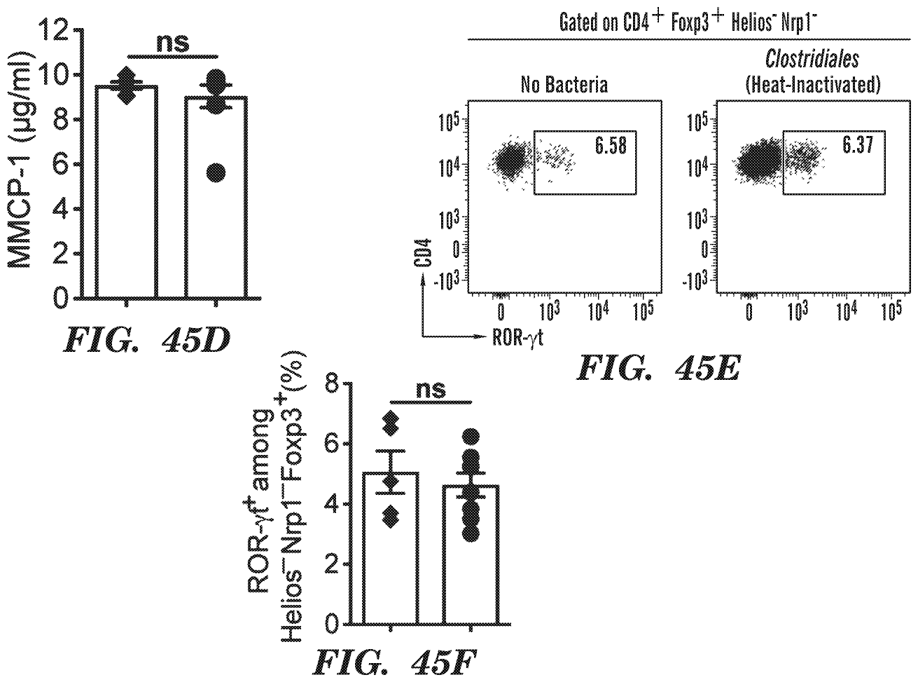

The apparent SCFA-independent protection against FA by the Clostridiales and Bacteroidales consortia prompted investigation of their persistence in Il4ra^{F709} mice, as detected by real-time PCR analysis of fecal samples using species-specific primers (see e.g., TABLE 11). None of the Clostridial species were detected at baseline, and they were only transiently detected for a day or less after they were individually introduced by a single gavage, except for C. sardiniense, which was not detected (see e.g., FIG. 43A). Furthermore, when employed as a consortium to prevent FA induction in Il4ra^{F709} mice, none of the Clostridial species were detected following the initial antibiotic treatment or at the end of the 8 week therapy course (see e.g., FIG. 43B). In contrast, while the Bacteroidales species were also not detected at baseline, several persisted for up to 3 weeks following a single gavage and also at the end of the eight-week FA treatment course, including B. fragilis, B. vulgatus and P. distasonis, as did two of the four Proteobacteria species, P. mirabilis and E. coli, albeit at low levels (see e.g., FIG. 44). Heat inactivation of the Clostridiales consortium abrogated its protection against FA, indicating a requirement for bacterial viability for therapeutic efficacy (see e.g., FIG. 45). Similar results were found for the Bacteroidales consortium.

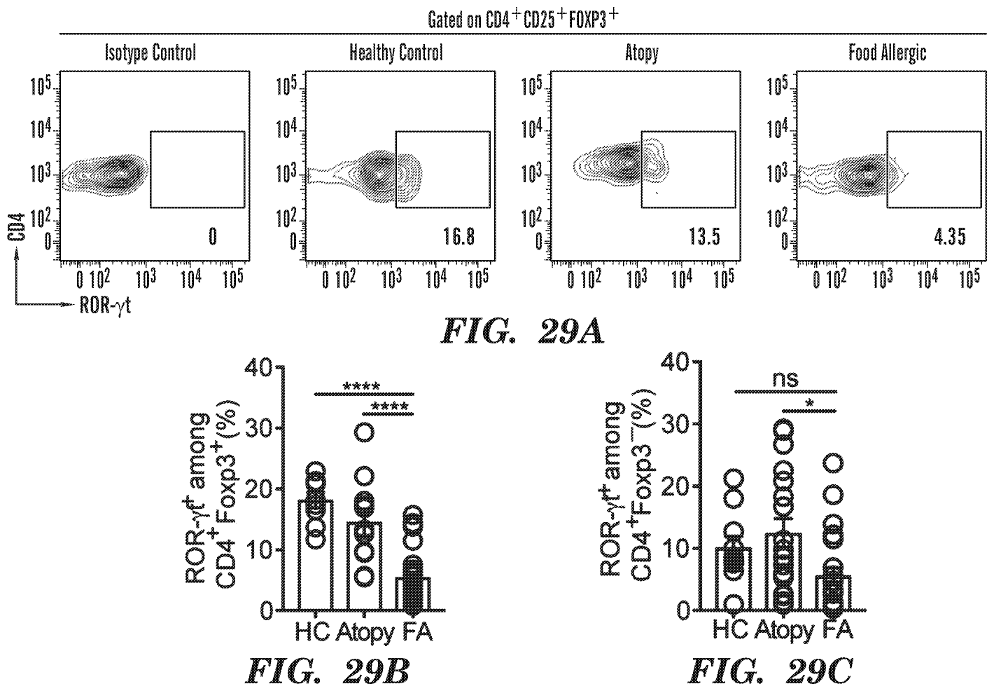

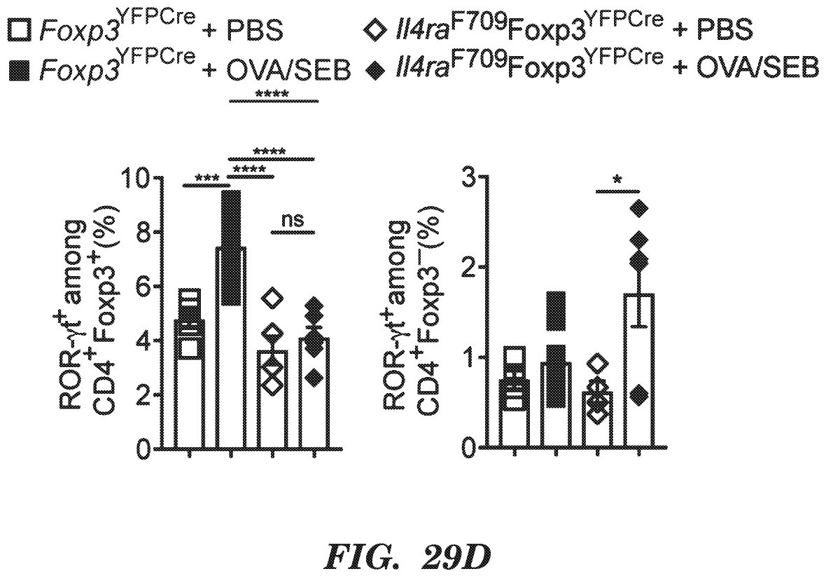

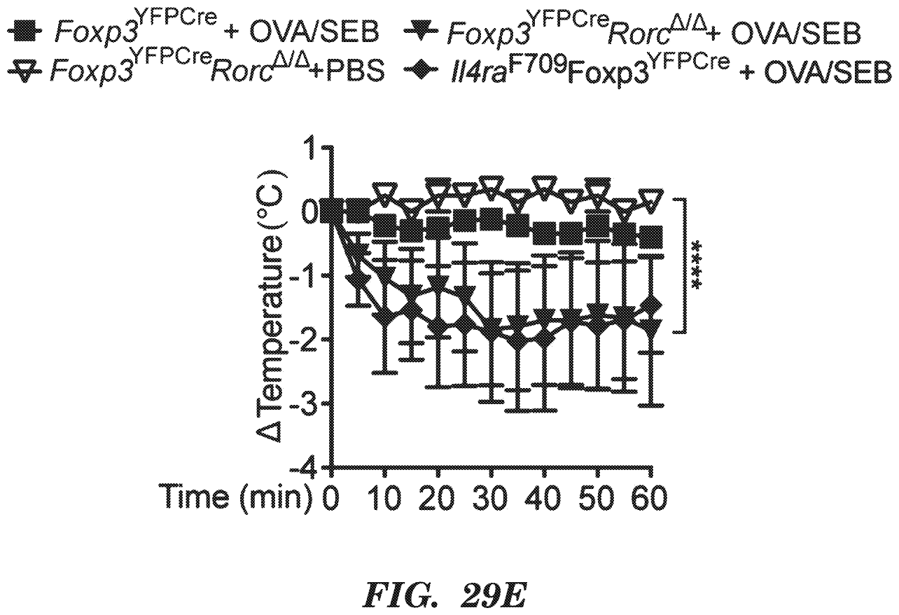

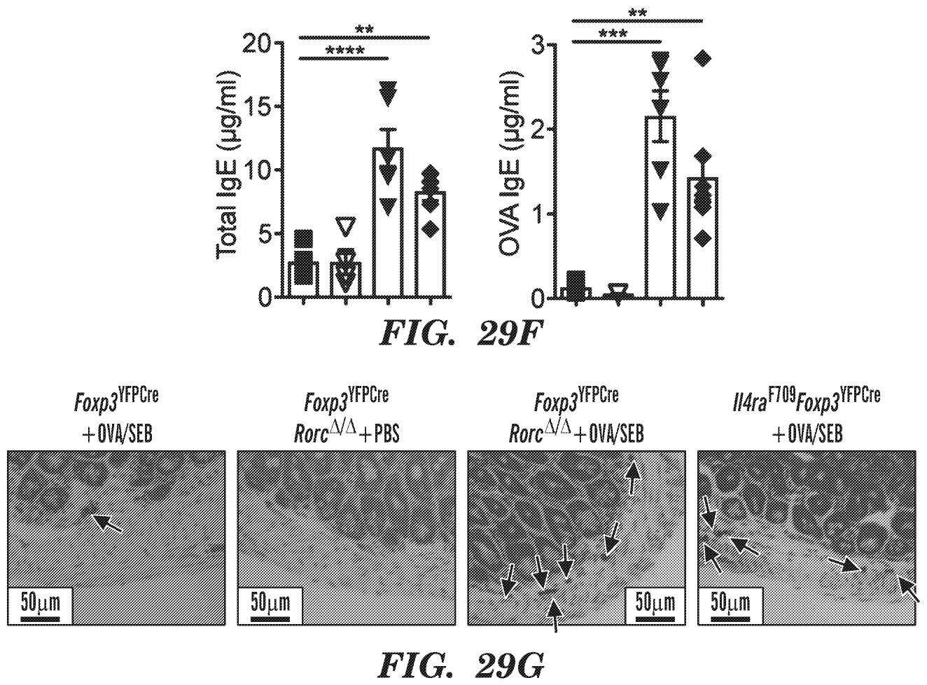

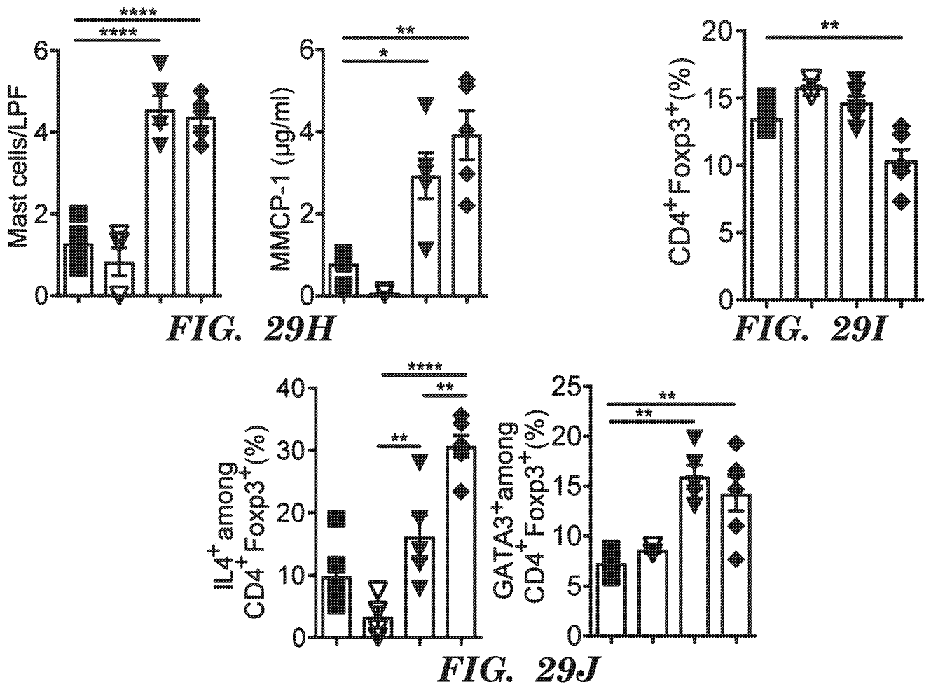

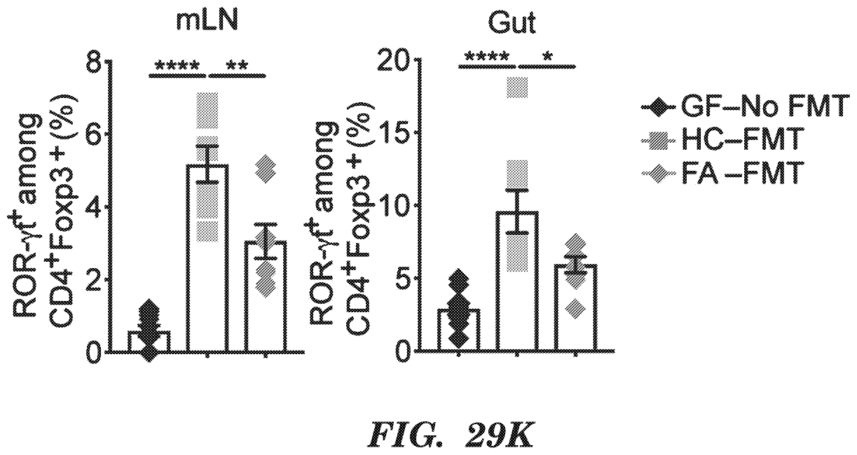

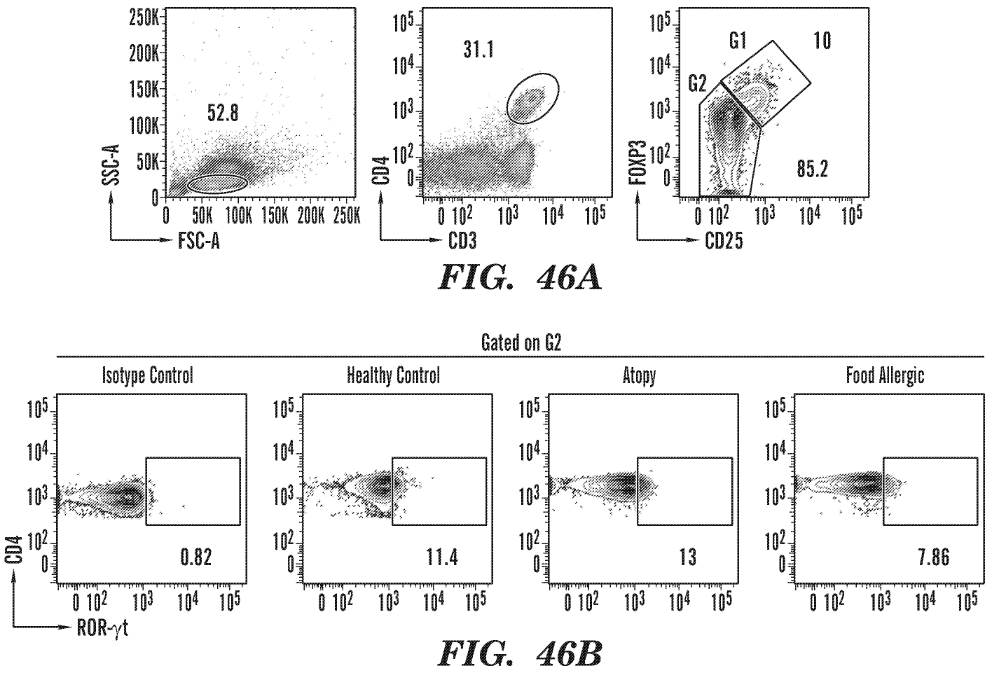

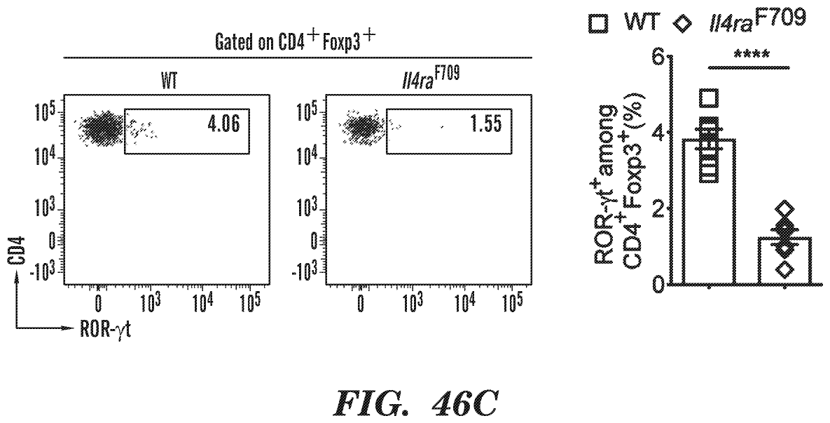

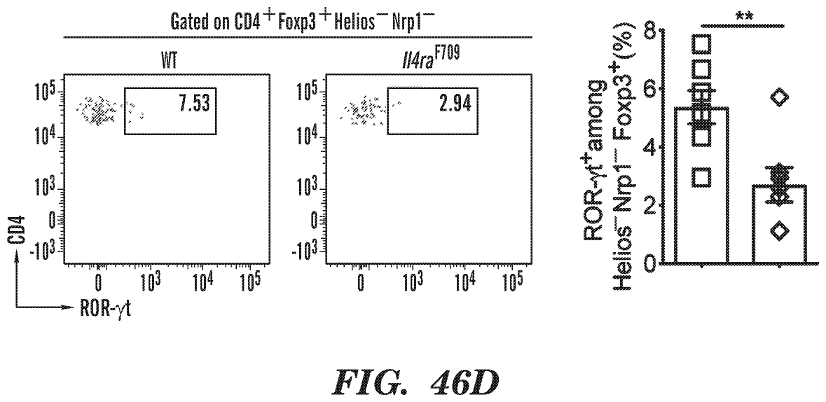

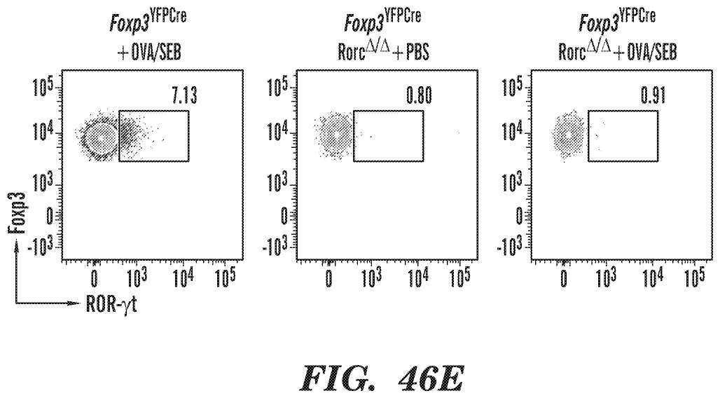

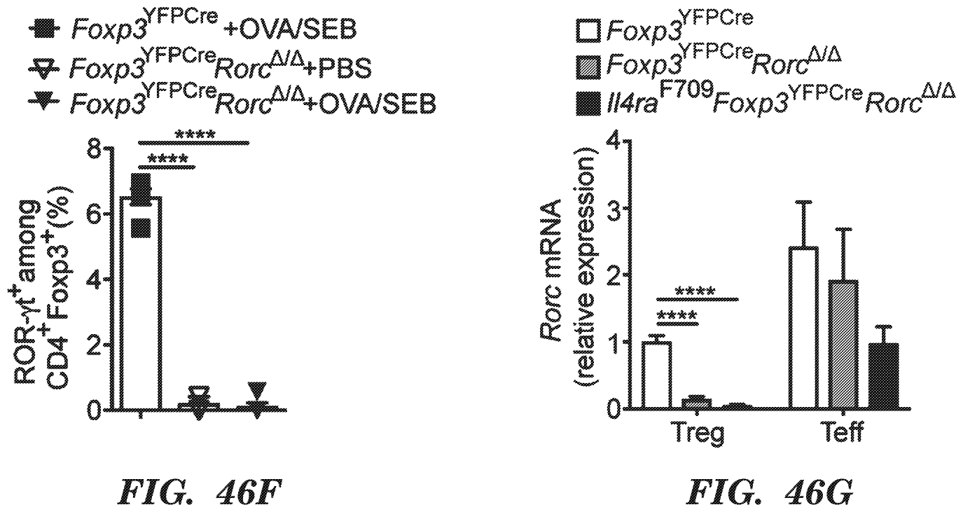

Protection against FA by the commensal bacteria is dependent on ROR-γt⁺ Treg cells. As both the Clostridiales and Bacteroidales consortia increased the frequencies and numbers of gut ROR-γt⁺ Treg cells in Il4ra^{F709} mice, the frequencies of circulating ROR-γt⁺ Treg cells were examined in human FA subjects as compared to subjects who were atopic but not allergic to foods and to non-atopic subjects (see e.g., TABLES 12-17). FA subjects had decreased circulating ROR-γt⁺ Treg cells as compared to those of the other two groups, which were otherwise similar (see e.g., FIGS. 29A-29B). In contrast, the frequencies of circulating ROR-γt⁺ T effector (Teff) cells were not significantly different between FA and control subjects and slightly increased in atopics (see e.g., FIG. 29C and FIGS. 46A-46B). ROR-γt⁺ Treg cells were similarly decreased at steady state in the peripheral blood of Il4ra^{F709} mice. Unlike the case in WT mice, OVA/SEB sensitization did not result in increased induction of ROR-γt+ Treg cells in the MLN of OVA/SEB-sensitized FA Il4ra^{F709} mice as compared to WT controls (see e.g., FIG. 29D and FIGS. 46C-46D).

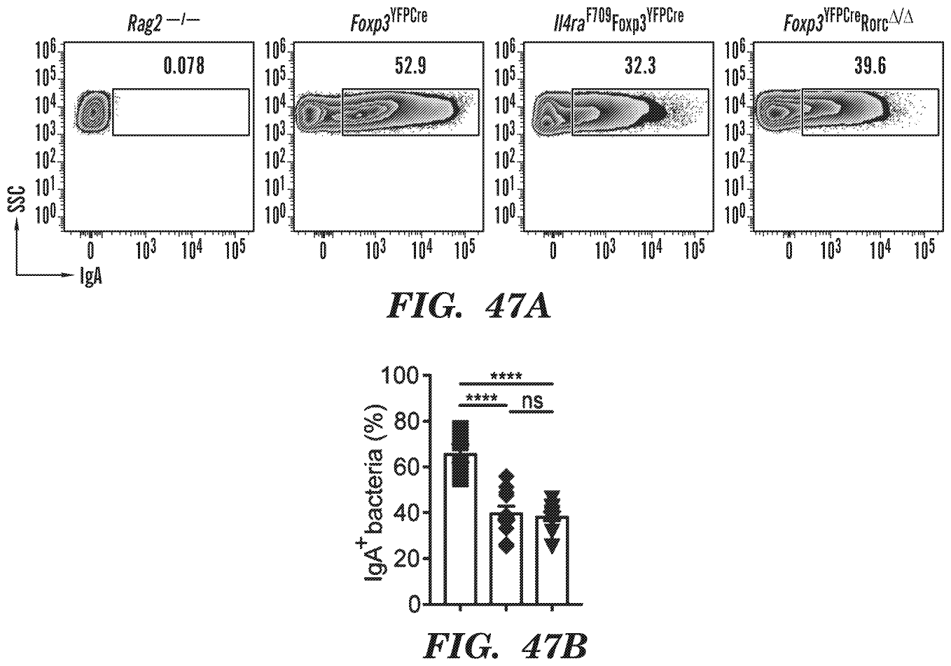

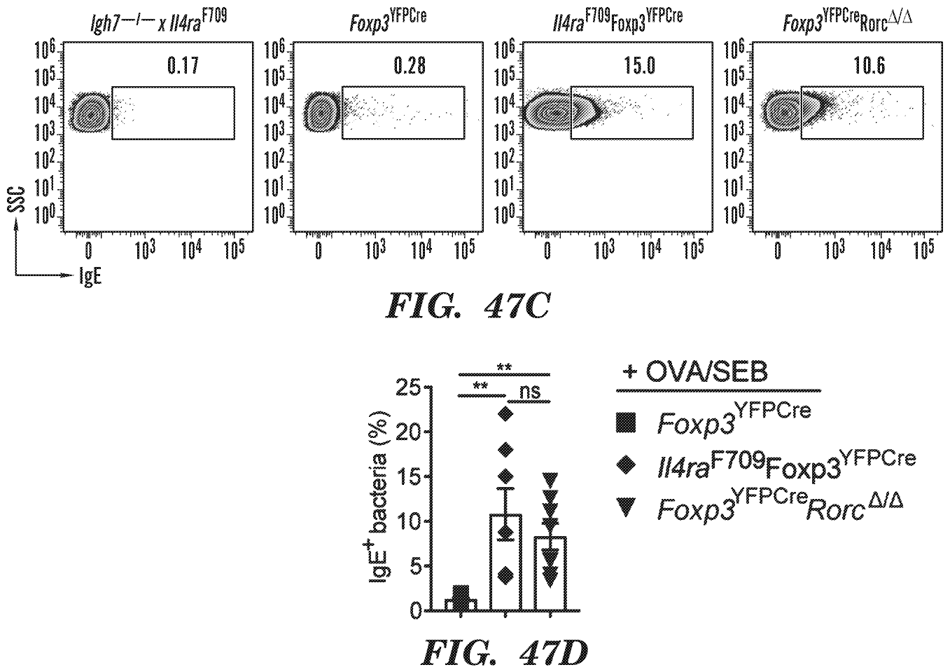

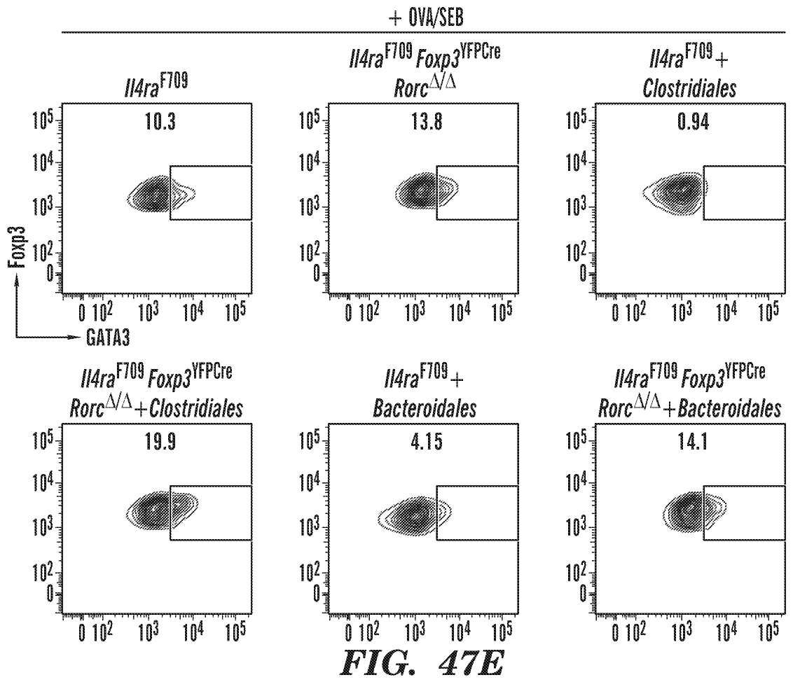

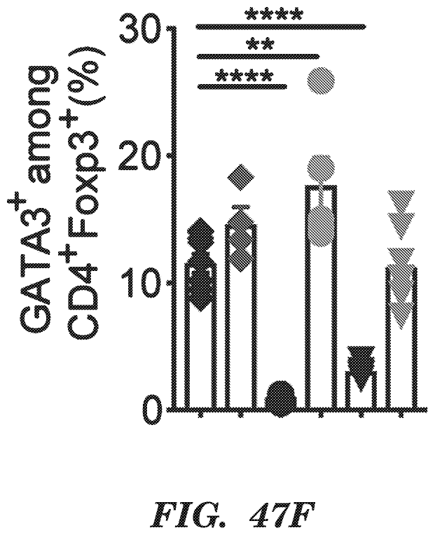

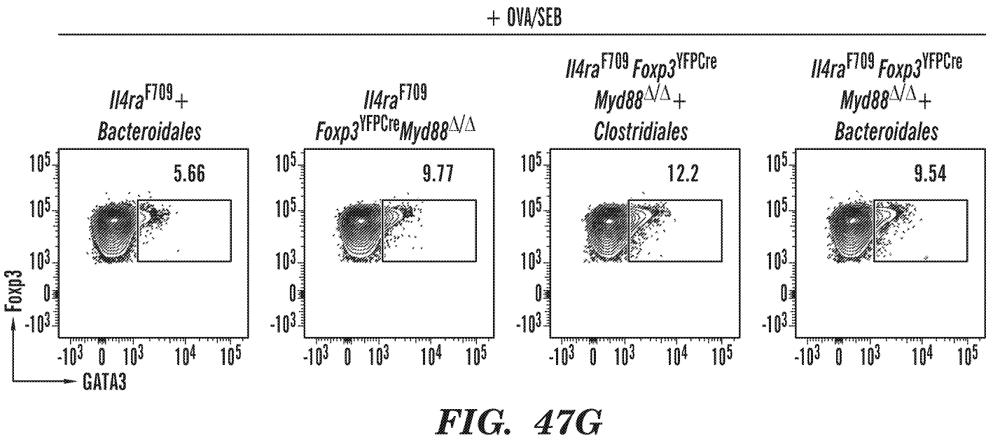

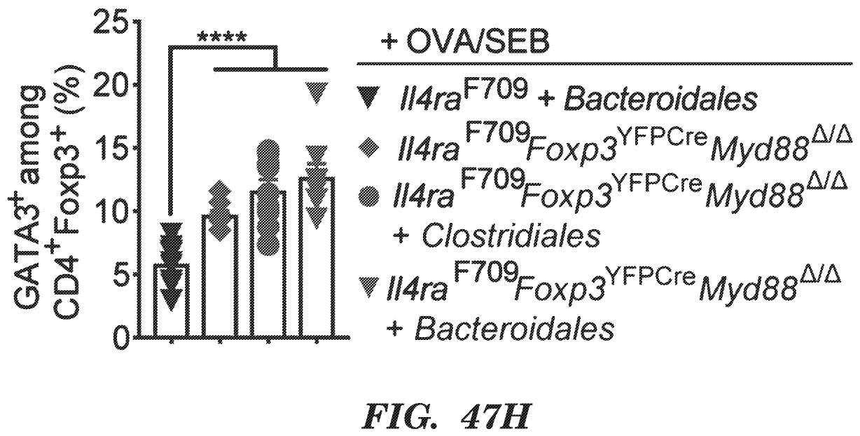

To establish the role of ROR-γt⁺ Treg cells in tolerance induction in FA, the consequences of Treg cell-specific deletion Rorc, the gene encoding ROR-γt, was examined in promoting FA. Mice expressing a Foxp3 allele that drove Treg cell-specific expression of a Cre recombinase and a yellow fluorescent protein (Foxp3^{YFPCre}) were crossed to homozygosity with a floxed Rorc allele. The Treg cells of Foxp3^{YFPCre}Rorc^{Δ/Δ} mice were profoundly deficient in ROR-γt expression as compared to those of Foxp3^{YFPCre} mice, reflecting the loss of Rorc mRNA specifically in Treg cells (see e.g., FIGS. 46E-46G). Foxp3^{YFPCre}Rorc^{Δ/Δ} mice sensitized with OVA/SEB and challenged with OVA developed a vigorous anaphylactic response that was comparable to that of similarly treated Il4ra^{F709} mice, with increased total and OVA-specific IgE, mast cell expansion and MMCP1 release. In contrast, Foxp3^{YFPCre} mice were resistant to FA induction (see e.g., FIGS. 29G-29H). Also, and similar to Il4ra^{F709} mice, Foxp3^{YFPCre}Rorc^{Δ/Δ} mice sensitized with OVA/SEB exhibited decreased sIgA and increased IgE binding to the commensal fecal bacteria, consistent with the dysregulation of the mucosal immune response (see e.g., FIGS. 47A-47D). Treg cell-specific Rorc deletion did not affect the frequency or numbers of Treg cells in the MLN of OVA/SEB sensitized Foxp3^{YFPC}reR_{orc}^{Δ/Δ} mice, but the Treg cells in those tissues did show evidence of disease-promoting Th2 cell-like skewing with increased GATA3 and IL-4 expression, similar to those of FA Il4ra^{F709} mice (see e.g., FIGS. 29F-29J)²⁵.

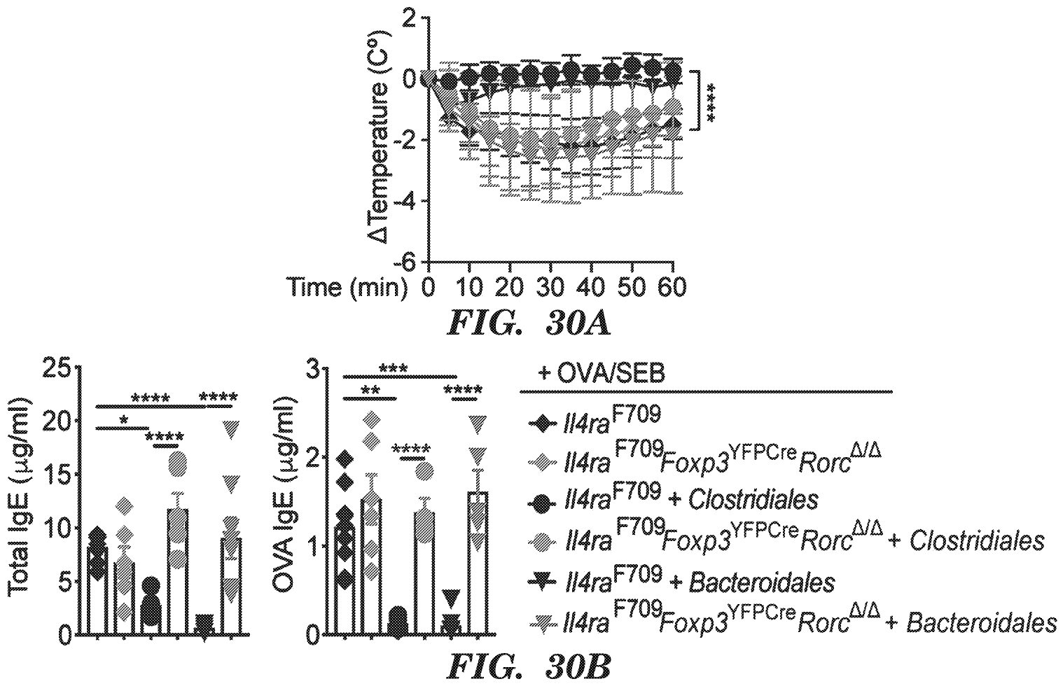

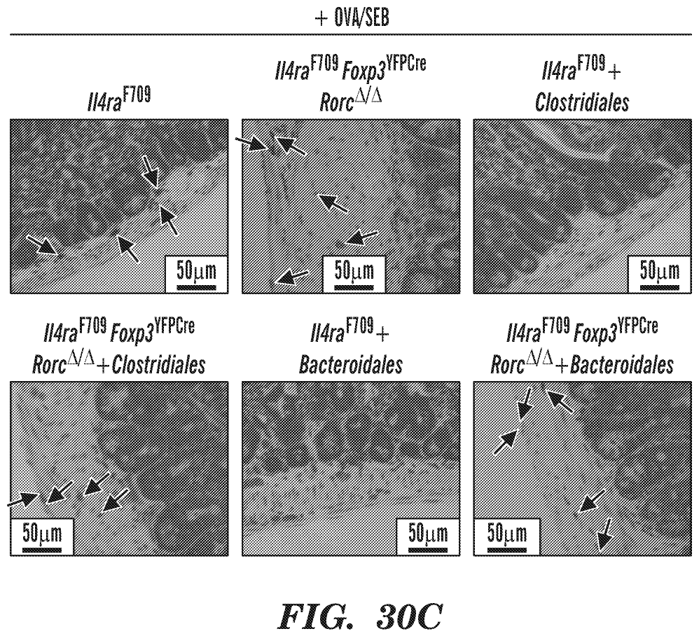

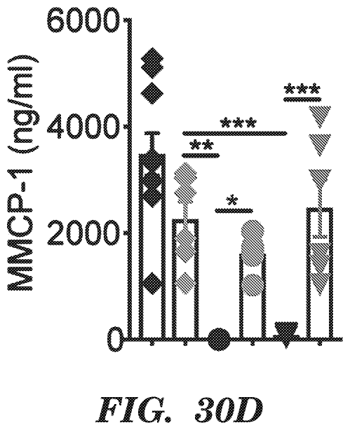

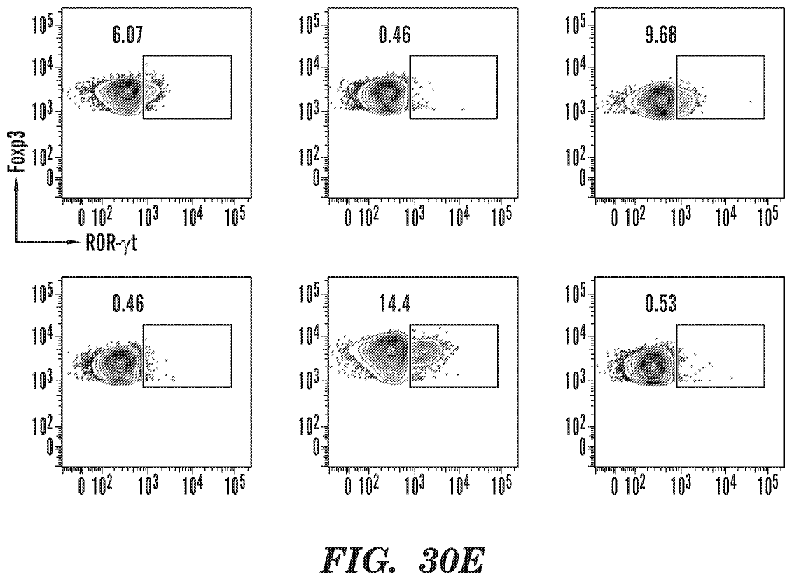

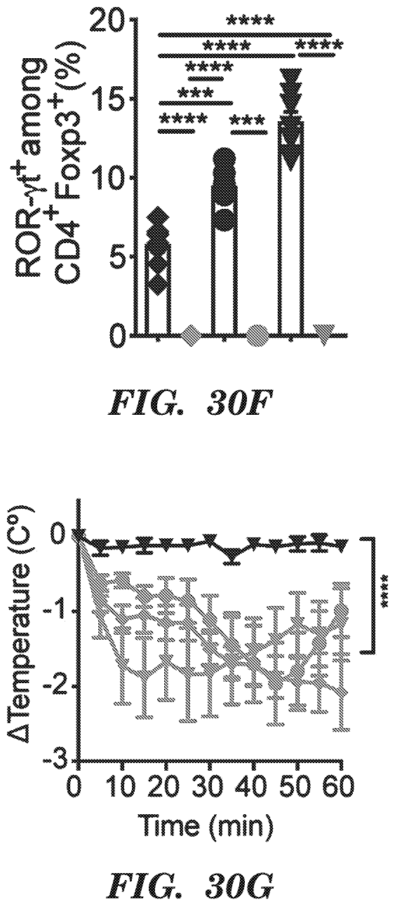

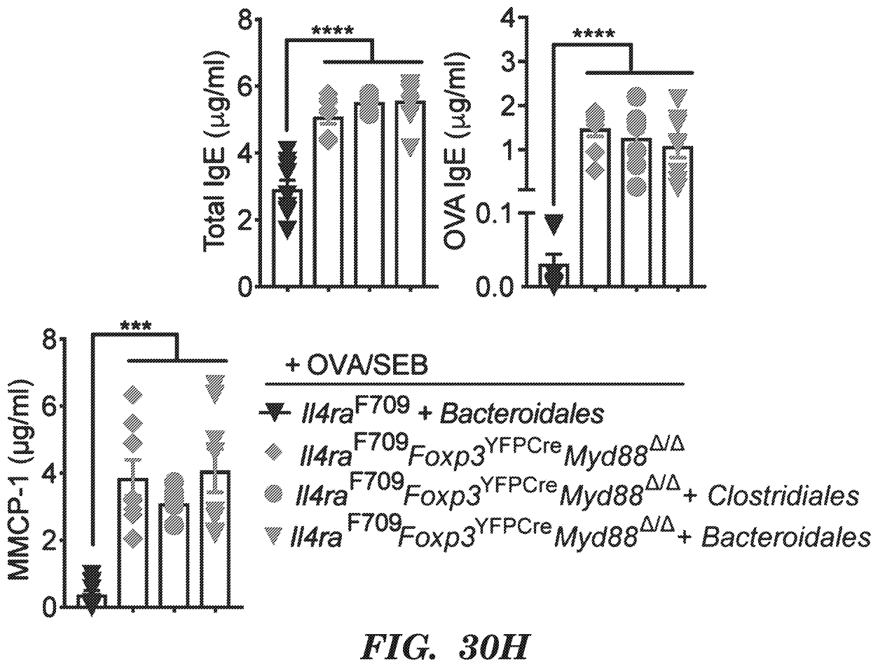

The role of ROR-γt⁺ Treg cells in mediating protection by the Clostridiales and Bacteroidales consortia was next examined in FA Il4ra^{F709} Whereas treatment of Rorc-sufficient Il4ra^{F709}Foxp3^{YFPCre} mice with either consortium prevented FA induction by OVA/SEB sensitization, Treg cell-specific deletion of Rorc in Il4raF709 mice (Il4ra^{F709}Foxp3^{YFPCre}Rorc^{Δ/Δ}), which depleted Rorc mRNA expression specifically in this cell compartment (see e.g., FIG. 46G), abrogated protection by both consortia. OVA/SEB-sensitized and bacterial consortia-treated Il4ra^{F709}Foxp3^{YFPCre}Rorc^{Δ/Δ} mice exhibited all the attributes of FA, including anaphylaxis, total and OVA-specific IgE responses and MMCP1 release (see e.g., FIGS. 30A-30D). As expected, the Clostridiales and Bacteroidales consortia induced ROR-γt⁺ Treg cells in Il4ra^{F709}Foxp3^{YFPCre} but not Il4ra^{F709}Foxp3^{YFPCre}Rorc^{Δ/Δ} mice (see e.g., FIGS. 30E-30F), with the latter mice showing Th2 cell-like skewing of their gut Treg cells (see e.g., FIGS. 47E-47F). These results established a requisite role for microbiota-induced ROR-γt⁺ Treg cells in enforcing oral tolerance in FA.

MyD88-dependent microbial sensing in nascent Treg cells is known to regulate the sIgA responses to gut commensals and promotes mucosal tolerance. Accordingly, the role of Treg cell-specific MyD88 signaling in mediating the effects of the consortia was examined in restoring immune tolerance. Treg cell-specific deletion of Myd88 in Il4ra^{F709} mice)(Il4ra^{F709}Foxp3^{YFPCre}Myd88^{Δ/Δ} abrogated the protection by both the Clostridiales and Bacteroidales consortia against FA and also their induction of ROR-γt⁺ iTreg cells (see e.g., FIGS. 30G-30J), with the gut Treg cells skewing towards a Th2 cell-like phenotype (see e.g., FIGS. 47G-47H). These results established a microbiota-responsive MyD88-ROR-γt pathway operative in nascent gut iTreg cells that mediates the therapeutic effects of bacteriotherapy in FA.

### Discussion

Several important aspects of the role of dysbiosis in FA emerged from these studies. First, an altered gut microbiota in FA infants was identified starting as early as 1-6 months of age, consistent with a dysbiotic process that begins very early in life. The dysbiosis evolved dynamically over time, with some of these differences found to be age group-specific, while others persisted across different age groups. This early life time window of disease vulnerability may be especially important given the malleability of the microbiota composition in infants and its susceptibility to pathogenic dysbiosis under environmental influences including diet and antibiotic usage. Importantly, the dysbiotic microbiota of FA infants failed to protect against FA when introduced in GF Il4ra^{F709} mice, whereas those of healthy human subjects did. In the same model, the microbiota of FA prone Il4ra^{F709} mice bred under SPF conditions were also non-protective as compared to those WT mice, indicating an essential role for dysbiosis in FA pathogenesis.

Oral administration of a defined consortium of six human-origin Clostridiales species, related to taxa impacted by dysbiosis in human FA infants, protected against FA in both GF and microbiota-sufficient FA-prone R4ra^{F709} mice and suppressed established disease in the latter mice. Immunologically, administration of the Clostridiales consortium normalized the sIgA antibody response and suppressed the IgE response to the gut commensals. In contrast, a consortium of Proteobacteria species associated with gut dysbiosis failed to protect the mice from FA or to normalize the gut anti-commensal IgA and IgE responses. These results show that a small consortium (e.g., less than 15) of human type strains Clostridiales species acted to both prevent and suppress FA. Intriguingly, monobacterial therapy with Subdoligranulum variabile, a Clostridiales species found deficient in FA subjects age one year and older was also protective against FA in SPF R4ra^{F709} mice, although less stringently than the Clostridales consortium, suggesting that the loss of protective species due to dysbiosis is a key feature of disease pathogenesis.

Critically, the protection against FA was not a unique attribute of Clostridiales species as a second unrelated consortium of five human origin Bacteroidales species was also effective in both preventing and curing FA in sensitized R4ra^{F709} mice. Both consortia required an intact ROR-γt⁺ iTreg cell response to promote therapeutic efficacy. Though related taxa of Bacteroidales (and Clostridales) were increased in FA subjects, the inability of the complex FA patient microbiota to provide protection in the GF R4ra^{F709} mouse model, thought its inability to induce protective ROR-γt⁺ iTreg cell responses, indicates that those taxa found in patients are either poorly immunomodulatory as compared to the species in the respective consortia, or that their in vivo activity fails to promote immunomodulatory effects.

ROR-γt⁺ Treg cells have been implicated in mediating tolerance induction by gut commensals, but it has been unclear which immune responses were regulated by this Treg cell subpopulation. These results indicate that the induction of ROR-γt⁺ iTreg cells by healthy commensal flora via a Treg cell-specific MyD88-dependent pathway plays a requisite role in forestalling the development of FA and in suppressing established disease. In contrast, the Proteobacteria consortium, which increased the frequencies of Treg cells in the gut but did not induce ROR-γt⁺ iTreg cells, failed to protect against FA. Notably, the frequencies of ROR-γt⁺ iTreg cells were decreased in both human FA allergic subjects and mice, consistent with their requirement in mediating oral tolerance in FA. These results provide a unifying mechanism for disease initiation in FA, involving dysbiotic gut microbial communities that fail to induce ROR-γt⁺ iTreg cell populations, and they support the use of immunomodulatory bacteria to restore ROR-γt⁺ iTreg populations and re-establish tolerance in FA.

MyD88-dependent microbial sensing by nascent gut Treg cells is instrumental in driving T follicular regulatory cell differentiation in the Peyer's patches and directing anti-IgA responses to gut luminal antigens including commensals and foods. Disruption of the MyD88-ROR-γt regulatory axis by dysbiosis in FA is reflected by the decreased IgA and increased IgE responses to the gut microbiota, reproduced upon Treg cell-specific deletion of Rorc. Under these conditions, a population of GATA3^{high}, Th2 cell-like reprogrammed natural Treg cells that expresses the cytokine IL-4 expands in the gut. This population, which is also increased in FA subjects, contributes to disease pathogenesis, evidenced by the down regulation of FA responses in Il4ra^{F709} mice upon Treg cell-specific deletion of a genetic cassette encompassing the Il4 and Il13 genes. These studies point to coordinated changes in gut Treg cell populations involving dysbiosis-induced deficiency of ROR-γt⁺ iTreg cells and a reciprocal expansion of GATA3^{high}, Th2 cell-like reprogrammed Treg cells as cardinal events in the pathogenesis of FA in early life.

The demonstration that the allergic response in FA extends beyond food allergens to involve the gut microbiota is indicative of a broader disturbance in oral tolerance than hitherto appreciated. Immune responses to the gut microbiota are known to arise in the context of gastrointestinal infections. These responses may contribute to recall immunity upon infectious re-challenge. Without wishing to be bound by theory, it is proposed that the presence of an anti-microbiota Th2 response can similarly act to aggravate pathogenic immune responses to foods and can play a critical role in disease initiation, persistence and outcome.

TABLE 6
Demographic characteristics of FA and control infants.

| | 1-6 months | | 7-12 months | | 13-18 months | | 19-24 months | | 25-30 months | |
|---|---|---|---|---|---|---|---|---|---|---|
| Demo- | | Food | | Food | | Food | | Food | | Food |
| graphic | Controls | allergic | Controls | allergic | Controls | allergic | Controls | allergic | Controls | allergic |
| charact- | (n = 32) | (n = 10) | (n = 61) | (n = 22) | (n = 56) | (n = 33) | (n = 33) | (n = 24) | (n = 9) | (n = 15) |
| eristics | n (%) | n (%) | n (%) | n (%) | n (%) | n (%) | n (%) | n (%) | n (%) | n (%) |

Age group in months

| | | | | | | | | | | | | | | | | | | | |
|---|---|---|---|---|---|---|---|---|---|---|---|---|---|---|---|---|---|---|---|
| 32 | (76.19) | 10 | (23.81) | 61 | (73.49) | 22 | (26.51) | 56 | (62.91) | 33 | (37.09) | 33 | (57.89) | 24 | (42.11) | 9 | (37.50) | 15 | (62.50) |

Gender

| | | | | | | | | | | | | | | | | | | | | |
|---|---|---|---|---|---|---|---|---|---|---|---|---|---|---|---|---|---|---|---|---|
| Female | 18 | (56.25) | 3 | (30) | 29 | (47.54) | 9 | (40.91) | 29 | (51.79) | 10 | (30.30) | 14 | (42.42) | 7 | (29.17) | 6 | (66.67) | 3 | (20) |
| Male | 14 | (43.75) | 7 | (70) | 32 | (52.46) | 13 | (59.09) | 27 | (48.21) | 23 | (69.70) | 19 | (57.58) | 17 | (70.83) | 3 | (33.33) | 12 | (80) |

| | | | | | |
|---|---|---|---|---|---|
| Ad- | 0.815 | 1.000 | 0.381 | 0.815 | 0.225 |
| justed | | | | | |
| p- | | | | | |
| values | | | | | |

Mode of delivery

| | | | | | | | | | | | | | | | | | | | | |
|---|---|---|---|---|---|---|---|---|---|---|---|---|---|---|---|---|---|---|---|---|
| C/S | 6 | (18.75) | 1 | (10) | 18 | (29.51) | 4 | (18.18) | 20 | (35.71) | 7 | (21.21) | 13 | (39.39) | 6 | (25) | 4 | (44.44) | 3 | (20) |
| NVD | 26 | (81.25) | 9 | (90) | 43 | (70.49) | 18 | (81.82) | 36 | (63.29) | 26 | (78.79) | 20 | (60.61) | 18 | (75) | 5 | (55.56) | 12 | (80) |

| | | | | | |
|---|---|---|---|---|---|
| Ad- | 1.000 | 0.815 | 0.815 | 0.815 | 0.815 |
| justed | | | | | |
| p- | | | | | |
| values | | | | | |

Breast feeding

| | | | | | | | | | | | | | | | | | | | | |
|---|---|---|---|---|---|---|---|---|---|---|---|---|---|---|---|---|---|---|---|---|
| No | 7 | (31.82) | 3 | (30) | 24 | (39.34) | 9 | (40.91) | 38 | (67. 86) | 21 | (63.63) | 30 | (90.91) | 22 | (91.67) | 8 | (88.89) | 12 | (80) |
| Yes | 25 | (68.18) | 7 | (70) | 37 | (60.66) | 13 | (59.09) | 18 | (32.14) | 12 | (36.37) | 3 | (9.09) | 2 | (8.33) | 1 | (11.11) | 3 | (20) |

| | | | | | |
|---|---|---|---|---|---|
| Ad- | 1.000 | 1.000 | 1.000 | 1.000 | 1.000 |
| justed | | | | | |
| p- | | | | | |
| values | | | | | |

Cow's Milk Proteins intake

| | | | | | | | | | | | | | | | | | | |
|---|---|---|---|---|---|---|---|---|---|---|---|---|---|---|---|---|---|---|
| No | 28 | (87.50) | 10 | (100) | 21 | (34.43) | 16 | (72.73) | 3 | (5.36) | 16 | (48.49) | 0 | 8 | (33.33) | 0 | 4 | (26.67) |

| | | | | | | | | | | | | | | | | | | | |
|---|---|---|---|---|---|---|---|---|---|---|---|---|---|---|---|---|---|---|---|
| Yes | 4 | (12.50) | 0 | 40 | (65.57) | 6 | (27.27) | 53 | (94.64) | 17 | (51.51) | 33 | (100) | 18 | (66.67) | 9 | (100) | 11 | (73.33) |

| | | | | | |
|---|---|---|---|---|---|
| Ad- | 0.994 | 0.022 | 0.0001 | 0.009 | 0.815 |
| justed | | | | | |
| p- | | | | | |
| values | | | | | |

TABLE 6 shows demographic characteristics of 56 FA and 98 controls subjects. Subjects were recruited ages 1-15 month and followed thereafter up to age 30 months. Samples that were collected from subjects were segregated by different age group: 1-6 months, 7-12 months-13-18 months, 19-24 months and 25-30 months. The attributes of patients contributing to the samples collected at the respective age groups, including gender, mode of delivery, breast-feeding and milk tolerance are shown. P-values were adjusted for multiple hypothesis using the Benjami-Hochberg method. A P value <0.05 was considered significant.

TABLE 7
Differences in bacterial genera between FA and control subjects

| | | | | | | | log2 | | |
|---|---|---|---|---|---|---|---|---|---|
| | Age | | OTU | Like−weight | P− | | Fold | Ifc | |
| Comparisons | Groups | Lineage | ID | ratio | value | FDR | Change | SE | Comments |
| Controls | 1 To 6 | Bacteria; Bacteroidetes; Bacteroidia; Bacteroidales; | Otu000024 | 1 | 5.68E−06 | 0.000112684 | −4.824930246 | 1.06325418 | Elevated |
| (n = 32) vs | | Porphyromonadaceae; Parabacteroides | | | | | | | in control |
| Food | | | | | | | | | group |
| Allergic | 1 To 6 | Bacteria; Firmicutes; Clostridia; Clostridiales; | Otu000033 | 1 | 0.021737196 | 0.078348234 | 2.327779198 | 1.01431427 | Elevated |
| (n = 10) | | Lachnospiraceae; Clostridium_XIVa | | | | | | | in control |
| | | | | | | | | | group |
| | 1 To 6 | Bacteria; Actinobacteria; Actinobacteria; Coriobacteriales; | Otu000197 | 0.403938 | 7.16E−07 | 2.00E−05 | 3.926314129 | 0.79210061 | Elevated |
| | | Coriobacteriaceae; Atopobium | | | | | | | in food |
| | | | | | | | | | allergic group |
| | 1 To 6 | Bacteria; Firmicutes; Bacilli; Lactobacillales; | Otu000122 | 0.822538 | 0.000192468 | 0.001959765 | 2.916655575 | 0.78221722 | Elevated |
| | | Lactobacillaceae; Lactobacillus | | | | | | | in food |
| | | | | | | | | | allergic group |
| | 1 To 6 | Bacteria; Firmicutes; Bacilli; Lactobacillales; | Otu000120 | 0.616971 | 3.41E−08 | 1.24E−06 | 5.120716702 | 0.9278032 | Elevated |
| | | Lactobacillaceae; Lactobacillus | | | | | | | in food |
| | | | | | | | | | allergic group |
| | 1 To 6 | Bacteria; Firmicutes; Clostridia; Clostridiales; | Otu000027 | 0.967685 | 4.98E−05 | 0.000625041 | 3.24682532 | 0.80039545 | Elevated |
| | | Clostridiaceae_1; Clostridium_sensu_stricto | | | | | | | in food |
| | | | | | | | | | allergic group |
| | 1 To 6 | Bacteria; Firmicutes; Clostridia; Clostridiales; | Otu000020 | 0.336472 | 2.61E−08 | 1.07E−06 | 6.018394601 | 1.0813377 | Elevated |
| | | Clostridiaceae_1; Clostridium_sensu_stricto | | | | | | | in food |
| | | | | | | | | | allergic group |
| | 1 To 6 | Bacteria; Firmicutes; Clostridia; Clostridiales; | Otu000070 | 1 | 8.20E−10 | 4.20E−08 | 5.786033105 | 0.94219611 | Elevated |
| | | Lachnospiraceae; Clostridium_XIVa | | | | | | | in food |
| | | | | | | | | | allergic group |
| | 1 To 6 | Bacteria; Firmicutes; Clostridia; | Otu000095 | 0.543413 | 0.001075162 | 0.007600283 | 2.840591855 | 0.86866066 | Elevated |
| | | Clostridiales; Lachnospiraceae; Dorea | | | | | | | in food |
| | | | | | | | | | allergic group |
| | 1 To 6 | Bacteria; Firmicutes; Clostridia; | Otu000089 | 1 | 1.87E−07 | 6.05E−06 | 4.169211312 | 0.7999795 | Elevated |
| | | Clostridiales; Peptostreptococcaceae; Clostridium_XI | | | | | | | in food |
| | | | | | | | | | allergic group |
| | 1 To 6 | Bacteria; Firmicutes; Clostridia; Clostridiales; | Otu000097 | 0.943417 | 2.63E−07 | 7.70E−06 | 4.203565724 | 0.81655895 | Elevated |
| | | unclassified; unclassified | | | | | | | in food |
| | | | | | | | | | allergic group |
| | 1 To 6 | Bacteria; Proteobacteria; Deltaproteobacteria; | Otu000068 | 1 | 1.37E−06 | 3.51E−05 | 4.504780092 | 0.93285968 | Elevated |
| | | Desulfovibrionales; Desulfovibrionaceae; Bilophila | | | | | | | in food |
| | | | | | | | | | allergic group |
| Controls | 7 To 12 | Bacteria; Actinobacteria; | Otu000004 | 0.315041 | 0.029210398 | 0.095049708 | −0.981972309 | 0.45031477 | Elevated |
| (n = 61) vs | | Actinobacteria; Bifidobacteriales; | | | | | | | in control |
| Food | | Bifidobacteriaceae; Bifidobacterium | | | | | | | group |
| Allergic | 7 To 12 | Bacteria; Bacteroidetes; Bacteroidia; | Otu000018 | 1 | 0.003305958 | 0.018773817 | −1.87652807 | 0.63876213 | Elevated |
| (n = 22) | | Bacteroidales; Bacteroidaceae; Bacteroides | | | | | | | in control |
| | | | | | | | | | group |
| | 7 To 12 | Bacteria; Firmicutes; Negativicutes; | Otu000023 | 1 | 0.002509367 | 0.014839045 | −1.774294356 | 0.58708515 | Elevated |
| | | Selenomonadales; Veillonellaceae; Dialister | | | | | | | in control |
| | | | | | | | | | group |
| | 7 To 12 | Bacteria; Proteobacteria; Betaproteobacteria; | Otu000074 | 1 | 0.001136112 | 0.00767812 | −2.007180265 | 0.61674898 | Elevated |
| | | Burkholderiales; Sutterellaceae; Sutterella | | | | | | | in control |
| | | | | | | | | | group |
| | 7 To 12 | Bacteria; Proteobacteria; Gammaproteobacteria; | Otu000005 | 0.143807 | 0.022366063 | 0.079052464 | −1.236873828 | 0.54151639 | Elevated |
| | | Enterobacteriales; Enterobacteriaceae; Escherichia/ | | | | | | | in control |
| | | Shigella | | | | | | | group |
| | 7 To 12 | Bacteria; Actinobacteria; Actinobacteria; Coriobacteriales; | Otu000112 | 0.675625 | 0.003437239 | 0.019217291 | 1.455400234 | 0.4974601 | Elevated |
| | | Coriobacteriaceae; Eggerthella | | | | | | | in food |
| | | | | | | | | | allergic group |
| | 7 To 12 | Bacteria; Bacteroidetes; Bacteroidia; | Otu000040 | 1 | 1.67E−11 | 1.03E−09 | 4.041213401 | 0.60023837 | Elevated |
| | | Bacteroidales; Bacteroidaceae; Bacteroides | | | | | | | in food |
| | | | | | | | | | allergic group |
| | 7 To 12 | Bacteria; Bacteroidetes; Bacteroidia; Bacteroidales; | Otu000035 | 0.418803 | 8.52E−09 | 3.74E−07 | 4.189706597 | 0.72765809 | Elevated |
| | | Porphyromonadaceae; Parabacteroides | | | | | | | in food |
| | | | | | | | | | allergic group |
| | 7 To 12 | Bacteria; Bacteroidetes; Bacteroidia; | Otu000008 | 1 | 1.62E−05 | 0.000243 | 2.878916004 | 0.66761025 | Elevated |
| | | Bacteroidales; Prevotellaceae; Prevotella | | | | | | | in food |
| | | | | | | | | | allergic group |
| | 7 To 12 | Bacteria; Bacteroidetes; Bacteroidia; | Otu000025 | 0.931276 | 1.07E−14 | 1.65E−12 | 4.882151003 | 0.63155586 | Elevated |
| | | Bacteroidales; Rikenellaceae; Alistipes | | | | | | | in food |
| | | | | | | | | | allergic group |
| | 7 To 12 | Bacteria; Firmicutes; Bacilli; | Otu000117 | 0.801837 | 0.000125262 | 0.001375645 | 2.003081085 | 0.52223517 | Elevated |
| | | Lactobacillales; Lactobacillaceae; Lactobacillus | | | | | | | in food |
| | | | | | | | | | allergic group |
| | 7 To 12 | Bacteria; Firmicutes; Bacilli; | Otu000013 | 0.312224 | 0.022937535 | 0.080609052 | 1.281113462 | 0.56325716 | Elevated |
| | | Lactobacillales; Streptococcaceae; Streptococcus | | | | | | | in food |
| | | | | | | | | | allergic group |
| | 7 To 12 | Bacteria; Firmicutes; Clostridia; | Otu000027 | 0.336472 | 0.001648387 | 0.01013758 | 1.826261035 | 0.58028009 | Elevated |
| | | Clostridiales; Clostridiaceae_1; Clostridium_sensu_stricto | | | | | | | in food |
| | | | | | | | | | allergic group |
| | 7 To 12 | Bacteria; Firmicutes; Clostridia; | Otu000091 | 1 | 0.000737497 | 0.00581488 | 1.787866174 | 0.52969947 | Elevated |
| | | Clostridiales; Lachnospiraceae; Anaerostipes | | | | | | | in food |
| | | | | | | | | | allergic group |
| | 7 To 12 | Bacteria; Firmicutes; Clostridia; | Otu000009 | 1 | 0.021946868 | 0.078348234 | 1.398909738 | 0.61053471 | Elevated |
| | | Clostridiales; Lachnospiraceae; Blautia | | | | | | | in food |
| | | | | | | | | | allergic group |
| | 7 To 12 | Bacteria; Firmicutes; Clostridia; | Otu000030 | 0.976048 | 0.002592811 | 0.015186464 | 1.891179265 | 0.62782022 | Elevated |
| | | Clostridiales; Lachnospiraceae; Blautia | | | | | | | in food |
| | | | | | | | | | allergic group |
| | 7 To 12 | Bacteria; Firmicutes; Clostridia; | Otu000080 | 0.268673 | 6.91E−13 | 6.07E−11 | 4.371235598 | 0.60870178 | Elevated |
| | | Clostridiales; Lachnospiraceae; Blautia | | | | | | | in food |
| | | | | | | | | | allergic group |
| | 7 To 12 | Bacteria; Firmicutes; Clostridia; | Otu000033 | 1 | 0.001185408 | 0.007924195 | 1.951055747 | 0.6017379 | Elevated |
| | | Clostridiales; Lachnospiraceae; Clostridium_XIVa | | | | | | | in food |
| | | | | | | | | | allergic group |
| | 7 To 12 | Bacteria; Firmicutes; Clostridia; | Otu000039 | 1 | 3.15E−12 | 2.42E−10 | 3.828189092 | 0.54915812 | Elevated |
| | | Clostridiales; Lachnospiraceae; Lachnospira | | | | | | | in food |
| | | | | | | | | | allergic group |
| | 7 To 12 | Bacteria; Firmicutes; Clostridia; | Otu000014 | 0.946 | 0.023356712 | 0.081615783 | 1.494636904 | 0.65914207 | Elevated |
| | | Clostridiales; Lachnospiraceae; Roseburia | | | | | | | in food |
| | | | | | | | | | allergic group |
| | 7 To 12 | Bacteria; Firmicutes; Clostridia; | Otu000065 | 0.176032 | 2.07E−14 | 2.55E−12 | 4.519073772 | 0.59104016 | Elevated |
| | | Clostridiales; Peptostreptococcaceae; Clostridium XI | | | | | | | in food |
| | | | | | | | | | allergic group |
| | 7 To 12 | Bacteria; Firmicutes; Clostridia; | Otu000006 | 1 | 0.000601476 | 0.004803997 | 2.202588576 | 0.64197671 | Elevated |
| | | Clostridiales; Ruminococcaceae; Faecalibacterium | | | | | | | in food |
| | | | | | | | | | allergic group |
| | 7 To 12 | Bacteria; Firmicutes; Clostridia; | Otu000050 | 0.847298 | 0.027166823 | 0.090802153 | 1.138196281 | 0.51522787 | Elevated |
| | | Clostridiales; Ruminococcaceae; Subdoligranulum | | | | | | | in food |
| | | | | | | | | | allergic group |
| | 7 To 12 | Bacteria; Firmicutes; Negativicutes; | Otu000021 | 0.2994 | 0.003986824 | 0.021698201 | 2.344484296 | 0.81428257 | Elevated |
| | | Selenomonadales; Veillonellaceae; Megasphaera | | | | | | | in food |
| | | | | | | | | | allergic group |
| | 7 To 12 | Bacteria; Firmicutes; Negativicutes; | Otu000012 | 0.892415 | 8.10E−06 | 0.000138375 | 3.42288433 | 0.76704119 | Elevated |
| | | Selenomonadales; Veillonellaceae; Veillonella | | | | | | | in food |
| | | | | | | | | | allergic group |
| | 7 To 12 | Bacteria; Proteobacteria; Betaproteobacteria; | Otu000028 | 1 | 4.21E−05 | 0.000550883 | 2.454349591 | 0.59929553 | Elevated |
| | | Burkholderiales; Sutterellaceae; Parasutterella | | | | | | | in food |
| | | | | | | | | | allergic group |
| | 7 To 12 | Bacteria; Proteobacteria; Deltaproteobacteria; | Otu000068 | 1 | 0.000270178 | 0.002479992 | 2.177805477 | 0.59791494 | Elevated |
| | | Desulfovibrionales; Desulfovibrionaceae; Bilophila | | | | | | | in food |
| | | | | | | | | | allergic group |
| Controls | 13 To 18 | Bacteria; Firmicutes; Clostridia; | Otu000020 | 0.967685 | 0.018370851 | 0.071506793 | −1.263124317 | 0.53566309 | Elevated |
| (n = 56) vs | | Clostridiales; Clostridiaceae_1; Clostridium_sensu_stricto | | | | | | | in control |
| Food | | | | | | | | | group |
| Allergic | 13 To 18 | Bacteria; Firmicutes; Clostridia; | Otu000095 | 0.543413 | 7.69E−05 | 0.000875806 | 2.033359363 | 0.51427533 | Elevated |
| (n = 33) | | Clostridiales; Lachnospiraceae; Dorea | | | | | | | in control |
| | | | | | | | | | group |
| | 13 To 18 | Bacteria; Firmicutes; Clostridia; | Otu000039 | 1 | 0.003477378 | 0.019266554 | 1.584619261 | 0.54229745 | Elevated |
| | | Clostridiales; Lachnospiraceae; Lachnospira | | | | | | | in control |
| | | | | | | | | | group |
| | 13 To 18 | Bacteria; Firmicutes; Clostridia; | Otu000059 | 0.699866 | 1.99E−09 | 9.41E−08 | −2.963520631 | 0.49402426 | Elevated |
| | | Clostridiales; Lachnospiraceae unclassified | | | | | | | in control |
| | | | | | | | | | group |
| | 13 To 18 | Bacteria; Firmicutes; Clostridia; | Otu000071 | 0.893897 | 0.026222412 | 0.088124499 | −1.221376347 | 0.54945129 | Elevated |
| | | Clostridiales; Lachnospiraceae unclassified | | | | | | | in control |
| | | | | | | | | | group |
| | 13 To 18 | Bacteria; Firmicutes; Clostridia; | Otu000121 | 1 | 0.024861429 | 0.084943216 | 0.990376209 | 0.44143283 | Elevated |
| | | Clostridiales; Lachnospiraceae unclassified | | | | | | | in control |
| | | | | | | | | | group |
| | 13 To 18 | Bacteria; Firmicutes; Clostridia; | Otu000050 | 0.847298 | 1.60E−05 | 0.000243 | −1.921399826 | 0.44537636 | Elevated |
| | | Clostridiales; Ruminococcaceae; Subdoligranulum | | | | | | | in control |
| | | | | | | | | | group |
| | 13 To 18 | Bacteria; unclassified; unclassified; | Otu000081 | 0.789286 | 0.008080704 | 0.037648735 | 1.283670113 | 0.4846454 | Elevated |
| | | unclassified; unclassified; unclassified | | | | | | | in control |
| | | | | | | | | | group |
| | 13 To 18 | Bacteria; Bacteroidetes; Bacteroidia; | Otu000010 | 0.238788 | 0.029847297 | 0.096610988 | 1.344857638 | 0.61914777 | Elevated |
| | | Bacteroidales; Bacteroidaceae; Bacteroides | | | | | | | in food |
| | | | | | | | | | allergic group |
| | 13 To 18 | Bacteria; Bacteroidetes; Bacteroidia; | Otu000018 | 1 | 0.006269803 | 0.03150596 | 1.580888306 | 0.57837508 | Elevated |
| | | Bacteroidales; Bacteroidaceae; Bacteroides | | | | | | | in food |
| | | | | | | | | | allergic group |
| | 13 To 18 | Bacteria; Bacteroidetes; Bacteroidia; | Otu000044 | 1 | 1.43E−18 | 8.79E−16 | 4.553081946 | 0.5177098 | Elevated |
| | | Bacteroidales; Bacteroidaceae; Bacteroides | | | | | | | in food |
| | | | | | | | | | allergic group |
| | 13 To 18 | Bacteria; Bacteroidetes; Bacteroidia; Bacteroides | Otu000040 | 0.577093 | 1.47E−17 | 4.52E−15 | 4.60134549 | 0.53944395 | Elevated |
| | | Bacteroidales; Bacteroidaceae; | | | | | | | in food |
| | | | | | | | | | allergic group |
| | 13 To 18 | Bacteria; Bacteroidetes; Bacteroidia; | Otu000035 | 1 | 0.011149891 | 0.047619326 | 1.165860488 | 0.4593681 | Elevated |
| | | Bacteroidales; Porphyromonadaceae; Parabacteroides | | | | | | | in food |
| | | | | | | | | | allergic group |
| | 13 To 18 | Bacteria; Bacteroidetes; Bacteroidia; | Otu000024 | 0.418803 | 0.000748862 | 0.005829748 | 2.045772488 | 0.60686767 | Elevated |
| | | Bacteroidales; Porphyromonadaceae; Parabacteroides | | | | | | | in food |
| | | | | | | | | | allergic group |
| | 13 To 18 | Bacteria; Bacteroidetes; Bacteroidia; | Otu000008 | 1 | 2.06E−05 | 0.000301643 | 2.512875344 | 0.59004316 | Elevated |
| | | Bacteroidales; Prevotellaceae; Prevotella | | | | | | | in food |
| | | | | | | | | | allergic group |
| | 13 To 18 | Bacteria; Bacteroidetes; Bacteroidia; Bacteroidales; | Otu000041 | 0.931276 | 0.003512503 | 0.019287405 | 1.540146801 | 0.52764333 | Elevated |
| | | Rikenellaceae; Alistipes | | | | | | | in food |
| | | | | | | | | | allergic group |
| | 13 To 18 | Bacteria; Bacteroidetes; Bacteroidia; | Otu000025 | 0.974259 | 0.001601642 | 0.010126295 | 1.813490413 | 0.5746882 | Elevated |
| | | Bacteroidales; Rikenellaceae; Alistipes | | | | | | | in food |
| | | | | | | | | | allergic group |
| | 13 To 18 | Bacteria; Firmicutes; Clostridia; | Otu000062 | 0.375712 | 2.33E−05 | 0.00032567 | 1.990728065 | 0.47056694 | Elevated |
| | | Clostridiales; Clostridiaceae_1; Clostridium_sensu_stricto | | | | | | | in food |
| | | | | | | | | | allergic group |
| | 13 To 18 | Bacteria; Firmicutes; Clostridia; | Otu000082 | 0.685072 | 0.002809375 | 0.016299676 | 1.221808873 | 0.4089243 | Elevated |
| | | Clostridiales; Lachnospiraceae; Blautia | | | | | | | in food |
| | | | | | | | | | allergic group |
| | 13 To 18 | Bacteria; Firmicutes; Clostridia; | Otu000052 | 0.181277 | 2.39E−06 | 5.65E−05 | 2.433901805 | 0.51595357 | Elevated |
| | | Clostridiales; Lachnospiraceae; Clostridium_XIVa | | | | | | | in food |
| | | | | | | | | | allergic group |
| | 13 To 18 | Bacteria; Firmicutes; Clostridia; | Otu000031 | 1 | 0.019735117 | 0.075707225 | 1.312314362 | 0.56289985 | Elevated |
| | | Clostridiales; Lachnospiraceae; Roseburia | | | | | | | in food |
| | | | | | | | | | allergic group |
| | 13 To 18 | Bacteria; Firmicutes; Clostridia; | Otu000036 | 0.739206 | 0.007261883 | 0.034092046 | 1.380851502 | 0.51436216 | Elevated |
| | | Clostridiales; Lachnospiraceae unclassified | | | | | | | in food |
| | | | | | | | | | allergic group |
| | 13 To 18 | Bacteria; Firmicutes; Clostridia; | Otu000042 | 1 | 0.006650541 | 0.032984538 | 1.484968037 | 0.54718046 | Elevated |
| | | Clostridiales; Ruminococcaceae; Ruminococcus | | | | | | | in food |
| | | | | | | | | | allergic group |
| | 13 To 18 | Bacteria; Firmicutes; Clostridia; | Otu00043 | 0.344761 | 1.91E−15 | 3.92E−13 | 4.260626919 | 0.53611824 | Elevated |
| | | Clostridiales; Ruminococcaceae; unclassified | | | | | | | in food |
| | | | | | | | | | allergic group |
| | 13 To 18 | Bacteria; Firmicutes; Erysipelotrichia; Erysipelotrichales; | Otu000049 | 0.425787 | 5.91E−13 | 6.06E−11 | 3.660394999 | 0.50820638 | Elevated |
| | | Erysipelotrichaceae unclassified | | | | | | | in food |
| | | | | | | | | | allergic group |
| | 13 To 18 | Bacteria; Firmicutes; Negativicutes; Selenomonadales; | Otu000003 | 0.274377 | 0.019950189 | 0.075707225 | 1.273540667 | 0.54722201 | Elevated |
| | | Veillonellaceae; Veillonella | | | | | | | in food |
| | | | | | | | | | allergic group |
| | 13 To 18 | Bacteria; Proteobacteria; Betaproteobacteria; | Otu000028 | 1 | 0.000883388 | 0.006707205 | 1.940314579 | 0.58350909 | Elevated |
| | | Burkholderiales; Sutterellaceae; Parasutterella | | | | | | | in food |
| | | | | | | | | | allergic group |
| | 13 To 18 | Bacteria; Proteobacteria; Gammaproteobacteria; | Otu000016 | 0.94753 | 0.017121567 | 0.067933959 | 1.205343535 | 0.50557873 | Elevated |
| | | Pasteurellales; Pasteurellaceae; unclassified | | | | | | | in food |
| | | | | | | | | | allergic group |
| | 13 To 18 | Bacteria; Verrucomicrobia; Verrucomicrobiae; | Otu000007 | 1 | 0.008663677 | 0.039177657 | 1.412386041 | 0.538042 | Elevated |
| | | Verrucomicrobiales; Verrucomicrobiaceae; Akkermansia | | | | | | | in food |
| | | | | | | | | | allergic group |
| Controls | 19 To 24 | Bacteria; Bacteroidetes; Bacteroidia; | Otu000038 | 0.983038 | 0.012729792 | 0.052192147 | −1.71740168 | 0.68937611 | Elevated |
| (n = 33) vs | | Bacteroidales; Bacteroidaceae; Bacteroides | | | | | | | in control |
| Food | | | | | | | | | group |
| Allergic | 19 To 24 | Bacteria; Firmicutes; Bacilli; | Otu000013 | 0.312224 | 0.010717103 | 0.046415622 | −1.573848798 | 0.61676262 | Elevated |
| (n = 24) | | Lactobacillales; Streptococcaceae; Streptococcus | | | | | | | in control |
| | | | | | | | | | group |
| | 19 To 24 | Bacteria; Firmicutes; Clostridia; | Otu000045 | 0.466853 | 0.027628612 | 0.091846467 | −1.311113913 | 0.59527906 | Elevated |
| | | Clostridiales; Lachnospiraceae; Blautia | | | | | | | in control |
| | | | | | | | | | group |
| | 19 To 24 | Bacteria; Firmicutes; Clostridia; | Otu000034 | 1 | 1.52E−05 | 0.000239692 | −2.46517666 | 0.56979639 | Elevated |
| | | Clostridiales; Lachnospiraceae; Clostridium_XIVa | | | | | | | in control |
| | | | | | | | | | group |
| | 19 To 24 | Bacteria; Firmicutes; Clostridia; | Otu000032 | 0.430722 | 1.99E−06 | 4.90E−05 | −2.31439115 | 0.4868233 | Elevated |
| | | Clostridiales; Lachnospiraceae; Clostridium_XIVa | | | | | | | in control |
| | | | | | | | | | group |
| | 19 To 24 | Bacteria; Firmicutes; Clostridia; | Otu000064 | 0.883838 | 7.94E−05 | 0.000887836 | −2.265947262 | 0.5742122 | Elevated |
| | | Clostridiales; Lachnospiraceae; Clostridium_XIVa | | | | | | | in control |
| | | | | | | | | | group |
| | 19 To 24 | Bacteria; Firmicutes; Clostridia; | Otu000055 | 1 | 0.000470451 | 0.003963389 | −2.064928208 | 0.59047886 | Elevated |
| | | Clostridiales; Lachnospiraceae; Clostridium_XIVa | | | | | | | in control |
| | | | | | | | | | group |
| | 19 To 24 | Bacteria; Firmicutes; Clostridia; | Otu000101 | 0.61158 | 0.018302102 | 0.071506793 | −1.508417883 | 0.63930941 | Elevated |
| | | Clostridiales; Lachnospiraceae unclassified | | | | | | | in control |
| | | | | | | | | | group |
| | 19 To 24 | Bacteria; Firmicutes; Clostridia; | Otu000022 | 0.794179 | 0.001232463 | 0.008063455 | −1.849982428 | 0.57252684 | Elevated |
| | | Clostridiales; Peptostreptococcaceae; Clostridium_XI | | | | | | | in control |
| | | | | | | | | | group |
| | 19 To 24 | Bacteria; Firmicutes; Clostridia; | Otu000050 | 0.847298 | 0.010959227 | 0.04713234 | −1.136215718 | 0.44662634 | Elevated |
| | | Clostridiales; Ruminococcaceae; Subdoligranulum | | | | | | | in control |
| | | | | | | | | | group |
| | 19 To 24 | Bacteria; Proteobacteria; Deltaproteobacteria; | Otu000068 | 1 | 0.006880289 | 0.033317935 | −1.600030909 | 0.59203753 | Elevated |
| | | Desulfovibrionales; Desulfovibrionaceae; Bilophila | | | | | | | in control |
| | | | | | | | | | group |
| | 19 To 24 | Bacteria; Actinobacteria; Actinobacteria; Bifidobacteriales; | Otu000004 | 0.315041 | 0.000397417 | 0.003532279 | 1.681768247 | 0.47483524 | Elevated |
| | | Bifidobacteriaceae; Bifidobacterium | | | | | | | in food |
| | | | | | | | | | allergic group |
| | 19 To 24 | Bacteria; Bacteroidetes; Bacteroidia; | Otu000040 | 1 | 0.0001707 | 0.001779331 | 2.659639027 | 0.7075682 | Elevated |
| | | Bacteroidales; Bacteroidaceae; Bacteroides | | | | | | | in food |
| | | | | | | | | | allergic group |
| | 19 To 24 | Bacteria; Bacteroidetes; Bacteroidia; | Otu000035 | 0.418803 | 0.002420081 | 0.014449998 | 2.04817611 | 0.67526173 | Elevated |
| | | Bacteroidales; Porphyromonadaceae; Parabacteroides | | | | | | | in food |
| | | | | | | | | | allergic group |
| | 19 To 24 | Bacteria; Bacteroidetes; Bacteroidia; | Otu000041 | 0.974259 | 0.01196073 | 0.04970762 | 1.750989958 | 0.69668937 | Elevated |
| | | Bacteroidales; Rikenellaceae; Alistipes | | | | | | | in food |
| | | | | | | | | | allergic group |
| | 19 To 24 | Bacteria; Firmicutes; Clostridia; | Otu000082 | 0.685072 | 0.000497564 | 0.00413516 | 2.048753035 | 0.58837309 | Elevated |
| | | Clostridiales; Lachnospiraceae; Blautia | | | | | | | in food |
| | | | | | | | | | allergic group |
| | 19 To 24 | Bacteria; Firmicutes; Clostridia; | Otu000099 | 1 | 0.00104955 | 0.007505503 | 2.075729284 | 0.63344375 | Elevated |
| | | Clostridiales; Lachnospiraceae; Dorea | | | | | | | in food |
| | | | | | | | | | allergic group |
| | 19 To 24 | Bacteria; Firmicutes; Clostridia; | Otu000067 | 0.344761 | 0.004188232 | 0.022288284 | 1.569751777 | 0.54816941 | Elevated |
| | | Clostridiales; Ruminococcaceae; unclassified | | | | | | | in food |
| | | | | | | | | | allergic group |
| | 19 To 24 | Bacteria; Firmicutes; Clostridia; | Otu000043 | 0.349307 | 0.000990192 | 0.00724962 | 2.459254913 | 0.74674516 | Elevated |
| | | Clostridiales; Ruminococcaceae; unclassified | | | | | | | in food |
| | | | | | | | | | allergic group |
| | 19 To 24 | Bacteria; Firmicutes; Erysipelotrichia; | Otu000049 | 0.425787 | 0.001819421 | 0.011078653 | 2.137381494 | 0.68544752 | Elevated |
| | | Erysipelotrichales; Erysipelotrichaceae unclassified | | | | | | | in food |
| | | | | | | | | | allergic group |
| | 19 To 24 | Bacteria; Firmicutes; Negativicutes; Selenomonadales; | Otu000037 | 1 | 0.00163676 | 0.01013758 | 2.094700866 | 0.66513745 | Elevated |
| | | Acidaminococcaceae; Phascolarctobacterium | | | | | | | in food |
| | | | | | | | | | allergic group |
| | 19 To 24 | Bacteria; Firmicutes; Negativicutes; | Otu0000023 | 1 | 0.02451234 | 0.084673375 | 1.37519272 | 0.61146786 | Elevated |
| | | Selenomonadales; Veillonellaceae; Dialister | | | | | | | in food |
| | | | | | | | | | allergic group |
| | 19 To 24 | Bacteria; Firmicutes; Negativicutes; | Otu000012 | 0.892415 | 0.000202127 | 0.001959765 | 2.747294159 | 0.73924661 | Elevated |
| | | Selenomonadales; Veillonellaceae; Veillonella | | | | | | | in food |
| | | | | | | | | | allergic group |
| | 19 To 24 | Bacteria; Proteobacteria; Betaproteobacteria; | Otu000028 | 1 | 1.36E−06 | 3.51E−05 | 2.988264692 | 0.61854184 | Elevated |
| | | Burkholderiales; Sutterellaceae; Parasutterella | | | | | | | in food |
| | | | | | | | | | allergic group |
| Controls | 25 To 30 | Bacteria; Bacteroidetes; | Otu000156 | 0.945399 | 0.004541224 | 0.023870536 | −3.02190124 | 1.0648393 | Elevated |
| (n = 9) vs | | Bacteroidia; Bacteroidales; Rikenellaceae; Alistipes | | | | | | | in control |
| Food | | | | | | | | | group |
| Allergic | 25 To 30 | Bacteria; Firmicutes; Clostridia; | Otu000030 | 0.976048 | 0.004199694 | 0.022288284 | −3.129927105 | 1.09332526 | Elevated |
| (n = 15) | | Clostridiales; Lachnospiraceae; Blautia | | | | | | | in control |
| | | | | | | | | | group |
| | 25 To 30 | Bacteria; Firmicutes; Clostridia; | Otu000052 | 0.181277 | 0.000259099 | 0.002414332 | −4.099438046 | 1.12218246 | Elevated |
| | | Clostridiales; Lachnospiraceae; Clostridium_XIVa | | | | | | | in control |
| | | | | | | | | | group |
| | 25 To 30 | Bacteria; Firmicutes; Clostridia; | Otu000011 | 1 | 0.025491337 | 0.086138309 | −2.372337233 | 1.06198325 | Elevated |
| | | Clostridiales; Lachnospiraceae unclassified | | | | | | | in control |
| | | | | | | | | | group |
| | 25 To 30 | Bacteria; Firmicutes; Clostridia; | Otu000065 | 0.176032 | 0.001613621 | 0.010126295 | −3.311556472 | 1.05014315 | Elevated |
| | | Clostridiales; Peptostreptococcaceae; Clostridium_XI | | | | | | | in control |
| | | | | | | | | | group |
| | 25 To 30 | Bacteria; Firmicutes; Clostridia; | Otu000096 | 1 | 0.001124447 | 0.00767812 | −3.206355025 | 0.98433472 | Elevated |
| | | Clostridiales; Ruminococcaceae; Oscillibacter | | | | | | | in control |
| | | | | | | | | | group |
| | 25 To 30 | Bacteria; Firmicutes; Clostridia; | Otu000050 | 0.847298 | 0.019233627 | 0.074394218 | −2.134849913 | 0.91195035 | Elevated |
| | | Clostridiales; Ruminococcaceae; Subdoligranulum | | | | | | | in control |
| | | | | | | | | | group |
| | 25 To 30 | Bacteria; Firmicutes; Clostridia; | Otu000067 | 0.349307 | 3.58E−06 | 7.59E−05 | −5.330500773 | 1.1502275 | Elevated |
| | | Clostridiales; Ruminococcaceae; unclassified | | | | | | | in control |
| | | | | | | | | | group |
| | 25 To 30 | Bacteria; Firmicutes; Erysipelotrichia; | Otu000026 | 0.747721 | 0.017528416 | 0.069102409 | −2.48867778 | 1.04767371 | Elevated |
| | | Erysipelotrichales; Erysipelotrichaceae; Clostridium_XVIII | | | | | | | in control |
| | | | | | | | | | group |
| | 25 To 30 | Bacteria; Firmicutes; Negativicutes; | Otu000023 | 1 | 7.27E−06 | 0.000132489 | 4.397863441 | 0.98040561 | Elevated |
| | | Selenomonadales; Veillonellaceae; Dialister | | | | | | | in control |
| | | | | | | | | | group |
| | 25 To 30 | Bacteria; Proteobacteria; Gammaproteobacteria; | Otu000005 | 0.143807 | 3.02E−06 | 6.88E−05 | 5.080957218 | 1.08817588 | Elevated |
| | | Enterobacteriales; Enterobacteriaceae; Escherichia/Shigella | | | | | | | in control |
| | | | | | | | | | group |
| | 25 To 30 | Bacteria; Bacteroidetes; Bacteroidia; | Otu000111 | 0.682567 | 0.001320438 | 0.008548099 | 3.462133078 | 1.07804226 | Elevated |
| | | Bacteroidales; Porphyromonadaceae; Butyricimonas | | | | | | | in food |
| | | | | | | | | | allergic group |
| | 25 To 30 | Bacteria; Bacteroidetes; | Otu000086 | 1 | 1.11E−05 | 0.0001845 | 5.174672394 | 1.17737066 | Elevated |
| | | Bacteroidia; Bacteroidales; Prevotellaceae; Prevotella | | | | | | | in food |
| | | | | | | | | | allergic group |
| | 25 To 30 | Bacteria; Firmicutes; Clostridia; | Otu000031 | 1 | 0.006301192 | 0.03150596 | 3.104089798 | 1.13632911 | Elevated |
| | | Clostridiales; Lachnospiraceae; Roseburia | | | | | | | in food |
| | | | | | | | | | allergic group |
| | 25 To 30 | Bacteria; Firmicutes; Clostridia; | Otu000061 | 0.822466 | 0.000203943 | 0.001959765 | 4.000610977 | 1.07714672 | Elevated |
| | | Clostridiales; Lachnospiraceae; Ruminococcus2 | | | | | | | in food |
| | | | | | | | | | allergic group |
| | 25 To 30 | Bacteria; Firmicutes; Clostridia; | Otu000069 | 0.739206 | 0.005730174 | 0.029367142 | 2.849512801 | 1.0313728 | Elevated |
| | | Clostridiales; Lachnospiraceae unclassified | | | | | | | in food |
| | | | | | | | | | allergic group |
| | 25 To 30 | Bacteria; Firmicutes; Clostridia; | Otu000036 | 0.778839 | 0.004755742 | 0.024786282 | 3.177062787 | 1.12536931 | Elevated |
| | | Clostridiales; Lachnospiraceae unclassified | | | | | | | in food |
| | | | | | | | | | allergic group |
| | 25 To 30 | Bacteria; Firmicutes; Clostridia; | Otu000053 | 1 | 0.001024965 | 0.007415923 | 3.499808332 | 1.06585042 | Elevated |
| | | Clostridiales; Lachnospiraceae unclassified | | | | | | | in food |
| | | | | | | | | | allergic group |
| | 25 To 30 | Bacteria; Firmicutes; Clostridia; | Otu000077 | 1 | 7.54E−06 | 0.000132489 | 4.478225722 | 1.0000825 | Elevated |
| | | Clostridiales; Lachnospiraceae unclassified | | | | | | | in food |
| | | | | | | | | | allergic group |
| | 25 To 30 | Bacteria; Firmicutes; Negativicutes; | Otu000046 | 0.638876 | 0.000528016 | 0.004272761 | 3.563052489 | 1.02796399 | Elevated |
| | | Selenomonadales; Veillonellaceae; Megamonas | | | | | | | in food |
| | | | | | | | | | allergic group |
| | 25 To 30 | Bacteria; Firmicutes; Negativicutes; Selenomonadales; | Otu000073 | 0.377832 | 0.000196175 | 0.001959765 | 3.592886987 | 0.96482035 | Elevated |
| | | Veillonellaceae; Megasphaera | | | | | | | in food |
| | | | | | | | | | allergic group |

TABLE 7 shows differences in bacterial genera between FA and control subjects. 16S rDNA sequencing data using DESeq2 software package showing significant log 2 fold differences (using DESeq2 software package) in a total of 77 OTUs in FA versus control subjects, some followed serially over time. Key covariates of interest (breastfeeding for subjects younger than 19 months, gender, mode of delivery, and breastfeeding) were controlled for using the multi-factorial model in DESeq2. P-values were adjusted for multiple hypothesis testing using the method of Benjamini and Hochberg (BH). FDR: false discovery rate. 1fcSE: Log-fold change Standard Error. Pplacer-derived like-weight_ratio values shown for each OTU. OTUs reported met the following criteria: (1) adjusted p-value ≤0.1; (2) absolute value of log 2 fold change ≥2. Subjects were subdivided into different age groups (1-6 months 7-12 months-13-18 months, 19-24 months and 25-30 months). Negative log 2 fold change values represent higher abundance in control subjects, and positive log 2 fold change values represent higher abundance in food allergic subjects. Some OTU changes persisted across several age group while others were only age dependent.

TABLE 8

| Differences in bacterial genera between FA and control subjects consuming Cow's Milk Proteins | | | | | | | | | |
|---|---|---|---|---|---|---|---|---|---|
| | | | | Like− | | | | | |
| | Age | | | weight | | | log2 Fold | Ifc | |
| Comparisons | Groups | Lineage | OTU_ID | ratio | P−value | FDR | Change | SE | Comments |
| Controls | 7 To 12 | Bacteria; Actinobacteria; Actinobacteria; Bifidobacteriales; | Otu000004 | 0.315041 | 0.028861404 | 0.09441364 | 1.70425631 | 0.77984637 | Elevated in |
| (n = 40) vs | | Bifidobacteriaceae; Bifidobacterium | | | | | | | control group |
| Food | 7 To 12 | Bacteria; Actinobacteria; Actinobacteria; Coriobacteriales; | Otu000112 | 0.675625 | 0.020392096 | 0.07570722 | −2.075014876 | 0.89476772 | Elevated in |
| Allergic | | Coriobacteriaceae; Eggerthella | | | | | | | control group |
| (n = 6) | 7 To 12 | Bacteria; Bacteroidetes; Bacteroidia; Bacteroidales; | Otu000018 | 1 | 0.000518029 | 0.00424784 | 3.939836243 | 1.1349885 | Elevated in |
| | | Bacteroidaceae; Bacteroides | | | | | | | control group |
| | 7 To 12 | Bacteria; Firmicutes; Bacilli; Lactobacillales; | Otu000060 | 0.214057 | 0.00712793 | 0.03409205 | 2.493119264 | 0.92653199 | Elevated in |
| | | Enterococcaceae; Enterococcus | | | | | | | control group |
| | 7 To 12 | Bacteria; Firmicutes; Bacilli; Lactobacillales; | Otu000051 | 0.143529 | 0.008744205 | 0.03925318 | −2.132589919 | 0.81337693 | Elevated in |
| | | Streptococcaceae; Streptococcus | | | | | | | control group |
| | 7 To 12 | Bacteria; Firmicutes; Clostridia; Clostridiales; | Otu000017 | 0.341747 | 0.006795068 | 0.03331793 | 3.202061905 | 1.18300265 | Elevated in |
| | | Lachnospiraceae unclassified | | | | | | | control group |
| | 7 To 12 | Bacteria; Firmicutes; Negativicutes; | Otu000023 | 1 | 0.001230984 | 0.00806345 | 3.659269115 | 1.13233909 | Elevated in |
| | | Selenomonadales; Veillonellaceae; Dialister | | | | | | | control group |
| | 7 To 12 | Bacteria; Firmicutes; Negativicutes; | Otu000012 | 0.892415 | 3.56E−06 | 7.59E−05 | −6.537912484 | 1.41046112 | Elevated in |
| | | Selenomonadales; Veillonellaceae; Veillonella | | | | | | | control group |
| | 7 To 12 | Bacteria; Proteobacteria; Gammaproteobacteria; | Otu000016 | 0.94753 | 0.007240825 | 0.03409205 | 2.897325042 | 1.07512937 | Elevated in |
| | | Pasteurellales; Pasteurellaceae unclassified | | | | | | | control group |
| | 7 To 12 | Bacteria; Verrucomicrobia; Verrucomicrobiae; | Otu000007 | 1 | 5.46E−05 | 0.00067158 | 4.963841416 | 1.23025196 | Elevated in |
| | | Verrucomicrobiales; Verrucomicrobiaceae; Akkermansia | | | | | | | control group |
| | 7 To 12 | Bacteria; Bacteroidetes; Bacteroidia; | Otu000001 | 0.523821 | 0.024644771 | 0.08467338 | 2.640721822 | 1.17525993 | Elevated in food |
| | | Bacteroidales; Bacteroidaceae; Bacteroides | | | | | | | allergic group |
| | 7 To 12 | Bacteria; Bacteroidetes; Bacteroidia; | Otu000040 | 1 | 7.73E−12 | 5.28E−10 | 7.749867985 | 1.13244333 | Elevated in food |
| | | Bacteroidales; Bacteroidaceae; Bacteroides | | | | | | | allergic group |
| | 7 To 12 | Bacteria; Bacteroidetes; Bacteroidia; | Otu000008 | 1 | 6.18E−06 | 0.00011877 | 4.84488542 | 1.07182755 | Elevated in food |
| | | Bacteroidales; Prevotellaceae; Prevotella | | | | | | | allergic group |
| | 7 To 12 | Bacteria; Firmicutes; Clostridia; Clostridiales; | Otu000027 | 0.336472 | 0.025429169 | 0.08613831 | 2.220613554 | 0.99364314 | Elevated in food |
| | | Clostridiaceae_1; Clostridium_sensu_stricto | | | | | | | allergic group |
| | 7 To 12 | Bacteria; Firmicutes; Clostridia; | Otu000080 | 0.268673 | 0.019931507 | 0.07570722 | 2.446459532 | 1.05104959 | Elevated in food |
| | | Clostridiales; Lachnospiraceae; Blautia | | | | | | | allergic group |
| | 7 To 12 | Bacteria; Firmicutes; Clostridia; | Otu000039 | 1 | 3.42E−08 | 1.24E−06 | 5.082626334 | 0.92105838 | Elevated in food |
| | | Clostridiales; Lachnospiraceae; Lachnospira | | | | | | | allergic group |
| | 7 To 12 | Bacteria; Firmicutes; Clostridia; | Otu000014 | 0.946 | 0.000170403 | 0.00177933 | 4.407125202 | 1.17233229 | Elevated in food |
| | | Clostridiales; Lachnospiraceae; Roseburia | | | | | | | allergic group |
| | 7 To 12 | Bacteria; Firmicutes; Clostridia; | Otu000036 | 0.739206 | 0.020617232 | 0.07570722 | 2.231354768 | 0.96390086 | Elevated in food |
| | | Clostridiales; Lachnospiraceaeunclassified | | | | | | | allergic group |
| Controls | 7 To 12 | Bacteria; Proteobacteria; Deltaproteobacteria; | Otu000068 | 1 | 0.000880247 | 0.00670721 | 3.402745 | 1.02299913 | Elevated in food |
| (n = 53) vs | | Desulfovibrionales; Desulfovibrionaceae; Bilophila | | | | | | | allergic group |
| Food | 13 To 18 | Bacteria; Bacteroidetes; Bacteroidia; Bacteroidales; | Otu000035 | 0.418803 | 0.00843477 | 0.03842506 | −2.279776528 | 0.86546883 | Elevated in |
| Allergic | | Porphyromonadaceae; Parabacteroides | | | | | | | control group |
| (n = 17) | 13 To 18 | Bacteria; Firmicutes; Clostridia; | Otu000070 | 1 | 0.02192563 | 0.07834823 | 1.371261108 | 0.59837185 | Elevated in |
| | | Clostridiales; Lachnospiraceae; Clostridium_XIVa | | | | | | | control group |
| | 13 To 18 | Bacteria; Firmicutes; Clostridia; | Otu000095 | 0.5434131 | 0.022039422 | 0.07834823 | −1.458042207 | 0.63678647 | Elevated in |
| | | Clostridiales; Lachnospiraceae; Dorea | | | | | | | control group |
| | 13 To 18 | Bacteria; Firmicutes; Clostridia; | Otu000039 | 1 | 0.000201237 | 0.00195976 | −2.859589255 | 0.76921068 | Elevated in |
| | | Clostridiales; Lachnospiraceae; Lachnospira | | | | | | | control group |
| | 13 To 18 | Bacteria; Firmicutes; Clostridia; | Otu000059 | 0.699866 | 1.34E−05 | 0.00021687 | 3.105394755 | 0.7132062 | Elevated in |
| | | Clostridiales; Lachnospiraceae unclassified | | | | | | | control group |
| | 13 To 18 | Bacteria; Firmicutes; Clostridia; | Otu000085 | 0.470157 | 0.015970095 | 0.06419352 | −1.35790557 | 0.56354024 | Elevated in |
| | | Clostridiales; Lachnospiraceae unclassified | | | | | | | control group |
| | 13 To 18 | Bacteria; Firmicutes; Clostridia; | Otu000050 | 0.847298 | 0.004203969 | 0.02228828 | 1.742759233 | 0.6088376 | Elevated in |
| | | Clostridiales; Ruminococcaceae; Subdoligranulum | | | | | | | control group |
| | 13 To 18 | Bacteria; Firmicutes; Clostridia; | Otu000029 | 0.714544 | 0.011566122 | 0.04905631 | 1.69374541 | 0.67076034 | Elevated in |
| | | Clostridiales; Ruminococcaceae; unclassified | | | | | | | control group |
| | 13 To 18 | Bacteria; Proteobacteria; Betaproteobacteria; | Otu000074 | 1 | 0.00037532 | 0.00339444 | −2.795718243 | 0.78600826 | Elevated in |
| | | Burkholderiales; Sutterellaceae; Sutterella | | | | | | | control group |
| | 13 To 18 | Bacteria; Proteobacteria; Deltaproteobacteria; | Otu000068 | 1 | 0.000421266 | 0.00364899 | 2.414225365 | 0.6846166 | Elevated in |
| | | Desulfovibrionales; Desulfovibrionaceae; Bilophila | | | | | | | control group |
| | 13 To 18 | Bacteria; unclassified; unclassified; unclassified; | Otu000081 | 0.789286 | 0.000226012 | 0.00213842 | 2.598020946 | 0.70445001 | Elevated in |
| | | unclassified; unclassified | | | | | | | control group |
| | 13 To 18 | Bacteria; Bacteroidetes; Bacteroidia; Bacteroidales; | Otu000044 | 0.577093 | 2.77E−05 | 0.00037034 | 3.063649345 | 0.73090622 | Elevated in food |
| | | Bacteroidaceae; Bacteroides | | | | | | | allergic group |
| | 13 To 18 | Bacteria; Bacteroidetes; Bacteroidia; Bacteroidales; | Otu000025 | 0.931276 | 0.009858812 | 0.04330835 | 2.083046663 | 0.80676269 | Elevated in food |
| | | Rikenellaceae; Alistipes | | | | | | | allergic group |
| | 13 To 18 | Bacteria; Bacteroidetes; Bacteroidia; Bacteroidales; | Otu000041 | 0.974259 | 3.71E−06 | 7.61E−05 | 3.426561947 | 0.74054421 | Elevated in food |
| | | Rikenellaceae; Alistipes | | | | | | | allergic group |
| | 13 To 18 | Bacteria; Firmicutes; Clostridia; Clostridiales; | Otu000062 | 0.375712 | 0.020608278 | 0.07570722 | 1.469383995 | 0.63469979 | Elevated in food |
| | | Clostridiaceae_l; Clostridium_sensu_stricto | | | | | | | allergic group |
| | 13 To 18 | Bacteria; Firmicutes; Clostridia; | Otu000080 | 0.268673 | 0.006877404 | 0.03331793 | 1.693717511 | 0.62667078 | Elevated in food |
| | | Clostridiales; Lachnospiraceae; Blautia | | | | | | | allergic group |
| | 13 To 18 | Bacteria; Firmicutes; Clostridia; | Otu000031 | 1 | 0.010681116 | 0.04641562 | 1.874759738 | 0.73434691 | Elevated in food |
| | | Clostridiales; Lachnospiraceae; Roseburia | | | | | | | allergic group |
| | 13 To 18 | Bacteria; Firmicutes; Erysipelotrichia; Erysipelotrichales; | Otu000084 | 0.330651 | 0.016571966 | 0.06618025 | 1.561616933 | 0.6517412 | Elevated in food |
| | | Erysipelotrichaceae; Clostridium_XVIII | | | | | | | allergic group |
| | 13 To 18 | Bacteria; Firmicutes; Erysipelotrichia; Erysipelotrichales; | Otu000049 | 0.425787 | 3.05E−11 | 1.71E−09 | 4.956164931 | 0.74592831 | Elevated in food |
| | | Erysipelotrichaceae; unclassified | | | | | | | allergic group |
| | 13 To 18 | Bacteria; Firmicutes; Negativicutes; Selenomonadales; | Otu000003 | 0.274377 | 0.000904032 | 0.00678024 | 1.561616933 | 0.69646872 | Elevated in food |
| | | Veillonellaceae; Veillonella | | | | | | | allergic group |
| | 13 To 18 | Bacteria; Proteobacteria; Betaproteobacteria; | Otu000028 | 1 | 7.17E−05 | 0.00083199 | 3.168278495 | 0.797934 | Elevated in food |
| | | Burkholderiales; Sutterellaceae; Parasutterella | | | | | | | allergic group |
| Controls | 13 To 18 | Bacteria; Proteobacteria; Gammaproteobacteria; | Otu000016 | 0.94753 | 0.011962159 | 0.04970762 | 1.650222931 | 0.65660688 | Elevated in food |
| (n = 33) vs | | Pasteurellales; Pasteurellaceae unclassified | | | | | | | allergic group |
| Food | 19 to 24 | Bacteria; Firmicutes; Clostridia; | Otu000045 | 0.466853 | 0.020089738 | 0.07570722 | 1.650493987 | 0.70999137 | Elevated in food |
| Allergic | | Clostridiales; Lachnospiraceae; Blautia | | | | | | | allergic group |
| (n = 16) | 19 to 24 | Bacteria; Firmicutes; Clostridia; Clostridiales; | Otu000034 | 1 | 2.48E−05 | 0.00033893 | 2.801922554 | 0.66446875 | Elevated in food |
| | | Lachnospiraceae; Clostridium_XIVa | | | | | | | allergic group |
| | 19 to 24 | Bacteria; Firmicutes; Clostridia; | Otu000032 | 0.430722 | 0.000151433 | 0.00163388 | −2.272311045 | 0.59975877 | Elevated in |
| | | Clostridiales; Lachnospiraceae; Clostridium_XIVa | | | | | | | control group |
| | 19 to 24 | Bacteria; Firmicutes; Clostridia; | Otu000064 | 0.883838 | 0.001389574 | 0.00890196 | 2.148980946 | 0.67222711 | Elevated in |
| | | Clostridiales; Lachnospiraceae; Clostridium_XIVa | | | | | | | control group |
| | 19 to 24 | Bacteria; Firmicutes; Clostridia; | Otu000055 | 1 | 0.008385287 | 0.03842506 | 1.846927599 | 0.70061575 | Elevated in |
| | | Clostridiales; Lachnospiraceae; Clostridium_XIVa | | | | | | | control group |
| | 19 to 24 | Bacteria; Firmicutes; Clostridia; | Otu000061 | 0.822466 | 0.012558636 | 0.05183598 | 1.867545076 | 0.74820142 | Elevated in |
| | | Clostridiales; Lachnospiraceae; Ruminococcus2 | | | | | | | control group |
| | 19 to 24 | Bacteria; Firmicutes; Clostridia; | Otu000101 | 0.437782 | 0.006028732 | 0.0306419 | 2.113515557 | 0.7696105 | Elevated in |
| | | Clostridiales; Lachnospiraceae unclassified | | | | | | | control group |
| | 19 to 24 | Bacteria; Firmicutes; Clostridia; | Otu000063 | 0.470157 | 0.014606506 | 0.05909869 | 1.994711915 | 0.81683832 | Elevated in |
| | | Clostridiales; Lachnospiraceae unclassified | | | | | | | control group |
| | 19 to 24 | Bacteria; Firmicutes; Clostridia; | Otu000085 | 0.61158 | 0.01187688 | 0.04970762 | 1.517303607 | 0.60311423 | Elevated in |
| | | Clostridiales; Lachnospiraceae unclassified | | | | | | | control group |
| | 19 to 24 | Bacteria; Firmicutes; Clostridia; Clostridiales; | Otu000022 | 0.794179 | 0.012944452 | 0.05272078 | 1.660366323 | 0.66807645 | Elevated in |
| | | Peptostreptococcaceae; Clostridium_XI | | | | | | | control group |
| | 19 to 24 | Bacteria; Firmicutes; Clostridia; | Otu000050 | 0.847298 | 0.021493271 | 0.07821516 | 1.29358978 | 0.56262468 | Elevated in |
| | | Clostridiales; Ruminococcaceae; Subdoligranulum | | | | | | | control group |
| | 19 to 24 | Bacteria; Firmicutes; Clostridia; | Otu000029 | 0.714544 | 0.002846215 | 0.01635909 | 2.472879117 | 0.8287471 | Elevated in |
| | | Clostridiales; Ruminococcaceae; unclassified | | | | | | | control group |
| | 19 to 24 | Bacteria; Proteobacteria; Deltaproteobacteria; | Otu000068 | 1 | 4.56E−05 | 0.00058425 | −2.849006342 | 0.69874315 | Elevated in |
| | | Desulfovibrionales; Desulfovibrionaceae; Bilophila | | | | | | | control group |
| | 19 to 24 | Bacteria; Proteobacteria; Gammaproteobacteria; | Otu000005 | 0.143807 | 0.023630731 | 0.08210678 | 1.768329135 | 0.78138148 | Elevated in |
| | | Enterobacteriales; Enterobacteriaceae; Escherichia/Shigella | | | | | | | control group |
| | 19 to 24 | Bacteria; Actinobacteria; Actinobacteria; | Otu000004 | 0.315041 | 0.000937758 | 0.00694845 | 1.824894351 | 0.5515669 | Elevated in food |
| | | Bifidobacteriales; Bifidobacteriaceae; Bifidobacterium | | | | | | | allergic group |
| | 19 to 24 | Bacteria; Firmicutes; Clostridia; | Otu000053 | 0.778839 | 0.009257642 | 0.04096007 | 1.790024407 | 0.68783856 | Elevated in food |
| | | Clostridiales; Lachnospiraceae unclassified | | | | | | | allergic group |
| | 19 to 24 | Bacteria; Firmicutes; Erysipelotrichia; Erysipelotrichales; | Otu000049 | 0.425787 | 0.001945767 | 0.01173183 | 2.535037032 | 0.8181794 | Elevated in food |
| | | Erysipelotrichaceae; unclassified | | | | | | | allergic group |
| | 19 to 24 | Bacteria; Firmicutes; Negativicutes; | Otu000037 | 1 | 0.001113474 | 0.00767812 | 2.560878234 | 0.78550584 | Elevated in food |
| | | Selenomonadales; Acidaminococcaceae; Phascolarctobacterium | | | | | | | allergic group |
| Controls | 19 to 24 | Bacteria; Proteobacteria; Betaproteobacteria; | Otu000028 | 1 | 1.28E−07 | 4.37E−06 | 3.901064929 | 0.73862549 | Elevated in food |
| (n = 9) vs | | Burkholderiales; Sutterellaceae; Parasutterella | | | | | | | allergic group |
| Food | 25 To 30 | Bacteria; Bacteroidetes; | Otu000041 | 0.974259 | 0.028205348 | 0.09323415 | 2.887473076 | 1.31582617 | Elevated in |
| Allergic | | Bacteroidia; Bacteroidales; Rikenellaceae; Alistipes | | | | | | | control group |
| (n = 11) | 25 To 30 | Bacteria; Bacteroidetes; | Otu000156 | 0.945399 | 0.020680998 | 0.07570722 | 2.656885426 | 1.14829873 | Elevated in |
| | | Bacteroidia; Bacteroidales; Rikenellaceae; Alistipes | | | | | | | control group |
| | 25 To 30 | Bacteria; Firmicutes; Clostridia; | Otu000030 | 0.976048 | 0.004906245 | 0.0253558 | −3.38075013 | 1.20177709 | Elevated in |
| | | Clostridiales; Lachnospiraceae; Blautia | | | | | | | control group |
| | 25 To 30 | Bacteria; Firmicutes; Clostridia; | Otu000052 | 0.181277 | 0.001089655 | 0.0076152 | 4.030736152 | 1.23404008 | Elevated in |
| | | Clostridiales; Lachnospiraceae; Clostridium_XIVa | | | | | | | control group |
| | 25 To 30 | Bacteria; Firmicutes; Clostridia; Clostridiales; | Otu000065 | 0.176032 | 0.008932133 | 0.03980624 | 3.005021921 | 1.14930582 | Elevated in |
| | | Peptostreptococcaceae; Clostridium_XI | | | | | | | control group |
| | 25 To 30 | Bacteria; Firmicutes; Clostridia; | Otu000096 | 1 | 0.007212722 | 0.03409205 | −2.89018863 | 1.07567526 | Elevated in |
| | | Clostridiales; Ruminococcaceae; Oscillibacter | | | | | | | control group |
| | 25 To 30 | Bacteria; Firmicutes; Clostridia; | Otu000050 | 0.847298 | 0.020297822 | 0.07570722 | 2.355697715 | 1.0150384 | Elevated in |
| | | Clostridiales; Ruminococcaceae; Subdoligranulum | | | | | | | control group |
| | 25 To 30 | Bacteria; Firmicutes; Clostridia; | Otu000067 | 0.349307 | 7.52E−06 | 0.00013249 | 5.771812992 | 1.28878057 | Elevated in |
| | | Clostridiales; Ruminococcaceae; unclassified | | | | | | | control group |
| | 25 To 30 | Bacteria; Firmicutes; Erysipelotrichia; | Otu000026 | 0.747721 | 0.030303437 | 0.09757389 | −2.510482667 | 1.15898667 | Elevated in |
| | | Erysipelotrichales; Erysipelotrichaceae; Clostridium_XVIII | | | | | | | control group |
| | 25 To 30 | Bacteria; Firmicutes; Negativicutes; | Otu000023 | 1 | 6.73E−05 | 0.00079595 | 4.642569317 | 1.16478774 | Elevated in |
| | | Selenomonadales; Veillonellaceae; Dialister | | | | | | | control group |
| | 25 To 30 | Bacteria; Proteobacteria; Gammaproteobacteria; | Otu000005 | 0.143807 | 0.000462676 | 0.00395202 | 4.234972932 | 1.20947973 | Elevated in |
| | | Enterobacteriales; Enterobacteriaceae; Escherichia/Shigella | | | | | | | control group |
| | 25 To 30 | Bacteria; Bacteroidetes; Bacteroidia; Bacteroidales; | Otu000111 | 0.682567 | 2.24E−05 | 0.00032037 | 4.865154789 | 1.14753941 | Elevated in food |
| | | Porphyromonadaceae; Butyricimonas | | | | | | | allergic group |
| | 25 To 30 | Bacteria; Bacteroidetes; Bacteroidia; Bacteroidales; | Otu000086 | 1 | 2.63E−07 | 7.70E−06 | 6.317417378 | 1.2271563 | Elevated in food |
| | | Prevotellaceae; Prevotella | | | | | | | allergic group |
| | 25 To 30 | Bacteria; Firmicutes; Clostridia; | Otu000031 | 1 | 0.003327392 | 0.01877382 | 3.6162044386 | 1.2317809 | Elevated in food |
| | | Clostridiales; Lachnospiraceae; Roseburia | | | | | | | allergic group |
| | 25 To 30 | Bacteria; Firmicutes; Clostridia; | Otu000036 | 0.739206 | 0.00832908 | 0.03842506 | 3.241749445 | 1.22866589 | Elevated in food |
| | | Clostridiales; Lachnospiraceae unclassified | | | | | | | allergic group |
| | 25 To 30 | Bacteria; Firmicutes; Clostridia; | Otu000053 | 0.778839 | 0.000402048 | 0.00353228 | 4.095714721 | 1.1573947 | Elevated in food |
| | | Clostridiales; Lachnospiraceae unclassified | | | | | | | allergic group |
| | 25 To 30 | Bacteria; Firmicutes; Clostridia; | Otu000077 | 1 | 5.84E−05 | 0.00070424 | 4.404207069 | 1.09584907 | Elevated in food |
| | | Clostridiales; Lachnospiraceae unclassified | | | | | | | allergic group |
| | 25 To 30 | Bacteria; Proteobacteria; Gammaproteobacteria; | Otu000114 | 0.604395 | 0.028349247 | 0.09323415 | 2.328152002 | 1.06191003 | Elevated in food |
| | | Pasteurellales; Pasteurellaceae unclassified | | | | | | | allergic group |

TABLE 8 shows differences in bacterial genera between FA and control subjects consuming Cow's Milk Proteins. 16S rDNA sequencing data in milk consuming subjects showing significant log 2 fold differences (using DESeq2 software package) of selected OTUs in FA (but milk tolerant) versus control subjects, some followed serially overtime. Subjects were subdivided into different age groups (1-6 months 7-12 months-13-18 months, 19-24 months and 25-30 months). Key covariates of interest (breastfeeding for subjects younger than 19 months, gender, mode of delivery, and breastfeeding) were controlled for using the multi-factorial model in DESeq2. Pplacer like-weight_ratio values shown for each OTU. P-values were adjusted for multiple hypothesis testing using the method of Benjamini and Hochberg (BH) (57). FDR: false discovery rate. 1fcSE: Log-fold change Standard Error. OTUs reported met the following criteria: (1) adjusted p-value ≤0.1; (2) absolute value of log 2 fold change ≥2. Negative log 2 fold change values represent higher abundance in control subjects, and positive log 2 fold change values represent higher abundance in food allergic subjects. Some OTU changes persisted across several age group while others were only age dependent.

TABLE 9
Strain composition of the bacterial consortia and their respective growth conditions.

| | | | | | Culture |
|---|---|---|---|---|---|
| Species | Strain# | Consortium | Culture Media | Temperature | Time |
| Bacteroides fragilis | ATCC | Bacteroidetes | BHI | 37° C. | 24 hours |
| | 25285 | | | | |
| Bacteroides ovatus | ATCC | Bacteroidetes | BHI | 37° C. | 24 hours |
| | 8483 | | | | |
| Bacteroides | ATCC | Bacteroidetes | BHI | 37° C. | 24 hours |
| vulgatus | 8482 | | | | |
| Bilophila | ATCC | Proteobacteria | BHI | 37° C. | 96 hours |
| wadsworthia | 49620 | | | | |
| Clostridium | ATCC | Clostridiales | BHI | 37° C. | 24 hours |
| bifermentans | 638 | | | | |
| Clostridium | DSM | Clostridiales | BHIS | 37° C. | 24 hours |
| hiranonis | 13275 | | | | |
| Clostridium leptum | ATCC | Clostridiales | Peptone-Yeast | 37° C. | 72 hours |
| | 29065 | | with Maltose | | |
| Clostridium | ATCC | Clostridiales | BHIS | 37° C. | 24 hours |
| ramosum | 25582 | | | | |
| Clostridium | ATCC | Clostridiales | BHIS | 37° C. | 24 hours |
| sardiniense | 27555 | | | | |
| Clostridium | ATCC | Clostridiales | BHIS | 37° C. | 24 hours |
| scindens | 35704 | | | | |
| Escherichia coli | Nissle | Proteobacteria | TSB | 37° C. | 12 hours |
| Klebsiella oxytoca | ATCC | Proteobacteria | TSB | 37° C. | 12 hours |
| | 700324 | | | | |
| Parabacteroides | ATCC | Bacteroidetes | BHI | 37° C. | 24 hours |
| distasonis | 8503 | | | | |
| Prevotella | ATCC | Bacteroidetes | BHI | 37° C. | 48 hours |
| melaninogenica | 25845 | | | | |
| Proteus mirabilis | ATCC | Proteobacteria | TSB | 37° C. | 12 hours |
| | 29906 | | | | |

TABLE 9 shows strain composition of the bacterial consortia and their respective growth conditions. BHI—Brain Heart Infusion with vitamin K (1.0 ug/mL), hemin (0.5 mg/mL) an 0.05% cysteine hydrochloride; BHIS—BHI (per above) with 0.5% yeast extract (Sigma™, St. Louis, Mo.); Peptone-Yeast with Maltose—Anaerobe Systems Inc™ (Morgan Hill, Calif.).

TABLE 10
SCFA production by the bacterial consortia in GF Il4ra^{F709} mice.

| Mouse Sample | | | | | | |
|---|---|---|---|---|---|---|
| (GF Il4ra^{F709} + | Acetate | Isobutyrate | Butyrate | Isovalerate | Isocaproate | Propionate |
| OVA/SEB) | (mM/g) | (mM/g) | (mM/g) | (mM/g) | (mM/g) | (mM/g) |
| No Bacteria 1 | N.D. | | | | | |
| No Bacteria 2 | N.D. | | | | | |
| No Bacteria 3 | N.D. | | | | | |
| No Bacteria 4 | N.D. | | | | | |
| No Bacteria 5 | N.D. | | | | | |
| +Clostridiales 1 | 5.687 | N.D. | N.D. | N.D. | N.D. | N.D. |
| +Clostridiales 2 | 18.844 | 0.578 | 0.762 | 0.471 | 0.529 | N.D. |
| +Clostridiales 3 | 19.147 | 0.542 | 0.594 | 0.790 | 0.697 | N.D. |
| +Clostridiales 4 | 11.535 | N.D. | N.D. | N.D. | N.D. | N.D. |
| +Proteobacteria 1 | 14.053 | N.D. | N.D. | N.D. | N.D. | N.D. |
| +Proteobacteria 2 | 19.145 | N.D. | N.D. | N.D. | N.D. | N.D. |
| +Proteobacteria 3 | 17.113 | N.D. | N.D. | N.D. | N.D. | N.D. |
| +Proteobacteria 4 | 17.377 | N.D. | N.D. | N.D. | N.D. | N.D. |
| +Proteobacteria 5 | 20.962 | N.D. | N.D. | N.D. | N.D. | N.D. |
| +Bacteroidales 1 | 14.615 | N.D. | N.D. | N.D. | N.D. | 0.648 |
| +Bacteroidales 2 | 19.792 | N.D. | N.D. | N.D. | N.D. | 1.909 |
| +Bacteroidales 3 | 16.458 | N.D. | N.D. | N.D. | N.D. | 2.755 |
| +Bacteroidales 4 | 16.467 | N.D. | N.D. | N.D. | N.D. | N.D. |
| +Bacteroidales 5 | 12.727 | N.D. | N.D. | N.D. | N.D. | 1.995 |
| +Bacteroidales 6 | 12.975 | N.D. | N.D. | N.D. | N.D. | 3.367 |

TABLE 10 shows SCFA production by the bacterial consortia in GF Il4ra^{F709} mice. The mice were colonized with the indicated consortium for two weeks. Thereafter their fecal pellets were collected and analyzed for the indicated SCFA. The results are reported for individual mice colonized with the respective consortia (n=4 mice colonized with the Clostridiales consortium, n=5 mice colonized with the Proteobacteria consortium and n=6 mice colonized with the Bacteroidales consortium). The SCFA abundance was normalized to mM/g of fecal pellets. N.D.: not detected. The assay thresholds for detection of the respective SCFA were as follows: Acetate: 2.5 mM; Proprionate: 0.3125 mM; IsoByturate: 0.3125 mM; Butyrate: 0.3125 mM; Valerate: 0.3125 mM; Caproate: 0.3125 mM; Isovalerate: 0.0156-0.3125 mM; Isocaproate: 0.0156-0.3125 mM.

TABLE 11
Demographic characteristics of subjects analyzed for their peripheral
blood ROR-γt⁺ Treg and Teff cells.

| Subject | Gender | Age | Condition |
|---|---|---|---|
| HC | F | 8 | HC |
| | M | 10 | HC |
| | F | 10 | HC |
| | M | 10 | HC |
| | F | 8 | HC |
| | M | 2 | HC |
| | M | 2 | HC |
| | M | 12 | HC |
| | M | 11 | HC |
| | F | 6 | HC |
| | M | 1 | HC |
| Atopic | M | 11 | AS, ED, AR |
| Controls | M | 9 | AS, AR, AD |
| | F | 8 | AR, AD |
| | M | 12 | FA, AS, AR, AD |
| | M | 11 | AR, AD |
| | M | 9 | AR |
| | M | 6 | AR, AD |
| | M | 10 | AR |
| | F | 8 | AS, AR, AD |
| | M | 9 | AS, AD |
| | M | 13 | AS |
| | F | 10 | FA, AS |
| | M | 14 | AS, AR, AD |
| | F | 17 | AS, AR |
| | M | 15 | AS, AR |
| | M | 4 | AS |
| | M | 13 | AR, AS |
| | M | 12 | AR |
| | F | 5 | AS |
| | F | 10 | AR |
| | M | 13 | AR, AD, AS |
| | F | 21 | AR, AS |
| | F | 8 | AS, AR |
| | M | 5 | AD |
| | M | 3 | AD |
| Food | F | 10 | FA (peanut) |
| Allergy | M | 11 | FA (peanut, treenut) |
| | M | 14 | FA (peanut) |
| | M | 17 | FA (peanut) |
| | M | 14 | FA (peanut) |
| | F | 4 | FA (peanut) |
| | M | 5 | FA (peanut) |
| | F | 10 | FA (peanut) |
| | F | 2 | FA (oat) |
| | M | 1 | FA (soy, egg, peanut, sesame, treenut, milk) |
| | M | 13 | FA (egg, peanut) |
| | M | 14 | FA (peanut) |
| | F | 1 | FA (milk, egg) |
| | F | 1 | FA (peanut) |
| | M | 1 | FA (peanut) |
| | F | 15 | FA (peanut, treenut, egg, legume, shellfish) |
| | M | 3 | FA (treenut, sesame) |
| | M | 8 | FA (fish) |
| | M | 1 | FA (peanut, milk, egg) |
| | M | 7 | FA (milk, banana) |
| | M | 23 | FA (peanut, treenut, chicken, pumpkin, turkey) |
| | M | 8 | FA (peanut) |
| | F | 2 | FA (peanut) |
| | M | 11 | FA (treenut, sesame) |
| | M | 11 | FA (egg) |
| | M | 16 | FA (shellfish, peanut, treenut) |
| | M | 19 | FA (peanut) |
| | F | 5 | FA (peanut, treenut, sesame) |
| | M | 6 | FA (peanut) |
| | F | 4 | FA (peanut, treenut, sesame) |
| | F | 1 | FA (egg) |
| | M | 3 | FA (milk) |
| | M | 3 | FA (peanut) |
| | F | 1 | FA (egg, peanut, sunflower, sesame, poppy) |
| | M | 2 | FA (egg, peanut) |

TABLE 11 shows demographic characteristics of subjects analyzed for their peripheral blood ROR-γt⁺ Treg and Teff cells. Characteristics of 35 FA, 11 healthy subjects and 25 atopic controls recruited for the analysis of ROR-γt⁺ Treg and Teff cells. Subjects were recruited ages 1-21 years of age. HC: Healthy Controls; FA: Food Allergy; AS: Asthma; AR: Allergic Rhinitis; AD: Eczema.

TABLE 12
qPCR amplification primers and TaqMan probes against gene-specific
targets in each Clostridales consortium strain.

| | SEQ | Primer/ | | Amplicon | | Gene |
|---|---|---|---|---|---|---|
| Organism | ID NO | Probe | Primer/Probe Sequence | Length | Tm | Target |
| C. | 17 | Forward | GAACGTTTGAAGTATCAGGAGGA | 102 | 62 | ABC |
| bifermentans | | | CAA | | | transporter |
| | 18 | Probe | ACTCAAAGGGCTGCAATAGCTAG | | 68 | family |
| | | | AGGA | | | protein |
| | 19 | Reverse | GATTTCCTGTAGGTTCATCGGCTA | | 62 | |
| | | | AT | | | |
| C. ramosum | 20 | Forward | GCTCTTAATGAATTACCATGTGCA | 104 | 62 | 2,3- |
| | | | ACAA | | | bisphospho- |
| | 21 | Probe | AGGCTCGTTTACCAGCCTGCATAT | | 68 | glycerate- |
| | | | CT | | | dependent |
| | 22 | Reverse | ACAACAATTGAGCGGGTTATTCCT | | 62 | phosphogly- |
| | | | | | | cerate mutase |
| C. scindens | 23 | Forward | GGCAGTTCGGCAAGTATCTGTT | 116 | 62 | HDIG |
| | 24 | Probe | TGCATGACATCGGGAAAGGCAGGC | | 68 | domain |
| | 25 | Reverse | GCAATCTATGCAGCGGCAATTC | | 62 | protein |
| C. | 26 | Forward | TTCTATGGCTTTGTATGAAACTCT | 124 | 62 | Integral |
| sardiniense | | | TGC | | | membrane |
| | 27 | Probe | TCACCACCAGGATGGTTGTTTGGCA | | 68 | protein |
| | 28 | Reverse | AGTCATATATACTCTGTATAGAGC | | 62 | |
| | | | AAACCC | | | |
| C. leptum | 29 | Forward | AATACTGACTCCGGCGCAAAT | 100 | 62 | Rod shape- |
| | 30 | Probe | AAGCTGAACGCCGGCTACAAAGG | | 68 | determining |
| | | | T | | | protein |
| | 31 | Reverse | CGCCTTGGTGTTTGGCTTTATG | | 62 | RodA |
| C. hiranonis | 32 | Forward | TGTTCAAGCTGCTATAGGATCAG | 108 | 62 | Na+/H+ |
| | 33 | Probe | ACTTGCGGCTGGACTAAATTGTGG | | 68 | antiporter |
| | | | A | | | cdu2 |
| | 34 | Reverse | GCTCCAAGTGGTGCAGTTA | | 62 | |

TABLE 13
qPCR amplification primers and TaqMan probes against gene-specific
targets in each Bacteroidetes consortium strain.

| | SEQ ID | Primer/ | | Amplicon | | Gene |
|---|---|---|---|---|---|---|
| Organism | NO | Probe | Primer/Probe Sequence | Length | Tm | Target |
| B. fragilis | 35 | Forward | AACCTAGCCAGGCATTGTGAAC | 134 | 62 | putative |
| | 36 | Probe | ACCCTGCCTCAAGGGAAACACATGC | | 68 | glycosyltrans- |
| | 37 | Reverse | CGTACTTCTTCCGAAACCGGATTATA | | 62 | ferase |
| | | | G | | | protein |
| B. ovatus | 38 | Forward | CACCGGACAATTCGGGATAA | 119 | 62 | ABC |
| | 39 | Probe | CGAATCTGTAGCTGTCTCGCTCCAA | | 67 | transporter |
| | 40 | Reverse | CGTTTCCGAGCAGGAATATCA | | 62 | substrate- |
| | | | | | | binding |
| | | | | | | protein |
| B. vulgatus | 41 | Forward | CTTGTCCCGTAATCTTCACCGTATTC | 113 | 62 | putative outer |
| | 42 | Probe | AACCCTGCACCTGTTTGCTCTGCA | | 68 | membrane |
| | 43 | Reverse | GGTATTGTCTTGTTTGCCGTTAGGA | | 62 | protein, |
| | | | | | | probably |
| | | | | | | involved in |
| | | | | | | nutrient |
| | | | | | | binding |
| P. distanonis | 44 | Forward | GTGATTATCGGCGGAGGCATTT | 142 | 62 | ROK family |
| | 45 | Probe | ACGGGAACAGGTCTGGAAATTCGTG | | 68 | transcriptional |
| | | | ATGA | | | repressor |
| | 46 | Reverse | GGCATCCGGCCTTATTTCCTAATC | | 62 | |
| P. | 47 | Forward | GTGCCAAGTGGACTGACATCTTT | 126 | 62 | MFS |
| melaninogenica | 48 | Probe | ACAGCAGCAAGAAACCTATCTGTGG | | 67 | Transporter |
| | | | CC | | | |
| | 49 | Reverse | CAACGAGGTAAGGACGCAACATAG | | 62 | |

TABLE 14
qPCR amplification primers and TaqMan probes against gene-specific
targets in each Proteobacteria consortium strain.

| | SEQ | Primer/ | | Amplicon | | |
|---|---|---|---|---|---|---|
| Organism | ID NO | Probe | Primer/Probe Sequence | Length | Tm | Gene Target |
| P. mirabilis | 50 | Forward | CGGTAGAGCCTACTGCAGGAT | 126 | 62 | Hypothetical |
| | | | AA | | | protein |
| | 51 | Probe | ATCACCGTTGCACCCGAACCGT | | 68 | |
| | | | T-JUN | | | |
| | 52 | Reverse | CCGGAGTTAAAGGTATCGGCTA | | 61 | |
| | | | TG | | | |
| E. coli Nissle | 53 | Forward | GTAGTGTGTTGGCGGCTCAAAT | 116 | 62 | HNH nuclease |
| | | | A | | | |
| | 54 | Probe | TGCCAACGCACCGATGTAAGA | | 68 | |
| | | | GCC-ABY | | | |
| | 55 | Reverse | GCCCGCAGAGGGAATATACAA | | 62 | |
| | | | AG | | | |
| B. | 56 | Forward | GCGCTTATGATCCAAGGCATGA | 105 | 62 | C4- |
| wadsworthia | 57 | Probe | AGGAACAGGGCACGCTCGTCT | | 68 | dicarboxylate |
| | | | ACA-FAM | | | ABC |
| | 58 | Reverse | CATGAAGACGTTGGAGACGAA | | 62 | transporter |
| | | | CAG | | | permease |
| K. oxytoca | 59 | Forward | TTACCGACCGAACTCTCCT | 96 | 62 | MurR/RpiR |
| | 60 | Probe | CGCCGCAGGATGTGGTGAATA | | 68 | family |
| | | | AGGT-VIC | | | transcriptional |
| | 61 | Reverse | GATCGACTGGCCTTCCATAAT | | 62 | regulator |

TABLE 15
gene-specific targets in each Clostridales consortium strain (qPCR
amplification primers and TaqMan probes in Table 12)

| | Gene | |
|---|---|---|
| Organism | Target | Target Sequence |
| C. | ABC | ATGGAAATTTTAAAAATTAATAATGTATCTAAAACTTATGAAGGGAAGGT |
| bifermentans | transporter | ATCTTATCAAGCCTTAAAAAATATAAACTTGTCTATAGAAGAAGGTGAAT |
| (SEQ ID | family | TTGTTGCTGTAATGGGGCCAAGTGGTAGTGGAAAGTCAACTTTATTAAAT |
| NO: 62) | protein | GTTATATCTACAATAGATAGACCAACTTCAGGTGAAGTAATATTAAACTC |
| | | TAAAAACCCGCATGAATTAAAAGGCCAGGATTTAGCAAATTTCAGAAGA |
| | | AATGAACTTGGGTTTGTATTTCAAAACTTTAACTTGTTAGATACTTTAACA |
| | | ATTGGTGAAAATATAGTACTTCCTTTAACATTAGATGGAGCTTCAGTAAA |
| | | AGATATGAATAATAGATTAAATGAAATATCTAAAAAGCTAGGTATAGAA |
| | | CAGATAATAAACAAAAGAACGTTTGAAGTATCAGGAGGACAAACTCAAA |
| | | GGGCTGCAATAGCTAGAGGAATAATAAATAAACCATCAATTTTATTAGCC |
| | | GATGAACCTACAGGAAATCTAGATTCAAAATCTACAGATGATGTTATGG |
| | | ATTTATTTACAAAAATAAATACTGAAAATAAAATGACTACGCTTATGGTA |
| | | ACGCATGAGCCTTATACTGCAAGTTTTTGTAACAGAATCATTTTTATAAA |
| | | AGACGGAGAAATTTACAAAGAACTTAATAAAAAAGGTAATAGAGAAGA |
| | | CTTCTATGAAGAAATACTATTAGTGCTATCTCAAATAGGAGGTGCTAGAT |
| | | AG |
| C. | 2,3- | AAGGATCAATGTTTCAATCATAATTAAACAGTAACTAGTTTTTTACCCTG |
| ramosum | bisphospho- | ATCAGCAACAGCATCGATCTTCTCTTTTAATAACGTTTCATCACCTAAATA |
| (SEQ ID | glycerate- | GTAATGTTTTATTACTTTAAATTCATCATCAAATTCATATACTAAAGGAAT |
| NO: 63) | dependent | TCCAGTTGGAATATTAATATTCATGATTGCTTCGTTACTTAACTTATCAAA |
| | phospho- | GTATTTAACCAACGCTCTTAATGAATTACCATGTGCAACAATTAAGGCTC |
| | glycerate | GTTTACCAGCCTGCATATCTTTTTTTACTGTTTCATTAAAATAAGGAATAA |
| | mutase | CCCGCTCAATTGTTGTTTTTAAGCTCTCACCTGCTGGTAAAAGAGCAGAG |
| | | TCAATATTTCGATACATTGCCTGCTTTTGCGCGCTGCGTTTATCATTTATA |
| | | TTTAAAGCTGGTGGCAGTACATCAAATGAACGACGCCAAATTTTCACC |
| C. scindens | HDIG | TCATATTCTAATCTCCTTATTCTTTTTTTCAGTACTCCAGTTACAGTCCGTT |
| (SEQ ID | domain | TCTGCCGGACAGGCGAAACAGGCGCGAGAGGCCTTGTCGCTTTCATATA |
| NO: 64) | protein | ATAGTGCTTCCAGAGGTTCAGCGTTCTCGCGAAGCTTCCGGTGTCCACGG |
| | | ATTGCCGTAAGAATCTGTTCGTTTTCATTCGAAGTAAACATAAGTTCTTCC |
| | | GGCAGTTCGGCAAGTATCTGTTCAGCAATATCAGCGCTTGCAAGTTCATG |
| | | TGGGATTCCTGACTCATACTGCCTGCCTTTCCCGATGTCATGCAGAATTGC |
| | | CGCTGCATAGATTGCTTCTTTGGAAATTCCAATTCCTCTTTCAAGGCTCAG |
| | | AATGTAAGCAATTCTGGCTGTGTCCAGCAGATGATTCATCTGATGGCAGC |
| | | AGAAGATACGTTCTTGCTCCAGTTCTTGCAGTCTTTGGTAAGAAGATATA |
| | | TATAGGGGATGCTTGCGGATATAAGAAATTCTTAACATCGTGTTCTCCAT |
| | | ATGATTCAT |
| C. | Integral | ATGAAAAATATATTTAAGGTAAATGACAAATTTAAGCTTATACCATTGAT |
| sardiniense | membrane | CATATCAATATTAATTCCAGTAGGTGGAGGCGTATTGGTAGGATATATTA |
| (SEQ ID | protein | CAAAAGATTCTATGGCTTTGTATGAAACTCTTGCAAAACCTAAATTTTCA |
| NO: 65) | | CCACCAGGATGGTTGTTTGGCATTGTATGGCCTATTCTATATATCATTATA |
| | | GGGTTTGCTCTATACAGAGTATATATGACTCTTAAAGAAGAAAAGAGGA |
| | | GTTATGGAATTTTAATAGTGTATTTTATTCAGCTTTTGATTAATTTCTTAT |
| | | GGCCTATATTATTCTTTAATTTAAAATTGTATGGATTATCAGCAATTAATA |
| | | TAATCATACTTATAATTTTAATAATAATATGTATAATAAAATTTATAAAG |
| | | ATTGATAAGATATCATCAGTATTATTAATTCCATATTTAATATGGTGCGGT |
| | | TATGCAGCTTATTTAAATATAGTAATTTGGATGATAAATGAAATGTAA |
| C. leptum | Rod | TTGACGCTTCTGTCTGATAGCGTCAAAGGCTTTTTTATTGGTTACAGCTGT |
| (SEQ ID | shape- | TATACAGCATACAGTGATTTGCTCAATAGATTTGACAAGGAACGAAGAA |
| NO: 66) | determining | GTCCTCTATTGCTGCGCGCCTGCCTGCGCGGCATGAAGATGGCTGAAATC |
| | protein | GATTTCCTTTTCAATGTTATGCCGATGCATATAAACGCTTTCCACAATTCC |
| | RodA | GACGCCAAGGTACAGACACGCCACGGAGGAGCCTCCTGAGCTGAAAAAG |
| | | GGAAGAGTAATGCCGATTACCGGGAGCAGACCCAGGCACATGCCTACGT |
| | | TGATGACAGTCTGAACCGCCAGCATAGCGAAAAAGCCAAAACAAAGGAA |
| | | TTTACCCAGGTCATCCTTGGCGGAAAGAGCGTTCATGACGCAGCGGAGC |
| | | ATCAGCAGCAAAAGCAGTCCCAGCAGGACGACGCACCCGATAAAGCCCA |
| | | GCTCTTCTCCGGCTACAGAGAAAATAAAATCATTTTCCTGATAGGGAACC |
| | | GACCCTGCCGCCACCCGGGGGCCTTCATAGTAGCCCCGGCCATACATTTC |
| | | GCCGGAGGCGATAGAAATTTTTCCTTGGAGCTGCTGGTAGCCGAACCCAT |
| | | TGGGATCGGATTCCAAATTAAACAGAGTCCAAAACCGCATTTTTTGATCC |
| | | TCATTTAATACAGAATTCCATAAAATCGGAATACTGACTCCGGCGCAAAT |
| | | GATTAAAGCTAAATAGTATCTAAGCTGAACGCCGGCTACAAAGGTCATA |
| | | ATCAGGAACATAAAGCCAAACACCAAGGCGGTGCCGTCGTCGCCCATAA |
| | | AATGGATTAATCCAA |
| C. | Na+/H+ | ATGTTAGTATCACTTGCTTTAATTTTTTTAGTTGGAATGAGCTTGGCATCT |
| hiranonis | antiporter | ATATGCGAAAAAATTAAAATTCCGAGAATTATAGGAATGCTTGTAACTG |
| (SEQ ID | cdu2 | GGATAATTTTAGGTCCTTATGTACTAGATTTTTTAGATAGCTCAATTCTAA |
| NO: 67) | | ACATATCATCAGAGCTTAGAAAAATGGCTCTTATCATCATCTTAATCAAA |
| | | GCTGGGTTATCACTAGACTTAAAGGACTTAAAAAAAGTTGGGAGACCTG |
| | | CGCTTTTAATGTCGTTTCTTCCAGCTACATTTGAAATAATTGCTTATGCA |
| | | ATATTTGCTCCAATTTTATTTGGTGTCAGCAGAGTAGAGGCTGCACTAAT |
| | | TGGAGCAGTACTTAGTGCAGTATCTCCAGCAGTTGTAGTTCCTAGAATGG |
| | | TTGATTTAATGGACAATAATCTCGGGACAAAGAAGGGAATACCTCAGAT |
| | | GATTTTAGCTGGGGCATCTTTTGACGATGTATTTGTAATTGTGCTATTCAG |
| | | TACATTTTTAGCTATGAATCAAGGAGAAGGTGTTAATCTTTCTAGTTTTGC |
| | | AGACATACCAATTTCGATAGTATCTGGAATTTTAATTGGGTCTGTTGTT |
| | | GGATTAATTCTTTATAGATTCTTTGAGTATAGATACAACAAAGAACATCT |
| | | GATAAGAAACAGCACAAAAGTCATAATTATCTTGGCTGTATCGTTCTTAC |
| | | TTGTTGCACTTGAAGATTATTTAAAAGGAAGAGTTGCTATGTCCGGACTT |
| | | CTTGCTGTAACAAGTATGGCATTAGTACTTGCTATGAAGAGTACAAATAT |
| | | TGTAAAGGTCAGACTTCAAGAGAAATTCGGTAAAATTTGGATAGCCGCA |
| | | GAAGTTGTTTTATTCGTACTTGTTGGGGCTGCTGTCGATATAAGATATAC |
| | | AATGGGAGCTGGATTTACAGCAGTTATTATGATATTTATCGCACTTGCAA |
| | | TTCGTTCAATAGGTGTATTTATTTGCATGATTGGAACAGAATTAAACACT |
| | | AAAGAAAGATTATTCTGTGTGTTCTCATATCTTCCAAAGGCAACTGTTCA |
| | | AGCTGCTATAGGATCAGTACCACTTGCGGCTGGACTAAATTGTGGAAAAC |
| | | TTGTACTATCAATTGCAGTACTTGCAATAATAATAACTGCACCACTTGGA |
| | | GCATTTTTAATAGATTTCTCAAAAGAAAAATTATTATAG |

TABLE 16
Gene-specific targets in each Bacteroidetes consortium strain (qPCR
amplification primers and TaqMan probes in Table 13)

| Organism | Gene Target | Target Sequence |
|---|---|---|
| B. fragilis | putative | CAACATGGGCAGTGAACCGGGTATGGCCTGGAACTGGTGCATAAACC |
| (SEQ ID | glycosyl- | TAGCCAGGCATTGTGAACTATACATTATCACTGAAGGTGAATTCAGAG |
| NO: 68) | transferase | ACAAAATAGAGGCAGTGCTCCCTACCCTGCCTCAAGGGAAACACATG |
| | protein | CACTTTTACTATAATCCGGTTTCGGAAGAAGTACGGAAGATGTGTTGG |
| | | AATCAAGGAGATTGGCGTTTCTATAAACACTATAAGAAATGGCAATG |
| | | GAAGACTTACGAGATGGCACAGGAAATAATAGTCAAACAACATATAG |
| | | ATATTGTACACCAATTAAATATGATTGGCTTTAGAGAACCCGGATACC |
| | | TTTGGAAACTAGATAAGCCATTTGTTTGGGGACCGGTAGATGCTAAAG |
| | | AAAAATTTCCGACAGCATATCTAAGAGATGCAGGGATAAAAGCAAAC |
| | | TTATTCATCAGATTAAAAAATCACATAACCGGTTTACAGTTACGATAT |
| | | TCACAACGAGTAAAAAAAGCTGTAAAAAAAGCCTCTGTAGTAACATC |
| | | CGCATCTTCTGAATCTCAGAAGAGTTTCAAGAAATATTTTCATATTGA |
| | | CGCTCCCTTATTAAATGAAACAGGGTGTTATCCTAAAACAACAATAAT |
| | | AAACAGTACAAAAGAAAAAGGTGATTTAAATTTGCTTTGGGTAGGTA |
| | | AATTAGATTTCAGAAAACAATTGCCTTTAGCGATAAAGGCCATAGCAC |
| | | GACTGGCTAATCCACATATAAAACTCCATATCGTAGGTGGTAACAATA |
| | | ATTCCTATCAAAAGTTAGCGATGGAATTGAACATATCACATCAATGTA |
| | | TCTGGCATGGGGTTATCTCACATAATGAAGTTCAGGAACTCATGCAGA |
| | | AAGCAGATATTTTCTTTTTTACCAGCATAGCTGAAGGAACTCCACATG |
| | | TTGTTTTAGAAGCCATCAACAATAACCTTCCTGTTATCTGTTTCGACAT |
| | | ATGTGGACATGGTGACTCAATTAATGAACAAGTAGGGATAAAAATTC |
| | | CCCTATCTACTCCGCAACAATCCATCAACGATTTTGCGGAGAAGATAA |
| | | CATATCTGTTTAACCATAGAGACGTACTTAAGCAAATGTCTGAAAATT |
| | | GCAGAGTCCGTCAAGAGGAACTATCGTGGGACAATAAAGCCAAACAG |
| | | ATGGTCAGTCTATATAAAAAAGTATTGTCACAAAAATGAGTAAAAGA |
| | | TACAAGCTA |
| B. ovatus | ABC | TTATCTGCGTAACGGAGTCCGTTCATCAATAATTCCGCGGCCTCCGTA |
| (SEQ ID | transporter | AAGTCTCATCGTAACAGGACTTCCGGCTTCTATGGTAATATCCTGATC |
| NO: 69) | substrate- | TGCGATGATAGGTTCTTCCTTATCGTCTACAATTACAGAACCAATCTTC |
| | binding | TCACCAGCCTTGATTATCTGTGGAATCAAGTTAGTCAGGATACCTTCG |
| | protein | GAAATTTTCCGGATACTGATATATTCCGGGGTTGCATTCTTGTCAGCTT |
| | | CGATTTTACCGTTGAAAAGATCCTGCACACCTTTGTTCTTCAAGAAAA |
| | | GTTTAACGATTGCTTTACCTTCGTACGGGTTGCCTTTTTCATCAAGTAG |
| | | AATAACGGGCGCAGTCATTCGCTTGAATGTAAGGGCTACATTGCTAGA |
| | | ACCTTTGCTGGCTTGGGCTGTGCCAAACAAATATTCTAAACCGCCTTT |
| | | GATGTCACGGTAGCGACTAAAGAGGGATCTAGTCGTTACATCACCGG |
| | | ACAATTCGGGATAATGAGCATAGAAAGTTACAGCTTCCGAATCTGTAG |
| | | CTGTCTCGCTCCAATTTTGGTTATCTACTCCCAAAGTAAAAGGCTGAT |
| | | ATTCCTGCTCGGAAACGGACATTGAAATGACGTCTCCAGCTGAAAAAC |
| | | TACTTTTTAAAGGGAGTGACAGGTTACTGCTGCTACGAGTAACAGCGT |
| | | CAGTGATACTTTCATTGTCGACAGTAGTAAGAAAGTTGATACCTTCTT |
| | | TACTGGGATCTTGCAGTTCTTCTTGCTGTTGACAACTGAACAGGCATA |
| | | CTGATGCCATAACAAATAAAAGGCTTTTTCTTTTCAT |
| B. | putative outer | TTATTTGAATGATAAATTAATACCAAATATTACATTGCGAGTCATTGC |
| vulgatus | membrane | ATCCGATGTATTGATGCTGACATTCAACTGTTCAGGATCAACCACATC |
| (SEQ ID | protein, | CTTTGCACAGAAAATAAATGGATTCTGAACTGTTGCATATAAACGCAA |
| NO: 70) | probably | ATTGCCTAATTTCATTTTCTCAAGAACCGATGGTTTAAAAGTATATCCC |
| | involved in | AAAGTAATATAAGAAATCTTAAGGAAATTACTGGAAAACATCGTATG |
| | nutrient | TGACATTTCCGATTTTGCATTTCCCCATGATCCTGCCTTGCTTCGATAA |
| | binding | GTACCCATGTTACTAGGCTGTGCAGAAGCATTGGTAGGATTTTCCGGA |
| | | GTCCAATAATCTTTTCTTAAATTATTAAAATTGAGATTGTTGTTTTCCA |
| | | ATGCATACGAAACATAGAACTGATTTCTAGCTCTTGCCCCTGCTTGGA |
| | | AAGCTGCTTGAAATGACAAATCAAATTCCTTATATGTAAATGTATTAG |
| | | TCATACCACCTGTCCAATCCGGAGTACGTTTACCATCAATCACACGAT |
| | | CTTTATCGTCAATCACACCATCCTCATTCAAATCCAAAGGTTTATATTG |
| | | ACCAGGTTTACAACCATATTTAGCTGCTTCTTCAGCTTCATCCAGCTGC |
| | | CATACTCCCAATGTCATCAAATTATAATTTATATCAATAGGCTGACCA |
| | | ATAATCCAAAGATTACTATAATCACCACTCATACCAGCTAAAGACAAT |
| | | CCACGGGAAGTCAAATTCTCCTTATACTGTAAATCTACAATTTTATTCT |
| | | TATTATAGGCAAAATTCAAACTAGTTCTCCATGAAAAATCTTTAGTAC |
| | | GGATATTATCCGAATTAATATTTACCTCAAACCCTTCATTTCTAACAGA |
| | | CCCTACATTTGCTTTTACAGAAGAATAACCGGTAGTAACAGGTACTGT |
| | | CTTATTCATAATCAAGTCTTCTGTCAAACGATTATAATATTCCACACTA |
| | | CCCGAAATCCGATTATTGAAAAATCCGAAGTCAAGACCTACATTATAT |
| | | TCTGTAGTACGTTCCCATCCCAAATCCAAATTCCGTAAATTATTGGGA |
| | | ACATATCCAATAGACTCACTAGAACCGAATGTATAATATTTGGCACCA |
| | | CTAATAGATCCTTGCGTCTGATACGCACTTACATTATCATTACCGGTCT |
| | | GACCATAACTTAAACGTAACTTCAAATTACTCAACCAAGACAAGCTCT |
| | | TCATAAATTCTTCTCCTGACACTCTCCATGCTACAGCTGCTGATGGAA |
| | | AACTTCCCCACTTATTGCCTTCAGCTAATTTTGACGAACCATCAAAAC |
| | | GAATACTTGCTGTTACAAAATACTTGTCCTTATACACATAATTGGCAC |
| | | GTGCTAAATAAGACATCAAATTCGTTTTGGTAAAACCGGAAGAAGAA |
| | | GTATTACTTGCCGATCCTCCTGCCAAATTATACCACAAAGAGTTATAT |
| | | GACAATCCATTACCAATTCCTTTTAACTTTTCATCTTGTGATTGCTGCA |
| | | TAGAGAACACTCCGGTCAAATCTACGCGATGATCCTTCAATTCAAAGT |
| | | TATAATTTACAAGATTATCCCAAACCCAATCTACATAGCTATTTTTCGC |
| | | ATAATTACTGGTTGCCTTATTTTGCCCTTTATTAGCTTTAGTGTATTTAC |
| | | CTCTGTACTGGCCAATTTCTTCCAAATTTATATCCGGCGAGAAAGTGG |
| | | TCTTCAAAGTCAATCCTTTAATTGGAGTTATTGCCAGATAAATGTTACT |
| | | CAGCAAATTGTACTTTTTGGTTTTATTCAGTTCATTCTTCATGGTAGTT |
| | | AACGCATTATACTGACCATTAGAATAAGCCCACATTTCTTCTCCTGTC |
| | | ACCAAATCAGTCGGATGATAAGTCGGACGCAGACGAAAAGCATCCTG |
| | | TAACAAGTCAGAGTTACCCGTATCACGTACTGAATGGGTTCCATACAT |
| | | ATTAATTCCAAATTTCATGTATTTGCTAGGTTCTACATCCACAACAGCA |
| | | CGCAGATTATAACGGGAATATTCTTGAGGCTCCAACATACCATCTTCA |
| | | AAATAATATCCGGCAGATAAAGCATAAGTAGCCATCTCATTACCTCCG |
| | | GTAGCCGATATAGTATGATTTGTCATAAAGGCCGGACTAGAAACAGC |
| | | ATCCACCCAATCAAAATAATTTCCATCTTGAATAGCCTTTAACTCTGAT |
| | | GCTGTGAAAATCTGACTATCATCCACATATTTATTATTATTTCCGGCTC |
| | | TTTTAGCCTCTCGTGCCAATTGCACATATTCTTCACCAGACATCATATC |
| | | GGGCATATTCGTATATTTCCTATATCCGGCATATCCACTATAATCTATT |
| | | TTTACTTTACCTATCTTACCTCTTTTAGTAGTAACCATGACTACACCAT |
| | | TAGTAGCACGGGAACCATAGATTGCGGTAGATGATGCATCTTTCAAAA |
| | | TATCAATTTTTTCTATATCATCCGGATTCAGGTTAGATAAAGAAGCTCC |
| | | TGGCACACCATCTACAACAATTAAGGGAGATGTACTTCCGCTAATTGT |
| | | ATTCAAACCACGAATCAGAATATTATATTCCCCTCCTGGTTTACTATTA |
| | | GAACGTTGAATTTGCACACCTGGCAAAGCCCCTTGCATAGCTCCTACT |
| | | GCATCCGTTCTACCACTTCTTATCAAATCCTGAGTAGAAACAGAAGCT |
| | | ACAGCACCTGTCAAATCCGATTTCTTTACCGAACCATAACCTACCACT |
| | | ACAACTTCGTCCAACAGCTCAGTATCTTCTTTTAGAACAATCTTGAAA |
| | | GAACGGTTAGAACCAATATTCAATTCTTGAGTTTGAAAACCAACGTAA |
| | | GATATCTGTAACTTATGAGAAGGAAAAACAGTCAAACTAAAATTACC |
| | | ATCCAAATCAGTTACAATACCGTTGGTAGTTCCTTTCTCCATTACTGTA |
| | | GCACCTATTATCGGTTCTCCATTTGGATCAATAACTTGTCCCGTAATCT |
| | | TCACCGTATTCTGCAATACTTCTTGATTAATCGGAGAGTACAACCCTG |
| | | CACCTGTTTGCTCTGCAACCGCATTTCCTAACGGCAAACAAGACAATA |
| | | CCATAAGTAATAAAAATTTGTCTTTCTGTTTCAT |
| P. | ROK family | ATGAAATACGCTATCGCATTAGACATTGGAGGAACAAGCATAAAATA |
| distanonis | transcriptional | TACTCTTGTGAACCAGAATGGCGACATTCTTTACGAATCGTCGGAAAC |
| (SEQ ID | repressor | CACACAATCAAAAGAGAATCCACGCCCATTATCCGATACCATAAAAA |
| NO: 71) | | GTATCGTACGGAAAATGACAGACTACGCCCGTTCCCGGGACTGGGGG |
| | | ATTTACGGAATTGGCATAGGTGTACCTTCCGTAGTAGATAAGGGGGTG |
| | | GTTCTCTTTGCCAATAATCTTCCTGAACTGGACAACCAACAATTGGAT |
| | | CTTGCGTTAGCGGAATTTAATCTACCGGTATTCATCGACAACGACGCA |
| | | AACCTTATGGGGTTGGGCGAGGTGATATATGGGGCGGCTAAGGGCCT |
| | | CTCCGATATCGTTTTCTTGACGGTGGGTACCGGCATAGGGGGCGCCTT |
| | | GTTCTTGAACGGCCGGCTTTATGGCGGCTACCGGAACCGGGGCACCGA |
| | | ACTTGGGCACTTAATTATTCATGGTCTGAATGGGAATCAATGTACTTG |
| | | CGGAGCGTCCGGTTGCCTAGAGGCACACGCTTCCGTAAGTGCGTTGAT |
| | | CGCCTTATATCGGCAATTATTGGAGAAGAACGGACGGGAGATACCTTC |
| | | CCGTATCGATGGAAAATATATAGTAGAGCGCTATAAAGCTCAAGAGA |
| | | AAGAGGCCGTACTCGCTATGGAGGATCATTTCCGGAATCTGTCCCTAG |
| | | GCGTAGCGAGCCTTATCAACATTTTCGCTCCGCAAAAGGTGATTATCG |
| | | GCGGAGGCATTTCGGAATCCGGAGATTTCTACATTAATAATATACGGG |
| | | AACAGGTCTGGAAATTCGTGATGAAGGAAACCTCGTACTTCACGACTA |
| | | TCGAACTTGCCCGATTAGGAAATAAGGCCGGATGCCTCGGGGCGGCG |
| | | GCATTGGTGTTTAATCATTGA |
| P. | MFS | TTACCTTTGGTACTTCCCTCAGCATGACATTCTAAGGAGAGGATTAAG |
| melaninogenica | Transporter | GGTAGGAGAAAAAGGATGCTGCAGCTAATTCAGCTCACTTCTCCTAAA |
| (SEQ ID | | GTCCTTTCTCATGATATGTAACACGATGCTCTACTTATATAATTCACTA |
| NO: 72) | | CATGTTCAACAGCATTTCATCAAAGAAACTAAGCTTTGGTACGTTCTT |
| | | CTGTCTATATATTGCACAGATGGTACCGTCATCCTTTCTTATGACAGCC |
| | | TTACAGGTTATCATGAGGGAAGGACAATATAGTCTTGCAACCATTGGG |
| | | TTGCTAAACCTTGTCCGAGTTCCATGGACGATAAAGTTCTTATGGTCG |
| | | CCTTTTGTTGACCGCCACTGCGTAACAGTGCGTGACTACAAACGTACG |
| | | ATTATCGCTACAGAGTTGATATATGCCGTTGCACTCTTAGCCACAGGG |
| | | CTGATTAATGTGCGTTCGGAAATCATGCTTGTAGTCATTCTGGCGTTTA |
| | | TCTCTATGCTTGCTTCGGCTACGCAAGACATTGCTACAGATGCGCTTG |
| | | CCATCCTTTCTTTTAAGAAGCACGACCATAGTATGCTTAATAGTATGC |
| | | AGTCTATGGGAGCTTTCGGAGGTGCTGTTATTGGTGGAGGAGTCTTGC |
| | | TTATCTTACTGAAAAGCTATGGCTGGAATGTTGTAGTACCCTGCTTAG |
| | | CCTTGTTTGTTTGTATGATGATTATTCCTTTAATGTTTAATCCTCATATC |
| | | AAGATAGAGAATGAGAAACCAAGAGAGCGTGCCAAGTGGACTGACAT |
| | | CTTTAGCTTCTTTGGACGTAAGGAGATATGGCCACAGATAGGTTTCTT |
| | | GCTGCTGTATTATATGGGAATCATCGGTATTCTTTCTATGTTGCGTCCT |
| | | TACCTCGTTGATAAGGGATACGATATGAAGG |

TABLE 17
Gene-specific targets in each Proteobacteria consortium strain (qPCR
amplification primers and TaqMan probes shown in Table 14)

| Organism | Gene Target | Target Sequence |
|---|---|---|
| P. mirabilis | Hypothetical | TTATTGATAAGAAAAAGTGAGACTGGCTAATCCGTTTGCTTTACCCG |
| (SEQ ID | protein | GTTTTAGTGTCCCAGTCGTGATATAGCGAGTTGACAGGTTTAATATGT |
| NO: 73) | | AGTTTTCGTTGTTAGAAGTTGTTATATGGAGTAAACGTTGGTTAGGTT |
| | | GTCCTATCGCACTGATATCAGGGCCATACACCACGGGAGTTGGATTA |
| | | TCATTAAAGAAAAATTGAATACCAATACCAGAAGCATCAGAATCTGA |
| | | TGTCAATGTGAGGATATCTGTATTATTAGAATAGGTAGACTGATCGG |
| | | TCATGCTGGCATATAAATGCACATTACTATCGCATGTAACAGAGATG |
| | | GGTTTAGCCTGACTTTTTGATGTTGAACCAATGGCTGCCATATCTCGC |
| | | ATTGAGATATCACCTAATTCATAGGTGAGATTTTTGGTATTTACGGTA |
| | | CAGCCTCGAGCTTTAATAGTAATAGTGACGGGGTTAATTGCTACGAT |
| | | AGAGGTATTATTGCCTTTATGGCCTCCACCATATTCAGACCATGTATT |
| | | GGCGATAACTGTATATGGAATGTTATAAACGCCAGTCTCAAGATGTT |
| | | TATCTGTCACCACAAAGACGACCTTGCCTTTCAGTGAAACGCGATCC |
| | | ACATTATGGTTAACGGTAGAGCCTACTGCAGGATAAATCACCGTTGC |
| | | ACCCGAACCGTTATCAACATCAATAGGTACGTAAGCAACACTATCAT |
| | | TGTTATCTTTTAAACCAAAAGCATAGCCGATACCTTTAACTCCGGGG |
| | | AGTGAATAAACAGGATAACGATTACTACCTTCGTAATAGTAGATCCC |
| | | GCTAATTTTTGAACCAGTAGCATTAGCATAGAGGGTTTCAACCCAAC |
| | | ATTTTTGCAAAAATTTCTTCTCACATTTGAAACCATTATGGACAGAGG |
| | | TTTCACCTATCCAAGTTGACGTGATGGGCGTTCCTGCGGGAGTACCAT |
| | | CGGCGGCACCATCAAAATTCATAGGTGGAGGGGTTACCACTAATGGA |
| | | TCTGCAATAGCCCATGCCATTTGGCTCGGGATACAAAGAAGAGAAAC |
| | | CAGCAATGTGAAGAAAGAAAAAGAGCGTGTCAT |
| E. coli | HNH | GGCGATATTTCTTCAGGCTGTGGGACGTATGCGTCGGTTGGTAATCG |
| Nissle | nuclease | CTGGCAGGTTGGCCATGAAGATGAAATTTTTGCCTTTGCACTCACCA |
| (SEQ ID | | ACGCCATTACCAGTACTGGTAAAGGCGTTAATCTACAGGGGCTACAA |
| NO: 74) | | TTTTGCAAACTCATTGATAAAAGCTCACCGCTACTGTCTAATGCCATC |
| | | AATCAGAATGAGCGGTTATTTATTGAAATCGATTTGTATCGTATAAAT |
| | | AAAAGCGGGCGCTGGGAACGGTATTATTATATTCAGCTAAGAAATGC |
| | | TTCATTAACTGCTATTCATGTAAACATTTCTGACAATAATCTTCCTAC |
| | | CGAATGTGTAAATGTTAATTATGACTACATATTATGCAAACATCTAAT |
| | | AGCCAATACGGAATTTGACTGGTTGGCCTTTCCTGCTGGCTATAATAG |
| | | CTTATTTATTCCACCTAAAAACCCACCTGCCAGTAATCTTAACCCTGA |
| | | GCCGCTACCAGTTGTTAACCTTCCACTCTCTCCACCAGCGGTTAAACC |
| | | GGTCTATGCCAAATCCTGTCTGAAGGAGAAGGGATGTACAGATGCCG |
| | | GAACGGCAGAAGAACCCGCTGAAAACTTCGGGCAAGTAGCGATTTTT |
| | | GCTCTGCCAGTGGTTGATGACTGCTGTGGATACCACCATCCGGAGGC |
| | | TAACGATGTTGGGCAACCCGCAGAAGCTCAAACCATGCTACTGTTTC |
| | | CGGGTAGTGTGTTGGCGGCTCAAATATGGGGAAAATGGTCGCTCAGT |
| | | GGCATACTCAGTGCAACCCGCGGCTCTTACATCGGTGCGTTGGCATC |
| | | TGCTTTGTATATTCCCTCTGCGGGCGAGGGCAGTGCTCGTGTGCCTGG |
| | | ACGTGATGAGTTCTGGTATGAGGAAGAACTGCGCCAGAAAGCGCTTG |
| | | CAGGCAGTACCGCCACTACCCGGGTGCGTTTTTTCTGGGGAACTGAC |
| | | ATTCACAGCAAGCCCCAGGTATATGGTGTTCATACGGGTGAAGGTAC |
| | | GCCGTATGAAAACGTCCGCGTGGCGAACATGCTGTGGAACGAGGAG |
| | | AGGCAGCGTTATGAATTTACCCCCGCTCACGATGTCGATGGCCCCCT |
| | | GATTACCTGGACGCCGGAAAATCCGGAACATGGGAATGTTCCGGGCC |
| | | ATACCGGTAACGACAGGCCGCCGCTGGATCAGCCCACCATTCTGGTG |
| | | ACGCCGATTCCGGACGGCACCGATACCTATACCACGCCGCCATTCCC |
| | | GGTTCCTGATCCGAAAGAATTCAACGATTATATTCTGGTTTTTCCGGC |
| | | GGGATCCGGTATTAAGCCCATCTATGTTTACCTGAAGGAGGATCCGC |
| | | GAAAGCTGCCTGGTGTTGTAACAGGGCGCGGCGTCCTGCTTTCACCA |
| | | GGAACTCGCTGGCTGGATATGTCGGTATCCAATAACGGCAACGGCGC |
| | | ACCAATCCCGGCGCATATTGCT |
| B. | C4- | ATGTTCGATATGTTCATGACAGCCTTCAGTTCGGCCTGTAGTCTGGAA |
| wadsworthia | dicarboxylate | GCCCTCGTCGCCAACTTCATCGGCGTGGCGCTCGGCATCGTCTTCGGC |
| (SEQ ID | ABC | GCGCTGCCCGGCCTCACCGCCGTCATGGGCGTGGCCCTGCTCATTCC |
| NO: 75) | transporter | CTTGACCTTCGGCTTCCCCGCCGTCATCGCCTTTTCCTCCCTGCTCGG |
| | permease | CATGTACTGCGGGGCCATCTACGCGGGCAGCATCACCGCCATCCTCG |
| | | TCGGCACTCCGGGCACGGCGGCCGCCGCCGCGACCATGCTCGAAGG |
| | | GCCGCAGTTCACCGCCCGCGGGGAATCGCTCAAGGCGCTCGAAATGA |
| | | CCACCATCGCCTCCTTCATCGGCGGCATCTTCTCCTGCCTTGTGCTGG |
| | | CCACGGTCGCCCCCCAGCTCGCACACTTCGCCCTCGACTTCAGCGCC |
| | | CCGGAATATTTCTCCCTCGGCATCTTCGGCCTGACCATTGTGGCGACG |
| | | CTGTCCGAAGGTGCGCTGCTCAAGGGCTGCATCGCGGCGCTGCTCGG |
| | | CATGCTCATCTCCATGATCGGCATGGACCCGCTTTCCGGCAATCTGCG |
| | | CATGACCTTCGACTCGCCCGACCTCATCAACGGCGTATCCCTCGTCCC |
| | | GGCGCTCGTCGGCCTGTACGCCCTGTCGCAAGTGCTGATCACGGTTG |
| | | AAGACGTGTTCATGGGCCGCAAGCTCTCCACTGCCGAAATCTCGCGC |
| | | AAGCGTATGCCCCTTTCCGAAATCTGGACAAACCGGGCCGCCCTGCT |
| | | CCGCGGCTCGATCATCGGCACGTTCATCGGCATCGTCCCGGCCACGG |
| | | GCTCGGGCACGGCCTCATTCGCGGCCTACAGCGAAACCAAGCGCCAT |
| | | TCAAAGCATCCCGAACTTTTCGGCAAGGGCTCCATTGAAGGCATCGC |
| | | GGCCACGGAATCCGCCAACAACGCCGTCACCGGCGGCGCGCTCATCC |
| | | CCTTGCTGACCCTCGGCGTGCCCGGCGACGTGGTCACGGCGATCATG |
| | | CTCGGCGCGCTTATGATCCAGGGCATGACTCCCGGTCCGCTGCTGTTC |
| | | CAGGAACAGGGCACGCTCGTCTACAGCATTTTCATCGCCCTGTTCGT |
| | | CTCCAACGTCTTCATGCTGCTTCTGGGCTACTACGCGGTGCGCCTGTT |
| | | CGCCAAGGTCGTGCTCATTCCCGGCGGCATCCTCATGCCCCTCGTCAC |
| | | CACCCTGTGCGTGGTGGGCGGCTACGCCCTGAACAACTCCAACTTCG |
| | | ACCTCGCCGTCATGGCCGGTTTCGGGTTGCTCGGCTACATCATGACC |
| | | AAGGCGCGCTTCCCGCTCGCCCCCCTGCTCCTCGCCATGATCCTCTCC |
| | | GGAATCATCGAAACCAACTTCCGCAGGGCGCTCAGCATCTCCAATCA |
| | | GGATTTCTCCGTATTCTTCACCCGTCCTGTCTGCGCGGCGTTCCTCGC |
| | | CATCAGCCTCTTCATCCTGTTCAACCTGCTCTGGAAGGAATGGAAGA |
| | | AGTACCGCGCCGCCAGCGCCGCCTGA |
| K. oxytoca | MurR/RpiR | ATGAGTCAGACAGAATCCAGTTCGCTCCCTAACGGCATCGGCCTTGC |
| (SEQ ID | family | CCCCTGGCTTCGCATGAAGCAGGAAGGAATGACCGAAAACGAGAGC |
| NO: 76) | transcriptional | CGTATCGTCGAATGGTTGCTTACCCCCGGCAATCTCAGCGATGCGCC |
| | regulator | GGCAATCAAGGACGTCGCGGAAGCGCTGTCGGTATCAGAAGCCATG |
| | | ATCGTTAAGGTTTCTAAGCTGCTGGGGTTTAGCGGTTTTCGTAACCTG |
| | | CGCAGCGCGCTGGAGGCCTACTTTTCGCAGTCTGAACAGGTGTTACC |
| | | GACCGAACTCTCCTTTGATGATGCGCCGCAGGATGTGGTGAATAAGG |
| | | TGTTCAACATCACCCTGCGCACCATTATGGAAGGCCAGTCGATCGTG |
| | | AACGTTGACGAAATTCACCGCGCGGCGCGCTTTTTTGCCCAAGCCAA |
| | | CCAGCGCGACCTGTACGGCGCGGGCGGCTCCAACGCCATCTGCGCCG |
| | | ACGTGCAGCATAAGTTTTTACGCATCGGCGTACGCTGCCAGGCCTAC |
| | | CCGGACGCGCATATCATGATGATGTCCGCCTCGCTGCTGAAGGAAGG |
| | | CGATGTCGTGCTGGTGGTCTCTCACTCCGGGCGCACCAGCGATATCA |
| | | AATCAGCGGTGGAGCTGGCGAAAAAGAACGGGGCGAAAATTATCTG |
| | | TATCACCCATAGCTATCATTCGCCGATTGCCAAGTTAGCTGATTTTAT |
| | | TATTTGTTCGCCAGCACCAGAGACGCCTTTATTAGGGCGTAACGCCTC |
| | | AGCGCGTATATTACAACTCACATTATTAGATGCGTTTTTTGTTTCCGT |
| | | TGCACAGCTCAACATTGAGCAAGCGAATTTAAATATGCAAAAAACTG |
| | | GCGCGATTGTTAATTTCTTTTCACCCGGCGCGCTTAAATAA |

### Methods

Subjects. Subject Demographics. TABLE 6 summarizes subject demographics and FIG. 31 summarizes the distribution of samples collected by subject age. One hundred and fifty four subjects were enrolled, 56 (36%) with food allergies to at least one of the major food allergens including milk, soy, egg, tree nuts, fish, shellfish, wheat, or peanuts, and 98 (64%) healthy controls. Seventy-eight percent of FA subjects were poly-sensitized, as defined by a positive skin test and/or specific IgE to at least 2 foods. Other FA included sesame, oat, pea, avocado, apple, grape and cantaloupe. 26 FA infants (16.8%) were diagnosed with cow' milk allergy and were avoiding milk products at the time of stool sampling.

Healthy control subjects or subjects with FA age 1-15 months were enrolled. FA criteria included a history of allergic reactions to one or more of the major food allergens (e.g. milk, soy, egg, tree nuts, fish, shellfish, wheat, or peanuts), such as urticaria, angioedema, wheezing, diarrhea, vomiting, and moderate to severe eczema that was clearly triggered by food exposure and improving markedly after food avoidance, and a confirmatory positive food-specific skin prick test (SPT) ≥3 mm compared to saline control and/or serum food-specific IgE ≥0.35. Exclusion criteria included: 1) prematurity, defined as delivery before 37 weeks of gestation, 2) recurrent or chronic infections necessitating frequent systemic (including oral) antibiotic administration, 3) history of chronic immunosuppressive therapies, 4) history of gastroenterological conditions, including non-IgE mediated colitis, eosinophilic esophagitis and food protein induced enterocolitis, allergic colitis, GE reflux or constipation necessitating medication, and 5) history of other chronic diseases, except for atopic conditions.

For studies on circulating ROR-γt⁺ Treg and Teff cells, demographic details on the human subjects involved, including FA, atopic but not FA and healthy controls are detailed in TABLE 10. The diagnosis of FA was ascertained as detailed herein. Subjects who were atopic but not FA carried a diagnosis of allergic rhinitis, asthma and/or eczema, as detailed in TABLE 10, while healthy control subjects did not have a history of FA or atopic diseases.

Sample collection. Parents were asked to collect stools from subjects every 4-6 months for up to 30 months of age, using a clean wood stick in Eppendorf tubes and RNA-later tubes (Thermofisher™) and to freeze the sample in their home freezer immediately. Samples were collected from subjects' homes within 1-3 weeks of the specimen collection. Overall, 60 subjects gave only one stool sample, 31 subjects gave 2 serial samples, 30 gave 3 samples, 17 gave 4 samples and 16 gave 5 samples. Each sample collection was spaced by 4-6 months. If systemic antibiotics were prescribed, sample collection was delayed for 4-6 weeks after the last dose of antibiotic administered, as guided by published studies tracking the recovery of gut microbiota following antibiotic treatment.

If a patient became tolerant to the food that they were initially allergic to (defined by being able to tolerate at least 4 grams of protein of that food), subsequent samples were not included in the analysis unless the subject had another FA confirmed by a history and positive skin and/or specific IgE. Samples from sensitized patients who lack a confirmatory history of an allergic food reaction were excluded from analysis. Parents completed a questionnaire with each stool collection that included information regarding diet, breastfeeding, age, FA, infection, and use of antibiotics.

16S rDNA gene phylotyping. A multiplexed amplicon library covering the 16S rDNA gene V4 region was generated from human stool sample DNA. Briefly, bacterial genomic DNA was extracted using the Mo Bio Power Fecal DNA Isolation kit (Mo Bio Laboratories™) according to the manufacturer's instructions. To increase the DNA yields, the following modifications were used. An additional bead beater step using the Faster Prep FP120 (Thermo™) at 6 meters/second for 1 min was used instead of vortex agitation. Incubation with buffers C2 and C3 was done for 10 min at 4° C. Starting nucleic acid concentrations were determined by a Qubit Fluromoter (Life Technologies™). The amplicon library was prepared using dual-index barcodes. The aggregated library pool was size selected from 300-500 base pairs (bp) on a pippin prep 1.5% agarose cassette (Sage Sciences™) according to the manufacturer's instructions. The concentration of the pool was measured by qPCR (Kapa Biosystems™) and loaded onto the MiSeq Illumina™ instrument (300 bp kit) at 6-9 pM with 40% phiX spike-in to compensate for low base diversity according to Illumina's standard loading protocol.

16S rDNA gene sequencing data preprocessing. Sequencing of the 16S rDNA amplicons from the 368 human stool samples generated 14,279,132 total raw reads, with a mean of 38,802 reads per sample. Raw sequencing reads were processed using the mothur software package (v.1.35.1) and custom Python scripts, which perform de-noising, quality filtering, alignment against the ARB Silva reference database of 16S rDNA gene sequences, and clustering into Operational Taxonomic Units (OTUs) at 97% identity. In total, 16387 OTUs were generated. OTUs with extremely low abundance were filtered using the following parameters: removed if mean relative abundance <0.001 across all samples or if zero reads in >80% of samples in at least one cohort.

Since the composition of the microbiota is known to change throughout childhood, subjects were stratified by age into six month intervals (1-6, 7-12, 13-18, 19-24, and 25-30 months). This interval span was chosen to provide resolution with respect to age, while ensuring sufficient numbers of subjects in each group to support meaningful comparisons. Because insufficient numbers of subjects were available for the 30-36 month group, this group was omitted from analyses. After stratifying subjects by age groups segregated at six month intervals, 47, 53, 78, 74 and 79 OTUs were available after filtering in age group 1-6 months, 7-12 months, 13-18 months, 19-24 months, and 25-30 months, respectively.

16S rDNA gene sequencing data statistical analysis. To assess for differences in ecological (alpha) diversity, Shannon entropy was calculated for each sample with statistical testing using the Wilcoxon rank-sum test. To assess differences in overall microbial community structure, beta-diversity was calculated using the unweighted and weighted Unifrac measures with statistical testing using the Analysis of Molecular Variance (AMOVA) method.

To statistically test for differences in the relevant abundances of fecal microbiota OTUs between control and food allergic subjects, the DESeq2 software package was employed with an analysis design depicted in FIG. 30. Key covariates of interest (gender, mode of delivery, and breastfeeding only for younger than 18 months) were controlled for using the multi-factorial model in DESeq2. Since cow's milk protein (CMP) intake could directly alter the microbiota, and is highly correlated with FA status, a subset analysis removing subjects without CMP intake was also performed. P-values were adjusted for multiple hypothesis testing using the method of Benjamini and Hochberg (BH). OTUs reported met the following criteria: (1) adjusted p-value <=0.1; (2) absolute value of log 2 fold change ≥2.

16S rDNA gene sequencing data phylogenetic analysis. To more accurately identify the micro-organisms present in samples and their phylogenetic relationships to known species, the pplacer software package was used to perform phylogenetic placement. Pplacer uses a likelihood-based methodology to place short sequencing reads of 16S rDNA amplicons on a reference tree, and also generates taxonomic classifications of the short sequencing reads using a least common ancestor-based algorithm. The reference tree required for phylogenetic placement was generated using full-length or near full-length (>1,200 nt) 16S rDNA sequences of type strains from the Ribosomal Database Project (RDP). In these studies, the taxa were report that were phylogenetically placed with a like weight ratio of ≥0.8. The rest of the taxa can be seen in TABLE 7. For purposes of describing OTUs in the manuscript, the closest reference species (CRS) to the phylogenetically placed consensus sequence for the OTU is referenced. While CRS does not represent an unambiguous species identification, it provides a point of reference for understanding microbiologically driven mechanisms in FA, and for deeper characterization using metagenomic or culture-based methods in future studies.

Oral allergic sensitization of mice. The following mice on BALB/c background were used: BALB/cByJ (designated as WT mice), Rag2^{-/-} (C.129S6(B6)-Rag2^{Tm1Fwa}), Il4ra^{F709} (C. 129X1-Il4ra^{tm3.ITch}), Igh7^{-/-} Il4ra^{F709} and Foxp3^{EGFP/DTR+}. The following C57BL/6 congenic strains Rorc^{fl/fl} (ROR-gtf/fB6(Cg)-Rorc^{tm3Litt}), B6.129(Cg)-Foxp^{3tm4(YFP/cre)Ayr/J}) (FoXp3^{YFPCre}) and B6.129(Cg)-Il4ra^{F709 23} were crossed to generate Il4raF709Foxp3^{YFPCre}, Foxp3^{YFPCre}Rorc^{Δ/Δ70} matched for strain background. Mice were subjected to oral allergic sensitization with ovalbumin (OVA), mixed together with the mucosal adjuvant staphylococcal enterotoxin B (SEB) (Toxin Technology), as previously described^{12,25}. For antibiotic treatment, mice were treated with an antibiotic cocktail (Sigma-Aldrich) containing ampicillin 2.5 mg/ml), metronidazole (2.5 mg/ml), gentamycin (0.4 mg/ml), streptomycin (0.5 mg/ml), vancomycin (0.5 mg/ml), administered by oral gavage in a final total volume of 100 μl PBS once daily for 1 week, as indicated. For Treg cell depletion with DT, mice were injected intra-peritoneally (i.p.) DT (Sigma-Aldrich) at 250 ng/ml per injection/mouse (about 10 μg/kg), as indicated. For treatment with anti-CD25 or isotype control mAbs (BioXCell), mice were injected i.p. with the indicated antibody at 100 μg/injection/mouse, as indicated.

Percutaneous allergic sensitization of mice. Mice were treated with antibiotics for one week, as described above. They were then sensitized through the skin with OVA/SEB as follows. The back of the mouse was shaved then tape stripped six times with Tegaderm dressings⁴⁸. OVA/SEB (at a concentration of 5 mg/ml OVA and 10 μg SEB in a final volume of 100 μl PBS were applied directly on the skin. The sensitization was repeated twice weekly for 5 weeks. In subgroups of mice the Clostridiales consortium (see below) was given by gavage at 200 μl per mouse twice weekly for 5 weeks. At the end of the sensitization period, the mice are challenged with OVA in 150 mg/300 μl of PBS/mouse via gavage.

Detection of Fecal Bacteria-bound IgA and IgE by Flow Cytometry. 50 mg of fecal pellet was homogenized in 1 ml of sterile cold PBS and centrifuged at 40 g for 10 minutes at 4° C. to remove large particles. Supernatant containing the bacteria were collected, filtered through a 70 μm strainer and centrifuged at 8000 g for 5 minutes to pellet the bacteria. The pellets were then washed twice with 1 ml of sterile PBS and incubated on ice for 15 minutes with blocking buffer (50% fetal calf serum (FCS) in PBS for human fecal samples and 50% FCS+10 mg/ml OVA in PBS for mouse fecal samples). Samples were centrifuged at 8000 g for 5 minutes and subsequently stained with 5 μM SYTO-BC (eBioscience—ThermoFisher™) along with either anti-mouse IgA (clone mA-6E1, eBioscience—ThermoFisher™) or anti-mouse IgE (clone RME-1, Biolegend™). Similarly, human fecal samples were stained with 5 μM SYTO-BC along with either anti-human IgA (clone IS11-8E10, Miltenyi Biotech™) or anti-human IgE (clone G7-26, BD Biosciences™). Samples were then washed 3 times with 1 ml of PBS before flow cytometric analysis on a BD LSR Fortessa™.

Isolation of MLN and LP lymphocytes. MLNs were isolated and homogenized in PBS containing 2% FCS buffer. Cells were washed once PBS containing 2% FCS and used for experiments. Small intestines were dissected from mice and the fecal contents were flushed out using PBS containing 2% FCS. Payer's patch was excised and the intestines were cut into 1 cm pieces and treated with PBS containing 2% FCS, 1.5 mM DTT, and 10 mM EDTA at 37° C. for 30 min with constant stirring to remove mucous and epithelial cells. The tissues were then minced and the cells were dissociated in RPMI containing collagenase (2 mg ml-1 collagenase II; Worthington™), DNase I (100 μg ml-1; Sigma™), 5 mM MgCl2, 5 mM CaCl2, 5 mM HEPES, and 10% FBS with constant stirring at 37° C. for 45 min. Leukocytes were collected at the interface of a 40%/70% Percoll gradient (GE Healthcare™). The cells were washed with PBS containing 2% FCS and used for experiments.

Preparation of therapeutic bacterial consortia. Frozen stock cultures of the bacterial isolates (see e.g., TABLE 10) were stored at −80° C. in microbank tubes (Pro-Lab Diagnostics™) Obligately anaerobic species were plated from frozen stocks onto pre-reduced Brucella agar plates (BBL, Beckton Dickinson™) and incubated in a Coy anaerobic chamber (Coy Labs™). Facultative anaerobes were plated onto Trypticase Soy Agar (TSA) media (Remel™) and incubated in the Coy chamber. All plates were incubated until visible growth was detected. Purity of materials was confirmed by Gram stain and rapid ANA (Remel™) panels for anaerobes or API-20E strips (Remel™) for aerotolerant facultative species.

Growth curve studies in the appropriate liquid media (see e.g., TABLE 9) were performed to quantitate a given biomass of organisms to the optical density (OD) measured at 600 nm. To prepare aggregate cultures, tubes containing 5 ml of the appropriate broth to support growth were inoculated. After visible growth and confirmation of culture purity, materials were then added to a larger culture volume to obtain additional biomass for aggregate mixtures. Cultures were staged based on the calculated time of the growth curves to be able to process a maximum biomass of each species on the day aggregate materials would be prepared. The bacterial consortia were prepared by normalizing the bacterial components according to OD 600 so that the OD of each component was approximately the same as the other bacteria in the cocktail. Materials were aggregated to have approximately 5×10⁷ CFU/ml of each organism. After preparation of each aggregate mixture, lml aliquots were prepared in a Coy anaerobic chamber into cryovials, which were sealed, removed from the chamber and flash frozen in liquid nitrogen. Flash freezing by this method minimized loss of viable cells with the freeze/thaw to a ½ log-level or less. Frozen aliquots were stored at −80° C. until use. Representative aliquots were subjected to qPCR with the probes in TABLE 11 to confirm component species and relative abundance in the mixtures. Mixtures of obligate anaerobes were also plated aerobically to TSA agar media and incubated at 37° C. in 5% CO₂ for 72 hours to confirm absence of aerotolerant contaminants. The negative control consortium (NCC) of members of the Proteobacteria was plated to CNA sheep's blood agar (Remel™) incubated at 37° C. under anaerobic conditions and at 5% CO₂ under aerobic conditions for 72 hours to confirm absence of Gram positive contaminants.

Preparation of Subdoligranulum variabile therapeutic. Microbiologic stocks of S. variabile (DSM 15176) were prepared. Maximal growth, ranging from 5×10⁶-2×10⁷ CFU/mL in liquid media, was observed at 72 hours post-inoculation. For preparation of aliquots, a PRAS BHI tube (Thermo Fisher™) was inoculated and incubated for 72 hours at 37° C. in a Coy anaerobic chamber. Gram stain and culture to BHI media were done to confirm purity. 2.5 ml of the culture was added to separate 150 ml volume cultures of BHIS with hemin and vitamin K (Remel™) and incubated in the anaerobic chamber for 72 hours before preparing aliquots. The strain lost substantive viability with attempts to concentrate its biomass under anaerobic conditions. Aliquots were thus prepared in a Coy chamber with nominal handling by transferring 1 ml aliquots of the 72-hour culture into cryovials and sealing them in the chamber. Aliquots were then removed and snap frozen on liquid nitrogen for storage at −80° C. until use. Separate aliquots were serially diluted and plated to BHI, with counting of pinpoint-sized colonies at 72 hr to confirm S. variabile and at a biomass of 1.2×10⁷ CFU/ml for administration of 2.4×10⁶ CFU per mouse in 200 μl.

Heat killing of therapeutic consortia. Sealed aliquots of the Clostridial or Bacteroidales consortia were placed in a heating block at 85° C. for 1 hour to kill vegetative cells and spores. Control aliquots were transferred into a Coy anaerobe chamber and plated to BHI agar and broth with hemin and vitamin K to confirm killing by absence of growth. The Clostridiales consortium was also inoculated into BHI broth media+1% maltose, hemin and vitamin K to confirm killing of C. leptum. No heat killed aliquots demonstrated any signs of growth. Heat-treated aliquots were then used in studies to assess efficacy of preparations lacking viable bacteria.

Short Chain Fatty Acid Analyses. Samples of gut contents were kept frozen at −80° C. until analysis. The samples were removed from the freezer and thawed. 500 μl of HPLC water was added to each sample and vortexed for 10 minutes and then centrifuged at 5000 g for 10 minutes. 400 μl of the clear supernatant was transferred to a 2.0 ml Eppendorf tube. The pH of each sample was adjusted to 2-3 by adding 50 μl of 50% sulfuric acid. 50 μl of the internal standard (1% 2-methyl pentanoic acid solution) and 400 μl of ethyl ether anhydrous were added (Sigma-Aldrich™). The tubes were mixed end over end for 10 minutes and then centrifuged at 2500 g for 2 minutes. The upper ether layer was transferred to an Agilent sampling vial for analysis. 1 μl of the upper ether layer was injected into the chromatogram for analysis.

Chromatographic analysis was carried out using an Agilent 7890B system with a flame ionization detector (FID) (Agilent Technologies™). A high-resolution gas chromatography capillary column 30 m×0.25 mm coated with 0.25 um film thickness was used (DB-FFAP™) for the volatile acids (Agilent Technologies™). Nitrogen was used as the carrier gas. The oven temperature was 145° C. and the FID and injection port was set to 225° C. The injected sample volume was 1 μl and the run time for each analysis was 12 minutes. Chromatograms and data integration was carried out using the OpenLab Chem Station software (Agilent Technologies™).

Standard Solutions: A volatile acid mix containing 10 mM of acetic, propionic, isobutyric, butyric, isovaleric, valeric, isocaproic, caproic, and heptanoic acids was used (Supelco™ and Sigma-Aldrich™). A standard stock solution containing 1% 2-methyl pentanoic acid (Sigma-Aldrich™) was prepared as an internal standard control for the volatile acid extractions

Quantification of Acids: 400 μl of the standard mix was used and the extracts prepared as described for the samples except that 400 μl of ethyl ether was added. The retention times and peak heights of the acids in the standard mix were used as references for the sample unknowns. These acids were identified by their specific retention times and the concentrations determined and expressed as mM concentrations per gram of sample.

Quantitative real-time PCR for host immunological Targets. RNA was extracted from cells using Quick-RNA MiniPrep kit (Zymo Research™) according to the manufacturer protocol. Reverse transcription was performed with the SuperScript III RT-PCR system and random hexamer primers (Invitrogen™) and quantitative real-time reverse transcription (RT)-PCR with Taqman® Fast Universal PCR master mix, internal house keeping gene mouse (Hprt VIC-MGB dye) and specific target gene primers for murine Rorc, as indicated (FAM Dye) (Applied Biosystems™) on Step-One-Plus machine. Relative expression was normalized to Hprt and calculated as fold change compared to Foxp3^{YFPCre} Treg cells.

Flow cytometry. The following anti-mouse antibodies were used: CD4 (RM4-5), CD3 (145-2C11), Foxp3 (FJK-16S), GATA-3 (TWAJ), ROR-γt (B2), IgA (mA-6E1), rat IgG1 Isotype control (eBRG1), (eBioscience™), IL-4 (11B11) and rat IgG1 Isotype control (R3-34) (BD Biosciences™), Neuropilin-1 (3E12), Helios (22F6), IgE (RME-1), (Biolegend™). Anti-human antibodies used in this study included IgE (G7-26), mouse IgG2a isotype control (G155-178) (BD Biosciences™), IgA (IS11-8E10) (Miltenyi Biotech™) and mouse IgG1 (P3) (eBioscience™). For cytokines cells were stimulated during 4 hours with PMA (50 ng/ml; Sigma-Aldrich™) and ionomycin (500 ng/ml; Sigma-Aldrich™) in the presence of Golgi Plug (BD Biosciences™), then stained with the BD Cytofix/Cytoperm buffers (BD Biosciences™) and the indicated anti-cytokine antibody. For intracellular staining of nuclear factors, the Foxp3 Transcription Factor buffer set (eBioscience™) was used. Dead cells were routinely excluded from the analysis based on the staining of eFluor 506 fixable viability dye (eBioscience™), and analyses were restricted to single cells using FSC-H and FSC-A signals. Stained cells were analyzed on an LSR Fortessa™ (BD Biosciences™) and data were processed using Flowjo™ (Tree Star Inc.™).

ELISA. Total, OVA-specific IgE and Murine mast cell protease 1 (MMCP-1) concentrations were measured in the sera of treated mice by ELISAs.

Histology. Intestinal mast cells were counted by microscopic examination of jejunal sections fixed in 10% formaldehyde and stored in ethanol 70% before staining with toluidine blue.

Statistical analysis. Anaphylaxis-related Core body temperature measurements were analyzed using repeat measures 2-way ANOVA with the indicated post-test analysis. Student unpaired 2-tailed t-tests were used for 2-group comparisons. For more than 2 groups, 1-way ANOVA with the indicated post-test analysis was used. Results are presented as means and SEMs, where each point represents 1 sample. In cases in which values were spread across multiple orders of magnitude, data were log-transformed for analysis with parametric tests.

For any additional details, see e.g., US Patent Application 20180117098; International Patent Application PCT/US2019/060504 filed Nov. 8, 2019; Abdel-Gadir et al., Microbiota therapy acts via a regulatory T cell MyD88/RORγt pathway to suppress food allergy, 24 Jun. 2019, Nature Medicine volume 25, pages 1164-1174; the contents of each of which is incorporated herein by reference in its entirety.

1. A pharmaceutical composition comprising:
(i) a preparation comprising a species of viable gut bacteria comprising a 16S rDNA sequence at least 97% identical to SEQ ID NO: 16, in an amount sufficient to treat or prevent a dysbiosis when administered to an individual in need thereof, and
(ii) a pharmaceutically acceptable carrier,
wherein the pharmaceutical composition is formulated to deliver the viable bacteria to the small intestine.

2.-3. (canceled)

4. The pharmaceutical composition of claim 1, wherein the pharmaceutically acceptable carrier comprises an enteric coating composition that encapsulates the species of viable gut bacteria.

5. The composition of claim 4, wherein the enteric-coating composition is in the form of a capsule, gel, pastille, tablet or pill.

6. The composition of claim 1, wherein the composition is formulated to deliver:
(a) a dose of at least 5×10⁶ colony forming units per mL (CFU/mL)-2×10⁷ CFU/mL; or
(b) at least 5×10⁶ CFU/mL-2×10⁷ CFU/mL in less than 30 capsules per one time dose.

7. (canceled)

8. The composition of claim 1, wherein the composition is frozen for storage.

9. The composition of claim 1, wherein the species of viable gut bacteria are encapsulated under anaerobic conditions.

10. The composition of claim 9, wherein anaerobic conditions comprise one or more of the following:
(i) oxygen impermeable capsules,
(ii) addition of a reducing agent including N-acetylcysteine, cysteine, or methylene blue to the composition, or
(iii) use of spores for organisms that sporulate.

11.-12. (canceled)

13. The composition of claim 1, wherein the species of viable gut bacteria is encapsulated, lyophilized, formulated in a food item, or is formulated as a liquid, gel, fluid-gel, or nanoparticles in a liquid.

14. The composition of claim 1, further comprising a pre-biotic composition.

15. The pharmaceutical composition of claim 1, wherein the dysbiosis is associated with:
(a) an inflammatory disease or a metabolic disorder; or
(b) an atopic disease or disorder.

16. (canceled)

17. The pharmaceutical composition of claim 15, wherein the atopic disease or disorder is selected from the group consisting of: food allergy, eczema, asthma, and rhinoconjunctivitis.

18. A method for treating or preventing the onset of a dysbiosis in a subject, the method comprising: administering to a subject a pharmaceutical composition of claim 1, thereby treating or preventing dysbiosis in the subject.

19. A method for the treatment, or prevention of gut inflammation or a metabolic disease or disorder, the method comprising: administering to a subject a pharmaceutical composition of claim 1, thereby treating, or preventing the gut inflammation or metabolic disease or disorder in the subject.

20. A method for the treatment, or prevention of an atopic disease or disorder, the method comprising: administering to a subject a pharmaceutical composition of claim 1, thereby treating, or preventing the atopic disease or disorder in the subject.

21.-38. (canceled)

39. The method of claim 20, wherein the atopic disease is a food allergy, and wherein the food allergy comprises allergy to soy, wheat, eggs, dairy, peanuts, tree nuts, shellfish, fish, mushrooms, stone fruits and/or other fruits.

40. The method of claim 20, wherein the pharmaceutical composition is administered before a first exposure to a potential food allergen.

41.-43. (canceled)

44. A method for reducing or eliminating a subject's immune reaction to an allergen, the method comprising: administering to a subject a pharmaceutical composition of claim 1, thereby reducing or eliminating a subject's immune reaction to the allergen.

45.-56. (canceled)

57. The method of claim 44, wherein the pharmaceutical composition is administered after an initial exposure and/or reaction to a potential allergen.

58.-60. (canceled)

61. A method of monitoring a subject's microbiome, the method comprising: determining the presence and/or biomass in a biological sample obtained from a subject, and wherein if at least one or more species selected from the group consisting of Subdoligranulum variabile, Bacteroides fragilis, Bacteroides ovatus, Bacteroides vulgatus, Parabacteroides distasonis, and Prevotella melaninogenica, are absent or low relative to a reference, the subject is treated with the pharmaceutical composition of claim 1.

62.-67. (canceled)

68. A method of reducing the number or activity of Th2 cells in a tissue of an individual in need thereof, the method comprising administering a pharmaceutical composition of claim 1 to the individual.

69.-83. (canceled)
